(19) United States
(12) Patent Application Publication
(10) Pub. No.: US 2024/0398933 A1
(43) Pub. Date: Dec. 5, 2024

(54) NOVEL LIPID NANOPARTICLES FOR DELIVERY OF NUCLEIC ACIDS COMPRISING PHOSPHATIDYLSERINE

(71) Applicant: CureVac SE, Tübingen (DE)

(72) Inventors: Patrick BAUMHOF, Tübingen (DE); Gemma NAVARRO, Tübingen (DE); Livia PALMERSTON MENDES, Tübingen (DE); Christine RÜCKERT, Tübingen (DE); Sandra LAZZARO, Tübingen (DE)

(73) Assignee: CureVac SE, Tübingen (DE)

(21) Appl. No.: 18/688,748

(22) PCT Filed: Sep. 2, 2022
(86) PCT No.: PCT/EP2022/074435

§ 371 Date: Mar. 1, 2024

(30) Foreign Application Priority Data
Sep. 3, 2021 (WO) PCT/EP2021/074342

(51) Int. Cl. A61K 39/205; A61K 39/00; A61K 39/015

(52) U.S. Cl. CPC A61K 39/205; A61K 39/015; A61K 2039/53; A61K 2039/55555; A61K 2039/6093; C12N 2760/20134

(57) ABSTRACT

The invention relates to a vaccine composition comprising a) at least one nucleic acid encoding at least one antigen or fragment or variant thereof; and b) a carrier composition, wherein the carrier composition comprises the phospholipid phosphatidylserine. The present invention further relates to a pharmaceutical composition comprising the vaccine composition and a pharmaceutically acceptable carrier, diluent or excipient, and to the vaccine composition or pharmaceutical composition for use in the treatment or prophylaxis of (as well as a corresponding method of treatment thereof) infectious diseases; cancer or tumor diseases, disorders or conditions; specific liver diseases; allergies; or autoimmune disease, disorder or condition; in a subject. Still further, the present invention is concerned with a kit or kit of parts, comprising the vaccine composition or the pharmaceutical composition as well as a method of inducing an immune response in a subject. Finally, the present invention is concerned with a use of a vaccine composition or the pharmaceutical composition or the kit or kit of parts for (i) inducing an immune response and for (ii) inducing an antigen specific T-cell response in a subject.

### TECHNICAL FIELD

The invention relates to a vaccine composition comprising at least one nucleic acid encoding at least one antigen or fragment or variant thereof and a carrier composition, wherein the carrier composition comprises the phospholipid phosphatidylserine. The at least one antigen or fragment or variant thereof may be derived from a pathogenic antigen, a tumour antigen, an allergenic antigen or an autoimmune self-antigen such that the vaccine composition or a pharmaceutical composition comprising the vaccine composition can be used for the treatment and prevention of infectious diseases; cancer or tumor diseases, disorders or conditions; specific liver diseases; allergies; or autoimmune disease, disorder or condition; in a subject. The present invention is also concerned with corresponding kits or kits of parts, corresponding methods of inducing an immune response in a subject and the use of the same for inducing an immune response and for inducing an antigen specific T-cell response in a subject.

### BACKGROUND OF THE INVENTION

Commonly, vaccines may be subdivided into “first”, “second” and “third” generation vaccines. “First generation” vaccines are, typically, whole-organism vaccines. They are based on either live and attenuated or killed pathogens, e.g. viruses, bacteria or the like. The major drawback of live and attenuated vaccines is the risk for a reversion to life-threatening variants. Thus, although attenuated, such pathogens may still intrinsically bear unpredictable risks. Killed pathogens may not be as effective as desired for generating a specific immune response. In order to minimize these risks, “second generation” vaccines were developed. These are, typically, subunit vaccines, consisting of defined antigens or recombinant protein components which are derived from pathogens.

Genetic vaccines, i.e. vaccines for genetic vaccination, are usually understood as “third generation” vaccines. They are typically composed of genetically engineered nucleic acid molecules which allow expression of peptide or protein (antigen) fragments characteristic for a pathogen or a tumor antigen in vivo. Genetic vaccines are expressed upon administration to a patient after uptake by target cells. Expression of the administered nucleic acids results in production of the encoded proteins. In the event these proteins are recognized as foreign by the patient's immune system, an immune response is triggered.

DNA as well as RNA may be used as nucleic acid molecules for administration in the context of genetic vaccination. DNA is known to be relatively stable and easy to handle. However, the use of DNA bears the risk of undesired insertion of the administered DNA-fragments into the patient's genome potentially resulting mutagenic events such as in loss of function of the impaired genes. As a further risk, the undesired generation of anti-DNA antibodies has emerged. Another drawback is the limited expression level of the encoded peptide or protein that is achievable upon DNA administration because the DNA must enter the nucleus in order to be transcribed before the resulting mRNA can be translated. Among other reasons, the expression level of the administered DNA will be dependent on the presence of specific transcription factors which regulate DNA transcription. In the absence of such factors, DNA transcription will not yield satisfying amounts of RNA. As a result, the level of translated peptide or protein obtained is limited.

The use of messenger RNA (mRNA) for delivery of genetic information into target cells offers an attractive alternative to DNA. The advantages of using mRNA include transient expression and a non-transforming character—mRNA does not need to enter the nucleus in order to be expressed and moreover cannot integrate into the host genome, thereby eliminating the risk of oncogenesis. I.e. by using RNA instead of DNA for genetic vaccination, the risk of undesired genomic integration and generation of anti-DNA antibodies is minimized or avoided. Two main issues relating to the use of mRNA in vaccines are connected to the degradation and the intracellular access. Thus, free RNAs are susceptible to nuclease digestion in plasma, and free RNAs have limited ability to gain access to the intracellular compartment where the relevant translation machinery resides.

Lipid nanoparticles (LNPs) formed from cationic lipids with other lipid components, such as neutral lipids, cholesterol, and polymer conjugated lipids as well as mRNA have been used to block degradation of the RNAs in plasma and facilitate the cellular uptake of the oligonucleotides. WO2018078053 and WO2016176330 describe lipid nanoparticle compositions comprising unmodified and nucleoside-modified RNA encoding different antigens in this regard.

While the use of the afore-mentioned lipid nanoparticles was already a big step forward towards an effective use of mRNA-based vaccines, there is the need for further improving mRNA-based vaccines, e.g. by adding at least one further component to mRNA-based vaccines such that the vaccines result in a greater immune response to the antigen encoded by the mRNA.

Furthermore, standard LNPs are usually targeted to the liver. This may be disadvantageous in some cases, e.g. in immunotherapy, as an immune response against these organs may be triggered. Therefore, there is a need for mRNA formulations which can be administered systemically, which avoid liver targeting. Additionally, providing LNPs which are targeted to the spleen, macrophages or respectively DCs, would be advantageous. In this regard, nucleic acids like mRNA are of high interest for various therapeutic interventions in patients, e.g. in tumor therapy approaches, based on tumor antigen expression by coding mRNA in antigen presenting cells (APCs) in order to induce a T-cell response to the tumor. Target cells for such intervention are dendritic cells (DCs) which reside, for example, in the lymph nodes (LNs) or in the spleen. Thus, mRNA encoding polypeptides comprising one or more epitopes can be used to deliver epitopes derived from tumor-associated antigens encoded by excessively upregulated RNA transcripts to a patient. Dendritic cells (DCs) residing in the spleen represent antigen-presenting cells of particular interest for mRNA expression of epitopes. Thus, one object of the invention thus is the development of an injectable RNA formulation with high spleen selectivity, which fulfils the criteria for products for application to patients. The claims of the present invention provide a solution to the above described problem or object. According to the invention, LNPs comprising phosphatidylserine surprisingly lead to substantial mRNA expression in spleen or respectively dendritic cells after administration of those LNPs. A strong expression of reporter gene in the target cells (spleen) was determined while the expression in other organs was low. This was unexpected, because usually LNPs show major expression in liver upon administration.

Thus, notwithstanding all the prior art, however, there is still a requirement for alternative lipid nanoparticle formulations comprising alternative lipids, that offer one or more properties of reduced cell toxicity, better targeting ability, enhanced short-term and/or long-term immunity, or promotion of endosomal escape of molecules, e.g. nucleic acids. Despite the vast amount of work undertaken to date in the field of lipid nanoparticle formulations, therefore, it is nevertheless desired to develop further lipid nanoparticle formulations capable of ameliorating or obviating one or more of the problems described above or of in vivo efficacy of the transfection process, toxicity, cost and simplicity of design.

Thus, the object of the present invention can also be seen as to the provision of novel lipid nanoparticle formulations ameliorating or obviating one or more of the problems described above or of in vivo efficacy of the transfection process, toxicity, cost and simplicity of design. These problems and the further problems described under “Background of the Invention” were solved by the subject-matter and claims of the present invention. One exemplary solution to the problem of the invention is the provision of a a vaccine composition comprising
a) at least one nucleic acid encoding at least one antigen or fragment or variant thereof; and
b) a carrier composition, wherein the carrier composition comprises the phospholipid phosphatidylserine, wherein the amount of the phosphatidylserine is not more than 9 mol %, preferably not more than 5 mol %, of the total molar amount of all lipidic excipients in the carrier composition.

Furthermore, the above problems were solved by adding phospholipids with shorter alkyl chains to LNP formulations, specifically (07:0) PC (DHPC; 1,2-diheptanoyl-sn-glycero-3-phosphocholine), as disclosed herein.

### SUMMARY OF THE INVENTION

The inventors solved the above need in that they surprisingly found that the addition of the phospholipid phosphatidylserine to a vaccine composition, which comprises a carrier composition (preferably a lipid nanoparticle) comprising the nucleic acid encoding an antigen, results in a more effective vaccine, most likely—without wishing to be bound by theory—by targeting the vaccine compositions for phagocytosis by phagocytic cells of the immune system, ultimately resulting in the presence of the encoded antigen or fragment or variant thereof in phagocytic cells.

Further, surprisingly it was found that the above described addition of phosphatidylserine to vaccine compositions leads to a substantially increased targeting of the vaccine compositions to antigen-presenting cells (APCs), like e.g. DCs, macrophages or to the spleen as such, respectively, as when compared to vaccine compositions not comprising phosphatidylserine, preferably wherein the vaccine compositions are mRNA-LNP vaccine compositions. Also surprisingly it was found that the above described addition of phosphatidylserine to mRNA-LNP vaccine compositions leads to a substantially increased targeting of the mRNA-LNP vaccine compositions to lymph nodes, as when compared to mRNA-LNP vaccine compositions not comprising phosphatidylserine.

In the first aspect, the present invention relates to a vaccine composition comprising
a) at least one nucleic acid encoding at least one antigen or fragment or variant thereof; and
b) a carrier composition, wherein the carrier composition comprises the phospholipid phosphatidylserine.

In another aspect, the present invention relates to a method of delivering a vaccine composition comprising at least one nucleic acid encoding at least one antigen or fragment or variant thereof to the spleen or lymph nodes, wherein the carrier composition comprises the phospholipid phosphatidylserine, as when compared to vaccine compositions not comprising phosphatidylserine.

In an embodiment of the first aspect, the antigen is derived from a pathogenic antigen, a tumour antigen, an allergenic antigen or an autoimmune self-antigen.

In another embodiment of the first aspect, the amount of the phosphatidylserine is not more than 9 mol %, preferably not more than 5 mol %, of the total molar amount of all lipidic excipients in the composition.

In another embodiment of the first aspect, the carrier composition is a lipid nanoparticle composition. In yet another embodiment, the lipid nanoparticle composition further comprises
(i) a cationic or ionizable lipid;
(ii) a steroid;
(iii) a further phospholipid in addition to phosphatidylserine; and
(iv) a polymer conjugated lipid.

In the second aspect, the present invention is concerned with a pharmaceutical composition comprising the vaccine composition according to the first aspect and a pharmaceutically acceptable carrier, diluent or excipient, preferably wherein the pharmaceutical composition is a sterile solid composition for reconstitution with a sterile liquid carrier, and wherein the composition further comprises one or more inactive ingredients selected from pH-modifying agents, bulking agents, stabilizers, non-ionic surfactants and antioxidants, and wherein the sterile liquid carrier is an aqueous carrier.

In the third aspect, the present invention relates to the vaccine composition according to the first aspect or the pharmaceutical composition according to the second aspect for use in the treatment or prophylaxis of infectious diseases; cancer or tumor diseases, disorders or conditions; liver diseases selected from the group consisting of liver fibrosis, liver cirrhosis and liver cancer; allergies; or autoimmune disease, disorder or condition; in a subject.

In a very preferred embodiment of the third aspect of the invention, the present invention relates to a vaccine composition for use in the treatment or prophylaxis of a cancer or tumor disease.

In the fourth aspect, the present invention is concerned with a kit or kit of parts, comprising the vaccine composition according to the first aspect or the pharmaceutical composition according to the second aspect, optionally comprising a liquid vehicle for solubilizing, and, optionally, technical instructions providing information on administration and dosage of the components.

In the fifth aspect, the present invention relates to a method of treatment or prophylaxis of infectious diseases; cancer or tumor diseases, disorders or conditions; liver diseases selected from the group consisting of liver fibrosis, liver cirrhosis and liver cancer; allergies; or autoimmune disease, disorder or condition; in a subject comprising the steps:
a) providing the vaccine composition of the first aspect or the pharmaceutical composition according to the second aspect or the kit or kit of parts according to the fourth aspect; and
b) applying or administering the vaccine composition or the pharmaceutical composition or the kit or kit of parts to a tissue or an organism of the subject.

In the sixth aspect, the present invention relates to a method of inducing an immune response in a subject, the method comprising administering to the subject the vaccine composition of the first aspect or the pharmaceutical composition of second aspect in an amount effective to produce an antigen-specific immune response in the subject.

In the seventh aspect, the present invention is concerned with a use of a vaccine composition of the first aspect or the pharmaceutical composition according to the second aspect or the kit or kit of parts according to the fourth aspect for (i) inducing an immune response, for (ii) inducing an antigen specific T-cell response or preferably for (iii) inducing CD8+ T cells responses, in a subject.

### Definitions

For the sake of clarity and readability, the following scientific background information and definitions are provided. Any technical features mentioned herein or disclosed thereby can be part of or may be read on each and every embodiment of the invention. Additional definitions and explanations can be provided in the context of this disclosure.

Unless defined otherwise, or unless the specific context requires otherwise, all technical terms used herein have the same meaning as is commonly understood by a person skilled in the relevant technical field.

Unless the context indicates or requires otherwise, the words “comprise”, “comprises” and “comprising” and similar expressions are to be construed in an open and inclusive sense, as “including, but not limited to” in this description and in the claims. It also needs to be understood that for the purposes of the present invention, the term “consisting of” is considered to be a preferred embodiment of the term “comprising”. If hereinafter a group is defined to comprise at least a certain number of embodiments, this is also meant to encompass a group which preferably consists of these embodiments only.

The expressions, “one embodiment”, “an embodiment”, “a specific embodiment” and the like mean that a particular feature, property or characteristic, or a particular group or combination of features, properties or characteristics, as referred to in combination with the respective expression, is present in at least one of the embodiments of the invention. The occurrence of these expressions in various places throughout this description do not necessarily refer to the same embodiment. Moreover, the particular features, properties or characteristics may be combined in any suitable manner in one or more embodiments.

The singular forms “a”, “an” and “the” should be understood as to include plural references unless the context clearly dictates otherwise.

Percentages in the context of numbers should be understood as relative to the total number of the respective items. In other cases, and unless the context dictates otherwise, percentages should be understood as percentages by weight (wt-%).

As used herein, a “compound” means a chemical substance, which is a material consisting of molecules having essentially the same chemical structure and properties. For a small molecular compound, the molecules are typically identical with respect to their atomic composition and structural configuration. For a macromolecular or polymeric compound, the molecules of a compound are highly similar but not all of them are necessarily identical.

For example, a segment of a polymer that is designated to consist of 50 monomeric units may also contain individual molecules with e.g. 48 or 53 monomeric units.

The term “molecule” may either be used as a synonym for “compound” or for an individual (i.e. a single) molecule.

Any reference to a compound or moiety having a functional group which is ionizable under physiological conditions should be understood as including the ionized form of the respective compound or moiety. Vice versa, any reference to a compound or moiety having an ionized functional group which may also exist in the non-ionized form under physiological conditions should be understood as including the non-ionized form of the respective compound or moiety. For example, the disclosure of a compound having a carboxyl group should be interpreted as referring to the respective compound with non-ionized carboxyl group or with the ionized carboxylate group.

As used herein, “physiological conditions” refers to an aqueous environment having a pH that is within the pH range known from human physiology, including both extra- and intracellular conditions. An approximation of this pH range is from about pH 1 to about pH 9. Depending on the context, physiological conditions may also refer to approximately neutral conditions, such as from about pH 5 to about pH 8.5, or from about pH 5.5 to about pH 8.

A lipidoid compound, also simply referred to as lipidoid, is a lipid-like compound, i.e. an amphiphilic compound with lipid-like physical properties. In the context of the present invention, the term lipid is considered to encompass lipidoids.

In the context of the present invention, the term “selected from the group consisting of” followed by a certain group of elements (e.g. “A, B and C”) is meant within the context of the invention to be not limited to said group. In other words, such a term does not indicate that the disclosure is closed to unrecited elements, i.e. also alternative meanings are comprised within the group following this term. Therefore, in the context of the present invention, the term “selected from the group consisting of” followed by a certain group of elements (i.e. “A, B and C”) should be understood as “selected from A, B, and C” or alternatively “is A, B, or C” encompassing also other structurally and functionally related and unrelated but not mentioned elements.

The term “about” is used when parameters or values do not necessarily need to be identical, i.e. 100% the same. Accordingly, “about” means, that a parameter or values may diverge by 0.1% to 20%, preferably by 0.1% to 10%; in particular, by 0.5%, 1%, 2%, 3%, 4%, 5%, 6%, 7%, 8%, 9%, 10%, 11%, 12%, 13%, 14%, 15%, 16%, 17%, 18%, 19%, 20%. The skilled person will know that e.g. certain parameters or values may slightly vary based on the method how the parameter was determined. For example, if a certain parameter or value is defined herein to have e.g. a length of “about 1000 nucleotides”, the length may diverge by 0.1% to 20%, preferably by 0.1% to 10%; in particular, by 0.5%, 1%, 2%, 3%, 4%, 5%, 6%, 7%, 8%, 9%, 10%, 11%, 12%, 13%, 14%, 15%, 16%, 17%, 18%, 19%, 20%. Accordingly, the skilled person will know that in that specific example, the length may diverge by 1 to 200 nucleotides, preferably by 1 to 100 nucleotides; in particular, by 5, 10, 20, 30, 40, 50, 60, 70, 80, 90, 100, 110, 120, 130, 140, 150, 160, 170, 180, 190, 200 nucleotides.

The term “cationic” means, unless a different meaning is clear from the specific context, that the respective structure bears a positive charge, either permanently or not permanently but in response to certain conditions such as e.g. pH. Thus, the term “cationic” covers both “permanently cationic” and “cationisable”. The term “cationisable” as used herein means that a compound, or group or atom, is positively charged at a lower pH and uncharged at a higher pH of its environment. Also in non-aqueous environments where no pH value can be determined, a cationisable compound, group or atom is positively charged at a high hydrogen ion concentration and uncharged at a low concentration or activity of hydrogen ions. It depends on the individual properties of the cationisable or polycationisable compound, in particular the pKₐ of the respective cationisable group or atom, at which pH or hydrogen ion concentration it is charged or uncharged. In diluted aqueous environments, the fraction of cationisable compounds, groups or atoms bearing a positive charge may be estimated using the so-called Henderson-Hasselbalch equation which is well-known to a person skilled in the art. E.g., if a compound or moiety is cationisable, it is preferred that it is positively charged at a pH value of about 1 to 9, preferably 4 to 9, 5 to 8 or even 6 to 8, more preferably of a pH value of or below 9, of or below 8, of or below 7, most preferably at physiological pH values, e.g. about 7.3 to 7.4, i.e. under physiological conditions, particularly under physiological salt conditions of the cell in vivo. In embodiments, it is preferred that the cationisable compound or moiety is predominantly neutral at physiological pH values, e.g. about 7.0-7.4, but becomes positively charged at lower pH values. In some embodiments, the preferred range of pKₐ for the cationisable compound or moiety is about 5 to about 7. In some embodiments, the protonatable lipids have a pKₐ of the protonatable group in the range of about 4 to about 11, e.g., a pKₐ of about 5 to about 7.

Unless a different meaning is clear from the specific context, the term “cationic” means that the respective structure bears a positive charge, either permanently, or not permanently but in response to certain conditions such as pH. Thus, the term “cationic” covers both “permanently cationic” and “cationisable”. For example, a compound or moiety with a primary, secondary or tertiary amino group is cationic, and more specifically, cationisable, as it may exist predominantly in the positively charged state under physiological conditions.

As used herein, “permanently cationic” means that the respective compound, or group or atom, is positively charged at any pH value or hydrogen ion activity of its environment. Very often, the positive charge results from the presence of a quaternary nitrogen atom. Where a compound carries a plurality of such positive charges, it may be referred to as permanently polycationic, which is a subcategory of permanently cationic.

Similarly, the terms “anionic”, “anionizable” and “permanently anionic” are used to have the analog meaning as “cationic”, “cationisable” and “permanently cationic”, except that the charge of the respective compound, group or atom is negative rather than positive.

The expression “neutral”, when applied to a compound such as a lipid or a steroid, or to a group or moiety, either means that it is neither cationic nor anionic, such as a compound having no functional groups that are ionizable under physiological conditions as, for example, like a hydrocarbon; or it is both cationic and anionic, i.e. zwitterionic, under typical physiological conditions, such as a typical native phosphatidylcholine.

A “lipid”, as used herein, refers to a group of organic compounds that are derivatives of fatty acids (e.g., esters) and are generally characterized by being insoluble in water but soluble in many organic solvents. Lipids are usually divided in at least three classes: (1) “simple lipids” which include fats and oils as well as waxes; (2) “compound lipids” which include phospholipids and glycolipids; and (3) “derived lipids” such as steroids. Regarding glycolipids, in certain embodiments, the LNP comprises glycolipids (e.g., monosialoganglioside GM₁).

In this context, the prefix “poly-” refers to a plurality of atoms or groups having the respective property in a compound. If put in parenthesis, the presence of a plurality is optional. For example, (poly)cationic means cationic and/or polycationic. However, the absence of the prefix should not be interpreted such as to exclude a plurality. For example, a polycationic compound is also a cationic compound and may be referred to as such.

The term “nucleic acid” means any compound comprising, or consisting of, DNA or RNA. The term may be used for a polynucleotide and/or oligonucleotide. Wherever herein reference is made to a nucleic acid or nucleic acid sequence encoding a particular protein and/or peptide, said nucleic acid or nucleic acid sequence, respectively, preferably also comprises regulatory sequences allowing in a suitable host, e.g. a human being, its expression, i.e. transcription and/or translation of the nucleic acid sequence encoding the particular protein or peptide.

Preferably, a nucleic acid according to the present invention is not a Toll-like receptor (TLR9) ligand CpG oligonucleotide (ODN) or a cyclic dinucleotide such as cyclic guanosine monophosphate-adenosine monophosphate (cGAMP).

In a very preferred embodiment, the “nucleic acid” of the invention is an “artificial mRNA” or an “isolated mRNA”. The term “artificial mRNA” (sequence) may typically be understood to be an mRNA molecule, that does not occur naturally. In other words, an artificial mRNA molecule may be understood as a non-natural mRNA molecule. Such mRNA molecule may be non-natural due to its individual sequence (which does not occur naturally) and/or due to other modifications, e.g. structural modifications of nucleotides which do not occur naturally. Typically, artificial mRNA molecules may be designed and/or generated by genetic engineering methods to correspond to a desired artificial sequence of nucleotides (heterologous sequence). In this context an artificial sequence is usually a sequence that may not occur naturally, i.e. it differs from the wild type sequence by at least one nucleotide. The term “wild type” may be understood as a sequence occurring in nature. Further, the term “artificial nucleic acid molecule” is not restricted to mean “one single molecule” but is, typically, understood to comprise an ensemble of identical molecules. Accordingly, it may relate to a plurality of identical molecules contained in an aliquot.

In a very preferred embodiment, the nucleic acid of the invention is an “isolated” mRNA.

“Isolated”: As used herein, the term “isolated”, in regard to a nucleic acid molecule, preferably an isolated mRNA, or a polypeptide, means that the nucleic acid molecule, preferably isolated mRNA, or polypeptide is in a condition other than its native environment, such as apart from blood and/or animal tissue. In some embodiments, an isolated nucleic acid molecule, preferably isolated mRNA, or polypeptide is substantially free of other nucleic acid molecules or other polypeptides, particularly other nucleic acid molecules or polypeptides of animal origin. In some embodiments, the nucleic acid molecule, preferably isolated mRNA, or polypeptide can be in a highly purified form, i.e., greater than 95% pure or greater than 99% pure. When used in this context, the term “isolated” does not exclude the presence of the same nucleic acid molecule or polypeptide in alternative physical forms, such as dimers or alternatively phosphorylated or derivatized forms. Isolated substances may also have varying levels of purity in reference to the substances from which they have been associated. Isolated substances and/or entities may also be separated from at least about 10%, about 20%, about 30%, about 40%, about 50%, about 60%, about 70%, about 80%, about 90%, or more of the other components with which they were initially associated. In some embodiments, isolated agents are more than about 80%, about 85%, about 90%, about 91%, about 92%, about 93%, about 94%, about 95%, about 96%, about 97%, about 98%, about 99%, or more than about 99% pure. As used herein, a substance is “pure” if it is substantially free of other components. In the context of the present invention, description and claims, the term “mRNA” preferably means an “isolated mRNA” and vice versa.

In particularly preferred embodiments, the artificial nucleic acid, nucleic acid or RNA is an mRNA, more preferably an isolated mRNA. mRNA technology is specifically preferred in the context of the invention because mRNA allows for regulated dosage, transient and controlled expression as when compared to viral systems, complete degradation of the mRNA after protein synthesis, and does not pose the risk of insertional mutations.

In the context of the present invention, the term “nucleoside modification” refers to nucleic acids such as mRNA compounds or molecules comprising nucleosides which do not normally occur in native mRNA, preferably non-natural nucleosides. In particular, the term preferably refers to mRNA nucleosides other than adenine, guanine, cytosine, uracil and thymine.

The term “nucleoside” generally refers to compounds consisting of a sugar, usually ribose or deoxyribose, and a purine or pyrimidine base. The term “nucleotide” generally refers to a nucleoside comprising a phosphate group attached to the sugar.

A “peptide” means an oligomer or polymer of at least two amino acid monomers linked by peptide bonds. The term does not limit the length of the polymer chain of amino acids. A peptide may, for example, contain less than 50 monomer units. Longer peptides are also called polypeptides, typically having 50 to 600 monomeric units, more specifically 50 to 300 monomeric units.

A “protein” comprises or consists of one or more polypeptides folded into a 3-dimensional form, facilitating a biological function.

Immune system: The immune system may protect organisms from infection. If a pathogen breaks through a physical barrier of an organism and enters this organism, the innate immune system provides an immediate, but non-specific response. If pathogens evade this innate response, vertebrates possess a second layer of protection, the adaptive immune system. Here, the immune system adapts its response during an infection to improve its recognition of the pathogen. This improved response is then retained after the pathogen has been eliminated, in the form of an immunological memory, and allows the adaptive immune system to mount faster and stronger attacks each time this pathogen is encountered. According to this, the immune system comprises the innate and the adaptive immune system. Each of these two parts contains so called humoral and cellular components.

Immune response: An immune response may typically either be a specific reaction of the adaptive immune system to a particular antigen (so called specific or adaptive immune response) or an unspecific reaction of the innate immune system (so called unspecific or innate immune response). The invention relates to the core to specific reactions (adaptive immune responses) of the adaptive immune system. Particularly, it relates to adaptive immune responses to infections by viruses like e.g. Influenza viruses. However, this specific response can be supported by an additional unspecific reaction (innate immune response). Therefore, the invention also relates to a compound for simultaneous stimulation of the innate and the adaptive immune system to evoke an efficient adaptive immune response.

Adaptive immune system: The adaptive immune system is composed of highly specialized, systemic cells and processes that eliminate or prevent pathogenic growth. The adaptive immune response provides the vertebrate immune system with the ability to recognize and remember specific pathogens (to generate immunity), and to mount stronger attacks each time the pathogen is encountered. The system is highly adaptable because of somatic hypermutation (a process of increased frequency of somatic mutations), and V(D)J recombination (an irreversible genetic recombination of antigen receptor gene segments). This mechanism allows a small number of genes to generate a vast number of different antigen receptors, which are then uniquely expressed on each individual lymphocyte. Because the gene rearrangement leads to an irreversible change in the DNA of each cell, all of the progeny (offspring) of that cell will then inherit genes encoding the same receptor specificity, including the Memory B cells and Memory T cells that are the keys to long-lived specific immunity. Immune network theory is a theory of how the adaptive immune system works, that is based on interactions between the variable regions of the receptors of T cells, B cells and of molecules made by T cells and B cells that have variable regions.

Adaptive immune response: The adaptive immune response is typically understood to be antigen-specific. Antigen specificity allows for the generation of responses that are tailored to specific antigens, pathogens or pathogen-infected cells. The ability to mount these tailored responses is maintained in the body by “memory cells”. Should a pathogen infect the body more than once, these specific memory cells are used to quickly eliminate it. In this context, the first step of an adaptive immune response is the activation of naïve antigen-specific T cells or different immune cells able to induce an antigen-specific immune response by antigen-presenting cells (APCs). This occurs in the lymphoid tissues and organs through which naïve T cells are constantly passing. Cell types that can serve as antigen-presenting cells are inter alia dendritic cells, macrophages, and B cells. Each of these cells has a distinct function in eliciting immune responses. Dendritic cells take up antigens by phagocytosis and macropinocytosis and are stimulated by contact with e.g. a foreign antigen to migrate to the local lymphoid tissue, where they differentiate into mature dendritic cells. Macrophages ingest particulate antigens such as bacteria and are induced by infectious agents or other appropriate stimuli to express MHC molecules. The unique ability of B cells to bind and internalize soluble protein antigens via their receptors may also be important to induce T cells. Presenting the antigen on MHC molecules leads to activation of T cells which induces their proliferation and differentiation into armed effector T cells. The most important function of effector T cells is the killing of infected cells by CD8+ cytotoxic T cells and the activation of macrophages by Th1 cells which together make up cell-mediated immunity, and the activation of B cells by both Th2 and Th1 cells to produce different classes of antibody, thus driving the humoral immune response. T cells recognize an antigen by their T cell receptors which do not recognize and bind antigen directly, but instead recognize short peptide fragments e.g. of pathogen-derived protein antigens, which are bound to MHC molecules on the surfaces of other cells.

Cellular immunity/cellular immune response: Cellular immunity relates typically to the activation of macrophages, natural killer cells (NK), antigen-specific cytotoxic T-lymphocytes, and the release of various cytokines in response to an antigen. In a more general way, cellular immunity is not related to antibodies but to the activation of cells of the immune system. A cellular immune response is characterized e.g. by activating antigen-specific cytotoxic T-lymphocytes that are able to induce apoptosis in body cells displaying epitopes of an antigen on their surface, such as virus-infected cells, cells with intracellular bacteria, and cancer cells displaying tumor antigens; activating macrophages and natural killer cells, enabling them to destroy pathogens; and stimulating cells to secrete a variety of cytokines that influence the function of other cells involved in adaptive immune responses and innate immune responses.

Humoral immunity/humoral immune response: Humoral immunity refers typically to antibody production and the accessory processes that may accompany it. A humoral immune response may be typically characterized, e.g., by Th2 activation and cytokine production, germinal center formation and isotype switching, affinity maturation and memory cell generation. Humoral immunity also typically may refer to the effector functions of antibodies, which include pathogen and toxin neutralization, classical complement activation, and opsonin promotion of phagocytosis and pathogen elimination.

Innate immune system: The innate immune system, also known as non-specific immune system, comprises the cells and mechanisms that defend the host from infection by other organisms in a non-specific manner. This means that the cells of the innate system recognize and respond to pathogens in a generic way, but unlike the adaptive immune system, it does not confer long-lasting or protective immunity to the host. The innate immune system may be e.g. activated by ligands of pathogen-associated molecular patterns (PAMP) receptors, e.g. Toll-like receptors (TLRs) or other auxiliary substances such as lipopolysaccharides, TNF-alpha, CD40 ligand, or cytokines, monokines, lymphokines, interleukins or chemokines, IL-1, IL-2, IL-3, IL-4, IL-5, IL-6, IL-7, IL-8, IL-9, IL-10, IL-12, IL-13, IL-14, IL-15, IL-16, IL-17, IL-18, IL-19, IL-20, IL-21, IL-22, IL-23, IL-24, IL-25, IL-26, IL-27, IL-28, IL-29, IL-30, IL-31, IL-32, IL-33, IFN-alpha, IFN-beta, IFN-gamma, GM-CSF, G-CSF, M-CSF, LT-beta, TNF-alpha, growth factors, and hGH, a ligand of human Toll-like receptor TLR1, TLR2, TLR3, TLR4, TLR5, TLR6, TLR7, TLR8, TLR9, TLR10, a ligand of murine Toll-like receptor TLR1, TLR2, TLR3, TLR4, TLR5, TLR6, TLR7, TLR8, TLR9, TLR10, TLR11, TLR12 or TLR13, a ligand of a NOD-like receptor, a ligand of a RIG-1 like receptor, an immunostimulatory nucleic acid, an immunostimulatory RNA (isRNA), a CpG-DNA, an antibacterial agent, or an anti-viral agent. Typically a response of the innate immune system includes recruiting immune cells to sites of infection, through the production of chemical factors, including specialized chemical mediators, called cytokines; activation of the complement cascade; identification and removal of foreign substances present in organs, tissues, the blood and lymph, by specialized white blood cells; activation of the adaptive immune system through a process known as antigen presentation; and/or acting as a physical and chemical barrier to infectious agents.

Adjuvant/adjuvant component: An adjuvant or an adjuvant component in the broadest sense is typically a (e.g. pharmacological or immunological) agent or composition that may modify, e.g. enhance, the efficacy of other agents, such as a drug or vaccine. Conventionally the term refers in the context of the invention to a compound or composition that serves as a carrier or auxiliary substance for immunogens and/or other pharmaceutically active compounds. It is to be interpreted in a broad sense and refers to a broad spectrum of substances that are able to increase the immunogenicity of antigens incorporated into or co-administered with an adjuvant in question. In the context of the present invention an adjuvant will preferably enhance the specific immunogenic effect of the active agents of the present invention. Typically, “adjuvant” or “adjuvant component” has the same meaning and can be used mutually. Adjuvants may be divided, e.g., into immunopotentiators, antigenic delivery systems or even combinations thereof.

The term “adjuvant” is typically understood not to comprise agents which confer immunity by themselves. An adjuvant assists the immune system unspecifically to enhance the antigen-specific immune response by e.g. promoting presentation of an antigen to the immune system or induction of an unspecific innate immune response. Furthermore, an adjuvant may preferably e.g. modulate the antigen-specific immune response by e.g. shifting the dominating Th2-based antigen specific response to a more Th1-based antigen specific response or vice versa. Accordingly, an adjuvant may favorably modulate cytokine expression/secretion, antigen presentation, type of immune response etc.

The term “antibody” as used herein, includes both an intact antibody and an antibody fragment. Typically, an intact “antibody” is an immunoglobulin that specifically binds to a particular antigen. An antibody may be a member of any immunoglobulin class, including any of the human classes: IgG, IgM, IgE, IgA and IgD. Typically, an intact antibody is a tetramer. Each tetramer consists of two identical pairs of polypeptide chains, each pair having a “light” chain and a “heavy” chain. The term “antigen” in the context of the present invention refers typically to a substance which may be recognized by the immune system, preferably by the adaptive immune system, and is capable of triggering an antigen-specific immune response, e.g. by formation of antibodies and/or antigen-specific T cells as part of an adaptive immune response. Typically, an antigen may be or may comprise a peptide or protein which may be presented by the MHC to T-cells. In the sense of the present invention an antigen may be the product of translation of a provided nucleic acid molecule, preferably an mRNA as defined herein. In this context, also fragments, variants and derivatives of peptides and proteins comprising at least one epitope are understood as antigen. Accordingly, the term “antigen” as used herein will be recognized and understood by the person of ordinary skill in the art, and is e.g. intended to refer to a substance which may be recognized by the immune system, preferably by the adaptive immune system, and is capable of triggering an antigen-specific immune response, e.g. by formation of antibodies and/or antigen-specific T cells as part of an adaptive immune response. Typically, an antigen may be or may comprise a peptide or protein which may be presented by the MHC to T-cells. Also fragments, variants and derivatives of peptides or proteins derived from e.g. cancer antigens comprising at least one epitope may be understood as antigens. In the context of the present invention, an antigen may be the product of translation of a provided RNA (e.g. coding RNA, replicon RNA, mRNA). The term “antigenic peptide or protein” will be recognized and understood by the person of ordinary skill in the art, and is e.g. intended to refer to a peptide or protein derived from a (antigenic) protein which may stimulate the body's adaptive immune system to provide an adaptive immune response. Therefore an “antigenic peptide or protein” comprises at least one epitope or antigen of the protein it is derived from (e.g. a tumor antigen, a viral antigen, a bacterial antigen, a protozoan antigen).

The term “derived from” as used throughout the present specification in the context of a nucleic acid, i.e. for a nucleic acid “derived from” (another) nucleic acid, means that the nucleic acid, which is derived from (another) nucleic acid, shares e.g. at least about 50%, 60%, 70%, 80%, 90%, 91%, 92%, 93%, 94%, 95%, 96%, 97%, 98%, or about 99% sequence identity with the nucleic acid from which it is derived. The skilled person is aware that sequence identity is typically calculated for the same types of nucleic acids, i.e. for DNA sequences or for RNA sequences. Thus, it is understood, if a DNA is “derived from” an RNA or if an RNA is “derived from” a DNA, in a first step the RNA sequence is converted into the corresponding DNA sequence (in particular by replacing U by T throughout the sequence) or, vice versa, the DNA sequence is converted into the corresponding RNA sequence (in particular by replacing the T by U throughout the sequence). Thereafter, the sequence identity of the DNA sequences or the sequence identity of the RNA sequences is determined. Preferably, a nucleic acid “derived from” a nucleic acid also refers to nucleic acid, which is modified in comparison to the nucleic acid from which it is derived, e.g. in order to increase RNA stability even further and/or to prolong and/or increase protein production. In the context of amino acid sequences, the term “derived from” means that the amino acid sequence, which is derived from (another) amino acid sequence, shares e.g. at least about 70%, 80%, 90%, 91%, 92%, 93%, 94%, 95%, 96%, 97%, 98%, or about 99% sequence identity with the amino acid sequence from which it is derived.

Epitope (also called “antigen determinant”): T cell epitopes or parts of the proteins in the context of the present invention may comprise fragments preferably having a length of about 6 to about 20 or even more amino acids, e.g. fragments as processed and presented by MHC class I molecules, preferably having a length of about 8 to about 10 amino acids, e.g. 8, 9, or 10, (or even 11, or 12 amino acids), or fragments as processed and presented by MHC class II molecules, preferably having a length of about 13 or more amino acids, e.g. 13, 14, 15, 16, 17, 18, 19, 20 or even more amino acids, wherein these fragments may be selected from any part of the amino acid sequence. These fragments are typically recognized by T cells in form of a complex consisting of the peptide fragment and an MHC molecule.

B cell epitopes are typically fragments located on the outer surface of (native) protein or peptide antigens as defined herein, preferably having 5 to 15 amino acids, more preferably having 5 to 12 amino acids, even more preferably having 6 to 9 amino acids, which may be recognized by antibodies, i.e. in their native form.

Such epitopes of proteins or peptides may furthermore be selected from any of the herein mentioned variants of such proteins or peptides. In this context antigenic determinants can be conformational or discontinuous epitopes which are composed of segments of the proteins or peptides as defined herein that are discontinuous in the amino acid sequence of the proteins or peptides as defined herein but are brought together in the three-dimensional structure or continuous or linear epitopes which are composed of a single polypeptide chain.

A “tolerogenic composition” is a composition that promotes immune tolerance in cells or cellular systems to an antigen, wherein the antigen may be a self-antigen or a non-self antigen. In other words, there is no immune response or a reduced immune response to the antigen. Contrary thereto, a vaccine composition according to the present invention induces an immune response to a specific antigen or fragment or variant thereof, namely the antigen or fragment or variant thereof encoded by the at least one nucleic acid. The antigen may also be a self-antigen or a non-self antigen, and the overall aim of a vaccine composition of the present invention is to create a (strong) immune response to this antigen or fragment or variant thereof, wherein the overall aim of a tolerogenic composition is to at least partly, at best completely, suppress an immune response to this antigen.

A “tolerogenic nucleic acid” is a nucleic acid that promotes immune tolerance in cells or cellular systems to an antigen, wherein the nucleic acid may be a chemically modified mRNA and/or encode a tolerogenic polypeptide. Contrary thereto, the at least one nucleic acid used in the present invention encodes at least one antigen or fragment or variant thereof, against which a (strong) immune response is desired and induced upon administration.

A “tolerogenic polypeptide” is a polypeptide that promotes immune tolerance in cells or cellular systems, typically by decreasing the immune response via acting on underlying pathways, in particular by inhibiting underlying mediators in such pathways. Thus, a tolerogenic polypeptide may be an inhibitor of mTOR, IL-2, IL-10 or an antibody reactive to CD3 or CD40. Contrary thereto, the at least one antigen or fragment or variant thereof according to the present invention does not promote immune tolerance in cells or cellular systems but induces a (strong) immune response against itself.

A “tolerogenic composition” may in particular comprise a tolerogenic nucleic acid, wherein the tolerogenic nucleic acid promotes immune tolerance as described above. The tolerogenic composition may in addition comprise a specific antigen, with the result that there is no immune response to this specific antigen or that the immune response to this specific antigen is reduced due to the presence of the tolerogenic nucleic acid. Contrary thereto, the vaccine composition according to the present invention in a preferred embodiment does not comprise an antigen or fragment or variant thereof but of course still comprises the at least one nucleic acid encoding at least one antigen or fragment or variant thereof, since it is the overall aim of the vaccine composition of the present invention to elucidate a (strong) immune response towards the encoded at least one antigen or fragment or variant thereof (and not, as is the aim of the tolerogenic composition, to block or reduce an immune response towards the co-administered antigen). In yet another preferred embodiment, the vaccine composition according to the present invention comprises the at least one nucleic acid encoding at least one antigen or fragment or variant thereof as the sole payload, and therefore cannot comprise an antigen (as does the tolerogenic composition discussed in this paragraph in addition to the tolerogenic nucleic acid).

The term “vaccine” or “vaccine composition” is typically understood to be a prophylactic or therapeutic material providing at least one antigen or antigenic function or a nucleic acid encoding an antigen or a fragment or variant thereof. The antigen or antigenic function may stimulate the body's adaptive immune system to provide an adaptive immune response.

The term “antigen-providing mRNA” in the context of the invention may typically be an mRNA, having at least one open reading frame that can be translated by a cell or an organism provided with that mRNA. The product of this translation is a peptide or protein that may act as an antigen, preferably as an immunogen. The product may also be a fusion protein composed of more than one immunogen, e.g. a fusion protein that consist of two or more epitopes, peptides or proteins derived from the same or different virus-proteins, wherein the epitopes, peptides or proteins may be linked by linker sequences.

The terms “heterologous” or “heterologous sequence” as used throughout the present specification in the context of a nucleic acid sequence or an amino acid sequence refers to a sequence (e.g. DNA, RNA, amino acid) will be recognized and understood by the person of ordinary skill in the art, and is intended to refer to a sequence that is derived from another gene, from another allele, from another species. Two sequences are typically understood to be “heterologous” if they are not derivable from the same gene or in the same allele. I.e., although heterologous sequences may be derivable from the same organism, they naturally (in nature) do not occur in the same nucleic acid molecule, such as e.g. in the same RNA or protein.

Bi-/multicistronic mRNA: mRNA, that typically may have two (bicistronic) or more (multicistronic) open reading frames (ORF) (coding regions or coding sequences). An open reading frame in this context is a sequence of several nucleotide triplets (codons) that can be translated into a peptide or protein. Translation of such an mRNA yields two (bicistronic) or more (multicistronic) distinct translation products (provided the ORFs are not identical). For expression in eukaryotes such mRNAs may for example comprise an internal ribosomal entry site (IRES) sequence.

Monocistronic mRNA: A monocistronic mRNA may typically be an mRNA, that comprises only one open reading frame (coding sequence or coding region). An open reading frame in this context is a sequence of several nucleotide triplets (codons) that can be translated into a peptide or protein. 3-untranslated region (3′-UTR): A 3′-UTR is typically the part of an mRNA which is located between the protein coding region (i.e. the open reading frame) and the poly(A) sequence of the mRNA. A 3′-UTR of the mRNA is not translated into an amino acid sequence. The 3′-UTR sequence is generally encoded by the gene which is transcribed into the respective mRNA during the gene expression process. The genomic sequence is first transcribed into pre-mature mRNA, which comprises optional introns. The pre-mature mRNA is then further processed into mature mRNA in a maturation process. This maturation process comprises the steps of 5′-capping, splicing the pre-mature mRNA to excise optional introns and modifications of the 3-end, such as polyadenylation of the 3-end of the pre-mature mRNA and optional endo- or exonuclease cleavages etc. In the context of the present invention, a 3′-UTR corresponds to the sequence of a mature mRNA which is located 3′ to the stop codon of the protein coding region, preferably immediately 3′ to the stop codon of the protein coding region, and which extends to the 5′-side of the poly(A) sequence, preferably to the nucleotide immediately 5′ to the poly(A) sequence. The term “corresponds to” means that the 3′-UTR sequence may be an RNA sequence, such as in the mRNA sequence used for defining the 3′-UTR sequence, or a DNA sequence which corresponds to such RNA sequence. In the context of the present invention, the term “a 3′-UTR of a gene”, such as “a 3′-UTR of an albumin gene”, is the sequence which corresponds to the 3′-UTR of the mature mRNA derived from this gene, i.e. the mRNA obtained by transcription of the gene and maturation of the pre-mature mRNA. The term “3′-UTR of a gene” encompasses the DNA sequence and the RNA sequence of the 3′-UTR.

5′-untranslated region (5′-UTR): A 5′-UTR is typically understood to be a particular section of messenger RNA (mRNA). It is located 5′ of the open reading frame of the mRNA. Typically, the 5′-UTR starts with the transcriptional start site and ends one nucleotide before the start codon of the open reading frame. The 5′-UTR may comprise elements for controlling gene expression, also called regulatory elements. Such regulatory elements may be, for example, ribosomal binding sites or a 5′-Terminal Oligopyrimidine Tract. The 5′-UTR may be post-transcriptionally modified, for example by addition of a 5′-CAP. In the context of the present invention, a 5′-UTR corresponds to the sequence of a mature mRNA which is located between the 5′-CAP and the start codon. Preferably, the 5′-UTR corresponds to the sequence which extends from a nucleotide located 3′ to the 5′-CAP, preferably from the nucleotide located immediately 3′ to the 5′-CAP, to a nucleotide located 5′ to the start codon of the protein coding region, preferably to the nucleotide located immediately 5′ to the start codon of the protein coding region. The nucleotide located immediately 3′ to the 5′-CAP of a mature mRNA typically corresponds to the transcriptional start site. The term “corresponds to” means that the 5′-UTR sequence may be an RNA sequence, such as in the mRNA sequence used for defining the 5′-UTR sequence, or a DNA sequence which corresponds to such RNA sequence. In the context of the present invention, the term “a 5′-UTR of a gene”, such as “a 5′-UTR of a TOP gene”, is the sequence which corresponds to the 5′-UTR of the mature mRNA derived from this gene, i.e. the mRNA obtained by transcription of the gene and maturation of the pre-mature mRNA. The term “5′-UTR of a gene” encompasses the DNA sequence and the RNA sequence of the 5′-UTR.

5′-Terminal Oligopyrimidine Tract (TOP): The 5′-terminal oligopyrimidine tract (TOP) is typically a stretch of pyrimidine nucleotides located at the 5′-terminal region of a nucleic acid molecule, such as the 5′-terminal region of certain mRNA molecules or the 5′-terminal region of a functional entity, e.g. the transcribed region, of certain genes. The sequence starts with a cytidine, which usually corresponds to the transcriptional start site, and is followed by a stretch of usually about 3 to 30 pyrimidine nucleotides. For example, the TOP may comprise 3, 4, 5, 6, 7, 8, 9, 10, 11, 12, 13, 14, 15, 16, 17, 18, 19, 20, 21, 22, 23, 24, 25, 26, 27, 28, 29, 30 or even more nucleotides. The pyrimidine stretch and thus the 5′-TOP ends one nucleotide 5′ to the first purine nucleotide located downstream of the TOP. Messenger RNA that contains a 5′-terminal oligopyrimidine tract is often referred to as TOP mRNA. Accordingly, genes that provide such messenger RNAs are referred to as TOP genes. TOP sequences have, for example, been found in genes and mRNAs encoding peptide elongation factors and ribosomal proteins.

TOP motif: In the context of the present invention, a TOP motif is a nucleic acid sequence which corresponds to a 5′-TOP as defined above. Thus, a TOP motif in the context of the present invention is preferably a stretch of pyrimidine nucleotides having a length of 3-30 nucleotides. Preferably, the TOP motif consists of at least 3 pyrimidine nucleotides, preferably at least 4 pyrimidine nucleotides, preferably at least 5 pyrimidine nucleotides, more preferably at least 6 nucleotides, more preferably at least 7 nucleotides, most preferably at least 8 pyrimidine nucleotides, wherein the stretch of pyrimidine nucleotides preferably starts at its 5′-end with a cytosine nucleotide. In TOP genes and TOP mRNAs, the TOP motif preferably starts at its 5′-end with the transcriptional start site and ends one nucleotide 5′ to the first purine residue in said gene or mRNA. A TOP motif in the sense of the present invention is preferably located at the 5′-end of a sequence which represents a 5′-UTR or at the 5′-end of a sequence which codes for a 5′-UTR. Thus, preferably, a stretch of 3 or more pyrimidine nucleotides is called “TOP motif” in the sense of the present invention if this stretch is located at the 5′end of a respective sequence, such as the inventive mRNA, the 5′-UTR element of the inventive mRNA, or the nucleic acid sequence which is derived from the 5′-UTR of a TOP gene as described herein. In other words, a stretch of 3 or more pyrimidine nucleotides which is not located at the 5′-end of a 5′-UTR or a 5′-UTR element but anywhere within a 5′-UTR or a 5′-UTR element is preferably not referred to as “TOP motif”.

TOP gene: TOP genes are typically characterized by the presence of a 5′-terminal oligopyrimidine tract. Furthermore, most TOP genes are characterized by a growth-associated translational regulation. However, also TOP genes with a tissue specific translational regulation are known. As defined above, the 5′-UTR of a TOP gene corresponds to the sequence of a 5′-UTR of a mature mRNA derived from a TOP gene, which preferably extends from the nucleotide located 3′ to the 5′-CAP to the nucleotide located 5′ to the start codon. A 5′-UTR of a TOP gene typically does not comprise any start codons, preferably no upstream AUGs (uAUGs) or upstream open reading frames (uORFs). Therein, upstream AUGs and upstream open reading frames are typically understood to be AUGs and open reading frames that occur 5′ of the start codon (AUG) of the open reading frame that should be translated. The 5′-UTRs of TOP genes are generally rather short. The lengths of 5′-UTRs of TOP genes may vary between 20 nucleotides up to 500 nucleotides, and are typically less than about 200 nucleotides, preferably less than about 150 nucleotides, more preferably less than about 100 nucleotides. Exemplary 5′-UTRs of TOP genes in the sense of the present invention are the nucleic acid sequences extending from the nucleotide at position 5 to the nucleotide located immediately 5′ to the start codon (e.g. the ATG) in the sequences according to SEQ ID NO:1-1363, SEQ ID NO:1395, SEQ ID NO:1421 and SEQ ID NO:1422 of the international patent application WO2013143700 or homologs or variants thereof, whose disclosure is incorporated herewith by reference. In this context a particularly preferred fragment of a 5′-UTR of a TOP gene is a 5′-UTR of a TOP gene lacking the 5′-TOP motif. The term “5′-UTR of a TOP gene” preferably refers to the 5′-UTR of a naturally occurring TOP gene.

Stabilized nucleic acid, preferably mRNA: A stabilized nucleic acid, preferably mRNA typically, exhibits a modification increasing resistance to in vivo degradation (e.g. degradation by an exo- or endo-nuclease) and/or ex vivo degradation (e.g. by the manufacturing process prior to vaccine administration, e.g. in the course of the preparation of the vaccine solution to be administered). Stabilization of RNA can, e.g., be achieved by providing a 5′-CAP-Structure, a polyA-Tail, or any other UTR-modification. It can also be achieved by chemical modification or modification of the G/C content of the nucleic acid. Various other methods are known in the art and conceivable in the context of the invention.

RNA In vitro transcription: The terms “RNA in vitro transcription” or “in vitro transcription” relate to a process wherein RNA is synthesized in a cell-free system (in vitro). DNA, particularly plasmid DNA, is used as template for the generation of RNA transcripts. RNA may be obtained by DNA-dependent in vitro transcription of an appropriate DNA template, which according to the present invention is preferably a linearized plasmid DNA template. The promoter for controlling in vitro transcription can be any promoter for any DNA-dependent RNA polymerase. Particular examples of DNA-dependent RNA polymerases are the T7, T3, and SP6 RNA polymerases. A DNA template for in vitro RNA transcription may be obtained by cloning of a nucleic acid, in particular cDNA corresponding to the respective RNA to be in vitro transcribed, and introducing it into an appropriate vector for in vitro transcription, for example into plasmid DNA. In a preferred embodiment of the present invention the DNA template is linearized with a suitable restriction enzyme, before it is transcribed in vitro.

The cDNA may be obtained by reverse transcription of mRNA or chemical synthesis. Moreover, the DNA template for in vitro RNA synthesis may also be obtained by gene synthesis.

Methods for in vitro transcription are known in the art (see, e.g., Geall et al. (2013) Semin. Immunol. 25(2): 152-159; Brunelle et al. (2013) Methods Enzymol. 530:101-14). Reagents used in said method typically include:
1) a linearized DNA template with a promoter sequence that has a high binding affinity for its respective RNA polymerase such as bacteriophage-encoded RNA polymerases;
2) ribonucleoside triphosphates (NTPs) for the four bases (adenine, cytosine, guanine and uracil); 3) optionally, a CAP analogue as defined above (e.g. m7G(5′)ppp(5′)G (m7G));
4) a DNA-dependent RNA polymerase capable of binding to the promoter sequence within the linearized DNA template (e.g. T7, T3 or SP6 RNA polymerase);
5) optionally, a ribonuclease (RNase) inhibitor to inactivate any contaminating RNase;
6) optionally, a pyrophosphatase to degrade pyrophosphate, which may inhibit transcription;
7) MgCl₂, which supplies Mg²⁺ ions as a co-factor for the polymerase;
8) a buffer to maintain a suitable pH value, which can also contain antioxidants (e.g. DTT), and/or polyamines such as spermidine at optimal concentrations.

Full-length protein: The term “full-length protein” as used herein typically refers to a protein that substantially comprises the entire amino acid sequence of the naturally occurring protein. Nevertheless, substitutions of amino acids e.g. due to mutation in the protein are also encompassed in the term full-length protein.

Fragments of proteins: “Fragments” of proteins or peptides in the context of the present invention may, typically, comprise a sequence of a protein or peptide as defined herein, which is, with regard to its amino acid sequence (or its encoded nucleic acid molecule), N-terminally and/or C-terminally truncated compared to the amino acid sequence of the original (native) protein (or its encoded nucleic acid molecule). Such truncation may thus occur either on the amino acid level or correspondingly on the nucleic acid level. A sequence identity with respect to such a fragment as defined herein may therefore preferably refer to the entire protein or peptide as defined herein or to the entire (coding) nucleic acid molecule of such a protein or peptide.

The term “variant” in the context of nucleic acid sequences of genes refers to nucleic acid sequence variants, i.e. nucleic acid sequences or genes comprising a nucleic acid sequence that differs in at least one nucleic acid from a reference (or “parent”) nucleic acid sequence of a reference (or “parent”) nucleic acid or gene. Variant nucleic acids or genes may thus preferably comprise, in their nucleic acid sequence, at least one mutation, substitution, insertion or deletion as compared to their respective reference sequence. Preferably, the term “variant” as used herein includes naturally occurring variants, and engineered variants of nucleic acid sequences or genes. Therefore, a “variant” as defined herein can be derived from, isolated from, related to, based on or homologous to the reference nucleic acid sequence. “Variants” may preferably have a sequence identity of at least 5%, 10%, 20%, 30%, 40%, 50%, 60%, 70%, 80%, 85%, 86%, 87%, 88%, 89%, 90%, 91%, 92%, 93%, 94%, 95%, 96%, 97%, 98%, or 99%, preferably of at least 70%, more preferably of at least 80%, even more preferably at least 85%, even more preferably of at least 90% and most preferably of at least 95% or even 97%, to a nucleic acid sequence of the respective naturally occurring (wild-type) nucleic acid sequence or gene, or a homolog, fragment or derivative thereof.

Also, the term “variant” as used throughout the present specification in the context of proteins or peptides will be recognized and understood by the person of ordinary skill in the art, and is e.g. intended to refer to a proteins or peptide variant having an amino acid sequence which differs from the original sequence in one or more mutation(s), such as one or more substituted, inserted and/or deleted amino acid(s). Preferably, these fragments and/or variants have the same biological function or specific activity compared to the full-length native protein, e.g. its specific antigenic property. “Variants” of proteins or peptides as defined herein may comprise conservative amino acid substitution(s) compared to their native, i.e. non-mutated physiological, sequence. Those amino acid sequences as well as their encoding nucleotide sequences in particular fall under the term variants as defined herein. Substitutions in which amino acids, which originate from the same class, are exchanged for one another are called conservative substitutions. In particular, these are amino acids having aliphatic side chains, positively or negatively charged side chains, aromatic groups in the side chains or amino acids, the side chains of which can enter into hydrogen bridges, e.g. side chains which have a hydroxyl function. This means that e.g. an amino acid having a polar side chain is replaced by another amino acid having a likewise polar side chain, or, e.g., an amino acid characterized by a hydrophobic side chain is substituted by another amino acid having a likewise hydrophobic side chain (e.g. serine (threonine) by threonine (serine) or leucine (isoleucine) by isoleucine (leucine)). Insertions and substitutions are possible, in particular, at those sequence positions which cause no modification to the three-dimensional structure or do not affect the binding region. Modifications to a three-dimensional structure by insertion(s) or deletion(s) can easily be determined e.g. using CD spectra (circular dichroism spectra). A “variant” of a protein or peptide may have at least 70%, 75%, 80%, 85%, 90%, 95%, 98% or 99% amino acid identity over a stretch of at least 10, 20, 30, 50, 75 or 100 amino acids of such protein or peptide. Preferably, a variant of a protein comprises a functional variant of the protein, which means that the variant exerts the same effect or functionality or at least 40%, 50%, 60%, 70%, 80%, 90%, or 95% of the effect or functionality as the protein it is derived from.

Also, the term “fragment” in the context of nucleic acid sequences or genes refers to a continuous subsequence of the full-length reference (or “parent”) nucleic acid sequence or gene. In other words, a “fragment” may typically be a shorter portion of a full-length nucleic acid sequence or gene. Accordingly, a fragment, typically, consists of a sequence that is identical to the corresponding stretch within the full-length nucleic acid sequence or gene. The term includes naturally occurring fragments as well as engineered fragments. A preferred fragment of a sequence in the context of the present invention, consists of a continuous stretch of nucleic acids corresponding to a continuous stretch of entities in the nucleic acid or gene the fragment is derived from, which represents at least 20%, preferably at least 30%, more preferably at least 40%, more preferably at least 50%, even more preferably at least 60%, even more preferably at least 70%, and most preferably at least 80% of the total (i.e. full-length) nucleic acid sequence or gene from which the fragment is derived. A sequence identity indicated with respect to such a fragment preferably refers to the entire nucleic acid sequence or gene. Preferably, a “fragment” may comprise a nucleic acid sequence having a sequence identity of at least 5%, 10%, 20%, 30%, 40%, 50%, 60%, 70%, 80%, 85%, 86%, 87%, 88%, 89%, 90%, 91%, 92%, 93%, 94%, 95%, 96%, 97%, 98%, or 99%, preferably of at least 70%, more preferably of at least 80%, even more preferably at least 85%, even more preferably of at least 90% and most preferably of at least 95% or even 97%, to a reference nucleic acid sequence or gene that it is derived from.

Also, in this context a fragment of a protein may typically comprise an amino acid sequence having a sequence identity of at least 5%, 10%, 20%, 30%, 40%, 50%, 60%, 70%, 80%, 85%, 86%, 87%, 88%, 89%, 90%, 91%, 92%, 93%, 94%, 95%, 96%, 97%, 98%, or 99%, preferably of at least 70%, more preferably of at least 80%, even more preferably at least 85%, even more preferably of at least 90% and most preferably of at least 95% or even 97%, with an amino acid sequence of the respective naturally occurring full-length protein.

Fragments of proteins or peptides in the context of the present invention may furthermore comprise a sequence of a protein or peptide as defined herein, which has a length of for example at least 5 amino acids, preferably a length of at least 6 amino acids, preferably at least 7 amino acids, more preferably at least 8 amino acids, even more preferably at least 9 amino acids; even more preferably at least 10 amino acids; even more preferably at least 11 amino acids; even more preferably at least 12 amino acids; even more preferably at least 13 amino acids; even more preferably at least 14 amino acids; even more preferably at least 15 amino acids; even more preferably at least 16 amino acids; even more preferably at least 17 amino acids; even more preferably at least 18 amino acids; even more preferably at least 19 amino acids; even more preferably at least 20 amino acids; even more preferably at least 25 amino acids; even more preferably at least 30 amino acids; even more preferably at least 35 amino acids; even more preferably at least 50 amino acids; or most preferably at least 100 amino acids. For example such fragment may have a length of about 6 to about 20 or even more amino acids, e.g. fragments as processed and presented by MHC class I molecules, preferably having a length of about 8 to about 10 amino acids, e.g. 8, 9, or 10, (or even 6, 7, 11, or 12 amino acids), or fragments as processed and presented by MHC class II molecules, preferably having a length of about 13 or more amino acids, e.g. 13, 14, 15, 16, 17, 18, 19, 20 or even more amino acids, wherein these fragments may be selected from any part of the amino acid sequence. These fragments are typically recognized by T-cells in form of a complex consisting of the peptide fragment and an MHC molecule, i.e. the fragments are typically not recognized in their native form. Fragments of proteins or peptides may comprise at least one epitope of those proteins or peptides. Furthermore also domains of a protein, like the extracellular domain, the intracellular domain or the transmembrane domain and shortened or truncated versions of a protein may be understood to comprise a fragment of a protein.

Variants of proteins: “Variants” of proteins or peptides as defined in the context of the present invention may be generated, having an amino acid sequence which differs from the original sequence in one or more mutation(s), such as one or more substituted, inserted and/or deleted amino acid(s). Preferably, these fragments and/or variants have the same biological function or specific activity compared to the full-length native protein, e.g. its specific antigenic property. “Variants” of proteins or peptides as defined in the context of the present invention may comprise conservative amino acid substitution(s) compared to their native, i.e. non-mutated physiological, sequence. Those amino acid sequences as well as their encoding nucleotide sequences in particular fall under the term variants as defined herein. Substitutions in which amino acids, which originate from the same class, are exchanged for one another are called conservative substitutions. In particular, these are amino acids having aliphatic side chains, positively or negatively charged side chains, aromatic groups in the side chains or amino acids, the side chains of which can enter into hydrogen bridges, e.g. side chains which have a hydroxyl function. This means that e.g. an amino acid having a polar side chain is replaced by another amino acid having a likewise polar side chain, or, for example, an amino acid characterized by a hydrophobic side chain is substituted by another amino acid having a likewise hydrophobic side chain (e.g. serine (threonine) by threonine (serine) or leucine (isoleucine) by isoleucine (leucine)). Insertions and substitutions are possible, in particular, at those sequence positions which cause no modification to the three-dimensional structure or do not affect the binding region. Modifications to a three-dimensional structure by insertion(s) or deletion(s) can easily be determined e.g. using CD spectra (circular dichroism spectra) (Urry, 1985, Absorption, Circular Dichroism and ORD of Polypeptides, in: Modern Physical Methods in Biochemistry, Neuberger et al. (ed.), Elsevier, Amsterdam).

A “variant” of a protein or peptide may have at least 70%, 75%, 80%, 85%, 90%, 95%, 98% or 99% amino acid identity over a stretch of 10, 20, 30, 50, 75 or 100 amino acids of such protein or peptide.

Furthermore, variants of proteins or peptides as defined herein, which may be encoded by a nucleic acid molecule, may also comprise those sequences, wherein nucleotides of the encoding nucleic acid sequence are exchanged according to the degeneration of the genetic code, without leading to an alteration of the respective amino acid sequence of the protein or peptide, i.e. the amino acid sequence or at least part thereof may not differ from the original sequence in one or more mutation(s) within the above meaning.

Identity of a sequence: In order to determine the percentage to which two sequences are identical, e.g. nucleic acid sequences or amino acid sequences as defined herein, preferably the amino acid sequences encoded by a nucleic acid sequence of the polymeric carrier as defined herein or the amino acid sequences themselves, the sequences can be aligned in order to be subsequently compared to one another. Therefore, e.g. a position of a first sequence may be compared with the corresponding position of the second sequence. If a position in the first sequence is occupied by the same component (residue) as is the case at a position in the second sequence, the two sequences are identical at this position. If this is not the case, the sequences differ at this position. If insertions occur in the second sequence in comparison to the first sequence, gaps can be inserted into the first sequence to allow a further alignment. If deletions occur in the second sequence in comparison to the first sequence, gaps can be inserted into the second sequence to allow a further alignment. The percentage to which two sequences are identical is then a function of the number of identical positions divided by the total number of positions including those positions which are only occupied in one sequence. The percentage to which two sequences are identical can be determined using a mathematical algorithm. A preferred, but not limiting, example of a mathematical algorithm which can be used is the algorithm of Karlin et al. (1993), PNAS USA, 90:5873-5877 or Altschul et al. (1997), Nucleic Acids Res., 25:3389-3402. Such an algorithm is integrated in the BLAST program. Sequences which are identical to the sequences of the present invention to a certain extent can be identified by this program.

Derivative of a protein or peptide: A derivative of a peptide or protein is typically understood to be a molecule that is derived from another molecule, such as said peptide or protein. A “derivative” of a peptide or protein also encompasses fusions comprising a peptide or protein used in the present invention. For example, the fusion comprises a label, such as, for example, an epitope, e.g., a FLAG epitope or a V5 epitope. For example, the epitope is a FLAG epitope. Such a tag is useful for, for example, purifying the fusion protein.

Pharmaceutically effective amount: A pharmaceutically effective amount in the context of the invention is typically understood to be an amount that is sufficient to induce an immune response.

Carrier: A “carrier” or a “carrier composition” in the context of the invention is a compound or a plurality of compounds that facilitate transport and/or complexation of another compound, e.g. a nucleic acid. Said carrier may form a complex with said other compound, e.g. the nucleic acid. Preferably, the carrier or carrier composition is a lipid nanoparticle or a lipid nanoparticle composition, as described herein below, e.g. under paragraph “Lipid nanoparticle compositions”. A polymeric carrier is a carrier that is formed of a polymer, e.g. a cationic polymer comprising amino acids with a positive charge (e.g. peptides comprising amino acids G, H or R, preferably further comprising cysteine). Protamine as carrier is e.g. disclosed in PMIDs 27336830 or 23159882, EP1083232, WO2010037539, WO2012116811, WO2012116810, and WO2015024665.

Vehicle: An agent, e.g. a carrier that is typically used within a pharmaceutical composition or vaccine for facilitating administering of the components of the pharmaceutical composition or vaccine to an individual.

### BRIEF DESCRIPTION OF THE DRAWINGS

FIG. 1 (Chemical structures of cationic lipids)—shows the structures of HEXA1 (HEXA-C5DE-PipSS, see FIG. 1A), HEXA2 (HEXA-C5DE-PipC3SS, see FIG. 1B) and THIOETHER (VitE-C4DE-Pip-Thioether, see FIG. 1C)—full details can be seen in in Example 1).

FIG. 2 (LNPs for in vivo Malaria vaccination—IgGₜₒₜₐₗ titer)—shows that the tested mRNA Malaria vaccines encoding CSP induced very strong humoral immune responses in mice, using an ELISA assay (coating: [NANP]₇ peptide, IgG total detection signal at a fixed titer at day 35 post prime (full details can be found in Example 3).

FIG. 3 (LNPs for in vivo Malaria vaccination—Intracellular Staining (ICS)) shows that LNP formulated mRNA Malaria vaccine encoding CSP induced cellular immune responses in mice: CD8+ T-cell responses are shown for the re-stimulated setup in FIG. 3A, CD4+ T-cell responses are shown for the re-stimulated setup in FIG. 3B. An intracellular cytokine staining assay (day 35 post vaccination) was used. The full details can be found in Example 3.

FIG. 4 shows an exemplary PMOZ-lipid for Example 4.

FIG. 5 (Organ targeting of LNPs)—shows the results of the PpLuc expression in different tissue lysates after organ collection for an LNP8-formulated mRNA encoding PpLuc at two different time points, namely at 4 h and at 24 h, LNP8 comprising DPhyPS. At both time points, the highest expression is observed in the spleen (the expression levels are shown as pg of total luciferase protein/organ). The full details can be found in Example 5.

FIG. 6 (Organ targeting of LNPs)—shows the results of the PpLuc expression in spleen (see FIG. 6A) and liver (see FIG. 6B) for an LNP8-formulated mRNA encoding PpLuc at two different time points, namely at 4 h and at 24 h (the expression levels were normalized to total protein and are given as pg/mg of total protein; it is noted that the y-axes of FIG. 6A and FIG. 6B differ in order to account for the fact that the expression in the spleen is much higher than in the liver). The full details can be found in Example 5.

FIG. 7A (Immunogenicity of the LNPs/VNT analysis)—the figure shows that an i.m. immunization with 1 μg LNP-formulated RABV-G-mRNA induced very robust VNTs well above the protective titer of 0.5 IU/ml (indicated by a dashed line) in all animals at day 28, 7 days post boost vaccination (FIG. 7A). The full details can be found in Example 6.

FIG. 7B—shows the number of spots indicative of induction of RABV-G protein specific T-cells including cytotoxic T cells (CTL) in an experiment with mice after vaccination with mRNA coding for the RABV-G protein. One week after the last boost vaccination, on day 28 post prime, antigen specific cells were analyzed in spleens of vaccinated mice by ELISpot (Enzyme Linked Immuno Spot Assay) analysis. Splenocytes were re-stimulated with a RABV-G peptide mix (PepMix) of 129 peptides (final concentration of 5 μg/peptide). The y-axis shows the number of detected IFN-gamma spots per 1 mio. cells; bars represent the mean. IFN-gamma (Interferon-gamma) is considered to be a prototype proinflammatory cytokine being produced by a variety of immune cells under inflammatory conditions, notably by T-cells and natural killer (NK) cells. By promoting the development and activation of Type 1 helper T cells (Th1 cells—leading to an increased cell-mediated response), chemoattraction and activation of monocytes and macrophages and upregulation of antigen presentation molecules, it plays a key role in the host defense. IFN-gamma also exhibits antiviral, anti-proliferative, and apoptotic effects. Results: as can be seen, the addition of PS to the specific LNP formulations strongly increased the number of detected IFN-gamma spots indicative of induction of RABV-G protein specific T cells including cytotoxic T cell (CTL) in comparison to the control LNP formulations. The full details for this experiment can be found in Example 6.

FIG. 8A (LNPs for in vivo Malaria vaccination—long-term experiment)—shows that the tested mRNA Malaria vaccines encoding CSP induced a humoral immune responses in mice, using an ELISA assay (coating: [NANP]₇ peptide, IgGₜₒₜₐₗ titers at day 35 post prime (full details can be found in Example 7).

FIG. 8B (LNPs for in vivo Malaria vaccination—long-term experiment)—shows that the tested mRNA Malaria vaccines encoding CSP induced a very strong humoral immune responses in mice, using an ELISA assay (coating: [NANP]₇ peptide, IgGₜₒₜₐₗ titers at day 105 post prime (full details can be found in Example 7).

FIG. 9 (organ distribution of PpLuc after intramuscular LNP injection)—shows PpLuc expression in different tissue lysates after organ collection for LNP-formulated mRNA encoding PpLuc at two different time points (4 h and 24 h). Expression levels are shown as pg of total luciferase protein/organ. Full details can be found in Example 12; the following table is the corresponding figure legend to FIG. 9:

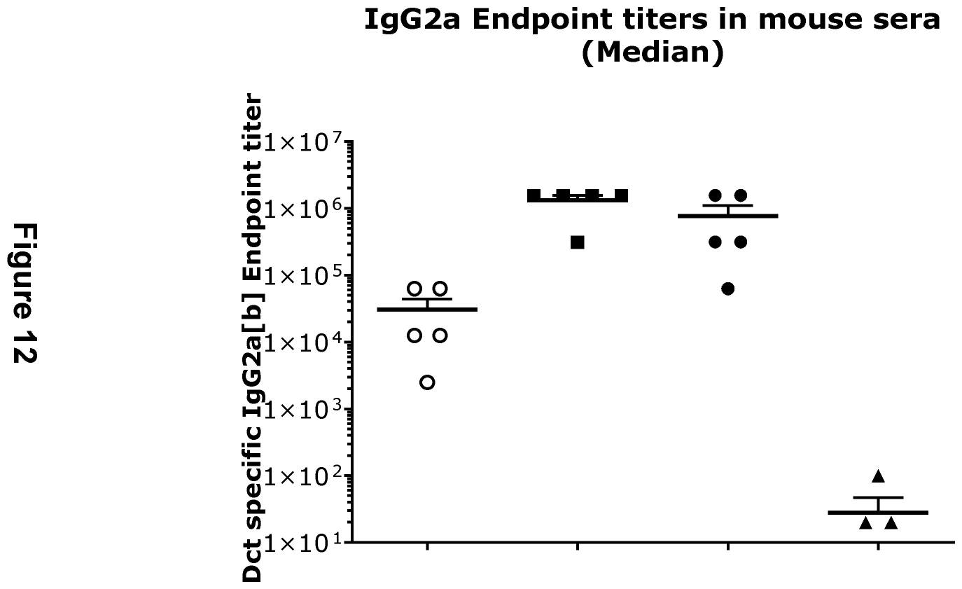

TABLE
figure legend to FIG. 9:

| | FIG. 9 | | | | | | Polymer |
|---|---|---|---|---|---|---|---|
| Group/ | legend/ | Time | | Ionizable | | Neutral | conjugated |
| Order | group | point | | lipid | Cholesterol | lipid(s) | lipid |
| 1 | open | 4 | h | 49 mol % | 38.5 mol % | 10 mol % DPhyPE | 2.5 mol % “PMOZ 4” |
| | circles | | | THIOETHER | | | (with with 50 |
| | | | | | | | monomer repeats |
| | | | | | | | (n = 50)) |
| 2 | open | 24 | h | 49 mol % | 38.5 mol % | 10 mol % DPhyPE | 2.5 mol % “PMOZ 4” |
| | squares | | | THIOETHER | | | (with with 50 |
| | | | | | | | monomer repeats |
| | | | | | | | (n = 50)) |
| 3 | filled | 4 | h | 59 mol % | 28.5 mol % | 5 mol % DPhyPE, | 2.5 mol % “PMOZ 4” |
| | circles | | | THIOETHER | | 5 mol % DPhyPS | (with with 50 |
| | | | | | | | monomer repeats |
| | | | | | | | (n = 50)) |
| 4 | filled | 24 | h | 59 mol % | 28.5 mol % | 5 mol % DPhyPE, | 2.5 mol % “PMOZ 4” |
| | squares | | | THIOETHER | | 5 mol % DPhyPS | (with with 50 |
| | | | | | | | monomer repeats |
| | | | | | | | (n = 50)) |

| | | | | |
|---|---|---|---|---|
| 5 | triangle | 4 | h | PBS buffer |
| | up | | | |
| 6 | triangle | 24 | h | PBS buffer |
| | down | | | |

FIG. 10 (organ distribution of PpLuc after intradermal LNP injection)—shows PpLuc expression in different tissue lysates after organ collection for LNP-formulated mRNA encoding PpLuc at two different time points (4 h and 24 h). Expression levels are shown as pg of total luciferase protein/organ; full details can be found in Example 13; the following table is the corresponding figure legend to FIG. 10:

TABLE
figure legend to FIG. 10:

| | FIG. 10 | | | | | | Polymer |
|---|---|---|---|---|---|---|---|
| Group/ | legend/ | Time | | Ionizable | | Neutral | conjugated |
| Order | group | point | | lipid | Cholesterol | lipid(s) | lipid |
| 1 | open | 4 | h | 49 mol % | 38.5 mol % | 10 mol % DPhyPE | 2.5 mol % “PMOZ 4” |
| | circles | | | THIOETHER | | | (with with 50 |
| | | | | | | | monomer repeats |
| | | | | | | | (n = 50)) |
| 2 | open | 24 | h | 49 mol % | 38.5 mol % | 10 mol % DPhyPE | 2.5 mol % “PMOZ 4” |
| | squares | | | THIOETHER | | | (with with 50 |
| | | | | | | | monomer repeats |
| | | | | | | | (n = 50)) |
| 3 | filled | 4 | h | 59 mol % | 28.5 mol % | 5 mol % DPhyPE, | 2.5 mol % “PMOZ 4” |
| | circles | | | THIOETHER | | 5 mol % DPhyPS | (with with 50 |
| | | | | | | | monomer repeats |
| | | | | | | | (n = 50)) |
| 4 | filled | 24 | h | 59 mol % | 28.5 mol % | 5 mol % DPhyPE, | 2.5 mol % “PMOZ 4” |
| | squares | | | THIOETHER | | 5 mol % DPhyPS | (with with 50 |
| | | | | | | | monomer repeats |
| | | | | | | | (n = 50)) |

| | | | | |
|---|---|---|---|---|
| 5 | triangle | 4 | h | PBS buffer |
| | up | | | |
| 6 | triangle | 24 | h | PBS buffer |
| | down | | | |

FIG. 11 (Mouse immunization with Tyrosinase-related protein 2 (Trp2) formulated in LNPs comprising PS)—shows that after peptide restimulation with a Trp2 immunodominant epitope, LNPs comprising DPhyPS (filled squares) and LNPs comprising DHPC (filled circles) showed a significantly higher CD8T cell response as when compared to LNPs not comprising DPhyPS or DHPC (open circles); full details can be found in Example 14.

FIG. 12 (Mouse immunization with Tyrosinase-related protein 2 (Trp2) formulated in LNPs comprising PS or DHPC)—shows that LNPs comprising DPhyPS (filled squares) and LNPs comprising DHPC (filled circles) showed significantly higher IgG2a endpoint titers as when compared to LNPs not comprising DPhyPS or DHPC (open circles); full details can be found in Example 14.

### DETAILED DESCRIPTION OF THE INVENTION

Although the present disclosure is described in detail below, it is to be understood that this disclosure is not limited to the particular methodologies, protocols and reagents described herein as these may vary. It is also to be understood that the terminology used herein is for the purpose of describing particular embodiments only, and is not intended to limit the scope of the present disclosure which will be limited only by the appended claims. Unless defined otherwise, all technical and scientific terms used herein have the same meanings as commonly understood by one of ordinary skill in the art.

The present invention is based on the inventors' surprising finding that phosphatidylserine-containing nucleic acid-based vaccines (in particular vaccines comprising a nucleic acid at least partly encapsulated by a lipid nanoparticle, wherein the lipid nanoparticle comprises phosphatidylserine) have improved properties compared to vaccines not comprising the phospholipid phosphatidylserine. Without wishing to be bound by theory, the inventors presently believe that this might be due to the role of the phospholipid phosphatidylserine during apoptosis. Thus, while phosphatidylserine is located in healthy cells exclusively in the inner lipid layer of the lipid bi-layer membrane of the cells, phosphatidylserine is translocated during apoptosis to the outer lipid layer of the lipid bi-layer membrane, such that the head-group of the phosphatidylserine is exposed to the cell surface in apoptotic cells. This exposed head-group of the phosphatidylserine serves as marker for the rapid uptake of apoptotic cells by cells of the immune system, namely phagocytic cells, in particular macrophages and dendritic cells. Accordingly, the marker phosphatidylserine might increase the process of phagocytosis of the vaccine compositions and thus increase the process of introducing the at least partly encapsulated nucleic acid into the phagocytic cells, where the nucleic acid will be translated, ultimately resulting in the presence of the encoded antigen or fragment or variant thereof in phagocytic cells. Again without wishing to be bound by theory, the inventors presently believe that this could inter alia explain the observed targeting of the phosphatidylserine-comprising vaccines to the spleen (see example 5 of the present application), which is in particular a center of activity of the mononuclear phagocyte system and serves as main storage for lymphocytes.

### The Phospholipid Phosphatidylserine

The term “phosphatidylserine” as used herein relates to a compound consisting of a head-group, which is a serine, bound via a phosphodiester to a carbon atom of glycerine, and one or more tail-groups. Preferably, said tail-group(s) is (are) a fatty acid, bound via an ester to another carbon atom of the glycerine. Preferably, the term “phosphatidylserine” as used herein relates to a compound consisting of a head-group, which is a serine, bound via a phosphodiester to a carbon atom of glycerine, and one or more tail-groups, wherein a tail-group is a fatty acid, which is bound via an ester to another carbon atom of the glycerine. A fatty acid can be a saturated fatty acid, preferably selected from the group consisting of caproic acid, caprylic acid, capric acid, lauric acid, myristic acid, palmitic acid, stearic acid, arachidic acid, behenic acid, lignoceric acid and cerotic acid. A fatty acid can also be an unsaturated fatty acid, preferably selected from the group consisting of myristoleic acid, palmitoleic acid, sapienic acid, oleic acid, elaidic acid, vaccenic acid, linoleic acid, linoelaidic acid, α-linolenic acid, arachidonic acid, eicosapentiaenoic acid, erucic acid and docosahexaenoic acid. A fatty acid can also be a branched chain fatty acid, such as in particular phytanic acid.

Examples are given below, wherein e.g. in the case of DPhyPS, WT-PS (i.e. 1-stearoyl-2-oleoyl-sn-glycero-3-phospho-L-serine or 18:0-18:1 PS, in accordance with the two different fatty acid/alkyl chains of WT-PS which is distributed widely among animals, plants and microorganisms), 16:0—PS, 14:0—PS, 10:0—PS, 6:0—PS and 18:1—PS DOPS, the serine is bound to a first carbon atom of the glycerine via a phosphodiester while the second and third carbon atoms of the glycerine are bound to a fatty acid, each via an ester. In this constellation, the two fatty acids may be identical (see e.g. DPhyPS, 16:0 PS, 14:0—PS, 10:0—PS, 6:0—PS and 18:1—PS DOPS) or may be different (see e.g. WT-PS or 18:0-18:1 PS). In other examples, e.g. in the case of 18:1-Lyso PS and 18:0-Lyso PS, the serine is again bound to a first carbon atom of the glycerine via a phosphodiester while only one further carbon atom of the glycerine is bound to a fatty acid via an ester, leaving a single OH-group at the remaining carbon atom of the glycerine. Such constellations are typically referred to as a “lysophosphatidylserine”, which is included in view of the above definition in the term “phosphatidylserine” as used herein.

In a preferred embodiment, the phosphatidylserine is selected from the group consisting of DPhyPS, WT-PS, 16:0—PS, 14:0—PS, 10:0—PS, 6:0—PS, 18:1—PS DOPS, 18:1-Lyso PS and 18:0-Lyso PS. It is most preferred that the phosphatidylserine is either DPhyPS or WT-PS (18:0-18:1 PS).

The structures of the phosphatidylserines mentioned above are as follows (it is noted that all of these lipids are commercially available, e.g. at Avanti Polar Lipids):

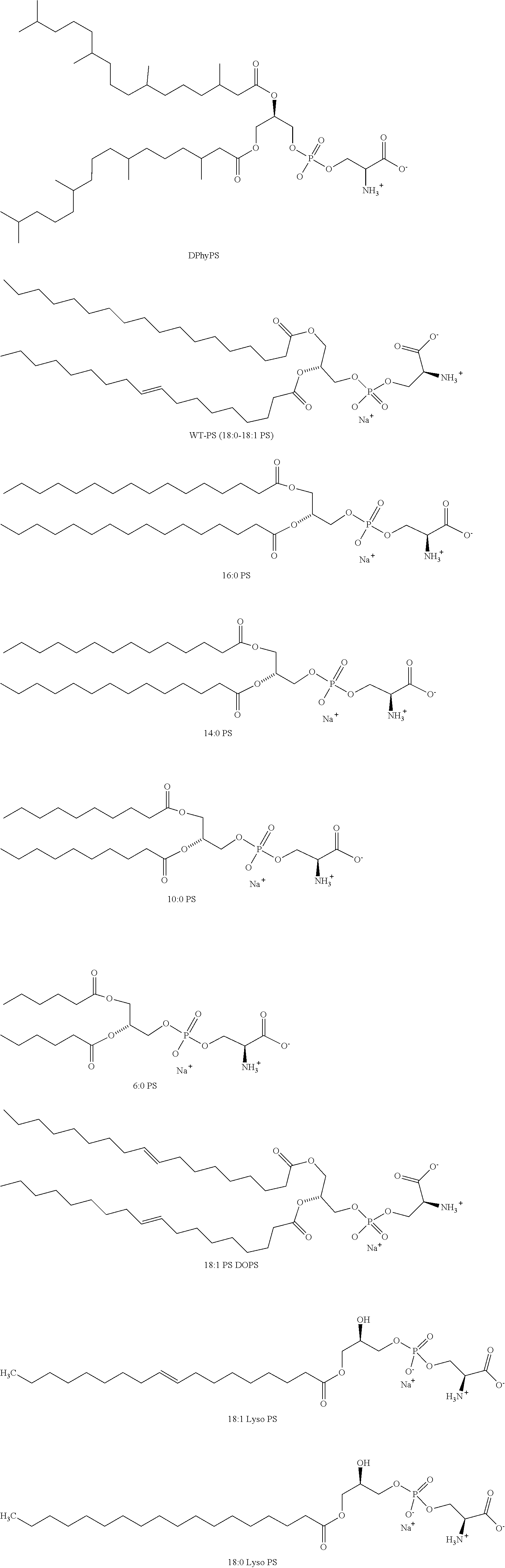

Further examples of saturated phosphatidylserine include 1,2-dilauroyl-sn-glycero-3-phosphoserine (DLPS), 1,2-dimyristoyl-sn-glycero-3-phosphoserine (dimyristoylphosphatidylserine; DMPS), 1,2-distearoyl-sn-glycero-3-phosphoserine (distearoylphosphatidylserine; DSPS), 1,2-dipalmitoyl-sn-glycero-3-phosphoserine (dipalmitoylphosphatidylserine; DPPS), 1-myristoyl-2-palmitoyl-sn-glycero-3-phosphoserine (MPPC), 1-palmitoyl-2-myristoyl-sn-glycero-3-phosphoserine (PMPS), 1-myristoyl-2-stearoyl-sn-glycero-3-phosphoserine (MSPS), 1-palmitoyl-2-stearoyl-sn-glycero-3-phosphoserine (PSPS), 1-stearoyl-2-palmitoyl-sn-glycero-3-phosphoserine (SPPS), and 1-stearoyl-2-myristoyl-sn-glycero-3-phosphoserine (SMPS). In some embodiments, the phosphatidylserine comprises a stearoyl (18:0) moiety, an oleoyl (18:1) moiety, an eicosatetraenoyl (20:4) moiety, a docosahexaenoyl (22:06) moiety, or a combination thereof. In other embodiments, the PS is L-a-phosphatidylserine (brain, porcine; CAS. Registry No. 383907-32-2).

### The Cationic or Ionizable Lipid

The cationic or ionizable lipid of an LNP may be cationisable, i.e. it becomes protonated as the pH is lowered below the pK of the ionizable group of the lipid, but is progressively more neutral at higher pH values. At pH values below the pK, the lipid is then able to associate with negatively charged nucleic acids. In certain embodiments, the cationic lipid comprises a zwitterionic lipid that assumes a positive charge on pH decrease.

Preferred cationic lipids are defined as a compound according to formula (Cat-I):

R^{a}-A-R^{b} formula (Cat-I)

wherein is
R^{a} is selected from:

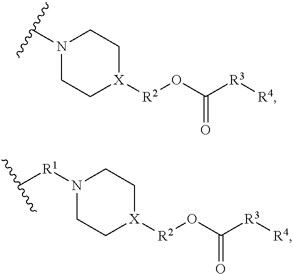

or
   —R¹—N(H)—C(O)—R³—R⁴;
R^{b} is selected from:

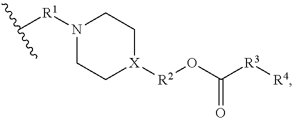

—R¹—N(H)—C(O)—R³—R⁴, or
   —R¹—N(CH₃)₂;
A is —S—, —S—S—, —NH—C(O)—, —NH—C(O)O—, —NH—C(O)—NH—, —S—C(O)—N(H)—, —C(O)O—, or —O—P(O)(OH)—O—;
R¹ is an optionally substituted ethanediyl, propanediyl, butanediyl, or linear or unbranched alkanediyl having 2 to 8 carbon atoms;
R² is an alkanediyl having 2 to 8 carbon atoms;
R³ is optional, and if present, is —R⁵—C(O)—O—, —R⁵—O—C(O)—, —R⁵—C(O)—NH—, —R⁵—OC(O)—NH—, or R⁵—NH—C(O)O—;
R⁴ is a lipophilic substituent with 12 to 36 carbon atoms;
R⁵ is an alkanediyl having 1 to 6 carbon atoms;
X is a carbon or nitrogen atom;
   wherein all selections are independent of one another,
   optionally provided that if R¹, R² and R⁵ are all linear unsubstituted ethanediyl, A is —S—S—, and R^{a} and R^{b} are identical, then R⁴ is not

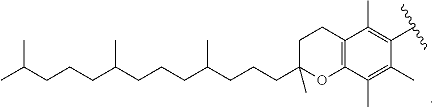 [SMILES: *c1c(C)c(C)c2c(c1C)CCC(C)(CCCC(C)CCCC(C)CCCC(C)C)O2]

In other embodiments, R⁴ is

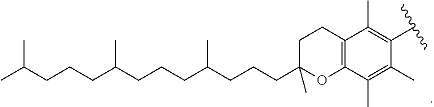 [SMILES: *c1c(C)c(C)c2c(c1C)CCC(C)(CCCC(C)CCCC(C)CCCC(C)C)O2]

In one preferred embodiment, A is —S—, and R^{a} and R^{b} are identical, and R⁴ is

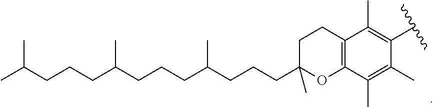 [SMILES: *c1c(C)c(C)c2c(c1C)CCC(C)(CCCC(C)CCCC(C)CCCC(C)C)O2]

In another preferred embodiment, A is —S— and R⁴ is

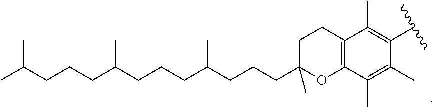 [SMILES: *c1c(C)c(C)c2c(c1C)CCC(C)(CCCC(C)CCCC(C)CCCC(C)C)O2]

R⁴ from formula (Cat-I) is defined as a lipophilic substituent with 12 to 36 carbon atoms. This “tail” end of R^{a} and optionally also of R^{b} (unless R^{b} is —R¹—N(CH₃)₂) is believed to provide the degree of lipophilicity which is typically required for molecules to be able to cross biological membranes. Therefore, R⁴ may in principle be of any structure that is substantially lipophilic. For example, a hydrocarbon structure is lipophilic. In one embodiment, R⁴, in at least one of its occurrences, may consist of only carbon and hydrogen atoms. In one preferred embodiment, R⁴ represents a linear or branched alkyl or alkenyl, preferably having 12 to 25 carbon atoms. The branched alkyl or alkenyl may optionally have a plurality of side chains, such as 2, 3, 4 or more methyl side chains. In another embodiment, R⁴ may be an alkyl or alkenyl comprising a single alkyl or alkenyl side chain with e.g. 2 to 10 carbon atoms. For example, R⁴ may be 1-n-hexyl-n-nonyl (or 7-n-pentadecyl), or 2-n-hexyl-n-decyl. In other embodiments, the lipophilic substituent may optionally include one or more heteroatoms such as O, S, or N. In other embodiments, the lipophilic substituent may optionally include one or more saturated, unsaturated, or aromatic ring structures that may optionally include one or more heteroatoms such as O, S, or N.

R⁴ may also include a small number of hetero atoms such as oxygen atoms, as long as the predominantly lipophilic character is maintained. In one embodiment, R⁴ comprises one or more oxygen atoms and no other hetero atoms. R⁴ may also comprise a cyclic structure, such as an aromatic or aliphatic ring structure optionally including one or more oxygen atoms. If present, it is preferred that the hetero atoms and/or the cyclic structure are located towards the optional R³ structure rather than towards the end of the “tail”. In one embodiment, R⁴ is a lipophilic group derived from tocopherol or tocotreinol. In one embodiment, R⁴ is a lipophilic group derived from alpha-tocopherol, in particular

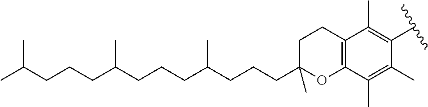 [SMILES: *c1c(C)c(C)c2c(c1C)CCC(C)(CCCC(C)CCCC(C)CCCC(C)C)O2]

in particular if not all of R¹, R² and R⁵ are linear unsubstituted ethanediyl, A is —S—S—, and R^{a} and R^{b} are identical.

A “lipophilic group derived from tocopherol or tocotreinol” as referred to herein includes derivatives of tocopherol and tocotreinol, in particular the derivatives with the structures shown in Scheme 1 below, i.e. the derivatives derived from alpha-tocopherol, beta-tocopherol, gamma-tocopherol, delta-tocopherol, alpha-tocotreinol, beta-tocotreinol, gamma-tocotreinol and delta-tocotreinol.

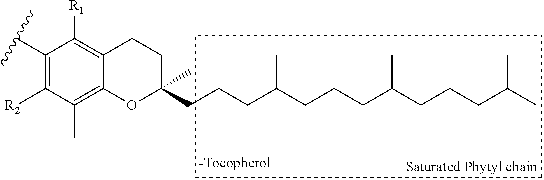

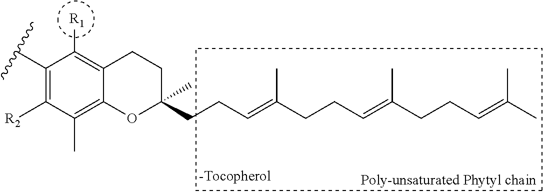

| Isoform | R₁ | R₂ |
|---|---|---|
| Alpha (α) | CH3 | CH3 |
| Beta (β) | CH3 | H |
| Gamma (γ) | H | CH3 |
| Delta (δ) | H | H |

Scheme 1: Derivatives of tocopherol have a saturated phytyl chain, whereas derivatives of tocotreinol have a poly-unsaturated phytyl chain. For both, derivatives of tocopherol and tocotreinol, the isoforms are defined by R₁ and R₂, which are selected from CH₃ and H. Thus, as shown, if e.g. R1 is CH₃ and R₂ is CH₃, the resulting derivative is the alpha isoform of tocopherol and tocotreinol, respectively (referred to as derivative of alpha- tocopherol and alpha-tocotreinol, respectively). The OH-group is of course not present in the derivatives since this is the point of attachment, as shown in the two structures on the left.

In other preferred embodiments, the at least one nucleic acid (e.g. DNA or RNA), preferably the at least one mRNA of the composition is complexed with one or more lipids thereby forming LNPs, wherein the cationic lipid of the LNP is selected from structures C1 to C23, or respectively C1 to C27 of Table 1 or a lipid derived from formula (I) of PCT patent application PCT/EP2019/086825 or the subsequent patent application thereof claiming the priority of PCT/EP2019/086825 i.e. WO2021123332. In other embodiments, the at least one nucleic acid (e.g. DNA or RNA), preferably the at least one mRNA of the composition is complexed with one or more lipids thereby forming LNPs, wherein the cationic lipid of the LNP is derived from structures C1 to C23, or respectively C1 to C27 of Table 1 of PCT patent application PCT/EP2019/086825 or the subsequent patent application thereof claiming the priority of PCT/EP2019/086825 i.e. WO2021123332, wherein the element “A” from formula (I) of PCT/EP2019/086825 is —S—. Accordingly, formulas C1 to C23, or respectively C1 to C27, of PCT patent application PCT/EP2019/086825 or the subsequent patent application thereof claiming the priority of PCT/EP2019/086825 i.e. WO2021123332, and the specific disclosure relating thereto, are herewith incorporated by reference.

In yet a further embodiment, the cationic lipid preferably is selected from the cationic lipids as listed herein in Table 1.

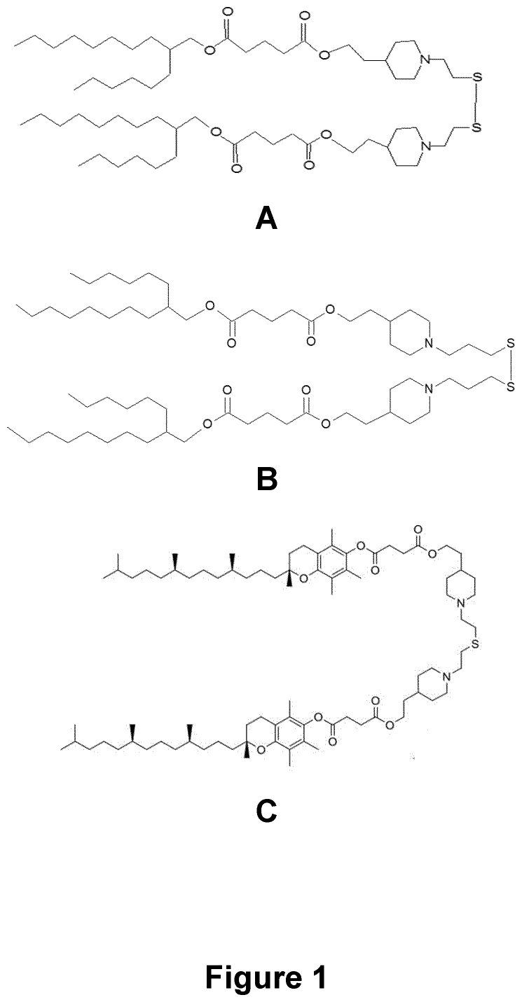

TABLE 1
Preferred cationic lipids according to formula (I) - when it is referred to specific lipids from this table, e.g.
lipid C1, reference is made e.g. to “Lipid C1”, “Lipid Compound 1”, “HEXA-C4DE-PipSS” or “C1”

| Cationic | | |
|---|---|---|
| Lipid | | |
| Compound | | |
| No. | Structure | Name |
| C1 | 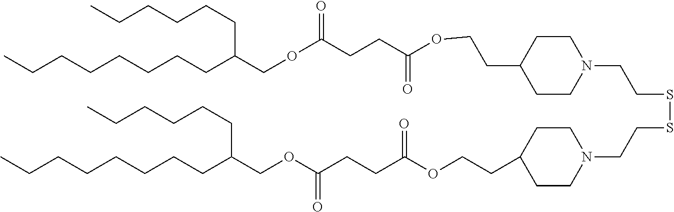 [SMILES: CCCCCCCCC(CCCCCC)COC(=O)CCC(=O)OCCC1CCN(CCSSCCN2CCC(CCOC(=O)CCC(=O)OCC(CCCCCC)CCCCCCCC)CC2)CC1] | HEXA-C4DE- PipSS |
| C2 | 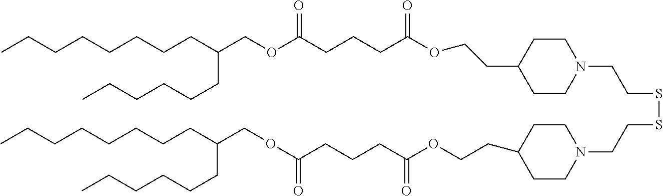 [SMILES: CCCCCCCCC(CCCCCC)COC(=O)CCCC(=O)OCCC1CCN(CCSSCCN2CCC(CCOC(=O)CCCC(=O)OCC(CCCCCC)CCCCCCCC)CC2)CC1] | HEXA-C5DE- PipSS (GN02- lipid) (see also FIG. 1A) |
| C3 | 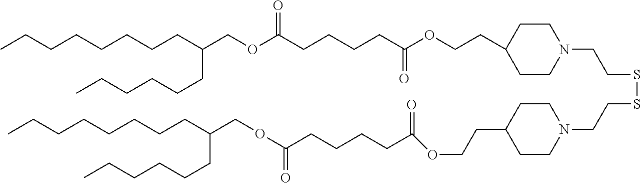 [SMILES: CCCCCCCCC(CCCCCC)COC(=O)CCCCC(=O)OCCC1CCN(CCSSCCN2CCC(CCOC(=O)CCCCC(=O)OCC(CCCCCC)CCCCCCCC)CC2)CC1] | HEXA-C6DE- PipSS |
| C4 | 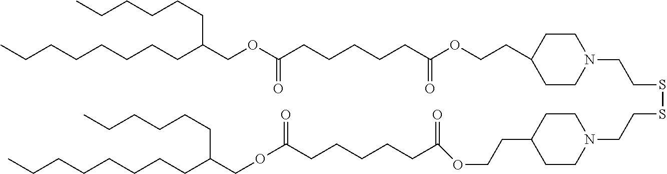 [SMILES: CCCCCCCCC(CCCCCC)COC(=O)CCCCCC(=O)OCCC1CCN(CCSSCCN2CCC(CCOC(=O)CCCCCC(=O)OCC(CCCCCC)CCCCCCCC)CC2)CC1] | HEXA-C7DE- PipSS |
| C5 | 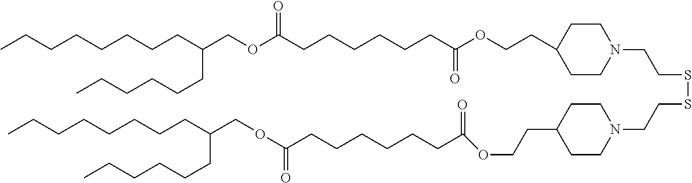 [SMILES: CCCCCCCCC(CCCCCC)COC(=O)CCCCCCC(=O)OCCC1CCN(CCSSCCN2CCC(CCOC(=O)CCCCCCC(=O)OCC(CCCCCC)CCCCCCCC)CC2)CC1] | HEXA-C8DE- PipSS |
| C6 | 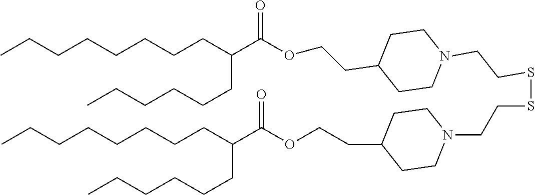 [SMILES: CCCCCCCCC(CCCCCC)C(=O)OCCC1CCN(CCSSCCN2CCC(CCOC(=O)C(CCCCCC)CCCCCCCC)CC2)CC1] | HEXACA -C3ME- PipSS |
| C7 | 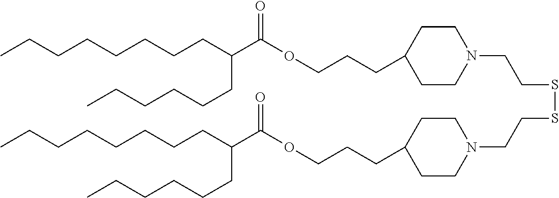 [SMILES: CCCCCCCCC(CCCCCC)C(=O)OCCCC1CCN(CCSSCCN2CCC(CCCOC(=O)C(CCCCCC)CCCCCCCC)CC2)CC1] | HEXACA- C4ME- PipSS |
| C8 | 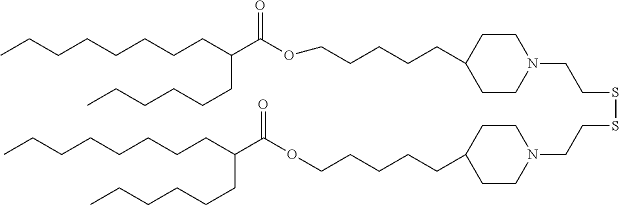 [SMILES: CCCCCCCCC(CCCCCC)C(=O)OCCCCCC1CCN(CCSSCCN2CCC(CCCCCOC(=O)C(CCCCCC)CCCCCCCC)CC2)CC1] | HEXACA- C6ME- PipSS |
| C9 | 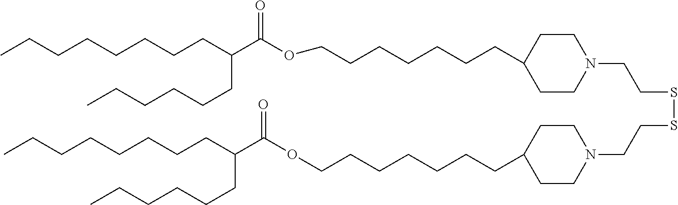 [SMILES: CCCCCCCCC(CCCCCC)C(=O)OCCCCCCCC1CCN(CCSSCCN2CCC(CCCCCCCOC(=O)C(CCCCCC)CCCCCCCC)CC2)CC1] | HEXACA- C8ME- PipSS |
| C10 | 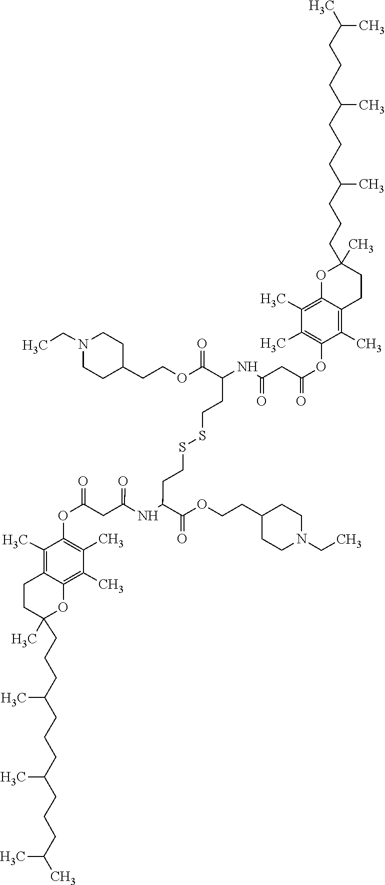 [SMILES: CCN1CCC(CCOC(=O)C(CCSSCCC(NC(=O)CC(=O)Oc2c(C)c(C)c3c(c2C)CCC(C)(CCCC(C)CCCC(C)CCCC(C)C)O3)C(=O)OCCC2CCN(CC)CC2)NC(=O)CC(=O)Oc2c(C)c(C)c3c(c2C)CCC(C)(CCCC(C)CCCC(C)CCCC(C)C)O3)CC1] | C10/Compound 10 |
| C11 | 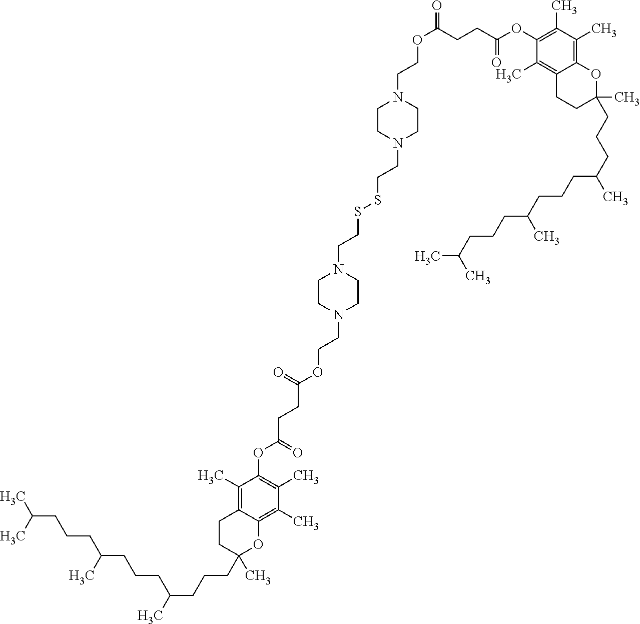 [SMILES: Cc1c(C)c2c(c(C)c1OC(=O)CCC(=O)OCCN1CCN(CCSSCCN3CCN(CCOC(=O)CCC(=O)Oc4c(C)c(C)c5c(c4C)CCC(C)(CCCC(C)CCCC(C)CCCC(C)C)O5)CC3)CC1)CCC(C)(CCCC(C)CCCC(C)CCCC(C)C)O2] | C11/Compound 11 |
| C12 | 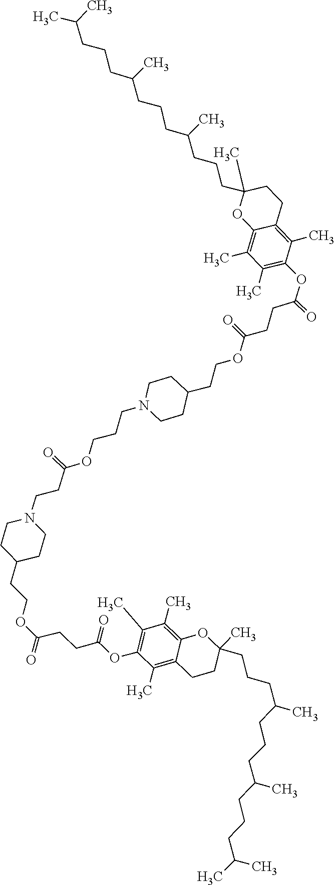 [SMILES: Cc1c(C)c2c(c(C)c1OC(=O)CCC(=O)OCCC1CCN(CCCOC(=O)CCN3CCC(CCOC(=O)CCC(=O)Oc4c(C)c(C)c5c(c4C)CCC(C)(CCCC(C)CCCC(C)CCCC(C)C)O5)CC3)CC1)CCC(C)(CCCC(C)CCCC(C)CCCC(C)C)O2] | C12/Compound 12 |
| C13 | 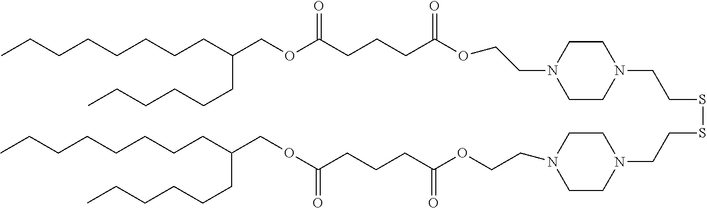 [SMILES: CCCCCCCCC(CCCCCC)COC(=O)CCCC(=O)OCCN1CCN(CCSSCCN2CCN(CCOC(=O)CCCC(=O)OCC(CCCCCC)CCCCCCCC)CC2)CC1] | HEXA-C5DE- PipAZSS |
| C14 | 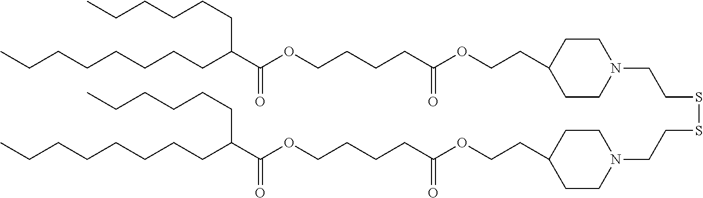 [SMILES: CCCCCCCCC(CCCCCC)C(=O)OCCCCC(=O)OCCC1CCN(CCSSCCN2CCC(CCOC(=O)CCCCOC(=O)C(CCCCCC)CCCCCCCC)CC2)CC1] | HEXACA- C5DE- PipSS |
| C15 | 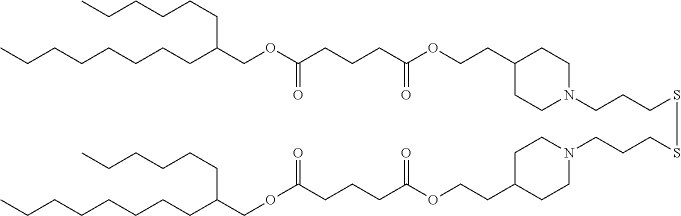 [SMILES: CCCCCCCCC(CCCCCC)COC(=O)CCCC(=O)OCCC1CCN(CCCSSCCCN2CCC(CCOC(=O)CCCC(=O)OCC(CCCCCC)CCCCCCCC)CC2)CC1] | HEXA- C5DE- PipC3SS (see also FIG. 1B) |
| C16 | 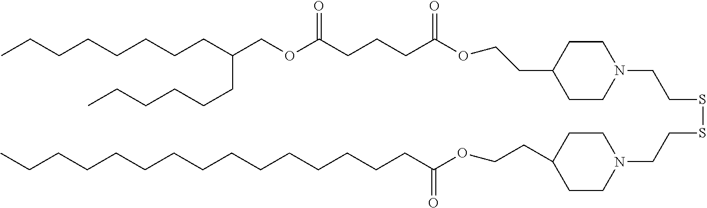 [SMILES: CCCCCCCCCCCCCCCC(=O)OCCC1CCN(CCSSCCN2CCC(CCOC(=O)CCCC(=O)OCC(CCCCCC)CCCCCCCC)CC2)CC1] | C16-HEXA- C5DE-PipSS |
| C17 | 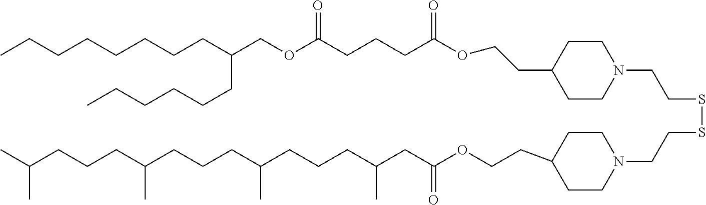 [SMILES: CCCCCCCCC(CCCCCC)COC(=O)CCCC(=O)OCCC1CCN(CCSSCCN2CCC(CCOC(=O)CC(C)CCCC(C)CCCC(C)CCCC(C)C)CC2)CC1] | “Phy-HEXA- C5DE- PipSS” or “DPhy-HEXA- C5DE-PipSS” |
| C18 | 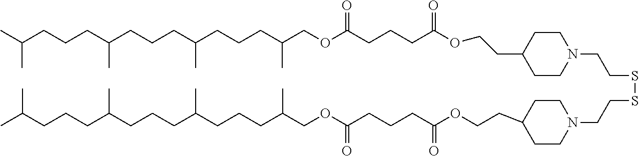 [SMILES: CC(C)CCCC(C)CCCC(C)CCCC(C)COC(=O)CCCC(=O)OCCC1CCN(CCSSCCN2CCC(CCOC(=O)CCCC(=O)OCC(C)CCCC(C)CCCC(C)CCCC(C)C)CC2)CC1] | “DPhy-C5DE- PipSS” or “2DPhy-C5DE- PipSS” |
| C19 | 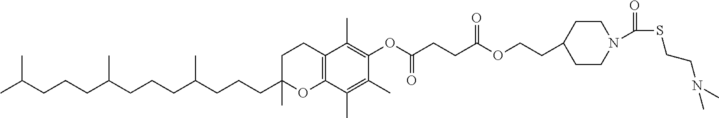 [SMILES: Cc1c(C)c2c(c(C)c1OC(=O)CCC(=O)OCCC1CCN(C(=O)SCCN(C)C)CC1)CCC(C)(CCCC(C)CCCC(C)CCCC(C)C)O2] | Vit E- C5DE-Pip- TEN |
| C20 | 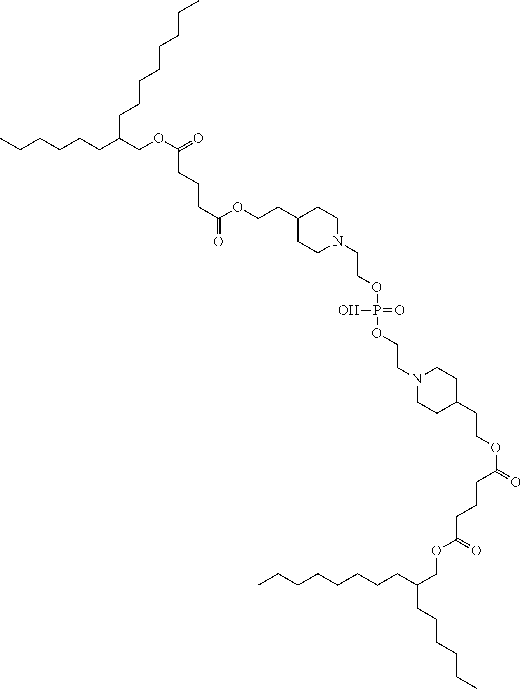 [SMILES: CCCCCCCCC(CCCCCC)COC(=O)CCCC(=O)OCCC1CCN(CCOP(=O)(O)OCCN2CCC(CCOC(=O)CCCC(=O)OCC(CCCCCC)CCCCCCCC)CC2)CC1] | HEXA- C5DE-Pip- Phosphate |
| C21 | 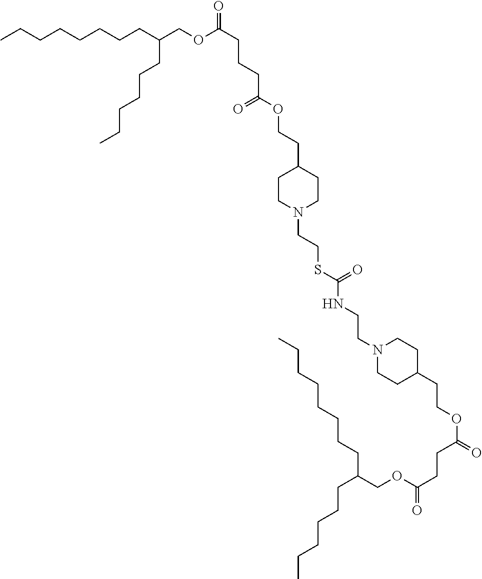 [SMILES: CCCCCCCCC(CCCCCC)COC(=O)CCCC(=O)OCCC1CCN(CCSC(=O)NCCN2CCC(CCOC(=O)CCC(=O)OCC(CCCCCC)CCCCCCCC)CC2)CC1] | HEXA- C5DE-Pip- Thiocarbamate |
| C22 | 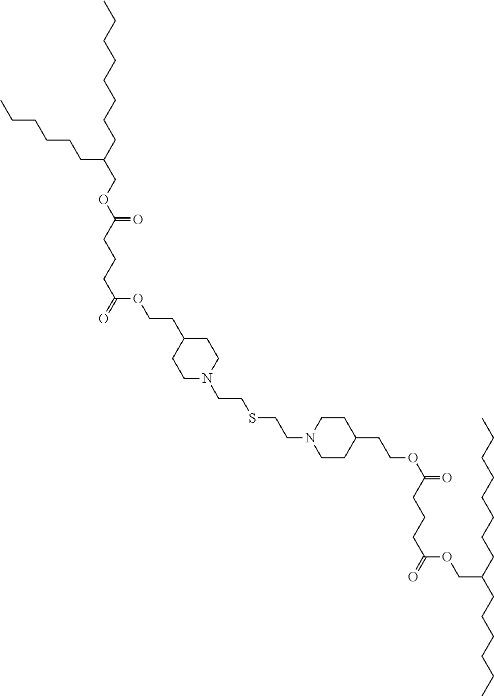 [SMILES: CCCCCCCCC(CCCCCC)COC(=O)CCCC(=O)OCCC1CCN(CCSCCN2CCC(CCOC(=O)CCCC(=O)OCC(CCCCCC)CCCCCCCC)CC2)CC1] | HEXA- C5DE-Pip- Thioether |
| C23 | 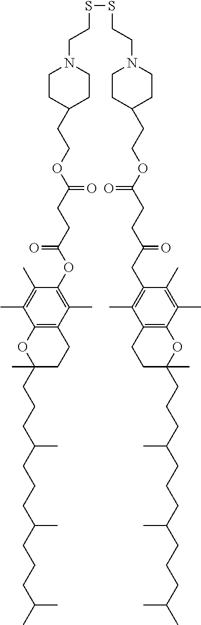 | COATSOME ® SS-EC |
| C24 | 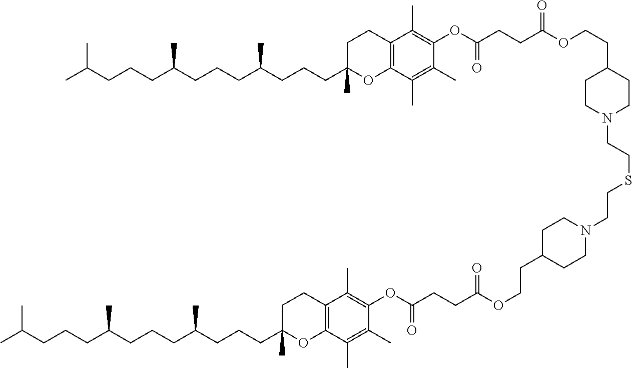 [SMILES: Cc1c(C)c2c(c(C)c1OC(=O)CCC(=O)OCCC1CCN(CCSCCN3CCC(CCOC(=O)CCC(=O)Oc4c(C)c(C)c5c(c4C)CC[C@@](C)(CCC[C@H](C)CCC[C@H](C)CCCC(C)C)O5)CC3)CC1)CC[C@@](C)(CCC[C@H](C)CCC[C@H](C)CCCC(C)C)O2] | THIOETHER or VitE-C4DE- Piperidine- Thioether (see also FIG. 1C) |
| C25 | 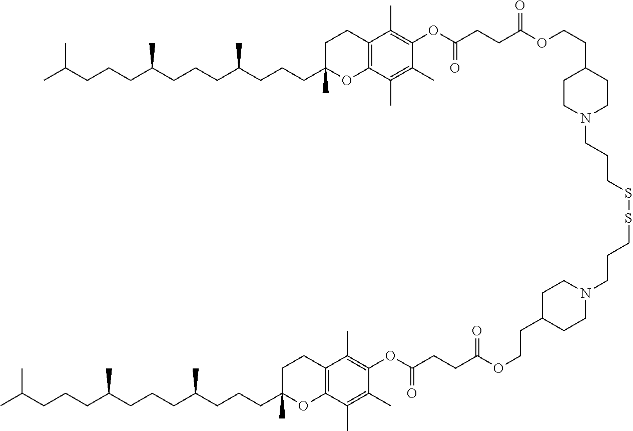 [SMILES: Cc1c(C)c2c(c(C)c1OC(=O)CCC(=O)OCCC1CCN(CCCSSCCCN3CCC(CCOC(=O)CCC(=O)Oc4c(C)c(C)c5c(c4C)CC[C@@](C)(CCC[C@H](C)CCC[C@H](C)CCCC(C)C)O5)CC3)CC1)CC[C@@](C)(CCC[C@H](C)CCC[C@H](C)CCCC(C)C)O2] | “C3SS” or “VitE- C4DE- Piperidine- C3SS” |
| C26 | 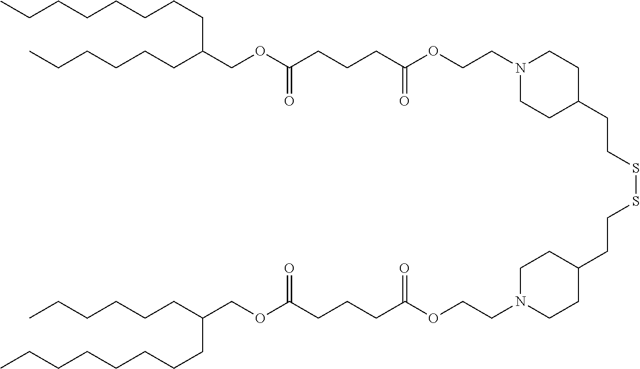 [SMILES: CCCCCCCCC(CCCCCC)COC(=O)CCCC(=O)OCCN1CCC(CCSSCCC2CCN(CCOC(=O)CCCC(=O)OCC(CCCCCC)CCCCCCCC)CC2)CC1] | HEXA-C5DE- inverted-PipSS |
| C27 | 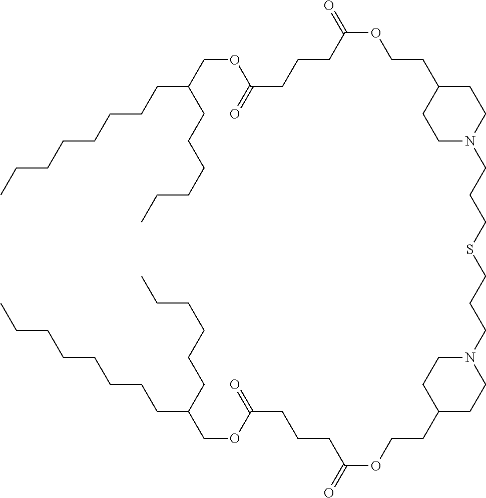 [SMILES: CCCCCCCCC(CCCCCC)COC(=O)CCCC(=O)OCCC1CCN(CCCSCCCN2CCC(CCOC(=O)CCCC(=O)OCC(CCCCCC)CCCCCCCC)CC2)CC1] | HEXA- C5DE-Pip- C3 thioether or HEXA-C5DE- piperidine-C3 thioether |

Accordingly, the invention encompasses a vaccine composition comprising the cationic lipid as described above. For example, the composition may comprise a cationic lipid selected from compounds C1 to C27 of Table 1.

In other preferred embodiments, the at least one nucleic acid (e.g. DNA or RNA), preferably the at least one mRNA of the composition is complexed with one or more lipids thereby forming LNPs, wherein the cationic lipid of the LNP has the structure “C24”, which in turn also is a very preferred structure of a cationic lipid

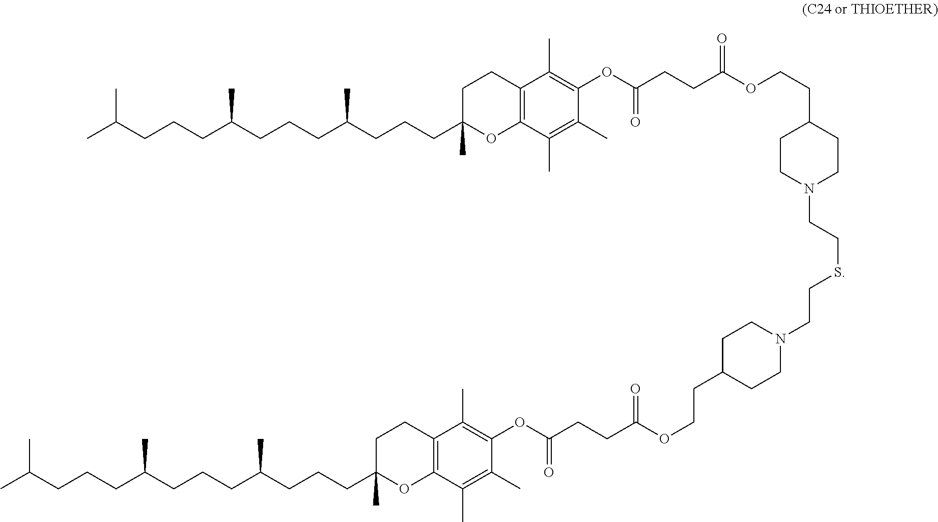 [SMILES: Cc1c(C)c2c(c(C)c1OC(=O)CCC(=O)OCCC1CCN(CCSCCN3CCC(CCOC(=O)CCC(=O)Oc4c(C)c(C)c5c(c4C)CC[C@@](C)(CCC[C@H](C)CCC[C@H](C)CCCC(C)C)O5)CC3)CC1)CC[C@@](C)(CCC[C@H](C)CCC[C@H](C)CCCC(C)C)O2]

Cationic, ionizable or cationisable lipids also include, but are not limited to, DSDMA, N,N-dioleyl-N,N-dimethylammonium chloride (DODAC), N,N-distearyl-N,N-dimethylammonium bromide (DDAB), 1,2-dioleoyltrimethyl ammonium propane chloride (DOTAP) (also known as N-(2,3-dioleoyloxy)propyl)-N,N,N-trimethylammonium chloride and 1,2-Dioleyloxy-3-trimethylaminopropane chloride salt), N-(1-(2,3-dioleyloxy)propyl)-N,N,N-trimethylammonium chloride (DOTMA), N,N-dimethyl-2,3-dioleyloxy)propylamine (DODMA), ckk-E12, ckk, 1,2-DiLinoleyloxy-N,N-dimethylaminopropane (DLinDMA), 1,2-Dilinolenyloxy-N,N-dimethylaminopropane (DLenDMA), 1,2-di-y-linolenyloxy-N,N-dimethylaminopropane (γ-DLenDMA), 98N12-5, 1,2-Dilinoleylcarbamoyloxy-3-dimethylaminopropane (DLin-C-DAP), 1,2-Dilinoleyoxy-3-(dimethylamino)acetoxypropane (DLin-DAC), 1,2-Dilinoleyoxy-3-morpholinopropane (DLin-MA), 1,2-Dilinoleoyl-3-dimethylaminopropane (DLinDAP), 1,2-Dilinoleylthio-3-dimethylaminopropane (DLin-S-DMA), 1-Linoleoyl-2-linoleyloxy-3-dimethylaminopropane (DLin-2-DMAP), 1,2-Dilinoleyloxy-3-trimethylaminopropane chloride salt (DLin-TMA·Cl), ICE (Imidazol-based), HGT5000, HGT5001, DMDMA, CLinDMA, CpLinDMA, DMOBA, DOcarbDAP, DLincarbDAP, DLinCDAP, KLin-K-DMA, DLin-K-XTC2-DMA, XTC (2,2-Dilinoleyl-4-dimethylaminoethyl-[1,3]-dioxolane) HGT4003, 1,2-Dilinoleoyl-3-trimethylaminopropane chloride salt (DLin-TAP·Cl), 1,2-Dilinoleyloxy-3-(N-methylpiperazino)propane (DLin-MPZ), or 3-(N,N-Dilinoleylamino)-1,2-propanediol (DLinAP), 3-(N,N-Dioleylamino)-1,2-propanedio (DOAP), 1,2-Dilinoleyloxo-3-(2-N,N-dimethylamino)ethoxypropane (DLin-EG-DM A), 2,2-Dilinoleyl-4-dimethylaminomethyl-[1,3]-dioxolane (DLin-K-DMA) or analogs thereof, (3aR,5s,6aS)—N,N-dimethyl-2,2-di((9Z,12Z)-octadeca-9,12-dienyl)tetrahydro-3aH-cyclopenta[d][1,3]dioxol-5-amine, (6Z,9Z,28Z,31Z)-heptatriaconta-6,9,28,31-tetraen-19-yl-4-(dimethylamino)butanoate (MC3), ALNY-100 ((3aR,5s,6aS)—N,N-dimethyl-2,2-di((9Z,12Z)-octadeca-9,12-dienyl)tetrahydro-3aH-cyclopenta[d] [1,3]dioxol-5-amine)), 1,1′-(2-(4-(2-((2-(bis(2-hydroxydodecyl)amino)ethyl)(2-hydroxydodecyl)amino)ethyl)piperazin-1-yl)ethylazanediyl)didodecan-2-ol (C12-200), 2,2-dilinoleyl-4-(2-dimethylaminoethyl)-[1,3]-dioxolane (DLin-K-C2-DMA), 2,2-dilinoleyl-4-dimethylaminomethyl-[1,3]-dioxolane (DLin-K-DMA), NC98-5 (4,7, 13-tris(3-oxo-3-(undecylamino)propyl)-N1,N16-diundecyl-4,7,10,13-tetraazahexadecane-1,16-diamide), (6Z,9Z,28Z,31Z)-heptatriaconta-6,9,28,31-tetraen-19-yl 4-(dimethylamino) butanoate (DLin-M-C3-DMA), 3-((6Z,9Z,28Z,31Z)-heptatriaconta-6,9,28,31-tetraen-19-yloxy)-N,N-dimethylpropan-1-amine (MC3 Ether), 4-((6Z,9Z,28Z,31Z)-heptatriaconta-6,9,28,31-tetraen-19-yloxy)-N,N-dimethylbutan-1-amine (MC4 Ether), LIPOFECTIN® (commercially available cationic liposomes comprising DOTMA and 1,2-dioleoyl-sn-3phosphoethanolamine (DOPE), from GIBCO/BRL, Grand Island, N.Y.); LIPOFECTAMINE® (commercially available cationic liposomes comprising N-(1-(2,3dioleyloxy)propyl)-N-(2-(sperminecarboxamido)ethyl)-N,N-dimethylammonium trifluoroacetate (DOSPA) and (DOPE), from GIBCO/BRL); and TRANSFECTAM® (commercially available cationic lipids comprising dioctadecylamidoglycyl carboxyspermine (DOGS) in ethanol from Promega Corp., Madison, Wis.) or any combination of any of the foregoing. Further suitable cationic lipids for use in the compositions and methods of the invention include those described in international patent publications WO2010053572 (and particularly, CI 2-200 described at paragraph [00225]) and WO2012170930, both of which are incorporated herein by reference, HGT4003, HGT5000, HGTS001, HGT5001, HGT5002 (see US20150140070).

In embodiments, the cationic lipid may be an amino lipid.

Representative amino lipids include, but are not limited to, 1,2-dilinoleyoxy-3-(dimethylamino)acetoxypropane (DLin-DAC), 1,2-dilinoleyoxy-3morpholinopropane (DLin-MA), 1,2-dilinoleoyl-3-dimethylaminopropane (DLinDAP), 1,2-dilinoleylthio-3-dimethylaminopropane (DLin-S-DMA), 1-linoleoyl-2-linoleyloxy-3dimethylaminopropane (DLin-2-DMAP), 1,2-dilinoleyloxy-3-trimethylaminopropane chloride salt (DLin-TMA·Cl), 1,2-dilinoleoyl-3-trimethylaminopropane chloride salt (DLin-TAP·Cl), 1,2-dilinoleyloxy-3-(N-methylpiperazino)propane (DLin-MPZ), 3-(N,Ndilinoleylamino)-1,2-propanediol (DLinAP), 3-(N,N-dioleylamino)-1,2-propanediol (DOAP), 1,2-dilinoleyloxo-3-(2-N,N-dimethylamino)ethoxypropane (DLin-EG-DMA), and 2,2-dilinoleyl-4-dimethylaminomethyl-[1,3]-dioxolane (DLin-K-DMA), 2,2-dilinoleyl-4-(2-dimethylaminoethyl)-[1,3]-dioxolane (DLin-KC2-DMA); dilinoleyl-methyl-4-dimethylaminobutyrate (DLin-MC3-DMA); MC3 (US20100324120).

In embodiments, the cationic lipid may an aminoalcohol lipidoid.

Aminoalcohol lipidoids which may be used in the present invention may be prepared by the methods described in U.S. Pat. No. 8,450,298, herein incorporated by reference in its entirety. Suitable (ionizable) lipids can also be the compounds as disclosed in Tables 1, 2 and 3 and as defined in claims 1-24 of WO2017075531, hereby incorporated by reference.

In another embodiment, suitable lipids can also be the compounds as disclosed in WO2015074085 (i.e. ATX-001 to ATX-032 or the compounds as specified in claims 1-26), U.S. Appl. Nos. 61/905,724 and Ser. No. 15/614,499 or U.S. Pat. Nos. 9,593,077 and 9,567,296 hereby incorporated by reference in their entirety.

In other embodiments, suitable cationic lipids can also be the compounds as disclosed in WO2017117530 (i.e. lipids 13, 14, 15, 16, 17, 18, 19, 20, or the compounds as specified in the claims), hereby incorporated by reference in its entirety.

In preferred embodiments, ionizable or cationic lipids may also be selected from the lipids disclosed in WO2018078053 (i.e. lipids derived from formula I, II, and III of WO2018078053, or lipids as specified in Claims 1 to 12 of WO2018078053), the disclosure of WO2018078053 hereby incorporated by reference in its entirety. In that context, lipids disclosed in Table 7 of WO2018078053 (e.g. lipids derived from formula I-1 to I-41) and lipids disclosed in Table 8 of WO2018078053 (e.g. lipids derived from formula II-1 to II-36) may be suitably used in the context of the invention. Accordingly, formula I-1 to formula I-41 and formula II-1 to formula II-36 of WO2018078053, and the specific disclosure relating thereto, are herewith incorporated by reference.

In preferred embodiments, cationic lipids may be derived from formula III of published PCT patent application WO2018078053. Accordingly, formula III of WO2018078053, and the specific disclosure relating thereto, are herewith incorporated by reference.

In particularly preferred embodiments, the at least one nucleic acid (e.g. DNA or RNA), preferably the at least one mRNA of the composition is complexed with one or more lipids thereby forming LNPs, wherein the cationic lipid of the LNP is selected from structures III-1 to III-36 of Table 9 of published PCT patent application WO2018078053. Accordingly, formula III-1 to III-36 of WO2018078053, and the specific disclosure relating thereto, are herewith incorporated by reference.

In particularly preferred embodiment of the second aspect, the at least one nucleic acid (e.g. DNA or RNA), preferably the at least one mRNA is complexed with one or more lipids thereby forming LNPs, wherein the LNPs comprise a cationic lipid according to

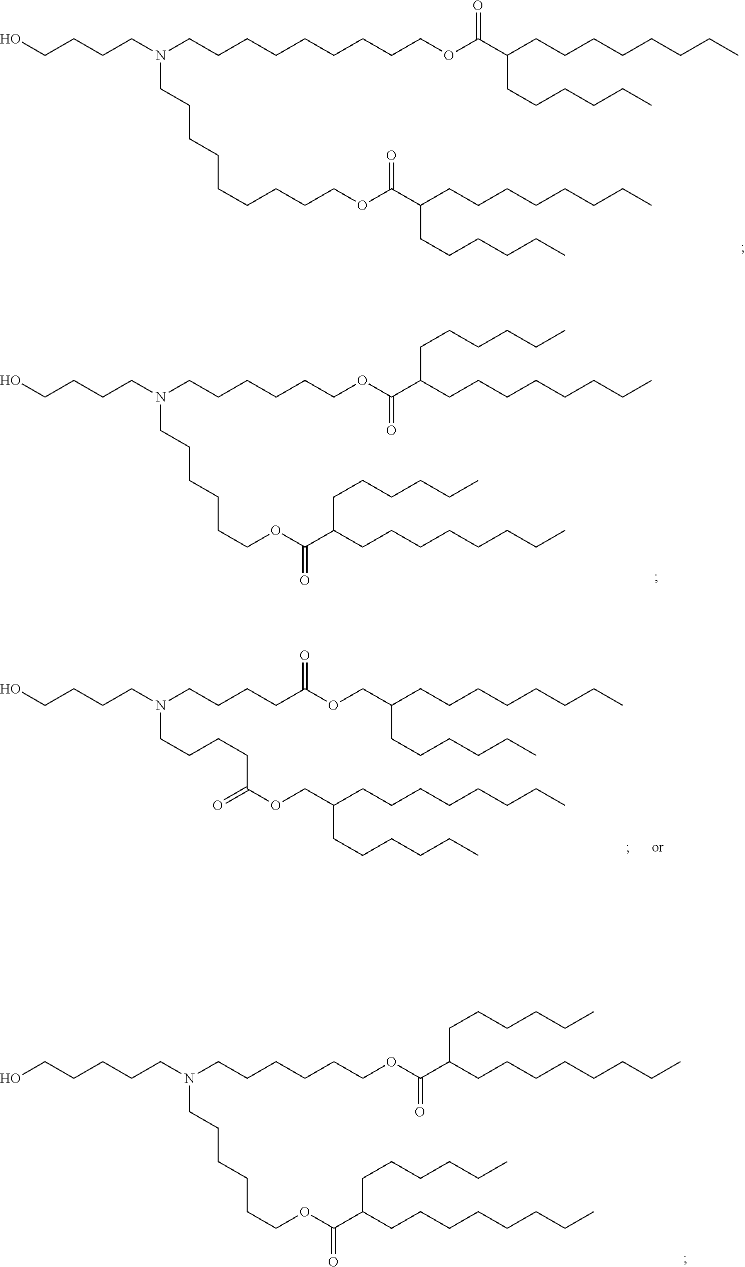 [SMILES: CCCCCCCCC(CCCCCC)C(=O)OCCCCCCCCCN(CCCCO)CCCCCCCCCOC(=O)C(CCCCCC)CCCCCCCC ; CCCCCCCCC(CCCCCC)C(=O)OCCCCCCN(CCCCO)CCCCCCOC(=O)C(CCCCCC)CCCCCCCC ; CCCCCCCCC(CCCCCC)COC(=O)CCCCN(CCCCO)CCCCC(=O)OCC(CCCCCC)CCCCCCCC ; CCCCCCCCC(CCCCCC)C(=O)OCCCCCCN(CCCCCO)CCCCCCOC(=O)C(CCCCCC)CCCCCCCC]

or most preferably formula III-3 of WO2018078053, i.e. (4-hydroxybutyl)azanediyl)bis(hexane-6,1-diyl)bis(2-hexyldecanoate):

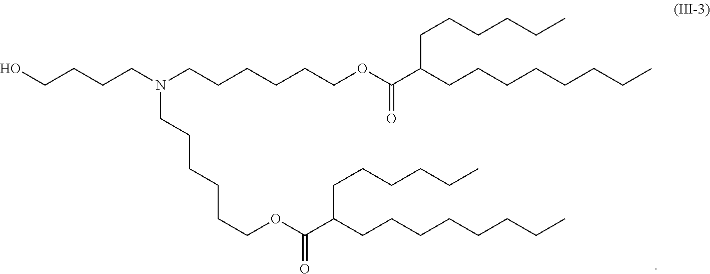 [SMILES: CCCCCCCCC(CCCCCC)C(=O)OCCCCCCN(CCCCO)CCCCCCOC(=O)C(CCCCCC)CCCCCCCC]

In certain embodiments, the cationic lipid as defined herein, more preferably cationic lipid compound III-3 ((4-hydroxybutyl) azanediyl)bis(hexane-6,1-diyl)bis(2-hexyldecanoate)), is present in the LNP in an amount from about 30 to about 80 mole percent, preferably about 30 to about 60 mole percent, more preferably about 40 to about 55 mole percent, more preferably about 47.4 mole percent, relative to the total lipid content of the LNP. If more than one cationic lipid is incorporated within the LNP, such percentages apply to the combined cationic lipids.

In embodiments, the cationic lipid is present in the LNP in an amount from about 30 to about 70 mole percent. In one embodiment, the cationic lipid is present in the LNP in an amount from about 40 to about 60 mole percent, such as about 40, 41, 42, 43, 44, 45, 46, 47, 48, 49, 50, 51, 52, 53, 54, 55, 56, 57, 58, 59 or 60 mole percent, respectively. In embodiments, the cationic lipid is present in the LNP in an amount from about 47 to about 48 mole percent, such as about 47.0, 47.1, 47.2, 47.3, 47.4, 47.5, 47.6, 47.7, 47.8, 47.9, 50.0 mole percent, respectively, wherein 47.4 mole percent are particularly preferred.

In some embodiments, the cationic lipid is present in a ratio of from about 20 mol % to about 70 or 75 mol % or from about 45 to about 65 mol % or about 20, 25, 30, 35, 40, 45, 50, 55, 60, 65, or about 70 mol % of the total lipid present in the LNP. In further embodiments, the LNPs comprise from about 25 mol % to about 75 mol % i.e. on a molar basis of cationic lipid, e.g., from about 20 to about 70 mol %, from about 35 to about 65 mol %, from about 45 to about 65 mol %, about 60 mol %, about 57.5 mol %, about 57.1 mol %, about 50 mol % or about 40 mol % i.e. on a molar basis (based upon 100% total moles of lipid in the lipid nanoparticle). In some embodiments, the ratio of cationic lipid to nucleic acid (e.g. coding RNA or DNA) is from about 3 to about 15, such as from about 5 to about 13 or from about 7 to about 11.

Other suitable (cationic or ionizable) lipids are disclosed in WO2009086558, WO2009127060, WO2010048536, WO2010054406, WO2010088537, WO2010129709, WO2011153493, WO 2013063468, US20110256175, US20120128760, US20120027803, U.S. Pat. No. 8,158,601, WO2016118724, WO2016118725, WO2017070613, WO2017070620, WO2017099823, WO2012040184, WO2011153120, WO2011149733, WO2011090965, WO2011043913, WO2011022460, WO2012061259, WO2012054365, WO2012044638, WO2010080724, WO201021865, WO2008103276, WO2013086373, WO2013086354, U.S. Pat. Nos. 7,893,302, 7,404,969, 8,283,333, 8,466,122 and 8,569,256 and US Patent Publication No. US20100036115, US20120202871, US20130064894, US20130129785, US20130150625, US20130178541, US20130225836, US20140039032 and WO2017112865. In that context, the disclosures of WO2009086558, WO2009127060, WO2010048536, WO2010054406, WO2010088537, WO2010129709, WO2011153493, WO 2013063468, US20110256175, US20120128760, US20120027803, U.S. Pat. No. 8,158,601, WO2016118724, WO2016118725, WO2017070613, WO2017070620, WO2017099823, WO2012040184, WO2011153120, WO2011149733, WO2011090965, WO2011043913, WO2011022460, WO2012061259, WO2012054365, WO2012044638, WO2010080724, WO201021865, WO2008103276, WO2013086373, WO2013086354, U.S. Pat. Nos. 7,893,302, 7,404,969, 8,283,333, 8,466,122 and 8,569,256 and US Patent Publication No. US20100036115, US20120202871, US20130064894, US20130129785, US20130150625, US20130178541, US20130225836 and US20140039032 and WO2017112865 specifically relating to (cationic) lipids suitable for LNPs are incorporated herewith by reference.

In other embodiments, the cationic or ionizable lipid is

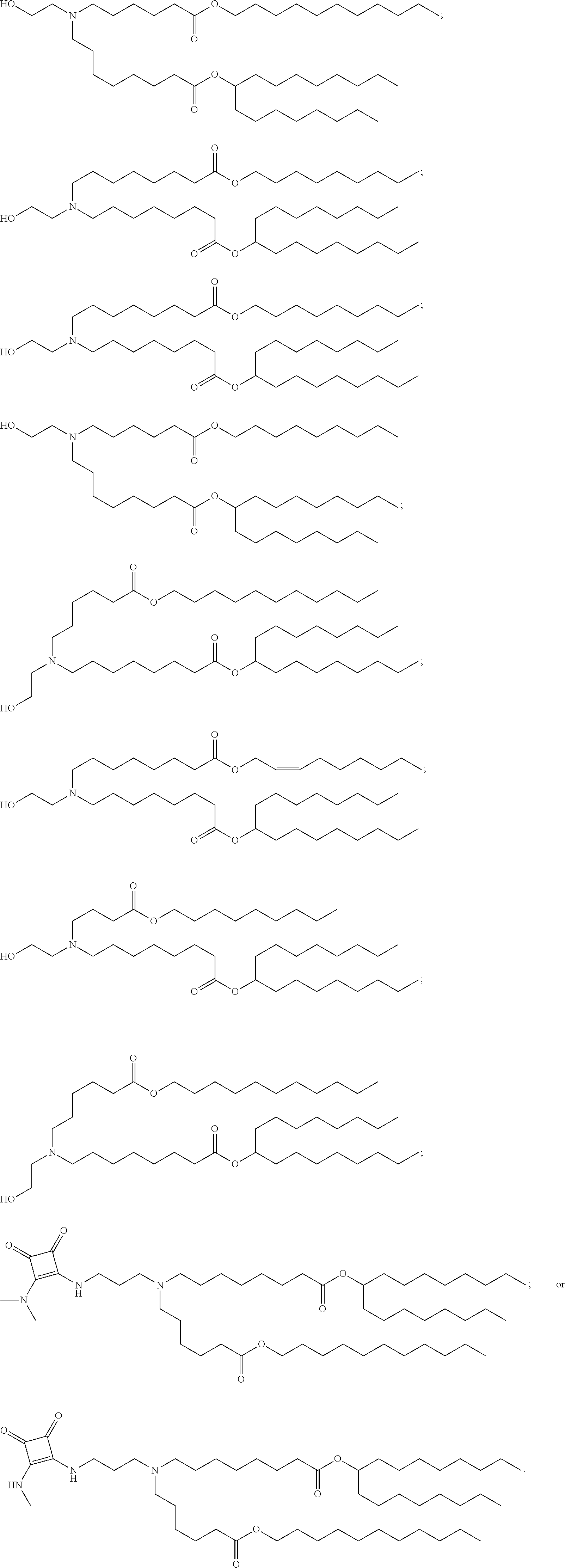

In embodiments, amino or cationic lipids as defined herein have at least one protonatable or deprotonatable group, such that the lipid is positively charged at a pH at or below physiological pH (e.g. pH 7.4), and neutral at a second pH, preferably at or above physiological pH. It will, of course, be understood that the addition or removal of protons as a function of pH is an equilibrium process, and that the reference to a charged or a neutral lipid refers to the nature of the predominant species and does not require that all of lipids have to be present in the charged or neutral form. Lipids having more than one protonatable or deprotonatable group, or which are zwitterionic, are not excluded and may likewise suitable in the context of the present invention. In some embodiments, the protonatable lipids have a pKa of the protonatable group in the range of about 4 to about 11, e.g., a pKa of about 5 to about 7.

LNPs can comprise two or more (different) cationic lipids as defined herein. Cationic lipids may be selected to contribute to different advantageous properties. For example, cationic lipids that differ in properties such as amine pKa, chemical stability, half-life in circulation, half-life in tissue, net accumulation in tissue, or toxicity can be used in the LNP. In particular, the cationic lipids can be chosen so that the properties of the mixed-LNP are more desirable than the properties of a single-LNP of individual lipids.

The amount of the permanently cationic lipid, lipidoid or preferably ionizable cationic lipid may be selected taking the amount of the nucleic acid cargo into account. In one embodiment, these amounts are selected such as to result in an N/P ratio of the nanoparticle(s) or of the composition in the range from about 0.1 to about 20, or
(i) at an amount such as to achieve an N/P ratio in the range of about 1 to about 20, preferably about 2 to about 15, more preferably about 3 to about 10, even more preferably about 4 to about 9, most preferably about 6;
(ii) at an amount such as to achieve an N/P ratio in the range of about 5 to about 20, more preferably about 10 to about 18, even more preferably about 12 to about 16, most preferably about 14;
(iii) at an amount such as to achieve a lipid:mRNA weight ratio in the range of 20 to 60, preferably from about 3 to about 15, 5 to about 13, about 4 to about 8 or from about 7 to about 11; or
(iv) at an amount such as to achieve an N/P ratio in the range of about 6 for a lipid nanoparticle according to the invention, especially a lipid nanoparticle comprising the cationic lipid III-3; or
(v) at an amount such as to achieve an N/P ratio preferably in the range of about 17 or about 17.5 for a lipid nanoparticle according to the invention.

In this context, the N/P ratio is defined as the mole ratio of the nitrogen atoms (“N”) of the basic nitrogen-containing groups of the lipid or lipidoid to the phosphate groups (“P”) of the nucleic acid which is used as cargo. The N/P ratio may be calculated on the basis that, for example, 1 μg RNA typically contains about 3 nmol phosphate residues, provided that the RNA exhibits a statistical distribution of bases. The “N”-value of the cationic lipid or lipidoid may be calculated on the basis of its molecular weight and the relative content of permanently cationic and—if present—cationisable groups. If more than one cationic lipid is present, the N-value should be calculated on the basis of all cationic lipids comprised in the lipid nanoparticles.

In other aspects, the ionizable lipids of the present disclosure may be one or more of compounds of Formula (Cat-II):

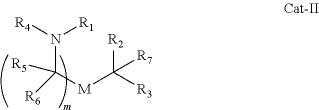

or their N-oxides, or salts or isomers thereof, wherein:
R₁ is selected from the group consisting of C5-30 alkyl, C5-20 alkenyl, —R*YR″, —YR″, and —R″M′R′;
R₂ and R₃ are independently selected from the group consisting of H, C₁₋₁₄ alkyl, C₂₋₁₄ alkenyl, —R*YR″, —YR″, and —R*OR″, or R₂ and R₃, together with the atom to which they are attached, form a heterocycle or carbocycle;
R₄ is selected from the group consisting of hydrogen, a C₃₋₆ carbocycle, —(CH₂)ₙQ, —(CH₂)ₙCHQR, —CHQR, —CQ(R)₂, and unsubstituted C₁₋₆ alkyl, where Q is selected from a carbocycle, heterocycle, —OR, —O(CH₂)ₙN(R)₂, —C(O)OR, —OC(O)R, —CX₃, —CX₂H, —CXH₂, —CN, —N(R)₂, —C(O)N(R)₂, —N(R)C(O)R, —N(R)S(O)₂R, —N(R)C(O)N(R)₂, —N(R)C(S)N(R)₂, —N(R)R₈, —N(R)S(O)₂RS, —O(CH₂)ₙOR, —N(R)C(═NR₉)N(R)₂, —N(R)C(═CHR₉)N(R)₂, —OC(O)N(R)₂, —N(R)C(O)OR, —N(OR)C(O)R, —N(OR)S(O)₂R, —N(OR)C(O)OR, —N(OR)C(O)N(R)₂, —N(OR)C(S)N(R)₂, —N(OR)C(═NR₉)N(R)₂, —N(OR)C(═CHR₉)N(R)₂, —C(═NR₉)N(R)₂, —C(═NR₉)R, —C(O)N(R)OR, and —C(R)N(R)₂C(O)OR, and each n is independently selected from 1, 2, 3, 4, and 5;
each R₅ is independently selected from the group consisting of C₁₋₃ alkyl, C₂₋₃ alkenyl, and H;
each R₆ is independently selected from the group consisting of C₁₋₃ alkyl, C₂₋₃ alkenyl, and H;
M and M′ are independently selected from —C(O)O—, —OC(O)—, —OC(O)-M″-C(O)O—, —C(O)N(R′)—, —N(R′)C(O)—, —C(O)—, —C(S)—, —C(S)S—, —SC(S)—, —CH(OH)—, —P(O)(OR′)O—, —S(O)₂—, —S—S—, an aryl group, and a heteroaryl group, in which M′ is a bond, C₁₋₁₃ alkyl or C₂₋₁₃ alkenyl;
R₇ is selected from the group consisting of C₁₋₃ alkyl, C₂₋₃ alkenyl, and H;
R₈ is selected from the group consisting of C₃₋₆ carbocycle and heterocycle;
R₉ is selected from the group consisting of H, CN, NO₂, C₁₋₆ alkyl, —OR, —S(O)₂R, —S(O)₂N(R)₂, C₂₋₆ alkenyl, C₃₋₆ carbocycle and heterocycle;
each R is independently selected from the group consisting of C₁₋₃ alkyl, C₂₋₃ alkenyl, and H;
each R′ is independently selected from the group consisting of C₁₋₁₈ alkyl, C₂₋₁₈ alkenyl, —R*YR″, —YR″, and H;
each R″ is independently selected from the group consisting of C₃₋₁₅ alkyl and C₃₋₁₅ alkenyl;
each R* is independently selected from the group consisting of C₁₋₁₂ alkyl and C₂₋₁₂ alkenyl;
each Y is independently a C₃₋₆ carbocycle;
each X is independently selected from the group consisting of F, Cl, Br, and I; and m is selected from 5, 6, 7, 8, 9, 10, 11, 12, and 13; and wherein when R₄ is —(CH₂)ₙQ, —(CH₂)ₙCHQR, —CHQR, or —CQ(R)₂, then (i) Q is not —N(R)₂ when n is 1, 2, 3, 4 or 5, or (ii) Q is not 5, 6, or 7-membered heterocycloalkyl when n is 1 or 2.

As used herein, the term “ionizable lipid” has its ordinary meaning in the art and may refer to a lipid comprising one or more charged moieties. In some embodiments, an ionizable lipid may be positively charged or negatively charged. An ionizable lipid may be positively charged, in which case it can be referred to as “cationic lipid”. In certain embodiments, an ionizable lipid molecule may comprise an amine group, and can be referred to as an ionizable amino lipid. As used herein, a “charged moiety” is a chemical moiety that carries a formal electronic charge, e.g., monovalent (+1, or −1), divalent (+2, or −2), trivalent (+3, or −3), etc. The charged moiety may be anionic (i.e., negatively charged) or cationic (i.e., positively charged). Examples of positively-charged moieties include amine groups (e.g., primary, secondary, and/or tertiary amines), ammonium groups, pyridinium group, guanidine groups, and imidazolium groups. In a particular embodiment, the charged moieties comprise amine groups. Examples of negatively-charged groups or precursors thereof, include carboxylate groups, sulfonate groups, sulfate groups, phosphonate groups, phosphate groups, hydroxyl groups, and the like. The charge of the charged moiety may vary, in some cases, with the environmental conditions, for example, changes in pH may alter the charge of the moiety, and/or cause the moiety to become charged or uncharged. In general, the charge density of the molecule may be selected as desired. It should be understood that the terms “charged” or “charged moiety” does not refer to a “partial negative charge” or “partial positive charge” on a molecule. The terms “partial negative charge” and “partial positive charge” are given its ordinary meaning in the art. A “partial negative charge” may result when a functional group comprises a bond that becomes polarized such that electron density is pulled toward one atom of the bond, creating a partial negative charge on the atom. Those of ordinary skill in the art will, in general, recognize bonds that can become polarized in this way. In some embodiments, the ionizable lipid is an ionizable amino lipid, sometimes referred to in the art as an “ionizable cationic lipid”. In one embodiment, the ionizable amino lipid may have a positively charged hydrophilic head and a hydrophobic tail that are connected via a linker structure.

A further particularly preferred embodiment for a lipid nanoparticle of the present invention is given when the following combination of excipients is used for formulating a lipid nanoparticle, i.e. 59 mol % C2 or C15 or C24 lipid as disclosed in Table 1 as cationic lipid (i.e. HEXA-C5DE-PipSS, cationic lipid compound C2 in Table 1, HEXA-C5DE-PipC3SS, cationic lipid compound C15 in Table 1, or respectively VitE-C4DE-Piperidine-Thioether, cationic lipid compound C24 in Table 1), 29.3 mol % cholesterol as steroid, a total of 10 mol % for the phosphatidylserine in combination with DPhyPE as further neutral lipid/phospholipid, and 1.7 mol % DMG-PEG2000 as polymer conjugated lipid.

### The Steroid

A “steroid” is an organic compound with four rings arranged in a specific molecular configuration. It comprises the following carbon skeleton:

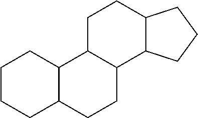 [SMILES: C1CCC2C(C1)CCC1C3CCCC3CCC21]

Steroids and neutral steroids include both naturally occurring steroids and analogues thereof (e.g. being amphipathic lipid cholesteryl hemisuccinate (CHEMS) which consists of succinic acid esterified to the beta-hydroxyl group of cholesterol as cholesterol derivate). Using the definition for “neutral” as provided herein, the neutral steroid may be a steroid either having no atoms or groups that are ionizable under physiological conditions, or it may be a zwitterionic steroid. In one of the preferred embodiments, the neutral steroid is free of atoms or groups that are ionizable under physiological conditions. In some preferred embodiments, the steroid or steroid analogue is cholesterol. The term “steroid” and “neutral steroid” is used herein interchangeably. In other embodiments, the sterol may be selected from the group consisting of a phytosterol, e.g. β-sitosterol, campesterol, stigmasterol, fucosterol, stigmastanol, dihydrocholesterol, ent-cholesterol, epi-cholesterol, desmosterol, cholestanol, cholestanone, cholestenone, cholesteryl-2′-hydroxyethyl ether, cholesteryl-4′-hydroxybutyl ether, 3β-[N—(N′N′-dimethylaminoethyl)carbamoyl cholesterol (DC-Chol), 24(S)-hydroxycholesterol, 25-hydroxy cholesterol, 25(R)-27-hydroxycholesterol, 22-oxacholesterol, 23-oxacholesterol, 24-oxacholesterol, cycloartenol, 22-ketosterol, 20-hydroxysterol, 7-hydroxycholesterol, 19-hydroxycholesterol, 22-hydroxycholesterol, 25-hydroxy cholesterol, 7-dehydrocholesterol, 5a-cholest-7-en-3β-ol, 3,6,9-trioxaoctan-1-ol-cholesteryl-3e-ol, dehydroergosterol, dehydroepiandrosterone, lanosterol, dihydrolanosterol, lanostenol, lumisterol, sitocalciferol, calcipotriol, coprostanol, cholecalciferol, lupeol, ergocalciferol, 22-dihydroegocalciferol, ergosterol, brassicasterol, tomatidine, tomatine, ursolic acid, cholic acid, chenodeoxycholic acid, zymosterol, diosgenin, fucosterol, fecosterol, or fecosterol, or a salt or ester thereof, cholesterol, cholesterol succinic acid, cholesterol sulfate, cholesterol hemisuccinate, cholesterol phthalate, cholesterol phosphate, cholesterol valerate, cholesterol acetate, cholesteryl oleate, cholesteryl linoleate, cholesteryl myristate, cholesteryl palmitate, cholesteryl arachidate, cholesteryl phosphorylcholine, and sodium cholate.

In a further embodiment, the steroid is an imidazole cholesterol ester or “ICE” as disclosed in paragraphs [0320] and [0339]-[0340] of WO2019226925; which is herein incorporated by reference in its entirety.

### The Further Phospholipid

The further phospholipid, which may also be referred to as “neutral lipid” or “helper lipid”, is an amphiphilic compound consisting of molecules that typically have two hydrophobic fatty acid “tails” and a hydrophilic “head” comprising a phosphate group. The phosphate group can be modified with simple organic molecules such as choline, ethanolamine or serine. Phospholipids occur abundantly in nature. For example, they represent a significant fraction of the excipients of biological membranes. As used herein, the expression “phospholipid” or “neutral phospholipid” covers both natural and synthetic phospholipids.

The terms “neutral lipid”, “neutral phospholipid” or “zwitterionic compound”, as used herein interchangeably, refer to any one of a number of lipid species that exist in either an uncharged or neutral zwitterionic form at physiological pH. Representative neutral lipids include diacylphosphatidylcholines, diacylphosphatidylethanolamines, ceramides, sphingomyelins, dihydro sphingomyelins, cephalins, and cerebrosides as further described herein below.

According to one of the preferred embodiments, the composition comprises a neutral lipid that is zwitterionic, such as a phosphatidylcholine or a phosphatidylethanolamine. Examples of suitable phosphatidylcholines include native or purified mixtures, sometimes referred to as “lecithin” or “phosphatidylcholine”, often derived from egg yolk or soy beans; or highly purified or semisynthetic compounds such as phosphatidylcholines having two fatty acyl moieties selected from myristoyl, palmitoyl, stearoyl, oleoyl and the like.

In another preferred embodiment, the neutral lipid or neutral phospholipid is a zwitterionic compound selected from, but not limited to the group of 1,2-diphytanoyl-sn-glycero-3-phosphoethanolamine (DPhyPE; also referred to as 1,2-di-(3,7,11,15-tetramethylhexadecanoyl)-sn-glycero-3-phosphoethanolamine), 1,2-diphytanoyl-sn-glycero-3-phosphocholine (DPhyPC), 1,2-dioleoyl-sn-glycero-3-phosphocholine (DOPC; also referred to as dioleoylphosphatidylcholine), 1,2-Dipalmitoyl-sn-glycero-3-phosphocholine (DPPC, also referred to as dipalmitoylphosphatidylcholine), 1,2-dioleoyl-sn-glycero-3-phosphoethanolamine (DOPE), phosphatidylethanolamines, distearoylphosphatidylcholines, dioleoyl-phosphatidylethanolamine (DOPEA), 1,2-distearoyl-sn-glycero-3-phosphoethanolamine (DSPE), palmitoyloleoylphosphatidylcholine (POPC), palmitoyl-oleoyl-phosphatidylethanolamine (POPE), 1,2-Dipalmitoyl-sn-glycero-3-phosphoethanolamine (DPPE), dioleoyl-phosphatidylethanolamine 4-(N-maleimidomethyl)-cyclohexane-1-carboxylate (DOPE-mal), 1,2-Dimyristoyl-sn-glycero-3-phosphoethanolamine (DMPE), 1,2-Dilinoleoyl-sn-glycero-3-phosphoethanolamine (DLoPE), distearoyl-phosphatidylethanolamine (DSPE), 1-Palmitoyl-2-oleoyl-sn-glycero-3-phosphoethanolamine (POPE), 1,2-Di-lauroyl-sn-glycero-3-phosphoethanolamine (DLPE), 16-O-monomethylphosphoethanolamine, 16-O-dimethyl phosphatidylethanolamine, 1,2-Dierucoyl-sn-glycero-3-phosphoethanolamine (DEPE), 18-1-trans phosphatidylethanolamine, 1-stearoyl-2-oleoylphosphatidyethanolamine (SOPE), 1,2-Disqualeoyl-sn-glycero-3-phosphoethanolamine (DSQPE), 1,2-dielaidoyl-sn-glycero-3-phosphoethanolamine (transDOPE), 1-Stearoyl-2-linoleoyl-sn-glycero-3-phosphoethanolamine (SLPE), 1-tridecanoyl-sn-glycero-3-phospho-L-serine (sodium salt), 1-oleoyl-2-hydroxy-sn-glycero-3-phospho-L-serine (sodium salt), 1-palmitoyl-2-oleoyl-sn-glycero-3-phospho-L-serine (sodium salt) (POPS), 1-1-stearoyl-2-oleoyl-sn-glycero-3-phospho-L-serine (sodium salt), 1,2-dioleoyl-sn-glycero-3-phospho-L-serine (sodium salt) (DOPS), 1,2-distearoyl-sn-glycero-3-phospho-L-serine (sodium salt), 1,2-diphytanoyl-sn-glycero-3-phospho-L-serine (sodium salt), 1-O-hexadecanyl-2-O-(9Z-octadecenyl)-sn-glycero-3-phosphoethanolamine, 1,2-distearoyl-sn-glycero-3-phosphatidylcholine or 1,2-distearoyl-sn-glycero-3-phosphocholine (DSPC), 1,2-di-O-phytanyl-sn-glycero-3-phosphoethanolamine, 1-palmitoyl-2-cholesteryl-hemisuccinoyl-sn-glycero-3-phosphocholine (PChemsPC), 1,2-dicholesterylhemisuccinoyl-sn-glycero-3-phosphocholine (DChemsPC), 2-((2,3-bis(oleoyloxy)propyl)dimethylammonio)ethyl hydrogen phosphate (DOCP), 2-((2,3-bis(oleoyloxy)propyl)dimtheylammonio)ethyl ethyl phosphate (DOCPe), and 1-O-octadecyl-2-O-methyl-sn-glycero-3-phosphocholine (Edelfosine).

In a preferred embodiment, the neutral lipid according to the invention is 1,2-Dipalmitoyl-sn-glycero-3-phosphocholine (DPPC), 1,2-dioleoyl-sn-glycero-3-phosphocholine (DOPC) or 1,2-dioleoyl-sn-glycero-3-phosphoethanolamine (DOPE). In a more preferred embodiment, the neutral lipid according to the invention is 1,2-diphytanoyl-sn-glycero-3-phosphocholine (DPhyPC). In an even more preferred particularly preferred embodiment, the neutral lipid according to the invention is 1,2-diphytanoyl-sn-glycero-3-phosphoethanolamine (DPhyPE). The inventive advantage connected with the use of DPhyPE is the high capacity for fusogenicity due to its bulky tails, whereby it is able to fuse at a high level with endosomal lipids. In other words, another inventive advantage connected with the use of DPhyPE is the high capacity for fusogenicity due to its bulky tails, whereby it is able to fuse at a high level with endosomal lipids. Therefore, in another embodiment, the invention is related to the use of a lipid with high fusogenicity in a lipid-based carrier or nucleic acid-lipid particle, preferably DPhyPE, as depicted here:

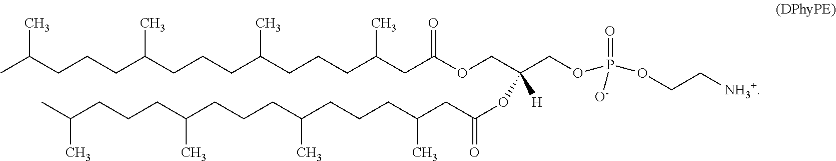

The term “fusogenic” or “fusogenicity” is meant to refer to a lipid which aids the fusion of a lipid-based carrier or nucleic acid-lipid particle with a cell membrane to help the nucleic acid contained in the lipid-based carrier or nucleic acid-lipid particle to enter the cell.

DSPC, DOPC or DOPE, which are routinely used in the art as phospholipid in LNPs, each have two C₁₈ chains side arms as apparent from the structures shown herein below:

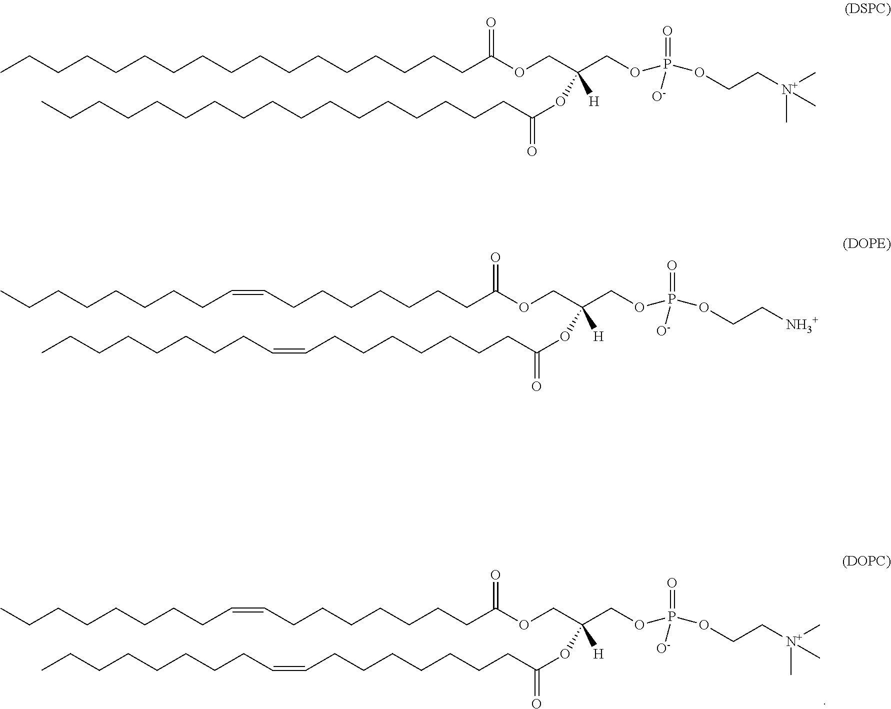

Surprisingly, in a further aspect of the invention, the inventors found that the addition of phospholipids with shorter alkyl chains than e.g. state of the art DSPC or DOPE, were highly beneficial for the efficacy of lipid nanoparticles of the invention, comprising polymer conjugated lipids according to formula (I). Specifically the advantageous use of (07:0) PC (DHPC; 1,2-diheptanoyl-sn-glycero-3-phosphocholine) with shorter alkyl chains than e.g. state of the art DSPC as disclosed herein, preferably in combination with the inventive polymer conjugated lipids as disclosed herein, for delivering mRNA vaccines in vivo, resulting in significantly enhanced immune responses, is a further very surprising finding made by the inventors and resembles specific aspects and embodiments of the present invention.

The structure of (07:0) PC (DHPC; 1,2-diheptanoyl-sn-glycero-3-phosphocholine) from is shown herein below:

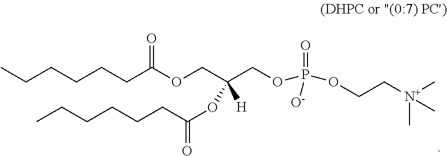 [SMILES: CCCCCCC(=O)OC[C@H](COP(=O)([O-])OCC[N+](C)(C)C)OC(=O)CCCCCC]

The inventors further surprisingly found that the addition of at least one further neutral lipid to the above neutral lipid, in particular a third neutral lipid, can also enhance the immune responses (see the corresponding examples). As noted above, it is preferred for the further (second) neutral lipid of the invention that it has two fatty acyl moieties selected from myristoyl, palmitoyl, stearoyl, oleoyl and the like, which in particular means that the fatty acyl moieties are rather long moieties starting from moieties with 14 carbon atoms. The inventors found that the addition of a neutral lipid with shorter fatty acyl moieties provides for beneficial effects, in particular if the additional neutral lipid has two fatty acid moieties selected from pentanoyl, hexanoyl, heptanoyl, octanoyl, nonaoyl and decanoyl, i.e. moieties with at most 10 carbon atoms. A particularly preferred additional neutral lipid is 1,2-diheptanoyl-sn-glycero-3-phosphocholine (DHPC), but related neutral lipids, such as e.g. 05:0 PC (1,2-dipentanoyl-sn-glycero-3-phosphocholine), 06:0 PC (1,2-dihexanoyl-sn-glycero-3-phosphocholine), 08:0 PC (1,2-dioctanoyl-sn-glycero-3-phosphocholine), 09:0 PC (1,2-dinonanoyl-sn-glycero-3-phosphocholine), and 10:0 PC (1,2-dihexanoyl-sn-glycero-3-phosphocholine) may be used as well.

Therefore, in one aspect of the invention, the lipid nanoparticles of the invention comprise a neutral lipid or phospholipid having at least one alkyl chain with a length of C₅, C₆, C₇, C₈, C₉, C₁₀, C₁₁, C₁₂, C₁₃ or C₁₄, preferably with a length of C₆, C₇, C₈, C₉, or C₁₀, more preferably with a length of C₆, C₇, C₈, most preferably with a length of C₇. In another embodiment of the invention, the lipid nanoparticles of the invention comprise a neutral lipid or phospholipid having at least two alkyl chains, whereby each alkyl chain independently has a length of C₅, C₆, C₇, C₈, C₉, C₁₀, C₁₁, C₁₂, C₁₃ or C₁₄, preferably with a length of C₆, C₇, C₈, C₉, or C₁₀, more preferably with a length of C₆, C₇, C₈, most preferably with a length of C₇. In a preferred embodiment, the lipid nanoparticles of the invention comprise additionally DHPC. In a further embodiment, one or more alkyl chains may comprise carbon double-bonds.

In other embodiment, the lipid nanoparticles comprise an additional phospholipid selected from the group consisting of 05:0 PC (1,2-dipentanoyl-sn-glycero-3-phosphocholine), 04:0 PC (1,2-dibutyryl-sn-glycero-3-phosphocholine), 06:0 PC (DHPC, 1,2-dihexanoyl-sn-glycero-3-phosphocholine), 08:0 PC (1,2-dioctanoyl-sn-glycero-3-phosphocholine), and 09:0 PC (1,2-dinonanoyl-sn-glycero-3-phosphocholine).

### The Polymer Conjugated Lipid

In some embodiments, the LNPs comprise a lipid-conjugate, preferably a polymer conjugated lipid. The term “polymer conjugated lipid” refers to a molecule comprising both a lipid portion and a polymer portion.

### PEG-Lipid

The polymer conjugated lipid can be a pegylated lipid or PEG-lipid. The terms “pegylated lipid” or “PEG-lipid” refer to a molecule comprising both a lipid portion and a polyethylene glycol portion. Pegylated lipids are known in the art and include PEG-DMG and the like.

In a specific embodiment, the polymer conjugated lipid is defined as a compound according to formula (I):

P-A-L formula (I)

wherein P is a hydrophilic polymer moiety, A is an optional linker or spacer, and L is a lipid moiety.

### Hydrophilic Polymer Moiety P

The hydrophilic polymer moiety P in the polymer conjugated lipid according to formula (I) may be a polyethylene glycol (“PEG”) moiety. In a specific embodiment, the PEG moiety has an average molecular mass of between 1 kDa and 3 kDa, e.g. between 1.5-2.5 kDa, between 1.7-2.3 kDa, between 1.8-2.2 kDa, between 1.9-2.1 kDa, or 2 kDa. Thus the PEG can be a PEG which is commonly known as “PEG 2000” or “PEG 2k”, although the shorter “PEG 1000” and longer “PEG 3000” can also be used. The PEG moiety usually comprises linear polymer chains but, in some embodiments, the PEG moiety may comprise branched polymer chains. Alternatively, contemplated PEG-modified lipids include, but are not limited to, a polyethylene glycol chain of up to 2 kDa, up to 3 kDa, up to 4 kDa or up to 5 kDa in length covalently attached to a lipid.

In another embodiment, the hydrophilic polymer moiety P in the polymer conjugated lipid may also be a substantially hydrophilic polymer which is different from the above describes hydrophilic polymer moieties, i.e. the hydrophilic polymer moiety P in the polymer conjugated lipid may be based on poly(propylene oxide), poly(vinyl pyrrolidone), poly(vinyl alcohol), Poly-N-(2-Hydroxypropyl) methacrylamide, a hesylation-process (in accordance with PMID 24681396), a PASylation-approach (i.e. proline-alanine-serine), an XTEN-approach as known in the art (i.e. peptide based PEG), polysarcosin or poly(vinyl acetate).

### Optional Linker or Spacer A

The optional linker or spacer A in the polymer conjugated lipid according to formula (I) may be any useful spacer structure, such as a spacer selected from those that have generally been found useful in pegylated lipids, for example, but not limited to, succinimide, amine, ether, ester, anhydride, aldehyde, ketone, amide, carbamate linkers or combinations thereof.

### Lipid Moiety L

The lipid moiety L in the polymer conjugated lipid according to formula (I) may be derived from a phospholipid, a sphingolipid or a ceramide. As used herein, the expression “derived from a phospholipid or a ceramide” includes radicals of phospholipids and ceramides. Examples are polymer conjugated lipids comprising a phosphatidylethanolamine or phosphatidylglycerol moiety.

In a specific embodiment, the polymer conjugated lipid is a pegylated lipid. In a more specific embodiment, the polymer conjugated lipid comprised in the composition of the invention is a polymer conjugated lipid selected from the group consisting of a pegylated diacylglycerol lipid (PEG-DAG); a pegylated ceramide lipid (PEG-Cer); a pegylated phosphatidylethanoloamine lipid (PEG-PE); a pegylated succinate diacylglycerol lipid (PEG-S-DAG); a pegylated dialkoxypropylcarbamate lipid; 1,2-dimyristoyl-rac-glycero-3-methoxypolyethylene glycol (“PEG-DMG” or “DMG-PEG”); 1,2-dicapryl-rac-glycero-3-methylpolyoxyethylene glycol (C₁₀ diacylglycerol PEG); N-octanoyl-sphingosine-1-{succinyl[methoxy(polyethylene glycol)2000]} (comprising N-octanoyl-D-erythro-sphingosine (d18:1/8:0), also named PEG-Ceramide8, C₃-ceramide-PEG, PEG-Cer8, C₈ PEG2000 Ceramide or Ceramide 8 PEG); 4-O-(2′,3′-di(tetradecanoyloxy)propyl-1-O-(ω-methoxy(polyethoxy)ethyl)butanedioate (PEG-S-DMG); 2-mPEG2000-n,n ditetradecylacetamide; N-[(methoxy poly(ethylene glycol)2000)carbamyl]-1,2-dimyristyloxlpropyl-3-amine (PEG-c-DMA); ω-methoxy(polyethoxy)ethyl-N-(2,3-di(tetradecanoxy)propyl)carbamate; a PEG-lipid as disclosed in WO2018126084, WO2020093061, or WO2020219941 (all three references are incorporated by reference herein), PEGylated cholesterol or a PEGylated cholesterol-derivate as disclosed herein, and 2,3-di(tetradecanoxy)propyl-N-(ω-methoxy(polyethoxy)ethyl)carbamate.

In a further preferred embodiment, the lipid moiety L comprises 1, 2, 3, 4, or more hydrophobic fatty acids (“tails”, corresponding to aliphatic chains comprising an even number of carbon atoms). In a more preferred embodiment, the lipid moiety L comprises 2 hydrophobic fatty acids (“tails”) having the same or different numbers of carbon atoms.

Preferably, lipid moiety L comprises a fatty acid (“tail”) comprising, 6, 7, 8, 9, 10, 11, 12, 13, 14, 15, 16, 17, 18, 19, 20, 21, 22, 23, 24, 25 or 26 carbon atoms or combinations thereof. More preferably, lipid moiety L comprises a fatty acid (“tail”) comprising, 8, 9, 10, 11, 12, 13, 14, 15, 16, 17 or 18 carbon atoms or combinations thereof. In a more specific embodiment, lipid moiety L comprises a fatty acid (“tail”) selected from the group consisting of caprylic acid or octanoic acid (8:0); capric acid (10:0); lauric acid (12:0); myristic acid (14:0); palmitic acid (16:0); stearic acid (18:0); arachidic acid (20:0); behenic acid (22:0); lignoceric acid (24:0); and cerotic acid (26:0).

In an even more preferred embodiment, lipid moiety L comprises at least one fatty acid (“tail”) comprising 8, 10 or 12 carbon atoms, preferably 8 or 10 carbon atoms.

In a further preferred embodiment, the composition comprises the polymer conjugated lipid

### 1,2-dimyristoyl-rac-glycero-3-methoxypolyethylene glycol 2000 (DMG-PEG2000)

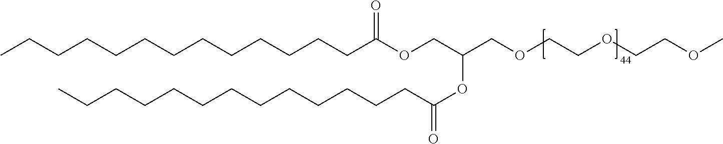

Preferably, and as used in the art, “DMG-PEG2000” is considered a mixture of 1,2-DMG PEG2000 and 1,3-DMG PEG2000 in ˜97:3 ratio.

In a further specific embodiment, the composition comprises a polymer conjugated lipid selected from the group consisting of

### 1,2-dicapryl-rac-glycero-3-methylpolyoxyethylene glycol 2000 (C₁₀-PEG2000)

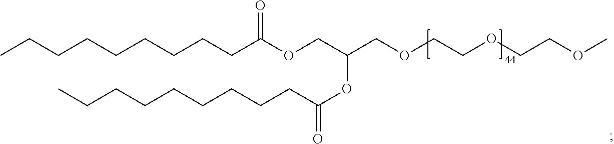

and

### N-octanoyl-sphingosine-1-{succinyl[methoxy(polyethylene glycol)2000]} (Cer8-PEG2000)

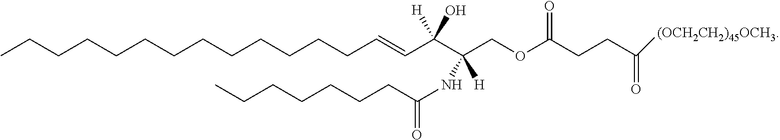

In a further embodiment, the composition comprises a polymer conjugated lipid selected from the group consisting of the following structure resembling “C₈-PEG 2000” having the following chemical structure:

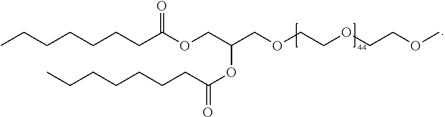

In specific embodiments of the invention, each composition as disclosed herein within the specification comprising “C₁₀-PEG 2000” can also be formulated with “C₈-PEG 2000” instead of “C₁₀-PEG 2000”.

Accordingly, as an example, a polymer conjugated lipid, or respectively lipid moiety L, may have two fatty acid tails, comprising saturated fatty acids, unsaturated fatty acids or a combination thereof (“tails”), f.e. like Cer8-PEG2000 comprising one saturated fatty acid chain (8:0; caprylic acid or respectively octanoic acid) and one unsaturated fatty acid chain of a different length with more than 8 carbon atoms.

### POZ-Lipid

The polymer conjugated lipid can be a POZ-lipid, which is defined as a compound according to formula (II):

[H]-[linker]-[M] formula (II)

wherein
[H] is a homopolymer moiety comprising at least one polyoxazoline (POZ) monomer unit

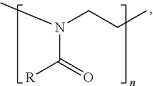

wherein R is C₁₋₉ alkyl or C₂₋₉ alkenyl and n has a mean value ranging from 2 to 200, preferably from 20 to 100, more preferably from 24 to 26 or 45 to 50
[linker] is an optional linker group, and
[M] is a lipid moiety.

In an embodiment, [H] is a heteropolymer moiety or homopolymer moiety comprising multiple monomer units selected from the group consisting of

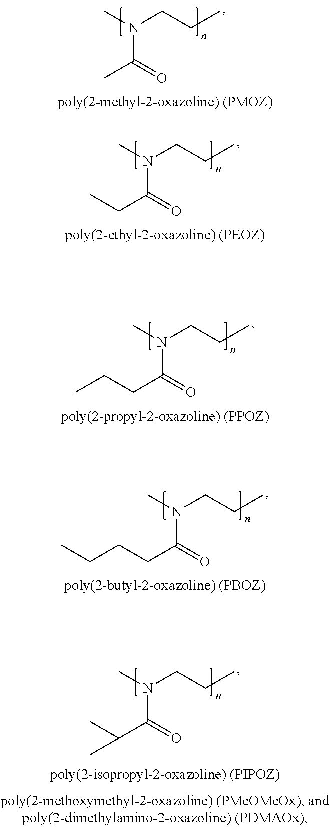

preferably wherein [H] is a homopolymer moiety comprising multiple PMOZ or PEOZ monomer units, more preferably wherein [H] comprises or preferably consists of multiple PMOZ monomer units,
wherein
(i) n has a mean value ranging from 2 to 200, preferably from 20 to 100, more preferably from 24 to 26 or 45 to 50 or wherein
(ii) n is selected such that the [H] moiety has an average molecular weight of 1.5 to 22 kDa, more preferably of 2 to 19 kDa, even more preferably of about 7.5 kDa or of about 15 kDa, preferably from 1 to 15 kDa, more preferably of 2 to 12.5 kDa, even more preferably of about 5 kDa or of about 10 kDa.

In another embodiment, [H] is a heteropolymer moiety or homopolymer moiety comprising multiple monomer units selected from the group consisting of

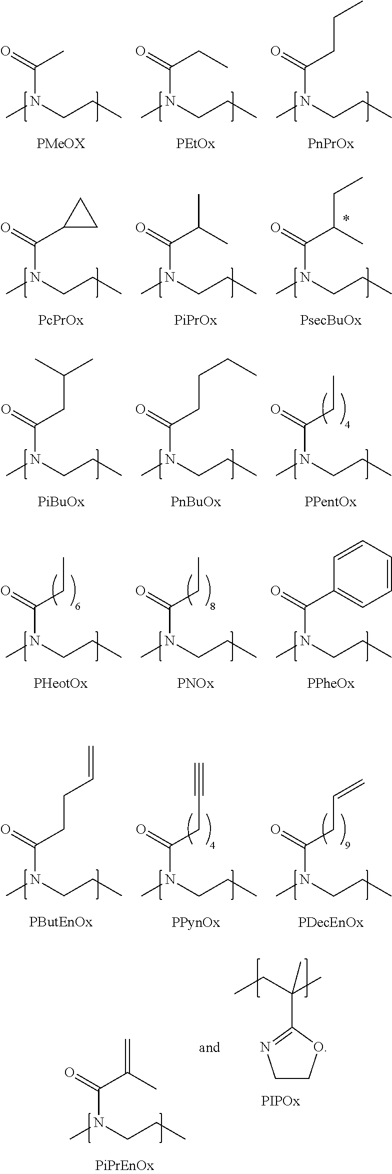

In yet another embodiment, the [H] from the polymer conjugated lipid according to formula (II) is selected from the group consisting of poly(2-methoxymethyl-2-oxazoline) (PMeOMeOx) and poly(2-dimethylamino-2-oxazoline) (PDMAOx).

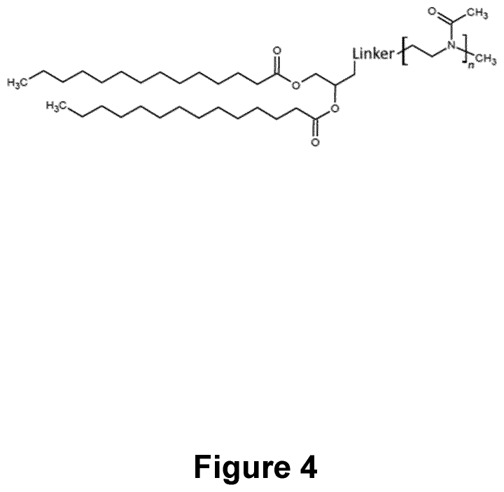

In yet a further embodiment, the polymer conjugated lipid according to formula (II) is selected from the group consisting of a POZ-monoacylglycerol conjugate, POZ-diacylglycerol conjugate, a POZ-dialkyloxypropyl conjugate, a POZ-steroid or POZ-sterol conjugate, a POZ-phospholipid conjugate, a POZ-ceramide conjugate, a PMOZ-lipid as shown in FIG. 4, and a mixture thereof.

In one embodiment, the lipid moiety [M] as shown in formula (II) comprises at least one straight or branched, saturated or unsaturated alkyl chain containing from 6 to 30 carbon atoms, preferably wherein the lipid moiety [M] comprises at least one straight or branched saturated alkyl chain, wherein the alkyl chain is optionally interrupted by one or more biodegradable group(s) and/or optionally comprises one terminal biodegradable group, wherein the biodegradable group is selected from the group consisting of but not limited to a pH-sensitive moiety, a zwitterionic linker, non-ester containing linker moieties and ester-containing linker moieties (—C(O)O— or —OC(O)—), amido (—C(O)NH—), disulfide (—S—S—), carbonyl (—C(O)—), ether (—O—), thioether (—S—), oxime (e.g., —C(H)=N—O— or —O—N═C(H)—), carbamate (—NHC(O)O—), urea (—NHC(O)NH—), succinyl (—(O)CCH2CH2C(O)—), succinamidyl (—NHC(O)CH2CH2C(O)NH—), —C(R⁵)=N—, —N═C(R⁵)—, —C(R⁵)=N—O—, —O—N═C(R⁵)—, —O—C(O)O—, —C(O)N(R⁵), —N(R⁵)C(O)—, —C(S)(NR⁵)—, (NR⁵)C(S)—, —N(R⁵)C(O)N(R⁵)—, —C(O)S—, —SC(O)—, —C(S)O—, —OC(S)—, —OSi(R⁵)₂O—, —C(O)(CR³R⁴)C(O)O—, or —OC(O)(CR³R⁴)C(O)—, carbonate (—OC(O)O—), succinoyl, phosphate esters (—O—(O)POH—O—), cyclic compound, heterocyclic compound, piperidine, pyrazine, pyridine, piperazine, and sulfonate esters, as well as combinations thereof, wherein R³, R⁴ and R⁵ are, independently H or alkyl (e.g. C₁-C₄ alkyl).

In another embodiment, the the lipid moiety [M] comprises at least one straight or branched, saturated or unsaturated alkyl chain comprising 6, 7, 8, 9, 10, 11, 12, 13, 14, 15, 16, 17, 18, 19, 20, 21, 22, 23, 24, 25, 26, 27, 28, 29, or 30 carbon atoms, preferably in the range of 10 to 20 carbon atoms, more preferably in the range of 12 to 18 carbon atoms, even more preferably 14, 16 or 18 carbon atoms, even more preferably 16 or 18 carbon atoms, most preferably 14 carbon atoms, wherein all selections are independent of one another.

In one embodiment, the linker group [linker] as shown in formula (II) is selected from the group consisting of but not limited to a pH-sensitive moiety, a zwitterionic linker, non-ester containing linker moieties and ester-containing linker moieties (—C(O)O— or —OC(O)—), amido (—C(O)NH—), disulfide (—S—S—), carbonyl (—C(O)—), ether (—O—), thioether (—S—), oxime (e.g., —C(H)═N—O— or —O—N═C(H)—), carbamate (—NHC(O)O—), urea (—NHC(O)NH—), succinyl (—(O)CCH2CH2C(O)—), succinamidyl (—NHC(O)CH2CH2C(O)NH—), —C(R⁵)═N—, —N═C(R⁵)—, —C(R⁵)═N—O—, —O—N═C(R⁵)—, —O—C(O)O—, —C(O)N(R⁵), —N(R⁵)C(O)—, —C(S)(NR⁵)—, (NR⁵)C(S)—, —N(R⁵)C(O)N(R⁵)—, —C(O)S—, —SC(O)—, —C(S)O—, —OC(S)—, —OSi(R⁵)₂O—, —C(O)(CR³R⁴)C(O)O—, or —OC(O)(CR³R⁴)C(O)—, carbonate (—OC(O)O—), succinoyl, phosphate esters (—O—(O)POH—O—), and sulfonate esters, as well as combinations thereof, wherein R³, R⁴ and R⁵ are, independently H or alkyl (e.g. C₁-C₄ alkyl).

In a very preferred embodiment, the polymer conjugated lipid has the structure of

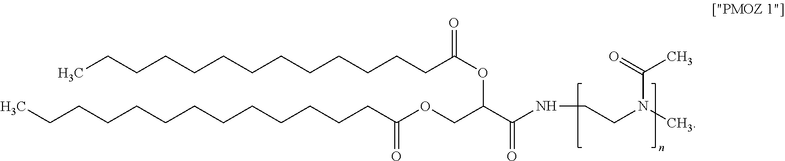

In another very preferred embodiment, the polymer conjugated lipid has the structure of

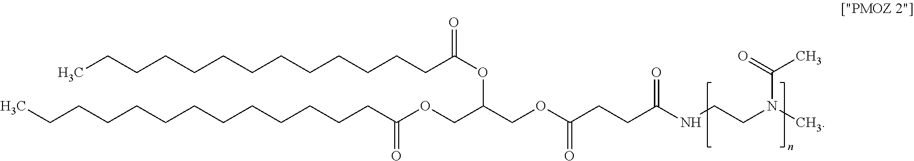

In another very preferred embodiment, the polymer conjugated lipid has the structure of “PMOZ 2” with n=50 i.e. having 50 monomer repeats.

In an even further preferred embodiment, the polymer conjugated lipid has the structure of

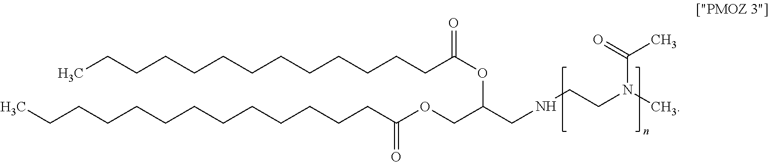

In another preferred embodiment, the polymer conjugated lipid has the structure of

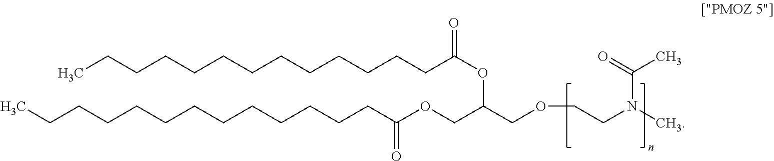

In an even further very preferred embodiment, the polymer conjugated lipid has the structure of

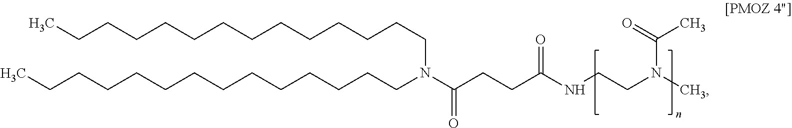

preferably with n=50 i.e. having 50 monomer repeats, i.e.

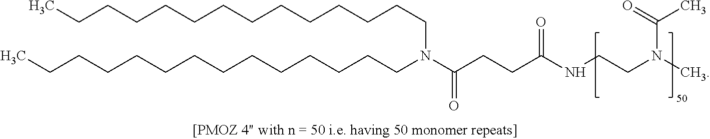

For “PMOZ 1” to “PMOZ 5”, preferably n has a mean value ranging from 2 to 200, preferably from 20 to 100, more preferably from 24 to 26, even more preferably about 100, or further even more preferably from 45 to 50, most preferably 50 or wherein n is selected such that the [P] moiety has an average molecular weight of about 4.2 kDa to about 4.4 kDa, or most preferably about 4.3 kDa.

In another very preferred embodiment, the linker group [linker] comprises preferably an amide linker moiety.

In a further very preferred embodiment, the linker group [linker] comprises preferably an ester linker moiety.

In a further very preferred embodiment, the linker group [linker] comprises preferably a succinate linker moiety.

In another very preferred embodiment, the linker group [linker] comprises both an ester linker and an amid linker moiety. In another preferred embodiment, the linker group [linker] comprises both an ester linker, an amine linker and an amid linker moiety.

It is noted herein, that all chemical compounds mentioned throughout the whole specification may be produced via processes known to a skilled worker; starting materials and/or reagents used in the processes are obtainable through routine knowledge of a skilled worker on the basis of common general knowledge (e.g. from text books or from e.g. patent applications WO2022173667, WO2009043027, WO2013067199, WO2010006282, WO2009089542, WO2016019340, WO2008106186, WO2020264505, and WO2020023947, the complete disclosure of said patent applications is incorporated by reference herein).

In yet a further embodiment, the lipid nanoparticle does not comprise a polyethylene glycol-(PEG)-lipid conjugate or a conjugate of PEG and a lipid-like material, and preferably do not comprise PEG and/or (ii) the polymer conjugated lipid of the invention does not comprise a sulphur group (—S—), a terminating nucleophile, and/or is covalently coupled to a biologically active ingredient is a nucleic acid compound selected from the group consisting of RNA, an artificial mRNA, chemically modified or unmodified messenger RNA (mRNA) comprising at least one coding sequence, self-replicating RNA, circular RNA, viral RNA, and replicon RNA; or any combination thereof, preferably wherein the biologically active ingredient is chemically modified mRNA or chemically unmodified mRNA, more preferably wherein the biologically active ingredient is chemically unmodified mRNA.

In another very preferred embodiment, the polymer conjugated lipid of the invention does not comprise sulphur (S) or a sulphur group (—S—).

### Lipid Nanoparticle Compositions

The terms “lipid nanoparticle composition” and “composition” are used in this section interchangeably. In the context of the present invention, lipid nanoparticles are not restricted to any particular morphology, and should be interpreted as to include any morphology generated when a cationic lipid and optionally one or more further lipids are combined, e.g. in an aqueous environment and/or in the presence of a nucleic acid compound. For example, a liposome, a lipid complex, a lipoplex and the like are within the scope of a lipid nanoparticle.

In the context of the invention, a “composition” refers to any type of composition in which the specified ingredients may be incorporated, optionally along with any further excipients, usually with at least one pharmaceutically acceptable carrier or excipient. Thus, the composition may be a dry composition such as a powder or granules, or a solid unit such as a lyophilized form or a tablet. Alternatively, the composition may be in liquid form, and each excipient may be independently incorporated in dissolved or dispersed (e.g. suspended or emulsified) form. In one of the preferred embodiments, the composition is formulated as a sterile solid composition, such as a powder or lyophilized form for reconstitution with an aqueous liquid carrier. Such formulation is also preferred for those versions of the composition which comprise a nucleic acid cargo as described in further detail below.

A “nanoparticle”, as used herein, is a submicron particle having any structure or morphology. Submicron particles may also be referred to as colloids, or colloidal. With respect to the material on which the nanoparticle is based, and to the structure or morphology, a nanoparticle may be classified, for example, as a nanocapsule, a vesicle, a liposome, a lipid nanoparticle, a micelle, a cross-linked micelle, a lipoplex, a polyplex, a mixed or hybrid complex, to mention only a few of the possible designations of specific types of nanoparticles. A “lipid nanoparticle” (LNP) is a nanoparticle formed by lipids, typically including at least one amphiphilic, membrane-forming lipid, and optionally other lipids, further optionally including a cargo material such as a nucleic acid compound. As used herein, the expression “lipid nanoparticles” or “LNP” includes any sub-types and morphologies of nanoparticles formed or co-formed by lipids, such as liposomes and lipoplexes.

As defined above, lipid nanoparticles include any type of nanoparticles formed or co-formed by lipids. In particular, lipid nanoparticles may co-formed by combinations of lipids comprising at least one amphiphilic, vesicle-forming lipid. Liposomes and lipoplexes are examples of lipid nanoparticles.

An LNP according to the present invention comprises the phospholipid phosphatidylserine, preferably in combination with the further lipids as outlined herein.

In principle, an LNP may comprise any lipid capable of forming a particle to which the one or more nucleic acid molecules are attached, or in which the one or more nucleic acid molecules are encapsulated. In some embodiments, the mRNA, or a portion thereof, is encapsulated in the lipid portion of the lipid nanoparticle or an aqueous space enveloped by some or all of the lipid portion of the lipid nanoparticle, thereby protecting it from enzymatic degradation or other undesirable effects induced by the mechanisms of the host organism or cells e.g. an adverse immune response. In some embodiments, the mRNA or a portion thereof is associated with the lipid nanoparticles.

As mentioned, a composition comprising the lipidic excipients as described herein will normally form lipid nanoparticles, at least in an aqueous environment. As defined herein, the nanoparticles have a predominantly submicron size. In certain embodiments, the mRNA, when present in the lipid nanoparticles, is resistant in aqueous solution to degradation with a nuclease. As used herein, the mean diameter may be represented by the z-average as determined by dynamic light scattering. In one embodiment, the composition is a sterile liquid composition comprising lipid nanoparticles having a mean hydrodynamic diameter (or mean size) as determined by dynamic laser scattering from about 30 nm to about 800 nm. In various embodiments, the lipid nanoparticles have a mean diameter of from about 30 nm to about 150 nm, from about 50 nm to about 200 nm, from about 60 nm to about 200 nm, from about 70 nm to about 200 nm, from about 80 nm to about 200 nm, from about 90 nm to about 200 nm, from about 90 nm to about 190 nm, from about 90 nm to about 180 nm, from about 90 nm to about 170 nm, from about 90 nm to about 160 nm, from about 90 nm to about 150 nm, from about 90 nm to about 140 nm, from about 90 nm to about 130 nm, from about 90 nm to about 120 nm, from about 90 nm to about 100 nm, from about 70 nm to about 90 nm, from about 80 nm to about 90 nm, from about 70 nm to about 80 nm, or about 30 nm, 35 nm, 40 nm, 45 nm, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, 150 nm, 160 nm, 170 nm, 180 nm, 190 nm, or 200 nm, and are substantially non-toxic. In another preferred embodiment of the invention the lipid nanoparticles have a hydrodynamic diameter in the range from about 50 nm to about 300 nm, or from about 60 nm to about 250 nm, from about 60 nm to about 150 nm, or from about 60 nm to about 120 nm, or from about 80 nm to about 160 nm, or from about 90 nm to about 140 nm, 50 nm to about 300 nm, or from about 60 nm to about 250 nm, or from about 60 nm to about 200 nm, or from about 70 nm to 200 nm, or from about 75 nm to about 160 nm, or from about 100 nm to about 140 nm, or from about 90 nm to about 140 nm. Also preferred is a range of about 50 nm to about 60 nm or a range of about 60 nm to about 80 nm.

Compositions comprising the lipidic excipients as described herein yielding lipid nanoparticles of the invention may be relatively homogenous. A polydispersity index (PDI) may be used to indicate the homogeneity of a nanoparticle composition, e.g., the particle size distribution of the nanoparticle compositions. A small (e.g., less than 0.3) polydispersity index generally indicates a narrow particle size distribution. A nanoparticle composition of the invention may have a polydispersity index from about 0 to about 0.35, such as 0.01, 0.02, 0.03, 0.04, 0.05, 0.06, 0.07, 0.08, 0.09, 0.10, 0.11, 0.12, 0.13, 0.14, 0.15, 0.16, 0.17, 0.18, 0.19, 0.20, 0.21, 0.22, 0.23, 0.24, 0.25, 0.26, 0.27, 0.28, 0.29, 0.30, 0.31, 0.32, 0.33, 0.34 or 0.35. In some embodiments, the polydispersity index (PDI) of a nanoparticle composition may be from about 0.1 to about 0.2.

Various optional features, selections and preferences relating to the composition of the invention in general have been described herein: all of these also apply to the lipid nanoparticles, as will be clearly understood by a person skilled in the art. Similarly, the options and preferences apply to compositions comprising such lipid nanoparticles.

With respect to the amounts of the respective excipients, it is preferred that the cationic lipid is incorporated in the lipid nanoparticles, or in the composition according to the invention, at a relatively high molar amount compared to the molar amount at which the polymer conjugated lipid according to formula (I) is present. Moreover, the molar amount of the cationic lipid is also preferably higher than the molar of amount of the neutral lipid in the composition or in the nanoparticles, respectively. Furthermore, the molar amount of the steroid is optionally higher than the molar amount of the polymer conjugated lipid according to formula (I).

In certain embodiments, the polymer conjugated lipid is present in the LNP in an amount from about 1 mol % to about 10 mol %, relative to the total lipid content of the nanoparticle. In one embodiment, the polymer conjugated lipid is present in the LNP in an amount from about 1 mol % to about 5 mol % percent. In one embodiment, the polymer conjugated lipid is present in the LNP in about 1 mol % or about 1.5 mol %. In a preferred embodiment, the polymer conjugated lipid is present in the LNP in an amount from about 1, 2, 3, 4, 5, 6, 7, 8, 9, or 10 mol %; preferably in an amount of 5 mol %, more preferably in an amount of 2.5 mol % or also preferably in an amount of 1.7 mol %, based upon a mol-percentage of the composition of 100% of all lipid components or excipients.

In various embodiments, the molar ratio of the cationic lipid to the polymer conjugated lipid ranges from about 100:1 to about 25:1, from about 50:1 to about 25:1, or from about 40:1 to about 25:1.

In certain embodiments, the LNP comprises one or more additional lipids which stabilize the formation of particles during their formation. Suitable stabilizing lipids include neutral lipids and anionic lipids. In various embodiments, the molar ratio of the cationic lipid to the neutral lipid ranges from about 2:1 to about 8:1, from about 3:1 to about 7:1, or from about 4:1 to about 6:1.

As used herein, references to molar amounts of lipidic excipients in the composition of the invention should be understood as also describing the molar amounts of the respective excipients in the lipid nanoparticles comprised in the composition, as the lipid nanoparticles are typically formed by these excipients and reflect the same quantitative ratios of excipients as the overall composition containing the nanoparticles.

The phospholipid phosphatidylserine may be present in the composition at an amount of in the range from about 1 mol % to about 15 mol %, or from about 2 mol % to about 10 mol %; such as about 1 mol %, 2 mol %, 3 mol %, 4 mol %, 5 mol %, 6 mol %, 7 mol %, 8 mol %, 9 mol %, 10 mol %, 11 mol %, 12 mol %, 13 mol %, 14 mol % or 15 mol %, respectively, using the same basis for the molar percentages. In some embodiments, the amount of phosphatidylserine is not more than 10 mol % or about 10 mol % of the total molar amount of all lipidic excipients in the composition. In some embodiments, the amount of phosphatidylserine is not more than 9 mol % or about 9 mol % of the total molar amount of all lipidic excipients in the composition. In some embodiments, the amount of phosphatidylserine is not more than 8 mol % or about 8 mol % of the total molar amount of all lipidic excipients in the composition. In some embodiments, the amount of phosphatidylserine is not more than 7 mol % or about 7 mol % of the total molar amount of all lipidic excipients in the composition. In some embodiments, the amount of phosphatidylserine is not more than 6 mol % or about 6 mol % of the total molar amount of all lipidic excipients in the composition. In other embodiments, the amount of phosphatidylserine is not more than 5 mol % or about 5 mol % of the total molar amount of all lipidic excipients in the composition.

In general, the amount of the cationic lipid in the composition (and thus in the lipid nanoparticles) is typically at least about 20 mol %, relative to the total molar amount of all lipidic excipients in the composition (or nanoparticles). In another embodiment, the amount of the cationic lipid is at least about 25 mol %, or at least 30 mol %, respectively. In other preferred embodiments, the amount of the cationic lipid in the composition is from about 30 mol % to about 70 mol %, or from about 40 mol % to about 70 mol %, or from about 45 mol % to about 65 mol %, respectively; such as about 30, 35, 40, 45, 50, 55, 60, 65, or 70 mol %, or from about 40 mol % to about 60 mol %, respectively; such as about 40, 41, 42, 43, 44, 45, 46, 47, 48, 49, 50, 51, 52, 53, 54, 55, 56, 57, 58, 59 or 60 mol %, respectively.

The amount of the steroid in the composition may optionally at least about 10 mol %, or it may be in the range from about 10 mol % to about 60 mol %, or from about 20 mol % to about 50 mol %, or from about 25 mol % to about 45 mol %, respectively; such as about 10, 15, 20, 25, 30, 35, 40, 45, 50, 55, or 60 mol %, respectively. Again, for the avoidance of doubt, the molar percentages are relative the total molar amount of all lipidic excipients in the composition.

The neutral lipid may optionally be present at an amount of at least about 5 mol %. In some embodiments, the amount of the neutral lipid in the composition is in the range from about 5 mol % to about 25 mol %, or from about 5 mol % to about 15 mol %, or from about 8 mol % to about 12 mol %, respectively; such as about 5 mol %, 6 mol %, 7 mol %, 8 mol %, 9 mol %, 10 mol %, 11 mol %, 12 mol %, 13 mol %, 14 mol %, 15 mol %, 16 mol %, 17 mol %, 18 mol %, 19 mol %, 20 mol %, 21 mol %, 22 mol %, 23 mol %, 24 mol % or 25 mol %, respectively, using the same basis for the molar percentages.

The amount of polymer conjugated lipid in the composition or in the lipid nanoparticles may, for example, be selected to be about 0.1 mol % and higher. In certain embodiments, the amount of the polymer conjugated lipid is in the range from about 1 mol % to about 15 mol %, or from about 2 mol % to about 12 mol %, respectively, using again the total molar amount of all lipidic excipients as basis for the molar percentages. In other certain embodiments, the composition or the lipid nanoparticles may comprise 0.1; 0.2; 0.3; 0.4; 0.5; 0.6; 0.7; 0.8; 0.9; 1.0; 1.1; 1.2; 1.3; 1.4; 1.5; 1.6; 1.7; 1.8; 1.9; 2.0; 2.1; 2.2; 2.3; 2.4; 2.5; 2.6; 2.7; 2.8; 2.9; 3.0; 3.1; 3.2; 3.3; 3.4; 3.5; 3.6; 3.7; 3.8; 3.9; 4.0; 4.1; 4.2; 4.3; 4.4; 4.5; 4.6; 4.7; 4.8; 4.9; 5.0; 5.1; 5.2; 5.3; 5.4; 5.5; 5.6; 5.7; 5.8; 5.9; 6; 6.1; 6.2; 6.3; 6.4; 6.5; 6.6; 6.7; 6.8; 6.9; 7; 7.1; 7.2; 7.3; 7.4; 7.5; 7.6; 7.7; 7.8; 7.9; 8; 8.1; 8.2; 8.3; 8.4; 8.5; 8.6; 8.7; 8.8; 8.9; 9; 9.1; 9.2; 9.3; 9.4; 9.5; 9.6; 9.7; 9.8; 9.9; 10; 10.1; 10.2; 10.3; 10.4; 10.5; 10.6; 10.7; 10.8; 10.9; 11; 11.1; 11.2; 11.3; 11.4; 11.5; 11.6; 11.7; 11.8; 11.9; or 12 mol % or more than 12 mol % polymer conjugated lipid. In a preferred embodiment, the content of the polymer conjugated lipid is about 1 mol % to 5 mol % of the overall lipid content of the formulation, preferably 1.7 mol % or 2.5 mol %. As a non-limiting preferred example, the lipid nanoparticle comprises 5 mol % polymer conjugated lipid. As another non-limiting example preferred, the lipid nanoparticle comprises 10 mol % polymer conjugated lipid. As another non-limiting example, the lipid nanoparticle comprises 7.5 mol % polymer conjugated lipid.

In one embodiment, the composition comprises lipid nanoparticles which comprise:
(a) the cationic lipid at an amount of 30-70 mol %;
(b) the steroid at an amount of 20-50 mol %;
(c) the phospholipid at an amount of 5-25 mol %; and
(d) polymer conjugated lipid according to formula (I) at an amount of 0.5-5 mol %;
each amount being relative to the total molar amount of all lipidic excipients of the lipid nanoparticles.

In another embodiment, the composition comprises lipid nanoparticles comprising:
(a) the cationic lipid at an amount of 40-60 mol %;
(b) the steroid at an amount of 20-40 mol %;
(c) the phospholipid at an amount of 10-20 mol %; and
(d) polymer conjugated lipid according to formula (I) at an amount of 1-2 mol %;
each amount being relative to the total molar amount of all lipidic excipients of the lipid nanoparticles.

In one embodiment, the composition comprises lipid nanoparticles which comprise:
(a) the cationic lipid at an amount of 30-70 mol %;
(b) cholesterol at an amount of 20-50 mol %;
(c) the phospholipid phosphatidylserine and a further phospholipid (preferably DPhyPE) at an amount of 5-25 mol %; and
(d) polymer conjugated lipid (preferably DMG-PEG2000 or DSG-PEG2000) at an amount of 0.5-5 mol %; each amount being relative to the total molar amount of all lipidic excipients of the lipid nanoparticles.

In a further embodiment, the composition comprises lipid nanoparticles which comprise:
(a) the cationic lipid at an amount of 40-60 mol %;
(b) cholesterol at an amount of 20-40 mol %;
(c) the phospholipid phosphatidylserine and a further phospholipid (preferably DPhyPE) at an amount of 10-20 mol %; and
(d) polymer conjugated lipid (preferably DMG-PEG2000 or DSG-PEG2000) at an amount of 1-2 mol %, preferably 1.7 mol %;
each amount being relative to the total molar amount of all lipidic excipients of the lipid nanoparticles.

In one embodiment, the composition comprises lipid nanoparticles which comprise:
(a) the cationic lipid at an amount of 30-70 mol %;
(b) cholesterol at an amount of 20-50 mol %;
(c) the phospholipid phosphatidylserine, DPhyPE and DHPC at an amount of 5-25 mol %; and
(d) polymer conjugated lipid (preferably DMG-PEG2000 or DSG-PEG2000) at an amount of 0.5-5 mol %; each amount being relative to the total molar amount of all lipidic excipients of the lipid nanoparticles.

In a further embodiment, the composition comprises lipid nanoparticles which comprise:
(a) the cationic lipid at an amount of 40-60 mol %;
(b) cholesterol at an amount of 20-40 mol %;
(c) the phospholipid phosphatidylserine, DPhyPE and DHP at an amount of 10-20 mol %; and
(d) polymer conjugated lipid (preferably DMG-PEG2000 or DSG-PEG2000) at an amount of 1-2 mol %, preferably 1.7 mol %;
each amount being relative to the total molar amount of all lipidic excipients of the lipid nanoparticles.

In these embodiments, the cationic lipid is preferably a compound selected according to any one of the preferences disclosed herein. For example, the cationic lipid may be selected from the compounds listed in Table 1. Moreover, these embodiments may also comprise a steroid, a phospholipid, and/or a polymer conjugated lipid selected according to any one of the preferences disclosed herein. In all embodiments which recite compositions or lipid nanoparticles as described herein and where mol %-values are given for each excipient, each amount should be seen being relative to the total molar amount of all lipidic excipients of the lipid nanoparticles.

In a further preferred embodiment, the composition comprises lipid nanoparticles which comprise:
(a) the cationic lipid “THIOETHER”, preferably at an amount of 30-70 mol %, more preferably at an amount of 40-60 mol %;
(b) cholesterol, preferably at an amount of 20-50 mol %, more preferably at an amount of 20-40 mol %;
(c) the phospholipid phosphatidylserine and a further phospholipid (preferably DPhyPE, optionally in combination with DHPC), preferably at an amount of 5-25 mol %; more preferably at an amount of 10-20 mol %; and
(d) the polymer conjugated lipid “PMOZ 2”, preferably at an amount of 0.5-5 mol %, more preferably at an amount of 2.5 mol %;
each amount being relative to the total molar amount of all lipidic excipients of the lipid nanoparticles.

In an especially preferred embodiment, the composition comprises lipid nanoparticles which comprise:
(a) the cationic lipid “THIOETHER”, preferably at an amount of 30-70 mol %, more preferably at an amount of 40-60 mol %;
(b) cholesterol, preferably at an amount of 20-50 mol %, more preferably at an amount of 20-40 mol %;
(c) the phospholipid phosphatidylserine and a further phospholipid (preferably DPhyPE, optionally in combination with DHPC), preferably at an amount of 5-25 mol %; more preferably at an amount of 10-20 mol %; and
(d) the polymer conjugated lipid “PMOZ 4”, preferably at an amount of 0.5-5 mol %, more preferably at an amount of 2.5 mol %;
each amount being relative to the total molar amount of all lipidic excipients of the lipid nanoparticles.

In a further preferred embodiment, the composition or the lipid nanoparticle as described herein comprises 59 mol % cationic lipid, 10 mol % phospholipid, 29.3 mol % steroid and 1.7 mol % polymer conjugated lipid. In a further preferred embodiment, the composition or the lipid nanoparticle as described herein comprises 58 mol % cationic lipid, 11 mol % phospholipid, 29.3 mol % steroid and 1.7 mol % polymer conjugated lipid. In a further preferred embodiment, the composition or the lipid nanoparticle as described herein comprises 49 mol % cationic lipid, 20 mol % phospholipid, 29.3 mol % steroid and 1.7 mol % polymer conjugated lipid.

In a further preferred embodiment, the composition or the lipid nanoparticle as described herein comprises 59 mol % cationic lipid, 10 mol % phosphatidylserine and DPhyPE, 29.3 mol % cholesterol and 1.7 mol % polymer conjugated lipid (preferably DMG-PEG2000 or DSG-PEG2000). In a further preferred embodiment, the composition or the lipid nanoparticle as described herein comprises 58 mol % cationic lipid, 11 mol % phosphatidylserine, DPhyPE and DHPC, 29.3 mol % cholesterol and 1.7 mol % polymer conjugated lipid (preferably DMG-PEG2000 or DSG-PEG2000). In a further preferred embodiment, the composition or the lipid nanoparticle as described herein comprises 49 mol % cationic lipid, 20 mol % phosphatidylserine, DPhyPE and DHPC, 29.3 mol % cholesterol and 1.7 mol % polymer conjugated lipid (preferably DMG-PEG2000 or DSG-PEG2000).

In these embodiments, the cationic lipid is preferably a compound selected according to any one of the preferences disclosed herein. For example, the cationic lipid may be selected from the compounds listed in Table 1.

In any of the above embodiments in this section disclosing specific compositions or lipid nanoparticles having distinct %-values for excipients, if 1,2-diphytanoyl-sn-glycero-3-phosphoethanolamine (DPhyPE) is mentioned as phospholipid, in further embodiments DPhyPE may be exchanged with another phospholipid, preferably 1,2-diphytanoyl-sn-glycero-3-phosphocholine (DPhyPC). Furthermore, In any of the above embodiments in this section disclosing specific compositions or lipid nanoparticles having distinct %-values for excipients, if 1,2-diphytanoyl-sn-glycero-3-phosphoethanolamine (DPhyPE) is mentioned as phospholipid, in even further embodiments DPhyPE may be exchanged with another phospholipid, preferably 1,2-dioleoyl-sn-glycero-3-phosphocholine (DOPC; also referred to as dioleoylphosphatidylcholine) or alternatively 1,2-dioleoyl-sn-glycero-3-phosphoethanolamine (DOPE).

Further preferred lipid compositions comprise the at least five lipid excipients as disclosed herein in Table E. For example, a preferred lipid composition comprises the excipients as disclosed in line “E1” which are “C1” as cationic lipid (as disclosed herein in Table 1), DPhyPE+DPhyPS as phospholipid combination, cholesterol as sterol and DMG-PEG2000 as polymer conjugated lipid (and thus in total five lipid excipients). As another example a preferred lipid composition comprises the excipients as disclosed in line “E56” which are “C2” as cationic lipid (as disclosed herein in Table 1), DPhyPE+DPhyPS+DHPC as phospholipid combination, cholesterol as sterol and DMG-PEG2000 as polymer conjugated lipid (and thus in total six lipid excipients).

TABLE E
Lipid excipient combinations for preferred compositions

| Excipient | | | | polymer | Excipient | | | | polymer |
|---|---|---|---|---|---|---|---|---|---|
| combination | cationic | | Phospholipid | conjugated | combination | cationic | | neutral | conjugated |
| [designation] | lipid | sterol | combination | lipid | [designation] | lipid | sterol | lipid | lipid |
| E1 | C1 | Chol | DPhyPE + | DMG-PEG | E36 | C9 | Chol | DPhyPE + | DMG-PEG |
| | | | DPhyPS | 2000 | | | | WT-PS | 2000 |
| E2 | C2 | Chol | DPhyPE + | DMG-PEG | E37 | C10 | Chol | DPhyPE + | DMG-PEG |
| | | | DPhyPS | 2000 | | | | WT-PS | 2000 |
| E3 | C3 | Chol | DPhyPE + | DMG-PEG | E38 | C11 | Chol | DPhyPE + | DMG-PEG |
| | | | DPhyPS | 2000 | | | | WT-PS | 2000 |
| E4 | C4 | Chol | DPhyPE + | DMG-PEG | E39 | C12 | Chol | DPhyPE + | DMG-PEG |
| | | | DPhyPS | 2000 | | | | WT-PS | 2000 |
| E5 | C5 | Chol | DPhyPE + | DMG-PEG | E40 | C13 | Chol | DPhyPE + | DMG-PEG |
| | | | DPhyPS | 2000 | | | | WT-PS | 2000 |
| E6 | C6 | Chol | DPhyPE + | DMG-PEG | E41 | C14 | Chol | DPhyPE + | DMG-PEG |
| | | | DPhyPS | 2000 | | | | WT-PS | 2000 |
| E7 | C7 | Chol | DPhyPE + | DMG-PEG | E42 | C15 | Chol | DPhyPE + | DMG-PEG |
| | | | DPhyPS | 2000 | | | | WT-PS | 2000 |
| E8 | C8 | Chol | DPhyPE + | DMG-PEG | E43 | C16 | Chol | DPhyPE + | DMG-PEG |
| | | | DPhyPS | 2000 | | | | WT-PS | 2000 |
| E9 | C9 | Chol | DPhyPE + | DMG-PEG | E44 | C17 | Chol | DPhyPE + | DMG-PEG |
| | | | DPhyPS | 2000 | | | | WT-PS | 2000 |
| E10 | C10 | Chol | DPhyPE + | DMG-PEG | E45 | C18 | Chol | DPhyPE + | DMG-PEG |
| | | | DPhyPS | 2000 | | | | WT-PS | 2000 |
| E11 | C11 | Chol | DPhyPE + | DMG-PEG | E46 | C19 | Chol | DPhyPE + | DMG-PEG |
| | | | DPhyPS | 2000 | | | | WT-PS | 2000 |
| E12 | C12 | Chol | DPhyPE + | DMG-PEG | E47 | C20 | Chol | DPhyPE + | DMG-PEG |
| | | | DPhyPS | 2000 | | | | WT-PS | 2000 |
| E13 | C13 | Chol | DPhyPE + | DMG-PEG | E48 | C21 | Chol | DPhyPE + | DMG-PEG |
| | | | DPhyPS | 2000 | | | | WT-PS | 2000 |
| E14 | C14 | Chol | DPhyPE + | DMG-PEG | E49 | C22 | Chol | DPhyPE + | DMG-PEG |
| | | | DPhyPS | 2000 | | | | WT-PS | 2000 |
| E15 | C15 | Chol | DPhyPE + | DMG-PEG | E50 | C23 | Chol | DPhyPE + | DMG-PEG |
| | | | DPhyPS | 2000 | | | | WT-PS | 2000 |
| E16 | C16 | Chol | DPhyPE + | DMG-PEG | E51 | C24 | Chol | DPhyPE + | DMG-PEG |
| | | | DPhyPS | 2000 | | | | WT-PS | 2000 |
| E17 | C17 | Chol | DPhyPE + | DMG-PEG | E52 | C25 | Chol | DPhyPE + | DMG-PEG |
| | | | DPhyPS | 2000 | | | | WT-PS | 2000 |
| E18 | C18 | Chol | DPhyPE + | DMG-PEG | E53 | C26 | Chol | DPhyPE + | DMG-PEG |
| | | | DPhyPS | 2000 | | | | WT-PS | 2000 |
| E19 | C19 | Chol | DPhyPE + | DMG-PEG | E54 | C27 | Chol | DPhyPE + | DMG-PEG |
| | | | DPhyPS | 2000 | | | | WT-PS | 2000 |
| E20 | C20 | Chol | DPhyPE + | DMG-PEG | E55 | C1 | Chol | DPhyPE + | DMG-PEG |
| | | | DPhyPS | 2000 | | | | DPhyPS + | 2000 |
| | | | | | | | | DHPC | |
| E21 | C21 | Chol | DPhyPE + | DMG-PEG | E56 | C2 | Chol | DPhyPE + | DMG-PEG |
| | | | DPhyPS | 2000 | | | | DPhyPS + | 2000 |
| | | | | | | | | DHPC | |
| E22 | C22 | Chol | DPhyPE + | DMG-PEG | E57 | C3 | Chol | DPhyPE + | DMG-PEG |
| | | | DPhyPS | 2000 | | | | DPhyPS + | 2000 |
| | | | | | | | | DHPC | |
| E23 | C23 | Chol | DPhyPE + | DMG-PEG | E58 | C4 | Chol | DPhyPE + | DMG-PEG |
| | | | DPhyPS | 2000 | | | | DPhyPS + | 2000 |
| | | | | | | | | DHPC | |
| E24 | C24 | Chol | DPhyPE + | DMG-PEG | E59 | C5 | Chol | DPhyPE + | DMG-PEG |
| | | | DPhyPS | 2000 | | | | DPhyPS + | 2000 |
| | | | | | | | | DHPC | |
| E25 | C25 | Chol | DPhyPE + | DMG-PEG | E60 | C6 | Chol | DPhyPE + | DMG-PEG |
| | | | DPhyPS | 2000 | | | | DPhyPS + | 2000 |
| | | | | | | | | DHPC | |
| E26 | C26 | Chol | DPhyPE + | DMG-PEG | E61 | C7 | Chol | DPhyPE + | DMG-PEG |
| | | | DPhyPS | 2000 | | | | DPhyPS + | 2000 |
| | | | | | | | | DHPC | |
| E27 | C27 | Chol | DPhyPE + | DMG-PEG | E62 | C8 | Chol | DPhyPE + | DMG-PEG |
| | | | DPhyPS | 2000 | | | | DPhyPS + | 2000 |
| | | | | | | | | DHPC | |
| E28 | C1 | Chol | DPhyPE + | DMG-PEG | E63 | C9 | Chol | DPhyPE + | DMG-PEG |
| | | | WT-PS | 2000 | | | | DPhyPS + | 2000 |
| | | | | | | | | DHPC | |
| E29 | C2 | Chol | DPhyPE + | DMG-PEG | E64 | C10 | Chol | DPhyPE + | DMG-PEG |
| | | | WT-PS | 2000 | | | | DPhyPS + | 2000 |
| | | | | | | | | DHPC | |
| E30 | C3 | Chol | DPhyPE + | DMG-PEG | E65 | C11 | Chol | DPhyPE + | DMG-PEG |
| | | | WT-PS | 2000 | | | | DPhyPS + | 2000 |
| | | | | | | | | DHPC | |
| E31 | C4 | Chol | DPhyPE + | DMG-PEG | E66 | C12 | Chol | DPhyPE + | DMG-PEG |
| | | | WT-PS | 2000 | | | | DPhyPS + | 2000 |
| | | | | | | | | DHPC | |
| E32 | C5 | Chol | DPhyPE + | DMG-PEG | E67 | C13 | Chol | DPhyPE + | DMG-PEG |
| | | | WT-PS | 2000 | | | | DPhyPS + | 2000 |
| | | | | | | | | DHPC | |
| E33 | C6 | Chol | DPhyPE + | DMG-PEG | E68 | C14 | Chol | DPhyPE + | DMG-PEG |
| | | | WT-PS | 2000 | | | | DPhyPS + | 2000 |
| | | | | | | | | DHPC | |
| E34 | C7 | Chol | DPhyPE + | DMG-PEG | E69 | C15 | Chol | DPhyPE + | DMG-PEG |
| | | | WT-PS | 2000 | | | | DPhyPS + | 2000 |
| | | | | | | | | DHPC | |
| E35 | C8 | Chol | DPhyPE + | DMG-PEG | E70 | C16 | Chol | DPhyPE + | DMG-PEG |
| | | | WT-PS | 2000 | | | | DPhyPS + | 2000 |
| | | | | | | | | DHPC | |
| | | | | | E71 | C17 | Chol | DPhyPE + | DMG-PEG |
| | | | | | | | | DPhyPS + | 2000 |
| | | | | | | | | DHPC | |
| | | | | | E72 | C18 | Chol | DPhyPE + | DMG-PEG |
| | | | | | | | | DPhyPS + | 2000 |
| | | | | | | | | DHPC | |
| | | | | | E73 | C19 | Chol | DPhyPE + | DMG-PEG |
| | | | | | | | | DPhyPS + | 2000 |
| | | | | | | | | DHPC | |
| | | | | | E74 | C20 | Chol | DPhyPE + | DMG-PEG |
| | | | | | | | | DPhyPS + | 2000 |
| | | | | | | | | DHPC | |
| | | | | | E75 | C21 | Chol | DPhyPE + | DMG-PEG |
| | | | | | | | | DPhyPS + | 2000 |
| | | | | | | | | DHPC | |
| | | | | | E76 | C22 | Chol | DPhyPE + | DMG-PEG |
| | | | | | | | | DPhyPS + | 2000 |
| | | | | | | | | DHPC | |
| | | | | | E77 | C23 | Chol | DPhyPE + | DMG-PEG |
| | | | | | | | | DPhyPS + | 2000 |
| | | | | | | | | DHPC | |
| | | | | | E78 | C24 | Chol | DPhyPE + | DMG-PEG |
| | | | | | | | | DPhyPS + | 2000 |
| | | | | | | | | DHPC | |
| | | | | | E79 | C25 | Chol | DPhyPE + | DMG-PEG |
| | | | | | | | | DPhyPS + | 2000 |
| | | | | | | | | DHPC | |
| | | | | | E80 | C26 | Chol | DPhyPE + | DMG-PEG |
| | | | | | | | | DPhyPS + | 2000 |
| | | | | | | | | DHPC | |
| | | | | | E81 | C27 | Chol | DPhyPE + | DMG-PEG |
| | | | | | | | | DPhyPS + | 2000 |
| | | | | | | | | DHPC | |
| | | | | | E82 | C1 | Chol | DPhyPE + | DMG-PEG |
| | | | | | | | | WT-PS + | 2000 |
| | | | | | | | | DHPC | |
| | | | | | E83 | C2 | Chol | DPhyPE + | DMG-PEG |
| | | | | | | | | WT-PS + | 2000 |
| | | | | | | | | DHPC | |
| | | | | | E84 | C3 | Chol | DPhyPE + | DMG-PEG |
| | | | | | | | | WT-PS + | 2000 |
| | | | | | | | | DHPC | |
| | | | | | E85 | C4 | Chol | DPhyPE + | DMG-PEG |
| | | | | | | | | WT-PS + | 2000 |
| | | | | | | | | DHPC | |
| | | | | | E86 | C5 | Chol | DPhyPE + | DMG-PEG |
| | | | | | | | | WT-PS + | 2000 |
| | | | | | | | | DHPC | |
| | | | | | E87 | C6 | Chol | DPhyPE + | DMG-PEG |
| | | | | | | | | WT-PS + | 2000 |
| | | | | | | | | DHPC | |
| | | | | | E88 | C7 | Chol | DPhyPE + | DMG-PEG |
| | | | | | | | | WT-PS + | 2000 |
| | | | | | | | | DHPC | |
| | | | | | E89 | C8 | Chol | DPhyPE + | DMG-PEG |
| | | | | | | | | WT-PS + | 2000 |
| | | | | | | | | DHPC | |
| | | | | | E90 | C9 | Chol | DPhyPE + | DMG-PEG |
| | | | | | | | | WT-PS + | 2000 |
| | | | | | | | | DHPC | |
| | | | | | E91 | C10 | Chol | DPhyPE + | DMG-PEG |
| | | | | | | | | WT-PS + | 2000 |
| | | | | | | | | DHPC | |
| | | | | | E92 | C11 | Chol | DPhyPE + | DMG-PEG |
| | | | | | | | | WT-PS + | 2000 |
| | | | | | | | | DHPC | |
| | | | | | E93 | C12 | Chol | DPhyPE + | DMG-PEG |
| | | | | | | | | WT-PS + | 2000 |
| | | | | | | | | DHPC | |
| | | | | | E94 | C13 | Chol | DPhyPE + | DMG-PEG |
| | | | | | | | | WT-PS + | 2000 |
| | | | | | | | | DHPC | |
| | | | | | E95 | C14 | Chol | DPhyPE + | DMG-PEG |
| | | | | | | | | WT-PS + | 2000 |
| | | | | | | | | DHPC | |
| | | | | | E96 | C15 | Chol | DPhyPE + | DMG-PEG |
| | | | | | | | | WT-PS + | 2000 |
| | | | | | | | | DHPC | |
| | | | | | E97 | C16 | Chol | DPhyPE + | DMG-PEG |
| | | | | | | | | WT-PS + | 2000 |
| | | | | | | | | DHPC | |
| | | | | | E98 | C17 | Chol | DPhyPE + | DMG-PEG |
| | | | | | | | | WT-PS + | 2000 |
| | | | | | | | | DHPC | |
| | | | | | E99 | C18 | Chol | DPhyPE + | DMG-PEG |
| | | | | | | | | WT-PS + | 2000 |
| | | | | | | | | DHPC | |
| | | | | | E100 | C19 | Chol | DPhyPE + | DMG-PEG |
| | | | | | | | | WT-PS + | 2000 |
| | | | | | | | | DHPC | |
| | | | | | E101 | C20 | Chol | DPhyPE + | DMG-PEG |
| | | | | | | | | WT-PS + | 2000 |
| | | | | | | | | DHPC | |
| | | | | | E102 | C21 | Chol | DPhyPE + | DMG-PEG |
| | | | | | | | | WT-PS + | 2000 |
| | | | | | | | | DHPC | |
| | | | | | E103 | C22 | Chol | DPhyPE + | DMG-PEG |
| | | | | | | | | WT-PS + | 2000 |
| | | | | | | | | DHPC | |
| | | | | | E104 | C23 | Chol | DPhyPE + | DMG-PEG |
| | | | | | | | | WT-PS + | 2000 |
| | | | | | | | | DHPC | |
| | | | | | E105 | C24 | Chol | DPhyPE + | DMG-PEG |
| | | | | | | | | WT-PS + | 2000 |
| | | | | | | | | DHPC | |
| | | | | | E106 | C25 | Chol | DPhyPE + | DMG-PEG |
| | | | | | | | | WT-PS + | 2000 |
| | | | | | | | | DHPC | |
| | | | | | E107 | C26 | Chol | DPhyPE + | DMG-PEG |
| | | | | | | | | WT-PS + | 2000 |
| | | | | | | | | DHPC | |
| | | | | | E108 | C27 | Chol | DPhyPE + | DMG-PEG |
| | | | | | | | | WT-PS + | 2000 |
| | | | | | | | | DHPC | |

(Chol = Cholesterol; table split into two tabulars)

In preferred embodiments, the polymer conjugated lipid DMG-PEG 2000 as shown in Table E is replaced with a PMOZ-lipid as shown in FIG. 4, preferably with the polymer conjugated lipid “PMOZ 2”, more preferably with the polymer conjugated lipid “PMOZ 4”, even more preferably with the polymer conjugated lipid “PMOZ 4” with n=50 i.e. having 50 monomer repeats.

Furthermore, preferred lipid formulations showing distinct mol-percentages of the at least five lipid excipients are shown in Table F. For example, a preferred lipid composition comprises the mol-percentages of lipids as disclosed in line “F1”, i.e. 59 mol % cationic lipid, 29.3 mol % sterol, 10 mol % phospholipid combination, and 1.7 mol % polymer conjugated lipid. As another example, a preferred lipid composition comprises the mol-percentages of lipids as disclosed in line “F9”, i.e. 49 mol % cationic lipid, 31 mol % sterol, 20 mol % phospholipid combination and 0 mol % polymer conjugated lipid. The phospholipid combinations may consist of two or three phospholipids in combination, wherein in each case at least one of the phospholipids is a phosphatidylserine.

TABLE F
Formulations incl. mol-percentages for
excipients of preferred compositions

| | | | | polymer | |
|---|---|---|---|---|---|
| | cationic | | Phospholipid | conjugated | |
| Formulation | lipid | sterol | combination | lipid | sum |
| [designation] | [mol %] | [mol %] | [mol %] | [mol %] | [mol %] |
| F1 | 59 | 29.3 | 10 | 1.7 | 100 |
| F2 | 59 | 29.5 | 10 | 1.5 | 100 |
| F3 | 59 | 31 | 10 | 0 | 100 |
| F4 | 58 | 29.3 | 11 | 1.7 | 100 |
| F5 | 58 | 29.5 | 11 | 1.5 | 100 |
| F6 | 58 | 31 | 11 | 0 | 100 |
| F7 | 49 | 29.3 | 20 | 1.7 | 100 |
| F8 | 49 | 29.5 | 20 | 1.5 | 100 |
| F9 | 49 | 31 | 20 | 0 | 100 |
| F10 | 30-70 | 5-25 | 8-25 | 0.5-5 | ** |
| F11 | 40-70 | 5-15 | 8-25 | 0.5-5 | ** |
| F12 | 20-60 | 5-25 | 8-25 | 0.5-15 | ** |
| F13 | 45-65 | 8-12 | 8-26 | 1-3 | ** |

** self-evidently, the sum [mol %] of the last four formulations in Table F, F10, F11, F12 and F13, is defined to be at 100 mol %. I.e. a skilled artisan naturally is able to select a value from the given ranges of the four excipients, so that the mol-percentages for each excipient of preferred compositions of the invention sums up to 100 mol %.

Accordingly, in a further preferred embodiment of the invention, a composition of the invention comprises excipients as disclosed in Table E selected from the group consisting of Excipient combination designation
E1, E2, E3, E4, E5, E6, E7, E8, E9, E10, E11, E12, E13, E14, E15, E16, E17, E18, E19, E20, E21, E22, E23, E24, E25, E26, E27, E28, E29, E30, E31, E32, E33, E34, E35, E36, E37, E38, E39, E40, E41, E42, E43, E44, E45, E46, E47, E48, E49, E50, E51, E52, E53, E54, E55, E56, E57, E58, E59, E60, E61, E62, E63, E64, E65, E66, E67, E68, E69, E70, E71, E72, E73, E74, E75, E76, E77, E78, E79, E80, E81, E82, E83, E84, E85, E86, E87, E88, E89, E90, E91, E92, E93, E94, E95, E96, E97, E98, E99, E100, E101, E102, E103, E104, E105, E106, E107 and E108;
   in distinct mol-percentages as disclosed in Table F selected from the group consisting of formulation designation
F1, F2, F3, F4, F5, F6, F7, F8, F9, F10, F11, F12 and F13.

The zeta potential of a nanoparticle composition may be used to indicate the electrokinetic potential of the composition. For example, the zeta potential may describe the surface charge of a nanoparticle composition. The lipid nanoparticles according to the invention may, due to the presence of both negatively and positively charged compounds, exhibit a relatively neutral zeta potential. The zeta potential (sometimes abbreviated as “charge”) may be determined along with the particle size of the particles, for example, by dynamic light scattering and Laser Doppler Microelectrophoresis, for example using a Malvern Zetasizer Nano (Malvern Instruments Ltd.; Malvern, UK). Depending on the amount and nature of charged compounds in the lipid nanoparticles, the nanoparticles may be characterized by a zeta potential. In a preferred embodiment, the zeta potential is in the range from about −50 mV to about +50 mV. In other preferred embodiments, the zeta potential is in the range from about −25 mV to about +25 mV. In some embodiments, the zeta potential of a lipid nanoparticle of the invention may be from about −10 mV to about +20 mV, from about −10 mV to about +15 mV, from about −10 mV to about +10 mV, from about −10 mV to about +5 mV, from about −10 mV to about 0 mV, from about −10 mV to about −5 mV, from about −5 mV to about +20 mV, from about −5 mV to about +15 mV, from about −5 mV to about +10 mV, from about −5 mV to about +5 mV, from about −5 mV to about 0 mV, from about 0 mV to about +20 mV, from about 0 mV to about +15 mV, from about 0 mV to about +10 mV, from about 0 mV to about +5 mV, from about +5 mV to about +20 mV, from about +5 mV to about +15 mV, or from about +5 mV to about +10 mV.

In certain embodiments, the LNP comprises one or more targeting moieties which are capable of targeting the LNP to a cell or cell population. For example, in one embodiment, the targeting moiety is a ligand which directs the LNP to a receptor found on a cell surface.

In certain embodiments, the LNP comprises one or more internalization domains. For example, in one embodiment, the LNP comprises one or more domains which bind to a cell to induce the internalization of the LNP. For example, in one embodiment, the one or more internalization domains bind to a receptor found on a cell surface to induce receptor-mediated uptake of the LNP. In certain embodiments, the LNP is capable of binding a biomolecule in vivo, where the LNP-bound biomolecule can then be recognized by a cell-surface receptor to induce internalization. For example, in one embodiment, the LNP binds systemic ApoE, which leads to the uptake of the LNP and associated cargo. In certain embodiments of the invention, ApoE may be supplemented to the medium or pharmaceutical composition used.

Preferably, the LNP of the invention comprises
(i) at least one cationic lipid;
(ii) at least one phospholipid;
(iii) at least one steroid or steroid analogue, preferably cholesterol; and
(iv) at least one polymer conjugated lipid, preferably a PEG-lipid;
wherein (i) to (iv) are in a molar ratio of about 20-60% cationic lipid, 5-25% neutral lipid, 25-55% sterol, and 0.5-15% polymer conjugated lipid.

Also, preferably, the LNP of the invention comprises
(i) at least one cationic lipid;
(ii) at least two phospholipids, preferably DPhyPE and phosphatidylserine;
(iii) at least one steroid or steroid analogue, preferably cholesterol; and
(iv) at least one polymer conjugated lipid, preferably a PEG-lipid;
wherein (i) to (iv) are in a molar ratio of about 20-60% cationic lipid, 5-25% neutral lipid, 25-55% sterol, and 0.5-15% polymer conjugated lipid.

Also, preferably, the LNP of the invention comprises
(i) at least one cationic lipid;
(ii) at least three phospholipids, preferably DPhyPE, DHPC and phosphatidylserine;
(iii) at least one steroid or steroid analogue, preferably cholesterol; and
(iv) at least one polymer conjugated lipid, preferably a PEG-lipid;
wherein (i) to (iv) are in a molar ratio of about 20-60% cationic lipid, 5-25% neutral lipid, 25-55% sterol, and 0.5-15% polymer conjugated lipid.

Also, preferably, the LNP of the invention comprises
(i) at least one cationic lipid;
(ii) at least two phospholipids, preferably DSPC and phosphatidylserine;
(iii) at least one steroid or steroid analogue, preferably cholesterol; and
(iv) at least one polymer conjugated lipid, preferably a PEG-lipid;
wherein (i) to (iv) are in a molar ratio of about 20-60% cationic lipid, 5-25% neutral lipid, 25-55% sterol, and 0.5-15% polymer conjugated lipid.

### Encapsulation/Complexation in LNPs

In preferred embodiments, the at least one nucleic acid (e.g. DNA or RNA), preferably the at least one mRNA is complexed, encapsulated, partially encapsulated, or associated with the phospholipid phosphatidylserine and one or more lipids (e.g. cationic lipids and/or neutral lipids), thereby forming liposomes, lipid nanoparticles (LNPs), lipoplexes, and/or nanoliposomes.

The liposomes, lipid nanoparticles (LNPs), lipoplexes, and/or nanoliposomes—incorporated nucleic acid (e.g. DNA or RNA) may be completely or partially located in the interior space of the liposomes, lipid nanoparticles (LNPs), lipoplexes, and/or nanoliposomes, within the lipid layer/membrane, or associated with the exterior surface of the lipid layer/membrane. The incorporation of a nucleic acid into liposomes/LNPs is also referred to herein as “encapsulation” wherein the nucleic acid, e.g. the RNA is entirely contained within the interior space of the liposomes, lipid nanoparticles (LNPs), lipoplexes, and/or nanoliposomes. The purpose of incorporating nucleic acid into liposomes, lipid nanoparticles (LNPs), lipoplexes, and/or nanoliposomes is to protect the nucleic acid, preferably RNA from an environment which may contain enzymes or chemicals or conditions that degrade nucleic acid and/or systems or receptors that cause the rapid excretion of the nucleic acid. Moreover, incorporating nucleic acid, preferably RNA into liposomes, lipid nanoparticles (LNPs), lipoplexes, and/or nanoliposomes may promote the uptake of the nucleic acid, and hence, may enhance the therapeutic effect of the nucleic acid, e.g. the RNA encoding antigenic SARS-CoV-2 (nCoV-2019) proteins. Accordingly, incorporating a nucleic acid, e.g. RNA or DNA, into liposomes, lipid nanoparticles (LNPs), lipoplexes, and/or nanoliposomes may be particularly suitable for a coronavirus vaccine (e.g. a SARS-CoV-2 vaccine), e.g. for intramuscular and/or intradermal administration.

In this context, the terms “complexed” or “associated” refer to the essentially stable combination of nucleic acid with one or more lipids into larger complexes or assemblies without covalent binding.

The term “lipid nanoparticle”, also referred to as “LNP”, is not restricted to any particular morphology, and include any morphology generated when a cationic lipid and optionally one or more further lipids are combined, e.g. in an aqueous environment and/or in the presence of a nucleic acid, e.g. an RNA. For example, a liposome, a lipid complex, a lipoplex and the like are within the scope of a lipid nanoparticle (LNP).

Liposomes, lipid nanoparticles (LNPs), lipoplexes, and/or nanoliposomes can be of different sizes such as, but not limited to, a multilamellar vesicle (MLV) which may be hundreds of nanometers in diameter and may contain a series of concentric bilayers separated by narrow aqueous compartments, a small unicellular vesicle (SUV) which may be smaller than 50 nm in diameter, and a large unilamellar vesicle (LUV) which may be between 50 nm and 500 nm in diameter.

LNPs of the invention are suitably characterized as microscopic vesicles having an interior aqua space sequestered from an outer medium by a membrane of one or more bilayers. Bilayer membranes of LNPs are typically formed by amphiphilic molecules, such as lipids of synthetic or natural origin that comprise spatially separated hydrophilic and hydrophobic domains. Bilayer membranes of the liposomes can also be formed by amphophilic polymers and surfactants (e.g., polymerosomes, niosomes, etc.). In the context of the present invention, an LNP typically serves to transport the at least one nucleic acid, preferably the at least one RNA to a target tissue. Accordingly, in preferred embodiments of the aspects of the invention, the at least one RNA is complexed with one or more lipids thereby forming lipid nanoparticles (LNP). LNPs typically comprise a cationic lipid and one or more excipients selected from neutral lipids, charged lipids, steroids and polymer conjugated lipids.

The mRNA

In one of the preferred embodiments, the nucleic acid compound is an mRNA or an mRNA compound. As has been found by the inventors, the vaccine compositions comprising a carrier composition comprising the phospholipid phosphatidylserine according to the present invention are particularly suitable for the in vivo delivery of mRNA compounds expressing antigens, and thus enable highly effective, potent, versatile and safe vaccines that can be rapidly developed at moderate cost. Specific antigens of interest for carrying out the present invention are described in more detail below. The mRNA compound according to the invention is preferably encapsulated in or associated with a lipid nanoparticle.

Advantages of the phosphatidylserine-containing vaccines comprising mRNA encoding at least one antigen or fragment or variant thereof are:
targeting to the spleen
Induction of a strong humoral immune response
Induction of B-cell memory
Faster onset of immune protection
Longevity of the induced immune responses
Induction of broad cellular T-cell responses
Induction of a (local and transient) pro-inflammatory environment
Good tolerability, no side-effects, non-toxic
Advantageous stability characteristics
Formulation compatible with many different antigens: larger antigen cocktails feasible based on the same (production) technology
No vector immunity, i.e. technology can be used to vaccinate the same subject multiple times against multiple (different) antigens
Speed, adaptability, simplicity and scalability of production
preferred patterns of induction of systemic cytokine or chemokine responses depending on the respective application.

In certain embodiments, the lipid nanoparticle composition comprises apart from the phospholipid phosphatidylserine:
(a) a cationic or ionizable lipid;
(b) a steroid;
(c) a further phospholipid in addition to phosphatidylserine;
(d) a polymer conjugated lipid; and/or
(e) an mRNA compound encoding an antigen or fragment or variant thereof.

In other embodiments, the lipid nanoparticle composition comprises apart from the phospholipid phosphatidylserine:
(a) a cationic or ionizable lipid;
(b) a steroid;
(c) a polymer conjugated lipid; and
(d) an mRNA compound encoding an antigen or fragment or variant thereof.

In other embodiments, the lipid nanoparticle composition comprises apart from the phospholipid phosphatidylserine:
(a) a cationic or ionizable lipid;
(b) a further phospholipid in addition to phosphatidylserine;
(c) a polymer conjugated lipid; and
(d) an mRNA compound encoding an antigen or fragment or variant thereof.

In certain preferred embodiments, the lipid nanoparticle composition comprises apart from the phospholipid phosphatidylserine:
(a) a cationic or ionizable lipid;
(b) a steroid;
(c) a further phospholipid in addition to phosphatidylserine;
(d) a polymer conjugated lipid; and
(e) an mRNA compound encoding an antigen or fragment or variant thereof.

With respect to the phospholipid phosphatidylserine, the cationic or ionizable lipid, the steroid, the phospholipid, the polymer conjugated lipid, and the mRNA compound encoding an antigen, the same options, preferences and alternatives apply as have been described with respect to these features herein above.

The amount of the cationic or ionizable lipid relative to that of the mRNA compound in the lipid nanoparticle may also be expressed as a weight ratio (abbreviated e.g. “m/m”). For example, the lipid nanoparticles comprise the mRNA compound at an amount such as to achieve a lipid to mRNA weight ratio in the range of about 20 to about 60, or about 10 to about 50. In other embodiments, the ratio of cationic or ionizable lipid to nucleic acid or mRNA is from about 3 to about 15, such as from about 5 to about 13, from about 4 to about 8 or from about 7 to about 11. In a very preferred embodiment of the present invention, the total lipid/mRNA mass ratio is about 40 or 40, i.e. about 40 or 40 times mass excess to ensure mRNA encapsulation. Another preferred RNA/lipid ratio is between about 1 and about 10, about 2 and about 5, about 2 and about 4, or preferably about 3.

Further, the amount of the cationic or ionizable lipid may be selected taking the amount of the nucleic acid cargo such as the mRNA compound into account. In one embodiment, the N/P ratio can be in the range of about 1 to about 50. In another embodiment, the range is about 1 to about 20, about 1 to about 10, about 1 to about 5. In one preferred embodiment, these amounts are selected such as to result in an N/P ratio of the lipid nanoparticles or of the composition in the range from about 10 to about 20. In a further very preferred embodiment, the N/P is 14 (i.e. 14 times mol excess of positive charge to ensure mRNA encapsulation). In other very preferred embodiments, the N/P is 17.5 (i.e. 17.5 times mol excess of positive charge to ensure mRNA encapsulation) or
(i) at an amount such as to achieve an N/P ratio in the range of about 1 to about 20, preferably about 2 to about 15, more preferably about 3 to about 10, even more preferably about 4 to about 9, most preferably about 6;
(ii) at an amount such as to achieve an N/P ratio in the range of about 5 to about 20, more preferably about 10 to about 18, even more preferably about 12 to about 16, most preferably about 14;
or
(iii) at an amount such as to achieve a lipid: mRNA weight ratio in the range of 20 to 60, preferably from about 3 to about 15, 5 to about 13, about 4 to about 8 or from about 7 to about 11

In other preferred embodiments, the N/P ratio can be in the range of about 1 to about 50. In other embodiments, the range is about 1 to about 20, and preferably about 1 to about 15. For the inventive lipid nanoparticles, a preferred N/P (lipid to RNA mol ratio) is about 14 or about 17. A further preferred N/P i.e. lipid to RNA mol ratio is about 6. Another preferred N/P ratio is about 4.85 or 5 (lipid to RNA mol ratio).

The total amount of mRNA in the lipid nanoparticles varies and may be defined depending on the mRNA to total lipid w/w ratio. In one embodiment of the invention the invention the mRNA to total lipid ratio is less than 0.06 w/w, preferably between 0.03 and 0.04 w/w.

Preferably, the mRNA compound or the coding sequence thereof has a length of about 50 to about 20000, or 100 to about 20000 nucleotides, preferably of about 250 to about 20000 nucleotides, more preferably of about 500 to about 10000, even more preferably of about 500 to about 5000.

### Pathogenic or Pathogen-Derived Antigens

Pathogenic antigens or pathogen-derived antigens are derived from pathogenic organisms, in particular bacterial, viral or protozoological (multicellular) pathogenic organisms, which evoke an immunological reaction by subject, in particular a mammalian subject, more particularly a human. More specifically, pathogenic antigens are preferably surface antigens, e.g. proteins (or fragments of proteins, e.g. the exterior portion of a surface antigen) located at the surface of the virus or the bacterial or protozoological organism.

Accordingly, in some preferred embodiments, the mRNA may encode in its at least one coding region at least one pathogenic antigen selected from a bacterial, viral, fungal or protozoal antigen. The encoded (poly-)peptide or protein may consist or comprise of a pathogenic antigen or a fragment or variant thereof.

Pathogenic antigens are peptide or protein antigens preferably derived from a pathogen associated with an infectious disease which are preferably selected from, but not limited to, the group of antigens derived from the pathogens disclosed on pages 21-35 in WO2018078053; WO2018078053 being incorporated herein by reference in its entirety. Furthermore, pathogenic antigens are peptide or protein antigens preferably derived from a pathogen associated with an infectious disease which are preferably selected from, but not limited to, the group of antigens derived from the pathogens disclosed on page 57 paragraph 3—page 63, paragraph 2 in WO2019077001; WO2019077001 being incorporated herein by reference in its entirety.

Even further pathogenic antigens are peptide or protein antigens preferably derived from a pathogen associated with infectious disease which are preferably selected from antigens derived from the pathogens selected from, but not limited to, the group of antigens derived from the pathogens disclosed on pages 32 line 26—page 34 line 27 in WO2013120628. Furthermore in this regard, the pathogenic antigen (antigen derived from a pathogen associated with infectious disease) may be preferably selected from the antigens preferably selected from antigens selected from, but not limited to, the group of antigens as disclosed on pages 34 line 29—page 59 line 5 (in brackets is the particular pathogen or the family of pathogens of which the antigen(s) is/are derived and the infectious disease with which the pathogen is associated) in WO2013120628; WO2013120628 being incorporated herein by reference in its entirety.

Among the preferred antigens expressed by the mRNA compound incorporated in the composition of the invention are pathogens selected from, but not limited to, the group consisting of a SARS coronavirus 2 (SARS-CoV-2), nCoV-2019 coronavirus, SARS coronavirus (SARS-CoV), Bunyavirales virus, Cytomegalovirus (CMV), Dengue viruses (DENV-1, DENV-2, DENV-3 and DENV-4), Ebola virus (EBOV), Flavivirus, Hepatitis B virus (HBV), Herpes simplex virus (HSV), Human immunodeficiency virus (HIV), Human metapneumovirus (HMPV), Human Papilloma virus (HPV), Human parainfluenza viruses (HPIV), Influenza virus, extraintestinal pathogenic E. coli (ExPEC), Lassa mammarenavirus (LASV), MERS coronavirus, Mycobacterium tuberculosis, Nipah virus, Norovirus, Rabies virus (RABV), Respiratory Syncytial virus (RSV), Rhinovirus, Rotavirus, Vaccinia virus, Yellow Fever virus (YFV), Zika virus (ZIKV), Chlamydia trachomatis (i.e. bacterium Chlamydia causing Chlamydia), and Malaria parasites (e.g. Plasmodium falciparum, Plasmodium vivax, Plasmodium malariae, or Plasmodium ovale). In another one of the preferred embodiments, the pathogenic antigen is derived from a SARS coronavirus 2 (SARS-CoV-2), nCoV-2019 coronavirus, SARS coronavirus (SARS-CoV), Malaria parasite, an Influenza virus or a Rabies virus (RABV).

Further, pathogenic antigens may further preferably be selected from antigens derived from the pathogens selected from, but not limited to, the group consisting of Acinetobacter baumannii, Anaplasma genus, Anaplasma phagocytophilum, Ancylostoma braziliense, Ancylostoma duodenale, Arcanobacterium haemolyticum, Ascaris lumbricoides, Aspergillus genus, Astroviridae, Babesia genus, Bacillus anthracis, Bacillus cereus, Bartonella henselae, BK virus, Blastocystis hominis, Blastomyces dermatitidis, Bordetella pertussis, Borrelia burgdorferi, Borrelia genus, Borrelia spp, Brucella genus, Brugia malayi, Bunyaviridae family, Burkholderia cepacia or other Burkholderia species, Burkholderia mallei, Burkholderia pseudomallei, Caliciviridae family, Campylobacter genus, Candida albicans, Candida spp, Chlamydia trachomatis, Chlamydophila pneumoniae, Chlamydophila psittaci, CJD prion, Clonorchis sinensis, Clostridium botulinum, Clostridium difficile, Clostridium perfringens, Clostridium perfringens, Clostridium spp, Clostridium tetani, Coccidioides spp, Coronaviruses, Coronaviridae family, Corynebacterium diphtheriae, Coxiella burnetii, Crimean-Congo haemorrhagic fever virus, Cryptococcus neoformans, Cryptosporidium genus, Cytomegalovirus (CMV), Dientamoeba fragilis, Ebola virus (EBOV—for example the envelope glycoprotein), Echinococcus genus, Ehrlichia chaffeensis, Ehrlichia ewingii, Ehrlichia genus, Entamoeba histolytica, Enterococcus genus, Enterovirus genus, Enteroviruses, mainly Coxsackie A virus or Enterovirus 71 (EV71), Epidermophyton spp, Epstein-Barr virus (EBV), Escherichia coli, Escherichia coli strains O157:H7, O111 or O104:H4, Fasciola hepatica or Fasciola gigantica, FFI prion, Feline immunodeficiency virus (FIV), Filarioidea superfamily, Flaviviruses, Francisella tularensis, Fusobacterium genus, Geotrichum candidum, Giardia intestinalis, Gnathostoma spp, GSS prion, Guanarito virus, Haemophilus ducreyi, Haemophilus influenzae, Helicobacter pylori, Henipavirus (Hendra virus, Nipah virus), Hepatitis A virus (HAV), Hepatitis B virus (HBV), Hepatitis C virus (HCV), Hepatitis D virus, Hepatitis E virus, Histoplasma capsulatum, Hortaea werneckii, Human bocavirus (HBoV), Human metapneumovirus (HMPV), Human parainfluenza viruses (HPIV), Japanese encephalitis virus, JC virus, Junin virus, Kingella kingae, Klebsiella granulomatis, Klebsiella pneumoniae, Kuru prion, Lassa virus, Legionella pneumophila, Leishmania genus, Leptospira genus, Listeria monocytogenes, Lymphocytic choriomeningitis virus (LCMV), Machupo virus, Malassezia spp, Marburg virus, Measles virus, Metagonimus yokagawai, Microsporidia phylum, Molluscum contagiosum virus (MCV), Mumps virus, Mycobacterium leprae or Mycobacterium lepromatosis, Mycobacterium tuberculosis, Mycobacterium ulcerans, Mycoplasma pneumoniae, Naegleria fowleri, Necator americanus, Neisseria gonorrhoeae, Neisseria meningitidis, Nocardia asteroides, Nocardia spp, Onchocerca volvulus, Orientia tsutsugamushi, Orthomyxoviridae family (Influenza), Paracoccidioides brasiliensis, Paragonimus spp, Paragonimus westermani, Parvovirus B19, Pasteurella genus, Plasmodium genus, Pneumocystis jirovecii, Poliovirus, Rhinovirus, rhinoviruses, Rickettsia akari, Rickettsia genus, Rickettsia prowazekii, Rickettsia rickettsii, Rickettsia typhi, Rift Valley fever virus, Rotavirus (preferably e.g. VP8 antigen), Rubella virus, Sabia virus, Salmonella genus, Sarcoptes scabiei, SARS coronavirus, Schistosoma genus, Shigella genus, Sin Nombre virus, Hantavirus, Sporothrix schenckii, Staphylococcus genus, Staphylococcus aureus, Streptococcus agalactiae, Streptococcus pneumoniae, Streptococcus pyogenes, Strongyloides stercoralis, Taenia genus, Taenia solium, Tick-borne encephalitis virus (TBEV), Toxocara canis or Toxocara cati, Toxoplasma gondii, Treponema pallidum, Trichinella spiralis, Trichomonas vaginalis, Trichophyton spp, Trichuris trichiura, Trypanosoma brucei, Trypanosoma cruzi, Ureaplasma urealyticum, Vaccinia virus (preferably e.g. immune evasion proteins E3, K3, or B18), Varicella zoster virus (VZV), Variola major or Variola minor, vCJD prion, Venezuelan equine encephalitis virus, Vibrio cholerae, West Nile virus, Western equine encephalitis virus, Wuchereria bancrofti, Yersinia enterocolitica, Yersinia pestis, or Yersinia pseudotuberculosis, Zika virus (ZIKV), Zika virus strains ZikaSPH2015-Brazil, Z1106033-Suriname, MR766-Uganda or Natal RGN, or an isoform, homolog, fragment, variant or derivative of any of these proteins, preferably from the Coronaviridae family.

In a further embodiment, antigens useful for treating infections—i.e. by administering nucleic acids, preferably mRNA, encoding said antigens—may be selected from the following antigens (the related infection and related pathogen are indicated in brackets after the respective antigens—naturally, also other antigens which may be derived from the following pathogens in brackets may be derived and used according to the invention):
spike protein (S), an envelope protein (E), a membrane protein (M) or a nucleocapsid protein (N), or an immunogenic fragment or variant of any of these (infectious disease is “COVID-19 disease”; pathogen: SARS coronavirus 2 (SARS-CoV-2), nCoV-2019 coronavirus, SARS coronavirus (SARS-CoV));
spike protein (S), a spike S1 fragment (S1), an envelope protein (E), a membrane protein (M) or a nucleocapsid protein (N) (infectious disease is MERS infection; pathogen: Middle East respiratory syndrome coronavirus (MERS coronavirus/MERS-CoV));
replication protein E1, regulatory protein E2, protein E3, protein E4, protein E5, protein E6, protein E7, protein E8, major capsid protein L1, minor capsid protein L2 (infectious disease is Human papillomavirus (HPV) infection; pathogen: Human papillomavirus (HPV) or HPV16);
fusion protein F, hemagglutinin-neuramidase HN, glycoprotein G, matrix protein M, phosphoprotein P, nucleoprotein N, polymerase L, hemagglutinin-neuraminidase, fusion (F) glycoprotein F0, F1 or F2, Recombinant HPIV-3/HPIV-1 fusion glycoprotein (F) and hemagglutinin (HN), C protein, phosphoprotein, D protein, matrix protein (M), nucleocapsid protein (N), viral replicase (L), non-structural V protein (infectious disease is Human parainfluenza virus infection; pathogen: Human parainfluenza viruses (HPIV/PIV) HPIV-1, HPIV-2, HPIV-3, or HPIV-4 serotype, preferably HPIV-3 serotype, preferably PIV3);
fusion (F) glycoprotein, glycoprotein G, phosphoprotein P, nucleoprotein N, nucleocapsid protein (infectious disease: HMPV infection; pathogen: Human metapneumovirus (HMPV));
hemagglutinin (HA), neuraminidase (NA), nucleoprotein (NP), M1 protein, M2 protein, NS1 protein, NS2 protein (NEP protein: nuclear export protein), PA protein, PB1 protein (polymerase basic 1 protein), PB1-F2 protein and PB2 protein, H10N8, H7N9, H10, H1N1, H3N2 (X31), H1, H2, H3, H4, H5, H6, H7, H8, H9, H10, H11, H12, H13, H14, H15, H16, H17, H18, antigenicsubdomains of HA: HA1, HA2, neuraminidase (NA), nucleoprotein (NP), matrix protein 1 (M1), matrix protein 2 (M2), non-structural protein 1 (NS1), nonstructural protein 2 (NS2), HA7 antigen, H7 or H10 and B, pathogen: Orthomyxoviridae family, Influenza virus (fFLU) or Influenza A virus (FLUAV) and/or Influenza B virus (FLUBV));
nucleoprotein N, large structural protein L, phosphoprotein P, matrix protein M, glycoprotein G, G protein (infectious disease is Rabies; pathogen: Rabies virus (RABV));
p24 antigen, envelope proteins (Gp120, Gp41, Gp160), polyprotein GAG, negative factor protein Nef, trans-activator of transcription Tat, Brec1 (infectious disease HIV; pathogen: Human immunodeficiency virus (HIV));
major outer membrane protein MOMP, probable outer membrane protein PMPC, outer membrane complex protein B OmcB, heat shock proteins Hsp60 HSP10, protein IncA, proteins from the type III secretion system, ribonucleotide reductase small chain protein NrdB, plasmid protein Pgp3, Chlamydial outer protein N CopN, antigen CT521, antigen CT425, antigen CT043, antigen TC0052, antigen TC0189, antigen TC0582, antigen TC0660, antigen TC0726, antigen TC0816, antigen TC0828 (infectious disease: infection with Chlamydia trachomatis; pathogen: Chlamydia trachomatis);
pp65 antigen, membrane protein pp15, capsid-proximal tegument protein pp150, protein M45, DNA polymerase UL54, helicase UL105, glycoprotein gM, glycoprotein gN, glycoprotein H, glycoprotein B gB, protein UL83, protein UL94, protein UL99, HCMV glycoprotein selected from gH gL, gB, gO, gN, and gM, HCMV protein selected from UL83, UL123, UL128, UL130 and UL131A, Tegument protein pp150 (pp150), Tegument protein pp65/lower matrix phosphoprotein (pp65), Envelope glycoprotein M (UL100), Regulatory protein IE1 (UL123), Envelopeprotein (UL128), Envelope glycoprotein (130), Envelopeprotein (UL131A), Envelope glycoprotein B (UL55), Structural glycoprotein N gpUL73 (UL73), Structural glycoprotein O gpUL74 (UL74) (infectious disease is Cytomegalovirus infection; pathogen: Cytomegalovirus (CMV/HCMV));
capsid protein C, premembrane protein prM, membrane protein M, envelope protein E (domain I, domain II, domain II), protein NS1, protein NS2A, protein NS2B, protein NS3, protein NS4A, protein 2K, protein NS4B, protein NS5 (infectious disease Dengue fever; pathogen: Dengue viruses (DENV-1, DENV-2, DENV-3 and DENV-4));
glycoprotein (GP), surface GP, wild type pro-GP, mature GP, secreted wild type pro-GP, secreted mature GP, nucleoprotein (NP), RNA polymerase L, and matrix protein selected from VP35, VP40, VP24, and VP30 (infectious disease: Ebola; pathogen: Ebola virus (EBOV));
hepatitis B surface antigen HBsAg, Hepatitis B core antigen HbcAg, polymerase, protein Hbx, preS2 middle surface protein, surface protein L, large S protein, virus protein VP1, virus protein VP2, virus protein VP3, virus protein VP4 (infectious disease is Hepatitis B; pathogen: Hepatitis B Virus (HBV));
fusionprotein F, F protein, nucleoprotein N, matrix protein M, matrix protein M2-1, matrix protein M2-2, phophoprotein P, small hydrophobic protein SH, major surface glycoprotein G, polymerase L, non-structural protein 1 NS1, non-structural protein 2 NS2, RSV attachment protein (G) (glycoprotein G), fusion (F) glycoprotein (glycoprotein F), nucleoprotein (N), phosphoprotein (P), large polymerase protein (L), matrix protein (M, M2), small hydrophobic protein (SH), nonstructural protein 1 (NS1), nonstructural protein 2 (NS2), membrane-bound RSV F protein, membrane-bound DS-Cavl (stabilized prefusion RSV F protein) (infectious disease is infection with Respiratory syncytial virus (RSV); pathogen: Respiratory syncytial virus (RSV));
secretory antigen SssA (Staphylococcus genus, Staphylococcal food poisoning); secretory antigen SssA (Staphylococcus genus e.g. aureus, Staphylococcal infection);
molecular chaperone DnaK, cell surface lipoprotein Mpt83, lipoprotein P23, phosphate transport system permease protein pstA, 14 kDa antigen, fibronectin-binding protein C FbpC1, Alanine dehydrogenase TB43, Glutamine synthetase 1, ESX-1 protein, protein CFP10, TB10.4 protein, protein MPT83, protein MTB12, protein MTB8, Rpf-like proteins, protein MTB32, protein MTB39, crystallin, heat-shock protein HSP65, protein PST-S (infectious disease is Tuberculosis; pathogen: Mycobacterium tuberculosis);
genome polyprotein, protein E, protein M, capsid protein C, protease NS3, protein NS1, protein NS2A, protein AS2B, protein NS4A, protein NS4B, protein NS5 (infectious disease is Yellow fever; pathogen: Yellow fever virus (YFV));
circumsporozoite protein (CSP) (infectious disease is Malaria; pathogen: P. falciparum and P. vivax); and
Zika virus proteins in accordance with WO2017140905, i.e. capsid protein (C), premembrane protein (prM), pr protein (pr), membrane protein (M), envelope protein (E), non-structural protein, prME antigen, capsid protein, premembrane/membrane protein, ZIKV non-structural protein 1, non-structural protein 2A, non-structural protein 2B, non-structural protein 3, non-structural protein 4A, non-structural protein 4B, non-structural protein 5, or a Zika virus envelope protein (E) wherein the fusion loop in domain II is mutated in accordance with WO2017140905; WO2017140905 being incorporated herein by reference in its entirety (infectious disease is Zika virus infection; pathogen: Zika virus (ZIKV)).

In another embodiment, antigens useful for treating infections—i.e. by administering nucleic acids, preferably mRNA, encoding said antigens—may be selected from a pathogenic antigen, preferably selected from the group consisting of a tumor antigen, a viral antigen, a bacterial antigen, and a protozoan antigen.

In some embodiments of the present invention, disclosure is provided for methods of inducing an antigen specific immune response in a subject, comprising administering to the subject any of the RNA (e.g. mRNA) vaccine as provided herein in an amount effective to produce an antigen-specific immune response.

In some embodiments, the RNA (e.g. mRNA) vaccine is a SARS coronavirus 2 (SARS-CoV-2), nCoV-2019 coronavirus, SARS coronavirus (SARS-CoV), Bunyavirales virus, Cytomegalovirus (CMV), Dengue viruses (DENV-1, DENV-2, DENV-3 and DENV-4), Ebola virus (EBOV), Flavivirus, Hepatitis B virus (HBV), Herpes simplex virus (HSV), Human immunodeficiency virus (HIV), Human metapneumovirus (HMPV), Human Papilloma virus (HPV), Human parainfluenza viruses (HPIV), Influenza virus, extraintestinal pathogenic E. coli (ExPEC), Lassa mammarenavirus (LASV), MERS coronavirus, Mycobacterium tuberculosis, Nipah virus, Norovirus, Rabies virus (RABV), Respiratory Syncytial virus (RSV), Rhinovirus, Rotavirus, Vaccinia virus, Yellow Fever virus (YFV), Zika virus (ZIKV), Chlamydia trachomatis (i.e. bacterium Chlamydia causing Chlamydia), or Malaria parasite (e.g. Plasmodium falciparum, Plasmodium vivax, Plasmodium malariae, or Plasmodium ovale) vaccine. In other embodiments, the RNA (e.g. mRNA) vaccine is a rabies, an influenza or a malaria vaccine.

In some embodiments, the RNA (e.g., mRNA) vaccine is a combination vaccine comprising a combination of influenza vaccines (a broad spectrum influenza vaccine). In some embodiments, an antigen-specific immune response comprises a T cell response or a B cell response.

In some embodiments, a method of producing an antigen-specific immune response comprises administering to a subject a single dose (i.e. no booster dose) of an SARS coronavirus 2 (SARS-CoV-2), nCoV-2019 coronavirus, SARS coronavirus (SARS-CoV), Bunyavirales virus, Cytomegalovirus (CMV), Dengue viruses (DENV-1, DENV-2, DENV-3 and DENV-4), Ebola virus (EBOV), Flavivirus, Hepatitis B virus (HBV), Herpes simplex virus (HSV), Human immunodeficiency virus (HIV), Human metapneumovirus (HMPV), Human Papilloma virus (HPV), Human parainfluenza viruses (HPIV), Influenza virus, extraintestinal pathogenic E. coli (ExPEC), Lassa mammarenavirus (LASV), MERS coronavirus, Mycobacterium tuberculosis, Nipah virus, Norovirus, Rabies virus (RABV), Respiratory Syncytial virus (RSV), Rhinovirus, Rotavirus, Vaccinia virus, Yellow Fever virus (YFV), Zika virus (ZIKV), Chlamydia trachomatis (i.e. bacterium Chlamydia causing Chlamydia), or Malaria parasite (e.g. Plasmodium falciparum, Plasmodium vivax, Plasmodium malariae, or Plasmodium ovale) RNA (e.g., mRNA) vaccine of the present disclosure.

In some embodiments, a method further comprises administering to the subject a second (booster) dose of an SARS coronavirus 2 (SARS-CoV-2), nCoV-2019 coronavirus, SARS coronavirus (SARS-CoV), Bunyavirales virus, Cytomegalovirus (CMV), Dengue viruses (DENV-1, DENV-2, DENV-3 and DENV-4), Ebola virus (EBOV), Flavivirus, Hepatitis B virus (HBV), Herpes simplex virus (HSV), Human immunodeficiency virus (HIV), Human metapneumovirus (HMPV), Human Papilloma virus (HPV), Human parainfluenza viruses (HPIV), Influenza virus, extraintestinal pathogenic E. coli (ExPEC), Lassa mammarenavirus (LASV), MERS coronavirus, Mycobacterium tuberculosis, Nipah virus, Norovirus, Rabies virus (RABV), Respiratory Syncytial virus (RSV), Rhinovirus, Rotavirus, Vaccinia virus, Yellow Fever virus (YFV), Zika virus (ZIKV), Chlamydia trachomatis (i.e. bacterium Chlamydia causing Chlamydia), or Malaria parasite (e.g. Plasmodium falciparum, Plasmodium vivax, Plasmodium malariae, or Plasmodium ovale) RNA (e.g. mRNA) vaccine may be administered.

In some embodiments, the subjects exhibit a seroconversion rate of at least 80% (e.g., at least 85%, at least 90%, or at least 95%) following the first dose or the second (booster) dose of the vaccine. Seroconversion is the time period during which a specific antibody develops and becomes detectable in the blood. After seroconversion has occurred, a virus can be detected in blood tests for the antibody. During an infection or immunization, antigens enter the blood, and the immune system begins to produce antibodies in response. Before seroconversion, the antigen itself may or may not be detectable, but antibodies are considered absent. During seroconversion, antibodies are present but not yet detectable. Any time after seroconversion, the antibodies can be detected in the blood, indicating a prior or current infection. In some embodiments, an RNA (e.g., mRNA) vaccine is administered to a subject by intradermal injection, intramuscular injection, or by intranasal administration. In some embodiments, an RNA (e.g. mRNA) vaccine is administered to a subject by intramuscular injection.

Some embodiments, of the present disclosure provide methods of inducing an antigen specific immune response in a subject, including administering to a subject an SARS coronavirus 2 (SARS-CoV-2), nCoV-2019 coronavirus, SARS coronavirus (SARS-CoV), Bunyavirales virus, Cytomegalovirus (CMV), Dengue viruses (DENV-1, DENV-2, DENV-3 and DENV-4), Ebola virus (EBOV), Flavivirus, Hepatitis B virus (HBV), Herpes simplex virus (HSV), Human immunodeficiency virus (HIV), Human metapneumovirus (HMPV), Human Papilloma virus (HPV), Human parainfluenza viruses (HPIV), Influenza virus, extraintestinal pathogenic E. coli (ExPEC), Lassa mammarenavirus (LASV), MERS coronavirus, Mycobacterium tuberculosis, Nipah virus, Norovirus, Rabies virus (RABV), Respiratory Syncytial virus (RSV), Rhinovirus, Rotavirus, Vaccinia virus, Yellow Fever virus (YFV), Zika virus (ZIKV), Chlamydia trachomatis (i.e. bacterium Chlamydia causing Chlamydia), or Malaria parasite (e.g. Plasmodium falciparum, Plasmodium vivax, Plasmodium malariae, or Plasmodium ovale) RNA (e.g., mRNA) vaccine in an effective amount to produce an antigen specific immune response in a subject.

Antigen-specific immune responses in a subject may be determined, in some embodiments, by assaying for antibody titer (for titer of an antibody that binds to an SARS coronavirus 2 (SARS-CoV-2), nCoV-2019 coronavirus, SARS coronavirus (SARS-CoV), Bunyavirales virus, Cytomegalovirus (CMV), Dengue viruses (DENV-1, DENV-2, DENV-3 and DENV-4), Ebola virus (EBOV), Flavivirus, Hepatitis B virus (HBV), Herpes simplex virus (HSV), Human immunodeficiency virus (HIV), Human metapneumovirus (HMPV), Human Papilloma virus (HPV), Human parainfluenza viruses (HPIV), Influenza virus, extraintestinal pathogenic E. coli (ExPEC), Lassa mammarenavirus (LASV), MERS coronavirus, Mycobacterium tuberculosis, Nipah virus, Norovirus, Rabies virus (RABV), Respiratory Syncytial virus (RSV), Rhinovirus, Rotavirus, Vaccinia virus, Yellow Fever virus (YFV), Zika virus (ZIKV), Chlamydia trachomatis (i.e. bacterium Chlamydia causing Chlamydia), or Malaria parasite (e.g. Plasmodium falciparum, Plasmodium vivax, Plasmodium malariae, or Plasmodium ovale) antigenic polypeptide) following administration to the subject of any of the Bunyavirales virus, Cytomegalovirus (CMV), Dengue viruses (DENV-1, DENV-2, DENV-3 and DENV-4), Ebola virus (EBOV), Flavivirus, Hepatitis B virus (HBV), Herpes simplex virus (HSV), Human immunodeficiency virus (HIV), Human metapneumovirus (HMPV), Human Papilloma virus (HPV), Human parainfluenza viruses (HPIV), Influenza virus, extraintestinal pathogenic E. coli (ExPEC), Lassa mammarenavirus (LASV), MERS coronavirus, Mycobacterium tuberculosis, Nipah virus, Norovirus, Rabies virus (RABV), Respiratory Syncytial virus (RSV), Rhinovirus, Rotavirus, Vaccinia virus, Yellow Fever virus (YFV), Zika virus (ZIKV), Chlamydia trachomatis (i.e. bacterium Chlamydia causing Chlamydia), or Malaria parasite (e.g. Plasmodium falciparum, Plasmodium vivax, Plasmodium malariae, or Plasmodium ovale) RNA (e.g., mRNA) vaccines of the present disclosure. In some embodiments, the anti-antigenic polypeptide antibody titer produced in the subject is increased by at least 1 log relative to a control. In some embodiments, the anti-antigenic polypeptide antibody titer produced in the subject is increased by 1-3 log relative to a control.

### Tumor Antigens

In a further preferred embodiment, the mRNA encodes a tumor antigen, preferably as defined herein, or a fragment or variant thereof, wherein the tumor antigen is preferably selected from, but not limited to, the group consisting of tumor antigens disclosed on pages 47-51 in WO2018078053; WO2018078053 being incorporated herein by reference in its entirety.

Furthermore, cytokines, chemokines, suicide enzymes and gene products, apoptosis inducers, endogenous angiogenesis inhibitors, heat shock proteins, tumor antigens, innate immune activators, antibodies directed against proteins associated with tumor or cancer development, useful for the present invention e.g. for cancer treatment, are selected from, but not limited to, the group of cytokines, chemokines, suicide enzymes and gene products, apoptosis inducers, endogenous angiogenesis inhibitors, heat shock proteins, tumor antigens, innate immune activators, antibodies directed against proteins associated with tumor or cancer development as disclosed in Table 1, Table 2, Table 3, Table 4, Table 5, Table 6, Table 7, Table 8, Table 9, Table 10, Table 11 and Table 12 of WO2016170176; WO2016170176 and especially Tables 1-12 being specifically incorporated herein by reference in its entirety.

### Other Antigens

Further antigens useful for the present invention are listed in WO2018078053 on pages 48-51; WO2018078053 being incorporated herein by reference in its entirety.

### Allergenic Antigens and Autoimmune Self-Antigens

As mentioned, the mRNA may encode an antigen that represents an allergen, or an allergenic antigen or a self-antigen, also referred to as autoantigen or autoimmune antigen. Such antigens and self-antigens associated with allergy or allergic disease (allergens or allergenic antigens) are derived from or preferably selected from, but not limited to, the group of antigens disclosed on pages 59-73 in WO2018078053; WO2018078053 being incorporated herein by reference in its entirety.

Molecular Therapy—Enzyme Replacement Therapy—mRNA Replacement Therapy

In other preferred embodiments, the nucleic acid compound is an mRNA comprising at least one coding region encoding a therapeutic protein replacing an absent, deficient or mutated protein; a therapeutic protein beneficial for treating inherited or acquired diseases; infectious diseases, or neoplasms e.g. cancer or tumor diseases); an adjuvant or immuno-stimulating therapeutic protein; a therapeutic antibody or an antibody fragment, variant or derivative; a peptide hormone; a gene editing agent; an immune checkpoint inhibitor; a T cell receptor, or a fragment, variant or derivative T cell receptor; and/or an enzyme. In another embodiment, the peptide or protein expressed by the nucleic acid compound is a therapeutic protein, or a fragment or variant thereof, wherein the therapeutic protein is beneficial for the treatment or prophylaxis of any inherited or acquired disease or which improves the condition of an individual. Particularly, therapeutic proteins play a key role in the design of new therapeutic agents that could modify and repair genetic deficiencies, destroy cancer cells or pathogen infected cells, treat or prevent immune system disorders, or treat or prevent metabolic or endocrine disorders, among other functions. Thusly, in one embodiment, the mRNA comprising at least one coding sequence may encode
(a) a peptide or protein, or a fragment or variant thereof, wherein the peptide or protein is an antigen, wherein the antigen preferably is derived from pathogenic antigens, tumour antigens, allergenic antigens or autoimmune self-antigens, or a fragment or variant thereof; or
(b) a therapeutic protein or a fragment or variant thereof. The therapeutic protein may, for example, be selected from the group consisting of
(i) therapeutic proteins for use in enzyme replacement therapy for the treatment of metabolic, endocrine or amino acid disorders or for use in replacing an absent, deficient or mutated protein;
(ii) therapeutic proteins for use in the treatment of blood disorders, diseases of the circulatory system, diseases of the respiratory system, infectious diseases or immune deficiencies;
(iii) therapeutic proteins for use in the treatment of cancer or tumour diseases;
(iv) therapeutic proteins for use in hormone replacement therapy;
(v) therapeutic proteins for use in reprogramming somatic cells into pluri- or omnipotent stem cells;
(vi) therapeutic proteins for use as adjuvant or immunostimulation;
(vii) therapeutic proteins being a therapeutic antibody;
(viii) therapeutic proteins being a gene editing agent; and
(ix) therapeutic proteins for use in treating or preventing a liver disease selected from the group consisting of liver fibrosis, liver cirrhosis and liver cancer.
   mRNA Elements

According to certain embodiments of the present invention, the mRNA sequence is mono-, bi-, or multicistronic, preferably as defined herein. The coding sequences in a bi- or multicistronic mRNA preferably encode distinct antigens as defined herein or a fragment or variant thereof. Preferably, the coding sequences encoding two or more antigens may be separated in the bi- or multicistronic mRNA by at least one IRES (internal ribosomal entry site) sequence, as defined below. Thus, the term “encoding two or more antigens” may mean, without being limited thereto, that the bi- or even multicistronic mRNA, may encode e.g. at least two, three, four, five, six or more (preferably different) antigens or their fragments or variants within the definitions provided herein. More preferably, without being limited thereto, the bi- or even multicistronic mRNA, may encode, for example, at least two, three, four, five, six or more (preferably different) antigens as defined herein or their fragments or variants as defined herein. In this context, a so-called IRES (internal ribosomal entry site) sequence can function as a sole ribosome binding site, but it can also serve to provide a bi- or even multicistronic mRNA as defined above, which encodes several antigens which are to be translated by the ribosomes independently of one another. Examples of IRES sequences, which can be used according to the invention, are those from Picornaviruses (e.g. FMDV), Pestiviruses (CFFV), Polioviruses (PV), Encephalomyocarditis viruses (ECMV), Foot and Mouth disease viruses (FMDV), Hepatitis C viruses (HCV), classical Swine fever viruses (CSFV), Mouse leukemia virus (MLV), Simian immunodeficiency viruses (SIV) or Cricket paralysis viruses (CrPV).

According to a further embodiment the at least one coding region or coding sequence of the mRNA sequence according to the invention may encode at least two, three, four, five, six, seven, eight and more antigens or fragments or variants thereof as defined herein linked with or without an amino acid linker sequence, wherein said linker sequence can comprise rigid linkers, flexible linkers, cleavable linkers (e.g., self-cleaving peptides) or a combination thereof. Therein, the antigens may be identical or different or a combination thereof. Particular antigen combinations can be encoded by said mRNA encoding at least two antigens as explained herein (also referred to herein as “multi-antigen-constructs/mRNA”).

In another preferred embodiment, the mRNA encodes a pathogenic antigen whose amino acid sequence is not modified with respect to the respective wild type amino acid sequence. In this case, the mRNA compound may also comprise a coding region with a nucleic acid sequence which is not modified with respect to the respective wild type mRNA sequence. For example, the mRNA compound may be a natural and non-modified mRNA. As used herein, natural and non-modified mRNA encompasses mRNA generated in vitro, without chemical modifications or changes in the sequence.

### Self-Replicating RNA

In one embodiment, the nucleic acid and in particular the mRNA sequence of the invention is capable of self-replication. Thusly, a polynucleotide may be capable of self-replication when introduced into a host cell. Examples of polynucleotides thus include self-replicating RNAs and DNAs and, for instance, selected from replicons, plasmids, cosmids, phagemids, transposons, viral vectors, artificial chromosomes (e.g., bacterial, yeast, etc.) as well as other self-replicating species. Polynucleotides include self-replicating polynucleotides within which natural or synthetic sequences derived from eucaryotic or prokaryotic organisms (e.g., genomic DNA sequences, genomic RNA sequences, cDNA sequences, etc.) have been inserted. Specific examples of self-replicating polynucleotides include RNA vector constructs and DNA vector constructs, among others. Sequences that may be expressed include native sequences and modifications, such as deletions, additions and substitutions (generally conservative in nature), to native sequences, among others. These modifications may be deliberate, as through site-directed mutagenesis, or may be accidental, such as through mutations of hosts that produce antigens. In one aspect, the self-replicating RNA molecule is derived from or based on an alphavirus. In other aspects, the self-replicating RNA molecule is derived from or based on a virus other than an alphavirus, preferably, a positive-stranded RNA virus, and more preferably a picornavirus, flavivirus, rubivirus, pestivirus, hepacivirus, calicivirus, or coronavirus. Suitable wild-type alphavirus sequences are well-known and are available from sequence depositories, such as the American Type Culture Collection, Rockville, Md. Representative examples of suitable alphaviruses include Aura (ATCC VR-368), Bebaru virus (ATCC VR-600, ATCC VR-1240), Cabassou (ATCC VR-922), Chikungunya virus (ATCC VR-64, ATCC VR-1241), Eastern equine encephalomyelitis virus (ATCC VR-65, ATCC VR-1242), Fort Morgan (ATCC VR-924), Getah virus (ATCC VR-369, ATCC VR-1243), Kyzylagach (ATCC VR-927), Mayaro (ATCC VR-66), Mayaro virus (ATCC VR-1277), Middleburg (ATCC VR-370), Mucambo virus (ATCC VR-580, ATCC VR-1244), Ndumu (ATCC VR-371), Pixuna virus (ATCC VR-372, ATCC VR-1245), Ross River virus (ATCC VR-373, ATCC VR-1246), Semliki Forest (ATCC VR-67, ATCC VR-1247), Sindbis virus (ATCC VR-68, ATCC VR-1248), Tonate (ATCC VR-925), Triniti (ATCC VR-469), Una (ATCC VR-374), Venezuelan equine encephalomyelitis (ATCC VR-69, ATCC VR-923, ATCC VR-1250 ATCC VR-1249, ATCC VR-532), Western equine encephalomyelitis (ATCC VR-70, ATCC VR-1251, ATCC VR-622, ATCC VR-1252), Whataroa (ATCC VR-926), and Y-62-33 (ATCC VR-375).

mRNA Modifications and Sequences

In another embodiment of the invention, the mRNA compound comprises an artificial mRNA. In this context, artificial mRNA encompasses mRNA with chemical modifications, sequence modifications or non-natural sequences.

### Chemical Modifications

According to another embodiment of the invention, the mRNA compound comprised in the composition comprises at least one chemical modification. In one embodiment, the chemical modification may be selected from the group consisting of base modifications, sugar modifications, backbone modifications and lipid modifications. A backbone modification in connection with the present invention is a modification in which phosphates of the backbone of the nucleotides contained in an mRNA compound comprising an mRNA sequence as defined herein are chemically modified. A sugar modification in connection with the present invention is a chemical modification of the sugar of the nucleotides of the mRNA compound comprising an mRNA sequence as defined herein. Furthermore, a base modification in connection with the present invention is a chemical modification of the base moiety of the nucleotides of the mRNA compound comprising an mRNA sequence. In this context, nucleotide analogues or modifications are preferably selected from nucleotide analogues, which are applicable for transcription and/or translation.

### Sugar Modifications

The modified nucleosides and nucleotides, which may be incorporated into a modified mRNA compound comprising an mRNA sequence as described herein, can be modified in the sugar moiety. For example, the 2′ hydroxyl group (OH) can be modified or replaced with a number of different “oxy” or “deoxy” substituents. Examples of “oxy”-2′ hydroxyl group modifications include, but are not limited to, alkoxy or aryloxy (—OR, e.g., R═H, alkyl, cycloalkyl, aryl, aralkyl, heteroaryl or sugar); polyethylene glycols (PEG), —O(CH2CH2O)nCH2CH2OR; “locked” nucleic acids (LNA) in which the 2′ hydroxyl is connected, e.g., by a methylene bridge, to the 4′ carbon of the same ribose sugar; and amino groups (—O-amino, wherein the amino group, e.g., NRR, can be alkylamino, dialkylamino, heterocyclyl, arylamino, diarylamino, heteroarylamino, or diheteroaryl amino, ethylene diamine, polyamino) or aminoalkoxy.

“Deoxy” modifications include hydrogen, amino (e.g. NH2; alkylamino, dialkylamino, heterocyclyl, arylamino, diaryl amino, heteroaryl amino, diheteroaryl amino, or amino acid); or the amino group can be attached to the sugar through a linker, wherein the linker comprises one or more of the atoms C, N, and O.

The sugar group can also contain one or more carbons that possess the opposite stereochemical configuration than that of the corresponding carbon in ribose. Thus, a modified mRNA can include nucleotides containing, for instance, arabinose as the sugar.

### Backbone Modifications

The phosphate groups of the backbone can be modified by replacing one or more of the oxygen atoms with a different substituent. Further, the modified nucleosides and nucleotides can include the full replacement of an unmodified phosphate moiety with a modified phosphate as described herein. Examples of modified phosphate groups include, but are not limited to, phosphorothioate, phosphoroselenates, borano phosphates, borano phosphate esters, hydrogen phosphonates, phosphoroamidates, alkyl or aryl phosphonates and phosphotriesters. Phosphorodithioates have both non-linking oxygens replaced by sulfur. The phosphate linker can also be modified by the replacement of a linking oxygen with nitrogen (bridged phosphoroamidates), sulfur (bridged phosphorothioates) and carbon (bridged methylene-phosphonates).

### Lipid Modifications

A lipid-modified mRNA typically comprises at least one linker covalently linked with that mRNA, and at least one lipid covalently linked with the respective linker. Alternatively, the lipid-modified mRNA comprises at least one mRNA as defined herein and at least one (bifunctional) lipid covalently linked (without a linker) with that mRNA. According to a third alternative, the lipid-modified mRNA comprises an mRNA molecule as defined herein, at least one linker covalently linked with that mRNA, and at least one lipid covalently linked with the respective linker, and also at least one (bifunctional) lipid covalently linked (without a linker) with that mRNA. In this context, it is particularly preferred that the lipid modification is present at the terminal ends of a linear mRNA sequence.

In another preferred embodiment, the mRNA compound does not comprise nucleoside modifications, in particular no base modifications. In a further embodiment, the mRNA compound does not comprise 1-methylpseudouridine, pseudouridine or 5-methoxy-uridine modifications. In one preferred embodiment, the mRNA comprises only naturally existing nucleosides. In a further preferred embodiment, the mRNA compound does not comprise any chemical modification and optionally comprises sequence modifications. In a further preferred embodiment of the invention the mRNA compound only comprises the naturally existing nucleosides adenine, uracil, guanine and cytosine.

### Base Modifications

In an alternative embodiment, the mRNA compound comprises at least one base modification.

Modified nucleosides and nucleotides, which may be incorporated into a modified mRNA compound comprising an mRNA sequence as described herein can further be modified in the nucleobase moiety. Examples of nucleobases found in mRNA include, but are not limited to, adenine, guanine, cytosine and uracil. For example, the nucleosides and nucleotides described herein can be chemically modified on the major groove face. In some embodiments, the major groove chemical modifications can include an amino group, a thiol group, an alkyl group, or a halo group.

In particularly preferred embodiments of the present invention, the nucleotide analogues/modifications are selected from base modifications, which are preferably selected from 2-amino-6-chloropurineriboside-5′-triphosphate, 2-Aminopurine-riboside-5′-triphosphate; 2-aminoadenosine-5′-triphosphate, 2′-Amino-2′-deoxy-cytidine-triphosphate, 2-thiocytidine-5′-triphosphate, 2-thiouridine-5′-triphosphate, 2′-Fluorothymidine-5′-triphosphate, 2′-O-Methyl-inosine-5′-triphosphate 4-thiouridine-5′-triphosphate, 5-aminoallylcytidine-5′-triphosphate, 5-aminoallyluridine-5′-triphosphate, 5-bromocytidine-5′-triphosphate, 5-bromouridine-5′-triphosphate, 5-Bromo-2′-deoxycytidine-5′-triphosphate, 5-Bromo-2′-deoxyuridine-5′-triphosphate, 5-iodocytidine-5′-triphosphate, 5-lodo-2′-deoxycytidine-5′-triphosphate, 5-iodouridine-5′-triphosphate, 5-lodo-2′-deoxyuridine-5′-triphosphate, 5-methyl-cytidine-5′-triphosphate, 5-methyluridine-5′-triphosphate, 5-Propynyl-2′-deoxycytidine-5′-triphosphate, 5-Propynyl-2′-deoxyuridine-5′-triphosphate, 6-azacytidine-5′-triphosphate, 6-azauridine-5′-triphosphate, 6-chloropurine-riboside-5′-triphosphate, 7-deazaadenosine-5′-triphosphate, 7-deazaguanosine-5′-triphosphate, 8-azaadenosine-5′-triphosphate, 8-azidoadenosine-5′-triphosphate, benzimidazole-riboside-5′-triphosphate, N1-methyladenosine-5′-triphosphate, N1-methylguanosine-5′-triphosphate, N6-methyladenosine-5′-triphosphate, 06-methylguanosine-5′-triphosphate, pseudouridine-5′-triphosphate, or puromycin-5′-triphosphate, xanthosine-5′-triphosphate. Particular preference is given to nucleotides for base modifications selected from the group of base-modified nucleotides consisting of 5-methylcytidine-5′-triphosphate, 7-deazaguanosine-5′-triphosphate, 5-bromocytidine-5′-triphosphate, and pseudouridine-5′-triphosphate. In some embodiments, modified nucleosides include pyridin-4-one ribonucleoside, 5-aza-uridine, 2-thio-5-aza-uridine, 2-thiouridine, 4-thio-pseudouridine, 2-thio-pseudouridine, 5-hydroxyuridine, 3-methyluridine, 5-carboxymethyl-uridine, 1-carboxymethyl-pseudouridine, 5-propynyl-uridine, 1-propynyl-pseudouridine, 5-taurinomethyluridine, 1-taurinomethyl-pseudouridine, 5-taurinomethyl-2-thio-uridine, 1-taurinomethyl-4-thio-uridine, 5-methyl-uridine, 1-methyl-pseudouridine, 4-thio-1-methyl-pseudouridine, 2-thio-1-methyl-pseudouridine, 1-methyl-1-deaza-pseudouridine, 2-thio-1-methyl-1-deaza-pseudouridine, dihydrouridine, dihydropseudouridine, 2-thio-dihydrouridine, 2-thio-dihydropseudouridine, 2-methoxyuridine, 2-methoxy-4-thio-uridine, 4-methoxy-pseudouridine, and 4-methoxy-2-thio-pseudouridine. In some embodiments, modified nucleosides include 5-aza-cytidine, pseudoisocytidine, 3-methyl-cytidine, N4-acetylcytidine, 5-formylcytidine, N4-methylcytidine, 5-hydroxymethylcytidine, 1-methyl-pseudoisocytidine, pyrrolo-cytidine, pyrrolo-pseudoisocytidine, 2-thio-cytidine, 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, and 4-methoxy-1-methyl-pseudoisocytidine. In other embodiments, modified nucleosides include 2-aminopurine, 2, 6-diaminopurine, 7-deaza-adenine, 7-deaza-8-aza-adenine, 7-deaza-2-aminopurine, 7-deaza-8-aza-2-aminopurine, 7-deaza-2,6-diaminopurine, 7-deaza-8-aza-2,6-diaminopurine, 1-methyladenosine, N6-methyladenosine, N6-isopentenyladenosine, N6-(cis-hydroxyisopentenyl)adenosine, 2-methylthio-N6-(cis-hydroxyisopentenyl) adenosine, N6-glycinylcarbamoyladenosine, N6-threonylcarbamoyl-adenosine, 2-methylthio-N6-threonyl carbamoyladenosine, N6,N6-dimethyladenosine, 7-methyladenine, 2-methyl-thio-adenine, and 2-methoxy-adenine. In other embodiments, modified nucleosides include inosine, 1-methyl-inosine, wyosine, wybutosine, 7-deaza-guanosine, 7-deaza-8-aza-guanosine, 6-thio-guanosine, 6-thio-7-deaza-guanosine, 6-thio-7-deaza-8-aza-guanosine, 7-methyl-guanosine, 6-thio-7-methyl-guanosine, 7-methylinosine, 6-methoxy-guanosine, 1-methylguanosine, N2-methylguanosine, N2,N2-dimethylguanosine, 8-oxo-guanosine, 7-methyl-8-oxo-guanosine, 1-methyl-6-thio-guanosine, N2-methyl-6-thio-guanosine, and N2,N2-dimethyl-6-thio-guanosine. In some embodiments, the nucleotide can be modified on the major groove face and can include replacing hydrogen on C-5 of uracil with a methyl group or a halo group. In specific embodiments, a modified nucleoside is 5′-O-(1-thiophosphate)-adenosine, 5′-O-(1-thiophosphate)-cytidine, 5′-O-(1-thiophosphate)-guanosine, 5′-O-(1-thiophosphate)-uridine or 5′-O-(1-thiophosphate)-pseudouridine.

In further specific embodiments, a modified mRNA may comprise nucleoside modifications selected from 6-aza-cytidine, 2-thio-cytidine, α-thio-cytidine, Pseudo-iso-cytidine, 5-aminoallyl-uridine, 5-iodo-uridine, N1-methyl-pseudouridine, 5,6-dihydrouridine, α-thio-uridine, 4-thio-uridine, 6-aza-uridine, 5-hydroxy-uridine, deoxy-thymidine, 5-methyl-uridine, Pyrrolo-cytidine, inosine, α-thio-guanosine, 6-methyl-guanosine, 5-methyl-cytdine, 8-oxo-guanosine, 7-deaza-guanosine, N1-methyl-adenosine, 2-amino-6-Chloro-purine, N6-methyl-2-aminopurine, Pseudo-iso-cytidine, 6-Chloro-purine, N6-methyl-adenosine, α-thio-adenosine, 8-azido-adenosine, 7-deazaadenosine.

In further embodiments, the chemical modification is selected from pseudouridine, N1-methylpseudouridine, N1-ethylpseudouridine, 2-thiouridine, 4′-thiouridine, 5-methylcytosine, 5-methyluridine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methoxyuridine and 2′-O-methyl uridine.

In a specific embodiment, the chemical modification is selected from the group consisting of pseudouracil (psi or ψ), N1-methylpseudouracil (N1MPU, N1Mpsi or N1ML), 1-ethylpseudouracil, 2-thiouracil (s2U), 4-thiouracil, 5-methylcytosine, 5-methyluracil, 5-methoxyuracil, and any combination thereof, most preferably the chemical modification is N1-methylpseudouracil (N1MPU, N1Mpsi or N1Mψ).

### Sequence Modifications

According to a further embodiment, the mRNA compound comprises a modified mRNA sequence. For example, a modification of the mRNA sequence may lead to the stabilization of the mRNA sequence. In one embodiment, the mRNA compound comprises a stabilized mRNA sequence comprising at least one coding region as defined herein. In particular, the composition of the invention as described herein may comprise an mRNA compound comprising a coding region encoding an antigen, such as defined in any of the embodiments described herein, wherein said coding region exhibits a sequence modification.

According to one embodiment, the mRNA compound comprises a “stabilized mRNA sequence”, that is to say as an mRNA that is essentially resistant to in vivo degradation (e.g. by an exo- or endo-nuclease). Such stabilization can be effected, for example, by a modified phosphate backbone of the mRNA of the present invention. A backbone modification in connection with the present invention is a modification in which phosphates of the backbone of the nucleotides contained in the mRNA are chemically modified. Nucleotides that may be preferably used in this connection contain e.g. a phosphorothioate-modified phosphate backbone, preferably at least one of the phosphate oxygens contained in the phosphate backbone being replaced by a sulfur atom. Stabilized mRNAs may further include, for example: non-ionic phosphate analogues, such as, for example, alkyl and aryl phosphonates, in which the charged phosphonate oxygen is replaced by an alkyl or aryl group, or phosphodiesters and alkylphosphotriesters, in which the charged oxygen residue is present in alkylated form.

Such backbone modifications typically include, without implying any limitation, modifications from the group consisting of methylphosphonates, phosphoramidates and phosphorothioates (e.g. cytidine-5-O-(1-thiophosphate)).

In the following, specific modifications are described which are preferably capable of “stabilizing” the mRNA as defined herein.

### G/C Content Modifications

According to one embodiment, the mRNA compound comprises an mRNA sequence which is modified, and thus stabilized, by a modification of its guanosine/cytosine (G/C) content. Such modification, or at least one of these modifications, is located in a coding region of the mRNA compound.

In one preferred embodiment, the G/C content of the coding region of the mRNA compound is increased compared to the G/C content of the coding region of the respective wild type mRNA, i.e. the unmodified mRNA. At the same time, the amino acid sequence encoded by the mRNA is preferably not modified as compared to the amino acid sequence encoded by the respective wild type mRNA. For example, the composition as described above may comprise an mRNA compound encoding a pathogenic antigen whose amino acid sequence is not modified with respect to the encoded amino acid sequence of the respective wild type nucleic acid.

This modification of the mRNA sequence of the present invention is based on the fact that the sequence of any mRNA region to be translated is important for efficient translation of that mRNA. Thus, the composition of the mRNA and the sequence of various nucleotides are important. In particular, sequences having an increased G (guanosine)/C (cytosine) content are more stable than sequences having an increased A (adenosine)/U (uracil) content. According to the invention, the codons of the mRNA are therefore varied compared to the respective wild type mRNA, while retaining the translated amino acid sequence, such that they include an increased amount of G/C nucleotides. In respect to the fact that several codons code for one and the same amino acid (so-called degeneration of the genetic code), the most favorable codons for the stability can be determined (so-called alternative codon usage). Depending on the amino acid to be encoded by the mRNA, there are various possibilities for modification of the mRNA sequence, compared to its wild type sequence. In the case of amino acids, which are encoded by codons, which contain exclusively G or C nucleotides, no modification of the codon is necessary. Thus, the codons for Pro (CCC or CCG), Arg (CGC or CGG), Ala (GCC or GCG) and Gly (GGC or GGG) require no modification, since no A or U is present. In contrast, codons which contain A and/or U nucleotides can be modified by substitution of other codons, which code for the same amino acids but contain no A and/or U. Examples of these are: the codons for Pro can be modified from CCU or CCA to CCC or CCG; the codons for Arg can be modified from CGU or CGA or AGA or AGG to CGC or CGG; the codons for Ala can be modified from GCU or GCA to GCC or GCG; the codons for Gly can be modified from GGU or GGA to GGC or GGG. In other cases, although A or U nucleotides cannot be eliminated from the codons, it is however possible to decrease the A and U content by using codons which contain a lower content of A and/or U nucleotides. Examples of these are: the codons for Phe can be modified from UUU to UUC; the codons for Leu can be modified from UUA, UUG, CUU or CUA to CUC or CUG; the codons for Ser can be modified from UCU or UCA or AGU to UCC, UCG or AGC; the codon for Tyr can be modified from UAU to UAC; the codon for Cys can be modified from UGU to UGC; the codon for His can be modified from CAU to CAC; the codon for Gln can be modified from CAA to CAG; the codons for Ile can be modified from AUU or AUA to AUC; the codons for Thr can be modified from ACU or ACA to ACC or ACG; the codon for Asn can be modified from AAU to AAC; the codon for Lys can be modified from AAA to AAG; the codons for Val can be modified from GUU or GUA to GUC or GUG; the codon for Asp can be modified from GAU to GAC; the codon for Glu can be modified from GAA to GAG; the stop codon UAA can be modified to UAG or UGA. In the case of the codons for Met (AUG) and Trp (UGG), on the other hand, there is no possibility of sequence modification. The substitutions listed above can be used either individually or in all possible combinations to increase the G/C content of the mRNA sequence of the present invention compared to its particular wild type mRNA (i.e. the original sequence). Thus, for example, all codons for Thr occurring in the wild type sequence can be modified to ACC (or ACG). Preferably, however, for example, combinations of the above substitution possibilities are used:
substitution of all codons coding for Thr in the original sequence (wild type mRNA) to ACC (or ACG) and
substitution of all codons originally coding for Ser to UCC (or UCG or AGC);
substitution of all codons coding for Ile in the original sequence to AUC and
substitution of all codons originally coding for Lys to AAG and
substitution of all codons originally coding for Tyr to UAC;
substitution of all codons coding for Val in the original sequence to GUC (or GUG) and
substitution of all codons originally coding for Glu to GAG and
substitution of all codons originally coding for Ala to GCC (or GCG) and
substitution of all codons originally coding for Arg to CGC (or CGG);
substitution of all codons coding for Val in the original sequence to GUC (or GUG) and
substitution of all codons originally coding for Glu to GAG and
substitution of all codons originally coding for Ala to GCC (or GCG) and
substitution of all codons originally coding for Gly to GGC (or GGG) and
substitution of all codons originally coding for Asn to AAC;
substitution of all codons coding for Val in the original sequence to GUC (or GUG) and
substitution of all codons originally coding for Phe to UUC and
substitution of all codons originally coding for Cys to UGC and
substitution of all codons originally coding for Leu to CUG (or CUC) and
substitution of all codons originally coding for Gln to CAG and
substitution of all codons originally coding for Pro to CCC (or CCG); etc.

Preferably, the G/C content of the coding region of the mRNA compound comprising an mRNA sequence of the present invention is increased by at least 7%, more preferably by at least 15%, particularly preferably by at least 20%, compared to the G/C content of the coding region of the wild type RNA, which codes for an antigen as defined herein or a fragment or variant thereof. According to a specific embodiment at least 5%, 10%, 20%, 30%, 40%, 50%, 60%, more preferably at least 70%, even more preferably at least 80% and most preferably at least 90%, 95% or even 100% of the substitutable codons in the region coding for a peptide or protein as defined herein or a fragment or variant thereof or the whole sequence of the wild type mRNA sequence are substituted, thereby increasing the G/C content of said sequence. In this context, it is particularly preferable to increase the G/C content of the mRNA sequence of the present invention, preferably of the at least one coding region of the mRNA sequence according to the invention, to the maximum (i.e. 100% of the substitutable codons) as compared to the wild type sequence. According to the invention, a further preferred modification of the mRNA sequence of the present invention is based on the finding that the translation efficiency is also determined by a different frequency in the occurrence of tRNAs in cells. Thus, if so-called “rare codons” are present in the mRNA sequence of the present invention to an increased extent, the corresponding modified mRNA sequence is translated to a significantly poorer degree than in the case where codons coding for relatively “frequent” tRNAs are present. According to the invention, in the modified mRNA sequence of the present invention, the region which codes for a peptide or protein as defined herein or a fragment or variant thereof is modified compared to the corresponding region of the wild type mRNA sequence such that at least one codon of the wild type sequence, which codes for a tRNA which is relatively rare in the cell, is exchanged for a codon, which codes for a tRNA which is relatively frequent in the cell and carries the same amino acid as the relatively rare tRNA. By this modification, the sequence of the mRNA of the present invention is modified such that codons for which frequently occurring tRNAs are available are inserted. In other words, according to the invention, by this modification all codons of the wild type sequence, which code for a tRNA which is relatively rare in the cell, can in each case be exchanged for a codon, which codes for a tRNA which is relatively frequent in the cell and which, in each case, carries the same amino acid as the relatively rare tRNA. Which tRNAs occur relatively frequently in the cell and which, in contrast, occur relatively rarely is known to a person skilled in the art; cf. e.g. Akashi, Curr. Opin. Genet. Dev. 2001, 11(6): 660-666. The codons, which use for the particular amino acid the tRNA which occurs the most frequently, e.g. the Gly codon, which uses the tRNA, which occurs the most frequently in the (human) cell, are particularly preferred. According to the invention, it is particularly preferable to link the sequential G/C content which is increased, in particular maximized, in the modified mRNA sequence of the present invention, with the “frequent” codons without modifying the amino acid sequence of the protein encoded by the coding region of the mRNA sequence. This preferred embodiment allows provision of a particularly efficiently translated and stabilized (modified) mRNA sequence of the present invention. The determination of a modified mRNA sequence of the present invention as described above (increased G/C content; exchange of tRNAs) can be carried out using the computer program explained in WO2002098443—the disclosure content of which is included in its full scope in the present invention. Using this computer program, the nucleotide sequence of any desired mRNA sequence can be modified with the aid of the genetic code or the degenerative nature thereof such that a maximum G/C content results, in combination with the use of codons which code for tRNAs occurring as frequently as possible in the cell, the amino acid sequence coded by the modified mRNA sequence preferably not being modified compared to the non-modified sequence. Alternatively, it is also possible to modify only the G/C content or only the codon usage compared to the original sequence. The source code in Visual Basic 6.0 (development environment used: Microsoft Visual Studio Enterprise 6.0 with Service Pack 3) is also described in WO02/098443. In a further preferred embodiment of the present invention, the A/U content in the environment of the ribosome binding site of the mRNA sequence of the present invention is increased compared to the A/U content in the environment of the ribosome binding site of its respective wild type mRNA. This modification (an increased A/U content around the ribosome binding site) increases the efficiency of ribosome binding to the mRNA. An effective binding of the ribosomes to the ribosome binding site (Kozak sequence: SEQ ID NO:1 or SEQ ID NO:2, the AUG forms the start codon, or a minimal Kozak binding site ACC) in turn has the effect of an efficient translation of the mRNA. According to a further embodiment of the present invention, the mRNA sequence of the present invention may be modified with respect to potentially destabilizing sequence elements. Particularly, the coding region and/or the 5′ and/or 3′ untranslated region of this mRNA sequence may be modified compared to the respective wild type mRNA such that it contains no destabilizing sequence elements, the encoded amino acid sequence of the modified mRNA sequence preferably not being modified compared to its respective wild type mRNA. It is known that, for example in sequences of eukaryotic mRNAs, destabilizing sequence elements (DSE) occur, to which signal proteins bind and regulate enzymatic degradation of mRNA in vivo. For further stabilization of the modified mRNA sequence, optionally in the region which encodes at least one peptide or protein as defined herein or a fragment or variant thereof, one or more such modifications compared to the corresponding region of the wild type mRNA can therefore be carried out, so that no or substantially no destabilizing sequence elements are contained there. According to the invention, DSE present in the untranslated regions (3′- and/or 5′-UTR) can also be eliminated from the mRNA sequence of the present invention by such modifications. Such destabilizing sequences are e.g. AU-rich sequences (AURES), which occur in 3′-UTR sections of numerous unstable mRNAs (Caput et al., Proc. Natl. Acad. Sci. USA 1986, 83: 1670-1674). The mRNA sequence of the present invention is therefore preferably modified compared to the respective wild type mRNA such that the mRNA sequence of the present invention contains no such destabilizing sequences. This also applies to those sequence motifs which are recognized by possible endonucleases, e.g. the sequence GAACAAG, which is contained in the 3′-UTR segment of the gene encoding the transferrin receptor (Binder et al., EMBO J. 1994, 13: 1969-1980). These sequence motifs are also preferably removed in the mRNA sequence of the present invention.

According to a further embodiment, the mRNA compound comprises an mRNA sequence comprising a coding region that comprises or consists of any one of the RNA sequences as disclosed in Tabs. 1-5, FIGS. 20-24 or in the sequence listing of WO2018078053; Tabs. 1-5 or FIGS. 20-24 of WO2018078053; WO2018078053 incorporated by reference in its entirety.

### Sequences Adapted to Human Codon Usage

A further preferred modification of the mRNA compound is based on the finding that codons encoding the same amino acid typically occur at different frequencies. According to this embodiment, the frequency of the codons encoding the same amino acid in the coding region of the mRNA compound differs from the naturally occurring frequency of that codon according to the human codon usage as e.g. shown in Table 2 (Human codon usage table). For example, in the case of the amino acid alanine (Ala), the wild type coding region is preferably adapted in a way that the codon “GCC” is used with a frequency of 0.40, the codon “GCT” is used with a frequency of 0.28, the codon “GCA” is used with a frequency of 0.22 and the codon “GCG” is used with a frequency of 0.10 etc. (see Table 2).

TABLE 2
Human codon usage table, most frequent
codons are marked with asterisks

| Amino | | | | Amino | | | |
|---|---|---|---|---|---|---|---|
| acid | codon | fraction | /1000 | acid | codon | fraction | /1000 |
| Ala | GCG | 0.10 | 7.4 | Pro | CCG | 0.11 | 6.9 |
| Ala | GCA | 0.22 | 15.8 | Pro | CCA | 0.27 | 16.9 |
| Ala | GCT | 0.28 | 18.5 | Pro | CCT | 0.29 | 17.5 |
| Ala | GCC* | 0.40 | 27.7 | Pro | CCC* | 0.33 | 19.8 |
| Cys | TGT | 0.42 | 10.6 | Gln | CAG* | 0.73 | 34.2 |
| Cys | TGC* | 0.58 | 12.6 | Gln | CAA | 0.27 | 12.3 |
| Asp | GAT | 0.44 | 21.8 | Arg | AGG | 0.22 | 12.0 |
| Asp | GAC* | 0.56 | 25.1 | Arg | AGA* | 0.21 | 12.1 |
| Glu | GAG* | 0.59 | 39.6 | Arg | CGG | 0.19 | 11.4 |
| Glu | GAA | 0.41 | 29.0 | Arg | CGA | 0.10 | 6.2 |
| Phe | TTT | 0.43 | 17.6 | Arg | CGT | 0.09 | 4.5 |
| Phe | TTC* | 0.57 | 20.3 | Arg | CGC | 0.19 | 10.4 |
| Gly | GGG | 0.23 | 16.5 | Ser | AGT | 0.14 | 12.1 |
| Gly | GGA | 0.26 | 16.5 | Ser | AGC* | 0.25 | 19.5 |
| Gly | GGT | 0.18 | 10.8 | Ser | TCG | 0.06 | 4.4 |
| Gly | GGC* | 0.33 | 22.2 | Ser | TCA | 0.15 | 12.2 |
| His | CAT | 0.41 | 10.9 | Ser | TCT | 0.18 | 15.2 |
| His | CAC* | 0.59 | 15.1 | Ser | TCC | 0.23 | 17.7 |
| Ile | ATA | 0.14 | 7.5 | Thr | ACG | 0.12 | 6.1 |
| Ile | ATT | 0.35 | 16.0 | Thr | ACA | 0.27 | 15.1 |
| Ile | ATC* | 0.52 | 20.8 | Thr | ACT | 0.23 | 13.1 |
| Lys | AAG* | 0.60 | 31.9 | Thr | ACC* | 0.38 | 18.9 |
| Lys | AAA | 0.40 | 24.4 | Val | GTG* | 0.48 | 28.1 |
| Leu | TTG | 0.12 | 12.9 | Val | GTA | 0.10 | 7.1 |
| Leu | TTA | 0.06 | 7.7 | Val | GTT | 0.17 | 11.0 |
| Leu | CTG* | 0.43 | 39.6 | Val | GTC | 0.25 | 14.5 |
| Leu | CTA | 0.07 | 7.2 | Trp | TGG* | 1 | 13.2 |
| Leu | CTT | 0.12 | 13.2 | Tyr | TAT | 0.42 | 12.2 |
| Leu | CTC | 0.20 | 19.6 | Tyr | TAC* | 0.58 | 15.3 |
| Met | ATG* | 1 | 22.0 | Stop | TGA* | 0.61 | 1.6 |
| Asn | AAT | 0.44 | 17.0 | Stop | TAG | 0.17 | 0.8 |
| Asn | AAC* | 0.56 | 19.1 | Stop | TAA | 0.22 | 1.0 |

*most frequent codon

### Codon-Optimized Sequences

In one embodiment, all codons of the wild type sequence which code for a tRNA, which is relatively rare in the cell, are exchanged for a codon which codes for a tRNA, which is relatively frequent in the cell and which, in each case, carries the same amino acid as the relatively rare tRNA. Therefore it is particularly preferred that the most frequent codons are used for each encoded amino acid (see Table 2). Such an optimization procedure increases the codon adaptation index (CAI) and ultimately maximizes the CAI. In the context of the invention, sequences with increased or maximized CAI are typically referred to as “codon-optimized” sequences and/or CAI increased and/or maximized sequences. According to a preferred embodiment, the mRNA compound comprising an mRNA sequence of the present invention comprises at least one coding region, wherein the coding region/sequence is codon-optimized as described herein. More preferably, the codon adaptation index (CAI) of the at least one coding sequence is at least 0.5, at least 0.8, at least 0.9 or at least 0.95. Most preferably, the codon adaptation index (CAI) of the at least one coding sequence is 1.

For example, in the case of the amino acid alanine (Ala) present in the amino acid sequence encoded by the at least one coding sequence of the RNA according to the invention, the wild type coding sequence is adapted in a way that the most frequent human codon “GCC” is always used for said amino acid, or for the amino acid Cysteine (Cys), the wild type sequence is adapted in a way that the most frequent human codon “TGC” is always used for said amino acid etc.

### C-Optimized Sequences

According to another embodiment, the mRNA compound comprising an mRNA sequence having a modified—in particular increased—cytosine (C) content, preferably of the coding region of the mRNA sequence, compared to the C content of the coding region of the respective wild type mRNA, i.e. the unmodified mRNA. At the same time, the amino acid sequence encoded by the at least one coding region of the mRNA sequence of the present invention is preferably not modified as compared to the amino acid sequence encoded by the respective wild type mRNA.

In a preferred embodiment of the present invention, the modified mRNA sequence is modified such that at least 10%, 20%, 30%, 40%, 50%, 60%, 70% or 80%, or at least 90% of the theoretically possible maximum cytosine-content or even a maximum cytosine-content is achieved.

In further preferred embodiments, at least 10%, 20%, 30%, 40%, 50%, 60%, 70%, 80%, 90% or even 100% of the codons of the target mRNA wild type sequence, which are “cytosine content optimizable” are replaced by codons having a higher cytosine-content than the ones present in the wild type sequence.

In a further preferred embodiment, some of the codons of the wild type coding sequence may additionally be modified such that a codon for a relatively rare tRNA in the cell is exchanged by a codon for a relatively frequent tRNA in the cell, provided that the substituted codon for a relatively frequent tRNA carries the same amino acid as the relatively rare tRNA of the original wild type codon. Preferably, all of the codons for a relatively rare tRNA are replaced by a codon for a relatively frequent tRNA in the cell, except codons encoding amino acids, which are exclusively encoded by codons not containing any cytosine, or except for glutamine (Gln), which is encoded by two codons each containing the same number of cytosines.

In a further preferred embodiment of the present invention, the modified target mRNA is modified such that at least 80%, or at least 90% of the theoretically possible maximum cytosine-content or even a maximum cytosine-content is achieved by means of codons, which code for relatively frequent tRNAs in the cell, wherein the amino acid sequence remains unchanged.

Due to the naturally occurring degeneracy of the genetic code, more than one codon may encode a particular amino acid. Accordingly, 18 out of 20 naturally occurring amino acids are encoded by more than one codon (with Tryp and Met being an exception), e.g. by 2 codons (e.g. Cys, Asp, Glu), by three codons (e.g. Ile), by 4 codons (e.g. Al, Gly, Pro) or by 6 codons (e.g. Leu, Arg, Ser). However, not all codons encoding the same amino acid are utilized with the same frequency under in vivo conditions. Depending on each single organism, a typical codon usage profile is established.

The term “cytosine content-optimizable codon” as used within the context of the present invention refers to codons, which exhibit a lower content of cytosines than other codons encoding the same amino acid. Accordingly, any wild type codon, which may be replaced by another codon encoding the same amino acid and exhibiting a higher number of cytosines within that codon, is considered to be cytosine-optimizable (C-optimizable). Any such substitution of a C-optimizable wild type codon by the specific C-optimized codon within a wild type coding region increases its overall C-content and reflects a C-enriched modified mRNA sequence. According to a preferred embodiment, the mRNA sequence of the present invention, preferably the at least one coding region of the mRNA sequence of the present invention comprises or consists of a C-maximized mRNA sequence containing C-optimized codons for all potentially C-optimizable codons. Accordingly, 100% or all of the theoretically replaceable C-optimizable codons are preferably replaced by C-optimized codons over the entire length of the coding region. In this context, cytosine-content optimizable codons are codons, which contain a lower number of cytosines than other codons coding for the same amino acid.

Any of the codons GCG, GCA, GCU codes for the amino acid Ala, which may be exchanged by the codon GCC encoding the same amino acid, and/or
the codon UGU that codes for Cys may be exchanged by the codon UGC encoding the same amino acid, and/or
the codon GAU which codes for Asp may be exchanged by the codon GAC encoding the same amino acid, and/or
the codon that UUU that codes for Phe may be exchanged for the codon UUC encoding the same amino acid, and/or
any of the codons GGG, GGA, GGU that code Gly may be exchanged by the codon GGC encoding the same amino acid, and/or
the codon CAU that codes for His may be exchanged by the codon CAC encoding the same amino acid, and/or
any of the codons AUA, AUU that code for Ile may be exchanged by the codon AUC, and/or
any of the codons UUG, UUA, CUG, CUA, CUU coding for Leu may be exchanged by the codon CUC encoding the same amino acid, and/or
the codon AAU that codes for Asn may be exchanged by the codon AAC encoding the same amino acid, and/or
any of the codons CCG, CCA, CCU coding for Pro may be exchanged by the codon CCC encoding the same amino acid, and/or
any of the codons AGG, AGA, CGG, CGA, CGU coding for Arg may be exchanged by the codon CGC encoding the same amino acid, and/or
any of the codons AGU, AGC, UCG, UCA, UCU coding for Ser may be exchanged by the codon UCC encoding the same amino acid, and/or
any of the codons ACG, ACA, ACU coding for Thr may be exchanged by the codon ACC encoding the same amino acid, and/or
any of the codons GUG, GUA, GUU coding for Val may be exchanged by the codon GUC encoding the same amino acid, and/or
the codon UAU coding for Tyr may be exchanged by the codon UAC encoding the same amino acid.

In any of the above instances, the number of cytosines is increased by 1 per exchanged codon. Exchange of all non C-optimized codons (corresponding to C-optimizable codons) of the coding region results in a C-maximized coding sequence. In the context of the invention, at least 70%, preferably at least 80%, more preferably at least 90%, of the non C-optimized codons within the at least one coding region of the mRNA sequence according to the invention are replaced by C-optimized codons.

It may be preferred that for some amino acids the percentage of C-optimizable codons replaced by C-optimized codons is less than 70%, while for other amino acids the percentage of replaced codons is higher than 70% to meet the overall percentage of C-optimization of at least 70% of all C-optimizable wild type codons of the coding region.

Preferably, in a C-optimized mRNA sequence, at least 50% of the C-optimizable wild type codons for any given amino acid are replaced by C-optimized codons, e.g. any modified C-enriched mRNA sequence preferably contains at least 50% C-optimized codons at C-optimizable wild type codon positions encoding any one of the above mentioned amino acids Ala, Cys, Asp, Phe, Gly, His, Ile, Leu, Asn, Pro, Arg, Ser, Thr, Val and Tyr, preferably at least 60%.

In this context, codons encoding amino acids which are not cytosine content-optimizable and which are, however, encoded by at least two codons, may be used without any further selection process. However, the codon of the wild type sequence that codes for a relatively rare tRNA in the cell, e.g. a human cell, may be exchanged for a codon that codes for a relatively frequent tRNA in the cell, wherein both code for the same amino acid.

Accordingly, the relatively rare codon GAA coding for Glu may be exchanged by the relative frequent codon GAG coding for the same amino acid, and/or the relatively rare codon AAA coding for Lys may be exchanged by the relative frequent codon AAG coding for the same amino acid, and/or the relatively rare codon CAA coding for Gln may be exchanged for the relative frequent codon CAG encoding the same amino acid.

In this context, the amino acids Met (AUG) and Trp (UGG), which are encoded by only one codon each, remain unchanged. Stop codons are not cytosine-content optimized, however, the relatively rare stop codons amber, ochre (UAA, UAG) may be exchanged by the relatively frequent stop codon opal (UGA).

The single substitutions listed above may be used individually as well as in all possible combinations in order to optimize the cytosine-content of the modified mRNA sequence compared to the wild type mRNA sequence.

Accordingly, the at least one coding sequence as defined herein may be changed compared to the coding region of the respective wild type mRNA in such a way that an amino acid encoded by at least two or more codons, of which one comprises one additional cytosine, such a codon may be exchanged by the C-optimized codon comprising one additional cytosine, wherein the amino acid is preferably unaltered compared to the wild type sequence.

According to a further preferred embodiment, the composition of the invention comprises an mRNA compound whose coding region has an increased G/C content compared to the G/C content of the corresponding coding region of the corresponding wild type mRNA, and/or an increased C content compared to the C content of the corresponding coding region of the corresponding wild type mRNA, and/or wherein the codons in the coding region are adapted to human codon usage, wherein the codon adaptation index (CAI) is preferably increased or maximized, and wherein the amino acid sequence encoded by the mRNA sequence is preferably not being modified compared to the amino acid sequence encoded by the corresponding wild type mRNA.

In one preferred embodiment of the invention, the composition comprises an mRNA compound comprising a coding region encoding a peptide or a protein, wherein the coding region exhibits a sequence modification selected from a G/C content modification, a codon modification, a codon optimization or a C-optimization of the sequence.

In another preferred embodiment, the composition or lipid nanoparticle as defined herein comprises an mRNA comprising a coding region encoding a peptide or protein as defined herein, wherein, compared with the coding region of the corresponding wild-type mRNA,
the G/C content of the coding region is increased;
the C content of the coding region is increased;
the codon usage in the coding region is adapted to the human codon usage; and/or the codon adaptation index (CAI) is increased or maximized in the coding region.

### 5′-CAP Structure

Suitably, the coding RNA may be modified by the addition of a 5′-CAP structure, which preferably stabilizes the coding RNA and/or enhances expression of the encoded antigen and/or reduces the stimulation of the innate immune system (after administration to a subject). A 5′-CAP structure is of particular importance in embodiments where the nucleic acid is an RNA, in particular a linear coding RNA, e.g. a linear mRNA or a linear coding replicon RNA.

Accordingly, in preferred embodiments, the RNA, in particular the coding RNA comprises a 5′-CAP structure, preferably CAP0, CAP1, CAP2, a modified CAP0, or a modified CAP1 structure.

The term “5′-CAP structure” as used herein will be recognized and understood by the person of ordinary skill in the art, and is e.g. intended to refer to a 5′ modified nucleotide, particularly a guanine nucleotide, positioned at the 5′-end of an RNA, e.g. an mRNA. Preferably, the 5′-CAP structure is connected via a 5′-5′-triphosphate linkage to the RNA.

5′-CAP structures which may be suitable in the context of the present invention are CAP0 (methylation of the first nucleobase, e.g. m7GpppN), CAP1 (additional methylation of the ribose of the adjacent nucleotide of m7GpppN), CAP2 (additional methylation of the ribose of the 2nd nucleotide downstream of the m7GpppN), CAP3 (additional methylation of the ribose of the 3rd nucleotide downstream of the m7GpppN), CAP4 (additional methylation of the ribose of the 4th nucleotide downstream of the m7GpppN), ARCA (anti-reverse CAP analogue), modified ARCA (e.g. phosphothioate modified ARCA), inosine, N1-methyl-guanosine, 2′-fluoro-guanosine, 7-deaza-guanosine, 8-oxo-guanosine, 2-amino-guanosine, LNA-guanosine, and 2-azido-guanosine.

A 5′-CAP (CAP0 or CAP1) structure may be formed in chemical RNA synthesis or in RNA in vitro transcription (co-transcriptional capping) using CAP analogues.

The term “CAP analogue” as used herein will be recognized and understood by the person of ordinary skill in the art, and is e.g. intended to refer to a non-polymerizable di-nucleotide or tri-nucleotide that has CAP functionality in that it facilitates translation or localization, and/or prevents degradation of a nucleic acid molecule, particularly of an RNA molecule, when incorporated at the 5′-end of the nucleic acid molecule. Non-polymerizable means that the CAP analogue will be incorporated only at the 5′-terminus because it does not have a 5′ triphosphate and therefore cannot be extended in the 3-direction by a template-dependent polymerase, particularly, by template-dependent RNA polymerase. Examples of CAP analogues include, but are not limited to, a chemical structure selected from the group consisting of m7GpppG, m7GpppA, m7GpppC; unmethylated CAP analogues (e.g. GpppG); dimethylated CAP analogue (e.g. m2,7GpppG), trimethylated CAP analogue (e.g. m2,2,7GpppG), dimethylated symmetrical CAP analogues (e.g. m7Gpppm7G), or anti reverse CAP analogues (e.g. ARCA; m7,2′OmeGpppG, m7,2′dGpppG, m7,3′OmeGpppG, m7,3′dGpppG and their tetraphosphate derivatives). Further CAP analogues have been described previously (WO2008016473, WO2008157688, WO2009149253, WO2011015347, and WO2013059475). Further suitable CAP analogues in that context are described in WO2017066793, WO2017066781, WO2017066791, WO2017066789, WO2017053297, WO2017066782, WO2018075827 and WO2017066797 wherein the disclosures referring to CAP analogues are incorporated herewith by reference.

In some embodiments, a modified CAP1 structure is generated using tri-nucleotide CAP analogue as disclosed in WO2017053297, WO2017066793, WO2017066781, WO2017066791, WO2017066789, WO2017066782, WO2018075827 and WO2017066797. In particular, any CAP structures derivable from the structure disclosed in claim 1-5 of WO2017053297 may be suitably used to co-transcriptionally generate a modified CAP1 structure. Further, any CAP structures derivable from the structure defined in claim 1 or claim 21 of WO2018075827 may be suitably used to co-transcriptionally generate a modified CAP1 structure.

In preferred embodiments, the (coding) RNA, in particular the mRNA comprises a CAP1 structure.

In preferred embodiments, the 5′-CAP structure may suitably be added co-transcriptionally using tri-nucleotide CAP analogue as defined herein in an RNA in vitro transcription reaction as defined herein.

In preferred embodiments, the CAP1 structure of the coding RNA of the invention is formed using co-transcriptional capping using tri-nucleotide CAP analogues m7G(5′)ppp(5′)(2′OMeA)pG or m7G(5′)ppp(5′)(2′OMeG)pG. A preferred CAP1 analogues in that context is m7G(5′)ppp(5′)(2′OMeA)pG.

In other preferred embodiments, the CAP1 structure of the RNA of the invention is formed using co-transcriptional capping using tri-nucleotide CAP analogue 3′OMe-m7G(5′)ppp(5′)(2′OMeA)pG.

In other embodiments, a CAP0 structure of the RNA of the invention is formed using co-transcriptional capping using CAP analogue 3′OMe-m7G(5′)ppp(5′)G.

In other embodiments, the 5′-CAP structure is formed via enzymatic capping using capping enzymes (e.g. vaccinia virus capping enzymes and/or CAP-dependent 2′-0 methyltransferases) to generate CAP0 or CAP1 or CAP2 structures. The 5′-CAP structure (CAP0 or CAP1) may be added using immobilized capping enzymes and/or CAP-dependent 2′-0 methyltransferases using methods and means disclosed in WO2016193226.

In preferred embodiments, about 70%, 75%, 80%, 85%, 90%, 95% of the RNA (species) comprises a CAP1 structure as determined using a capping assay. In preferred embodiments, less than about 20%, 15%, 10%, 5%, 4%, 3%, 2%, 1% of the RNA (species) does not comprises a CAP1 structure as determined using a capping assay. In other preferred embodiments, about 70%, 75%, 80%, 85%, 90%, 95% of the RNA (species) comprises a CAP0 structure as determined using a capping assay. In preferred embodiments, less than about 20%, 15%, 10%, 5%, 4%, 3%, 2%, 1% of the RNA (species) does not comprises a CAP0 structure as determined using a capping assay.

The term “RNA species” is not restricted to mean “one single molecule” but is understood to comprise an ensemble of essentially identical RNA molecules. Accordingly, it may relate to a plurality of essentially identical (coding) RNA molecules.

For determining the presence/absence of a CAP0 or a CAP1 structure, a capping assays as described in published PCT application WO2015101416, in particular, as described in claims 27 to 46 of published PCT application WO2015101416 can be used. Other capping assays that may be used to determine the presence/absence of a CAP0 or a CAP1 structure of an RNA are described in WO2020127959A1, or published PCT applications WO2014152673 and WO2014152659.

In preferred embodiments, the RNA comprises an m7G(5′)ppp(5′)(2′OMeA) CAP structure. In such embodiments, the coding RNA comprises a 5′-terminal m7G CAP, and an additional methylation of the ribose of the adjacent nucleotide of m7GpppN, in that case, a 2′O methylated Adenosine. Preferably, about 70%, 75%, 80%, 85%, 90%, 95% of the RNA (species) comprises such a CAP1 structure as determined using a capping assay.

In other preferred embodiments, the RNA comprises an m7G(5′)ppp(5′)(2′OMeG) CAP structure. In such embodiments, the coding RNA comprises a 5′-terminal m7G CAP, and an additional methylation of the ribose of the adjacent nucleotide, in that case, a 2′O methylated guanosine. Preferably, about 70%, 75%, 80%, 85%, 90%, 95% of the coding RNA (species) comprises such a CAP1 structure as determined using a capping assay.

Accordingly, the first nucleotide of said RNA or mRNA sequence, that is, the nucleotide downstream of the m7G(5′)ppp structure, may be a 2′O methylated guanosine or a 2′O methylated adenosine.

In one embodiment, the 5′-end of an mRNA is “GGGAGA”, preferably for an mRNA in which an mCap analog is used. In another embodiment, the 5′-end of an mRNA is “AGGAGA”, preferably for an mRNA in which a CleanCap® AG CAP analog is used. In a further embodiment, the 5′-end of an mRNA is “GGGAGA”, preferably for an mRNA in which a CleanCap® GG CAP analog is used.

In the context of the present invention, a 5′-CAP structure may also be formed in chemical RNA synthesis or RNA in vitro transcription (co-transcriptional capping) using CAP analogues, or a CAP structure may be formed in vitro using capping enzymes. Kits comprising capping enzymes are commercially available (e.g. ScriptCap™ Capping Enzyme and ScriptCap™ 2′-O-Methyltransferase (both from CellScript)). Therefore, the RNA transcript is preferably treated according to the manufacturer's instructions.

Thusly, a CAP analogue refers to a non-polymerizable di-nucleotide that has CAP functionality in that it facilitates translation or localization, and/or prevents degradation of the RNA molecule when incorporated at the 5′-end of the RNA molecule. Non-polymerizable means that the CAP analogue will be incorporated only at the 5′-terminus because it does not have a 5′ triphosphate and therefore cannot be extended in the 3-direction by a template-dependent RNA polymerase.

Further examples of 5′-CAP structures include glyceryl, inverted deoxy abasic residue (moiety), 4′,5′ methylene nucleotide, 1-(beta-D-erythrofuranosyl) nucleotide, 4′-thio nucleotide, carbocyclic nucleotide, 1,5-anhydrohexitol nucleotide, L-nucleotides, alpha-nucleotide, modified base nucleotide, threo-pentofuranosyl nucleotide, acyclic 3′,4′-seco nucleotide, acyclic 3,4-dihydroxybutyl nucleotide, acyclic 3,5 dihydroxypentyl nucleotide, 3′-3′-inverted nucleotide moiety, 3-3-inverted abasic moiety, 3-2′-inverted nucleotide moiety, 3-2′-inverted abasic moiety, 1,4-butanediol phosphate, 3-phosphoramidate, hexylphosphate, aminohexyl phosphate, 3-phosphate, 3′phosphorothioate, phosphorodithioate, or bridging or non-bridging methylphosphonate moiety. These modified 5′-CAP structures are regarded as at least one modification in this context and may be used in the context of the present invention to modify the mRNA sequence of the inventive composition.

Particularly preferred modified 5′-CAP structures are CAP1 (methylation of the ribose of the adjacent nucleotide of m7G), cap2 (additional methylation of the ribose of the 2nd nucleotide downstream of the m7G), CAP3 (additional methylation of the ribose of the 3rd nucleotide downstream of the m7G), CAP4 (methylation of the ribose of the 4th nucleotide downstream of the m7G), ARCA (anti-reverse CAP analogue, modified ARCA (e.g. phosphothioate modified ARCA), CleanCap or respectively m7G(5′)ppp(5′)(2′OMeA)pG or m7G(5′)ppp(5′)(2′OMeG)pG (TriLink) and or a CAP-structure as disclosed in WO2017053297 (herewith incorporated by reference), inosine, N1-methyl-guanosine, 2′-fluoro-guanosine, 7-deaza-guanosine, 8-oxo-guanosine, 2-amino-guanosine, LNA-guanosine, and 2-azido-guanosine. In particular, any CAP structures derivable from the structure disclosed in claim 1-5 of WO2017053297 may be suitably used to co-transcriptionally generate a modified CAP1 structure. Further, any CAP structures derivable from the structure defined in claim 1 or claim 21 of WO2018075827 may be suitably used to co-transcriptionally generate a modified CAP1 structure.

Furthermore, CAP analogues have been described previously (U.S. Pat. No. 7,074,596, WO2008016473, WO2008157688, WO2009149253, WO2011015347, and WO2013059475). The synthesis of N7-(4-chlorophenoxyethyl) substituted dinucleotide CAP analogues has been described recently (Kore et al. (2013) Bioorg. Med. Chem. 21(15): 4570-4). Further suitable CAP analogues in that context are described in WO2017066793, WO2017066781, WO2017066791, WO2017066789, WO2017066782, WO2018075827 and WO2017066797 wherein the specific disclosures referring to CAP analogues are incorporated herein by reference.

### Poly(A) Sequence/polyA-Tail

A polyA-tail also called “3′-poly(A) tail”, “polyA sequence” or “poly(A) sequence” is typically a long sequence of adenosine nucleotides of up to about 400 adenosine nucleotides, e.g. from 10 to 200, 10 to 100, 40 to 80, 50 to 70, about 25 to about 400, preferably from about 50 to about 400, more preferably from about 50 to about 300, even more preferably from about 50 to about 250, most preferably from about 60 to about 250 adenosine nucleotides, or about 40 to about 150 adenosine nucleotides, added to the 3-end of a RNA. In a particularly preferred embodiment, the poly(A) sequence comprises about 64 adenosine nucleotides. In another particularly preferred embodiment, the poly(A) sequence comprises about 100 adenosine nucleotides. Moreover, poly(A) sequences, or poly(A) tails may be generated in vitro by enzymatic polyadenylation of the RNA, e.g. using Poly(A)polymerases derived from E. coli or yeast. Suitably, the poly(A) sequence of the coding RNA may be long enough to bind at least 2, 3, 4, 5 or more monomers of PolyA Binding Proteins.

Polyadenylation is typically understood to be the addition of a poly(A) sequence to a nucleic acid molecule, such as an RNA molecule, e.g. to a premature mRNA. Polyadenylation may be induced by a so called polyadenylation signal. This signal is preferably located within a stretch of nucleotides at the 3-end of a nucleic acid molecule, such as an RNA molecule, to be polyadenylated. A polyadenylation signal typically comprises a hexamer consisting of adenine and uracil/thymine nucleotides, preferably the hexamer sequence AAUAAA. Other sequences, preferably hexamer sequences, are also conceivable. Polyadenylation typically occurs during processing of a pre-mRNA (also called premature-mRNA). Typically, RNA maturation (from pre-mRNA to mature mRNA) comprises the step of polyadenylation.

Thusly, according to a further preferred embodiment, the composition comprises an mRNA compound comprising an mRNA sequence containing a polyA tail on the 3-terminus of typically about 10 to 200 adenosine nucleotides, preferably about 10 to 100 adenosine nucleotides, more preferably about 40 to 80 adenosine nucleotides or even more preferably about 50 to 70 adenosine nucleotides. Preferably, the poly(A) sequence is derived from a DNA template by RNA in vitro transcription. Alternatively, the poly(A) sequence may also be obtained in vitro by common methods of chemical-synthesis without being necessarily transcribed from a DNA-progenitor. Moreover, poly(A) sequences, or poly(A) tails may be generated by enzymatic polyadenylation of the RNA according to the present invention using commercially available polyadenylation kits and corresponding protocols known in the art.

Alternatively, the mRNA as described herein optionally comprises a polyadenylation signal, which is defined herein as a signal, which conveys polyadenylation to a (transcribed) RNA by specific protein factors (e.g. cleavage and polyadenylation specificity factor (CPSF), cleavage stimulation factor (CstF), cleavage factors I and II (CF I and CF II), poly(A) polymerase (PAP)). In this context, a consensus polyadenylation signal is preferred comprising the NN(U/T)ANA consensus sequence. In a particularly preferred aspect, the polyadenylation signal comprises one of the following sequences: AA(U/T)AAA or A(U/T)(U/T)AAA (wherein uridine is usually present in RNA and thymidine is usually present in DNA).

### Poly(C) Sequence

A poly-(C)-sequence is typically a long sequence of cytosine nucleotides, typically about 10 to about 200 cytosine nucleotides, preferably about 10 to about 100 cytosine nucleotides, more preferably about 10 to about 70 cytosine nucleotides or even more preferably about 20 to about 50 or even about 20 to about 30 cytosine nucleotides. A poly(C) sequence may preferably be located 3′ of the coding region comprised by a nucleic acid.

Thusly, according to a further preferred embodiment, the composition of the invention comprises an mRNA compound comprising a poly(C) tail on the 3-terminus of typically about 10 to 200 cytosine nucleotides, preferably about 10 to 100 cytosine nucleotides, more preferably about 20 to 70 cytosine nucleotides or even more preferably about 20 to 60 or even 10 to 40 cytosine nucleotides.

In one preferred embodiment, the mRNA compound comprises, preferably in 5′- to 3-direction:
a) a 5′-CAP structure, preferably m7GpppN, more preferably CAP1 or m7G(5′)ppp(5′)(2′OMeA)pG;
b) optionally, a 5′-UTR element,
c) at least one coding region encoding at least one antigenic peptide or protein,
d) optionally, a poly(A) sequence, preferably comprising 64 adenosines or 100 adenosines;
e) optionally, a poly(C) sequence, preferably comprising 30 cytosines;
f) optionally, a histone stem-loop selected from SEQ ID NO:3 or 4; and/or
g) optionally, a 3-terminal sequence element selected from SEQ ID NO:41-70.

### UTRs

In a preferred embodiment, the composition comprises an mRNA compound comprising at least one 5′- or 3′-UTR element. In this context, an UTR element comprises or consists of a nucleic acid sequence, which is derived from the 5′- or 3′-UTR of any naturally occurring gene or which is derived from a fragment, a homolog or a variant of the 5′- or 3′-UTR of a gene. Preferably, the 5′- or 3′-UTR element used according to the present invention is heterologous to the at least one coding region of the mRNA sequence of the invention. Even if 5′- or 3′-UTR elements derived from naturally occurring genes are preferred, also synthetically engineered UTR elements may be used in the context of the present invention.

The term “3′-UTR element” typically refers to a nucleic acid sequence, which comprises or consists of a nucleic acid sequence that is derived from a 3′-UTR or from a variant of a 3′-UTR. A 3′-UTR element in the sense of the present invention may represent the 3′-UTR of an RNA, preferably an mRNA. Thus, in the sense of the present invention, preferably, a 3′-UTR element may be the 3′-UTR of an RNA, preferably of an mRNA, or it may be the transcription template for a 3′-UTR of an RNA. Thus, a 3′-UTR element preferably is a nucleic acid sequence which corresponds to the 3′-UTR of an RNA, preferably to the 3′-UTR of an mRNA, such as an mRNA obtained by transcription of a genetically engineered vector construct. Preferably, the 3′-UTR element fulfils the function of a 3′-UTR or encodes a sequence which fulfils the function of a 3′-UTR.

Preferably, the at least one 3′-UTR element comprises or consists of a nucleic acid sequence derived from the 3′-UTR of a chordate gene, preferably a vertebrate gene, more preferably a mammalian gene, most preferably a human gene, or from a variant of the 3′-UTR of a chordate gene, preferably a vertebrate gene, more preferably a mammalian gene, most preferably a human gene.

Preferably, the composition comprises an mRNA compound that comprises a 3′-UTR element, which may be derivable from a gene that relates to an mRNA with an enhanced half-life (that provides a stable mRNA), for example a 3′-UTR element as defined and described below. Preferably, the 3′-UTR element comprises or consists of a nucleic acid sequence derived from a 3′-UTR of a gene, which preferably encodes a stable mRNA, or from a homolog, a fragment or a variant of said gene.

In one preferred embodiment, the UTR-combinations which are disclosed in Table 1, claims 1 and claim 4, claims 6-8 and claim 9 of WO2019077001 are preferred UTR-combinations for mRNA compounds of the present invention. Further, preferably, the UTR-combinations as disclosed on page 24, second full paragraph after Table 1 and page 24, last paragraph to page 29, second paragraph of WO2019077001 are preferred UTR-combinations for mRNA compounds of the present invention. WO2019077001 is incorporated herein by reference in its entirety.

In a further preferred embodiment, that 3′-UTR element comprises or consists of a nucleic acid sequence which is derived from a 3′-UTR of a gene selected from the group consisting of a 3′-UTR of a gene selected from PSMB3 (SEQ ID NO:19, SEQ ID NO:20), ALB/albumin (SEQ ID NO:13-SEQ ID NO:18), alpha-globin (referred to as “muag” i.e. a mutated alpha-globin 3′-UTR; SEQ ID NO:11, SEQ ID NO:12), CASP1 (preferably SEQ ID NO:81 (DNA) or SEQ ID NO:82 (RNA)), COX6B1 (preferably SEQ ID NO:83 (DNA) or SEQ ID NO:84 (RNA)), GNAS (preferably SEQ ID NO:85 (DNA) or SEQ ID NO:86 (RNA)), NDUFA1 (preferably SEQ ID NO:87 (DNA) or SEQ ID NO:88 (RNA)) and RPS9 (preferably SEQ ID NO:79 (DNA) or SEQ ID NO:80 (RNA)), or from a homolog, a fragment or a variant of any one of these genes (for example, human albumin/alb 3′-UTR as disclosed in SEQ ID NO:1369 of WO2013143700, which is incorporated herein by reference), or from a homolog, a fragment or a variant thereof. In a further preferred embodiment, the 3′-UTR element comprises the nucleic acid sequence derived from a fragment of the human albumin gene according to SEQ ID NO:1376 of WO2013143700 (albumin/alb 3′-UTR). In a further preferred embodiment, the 3′-UTR element comprises or consists of a nucleic acid sequence which is derived from a 3′-UTR of an albumin gene, preferably a vertebrate albumin gene, more preferably a mammalian albumin gene, most preferably a human albumin gene such as from the 3′-UTR of the human albumin gene according to GenBank Accession number NM_000477.5, or a fragment or variant thereof. In another preferred embodiment, the 3′-UTR element comprises or consists of the center, α-complex-binding portion of the 3′-UTR of an α-globin gene, such as of a human α-globin gene, or a homolog, a fragment, or a variant of an α-globin gene, preferably (according to SEQ ID NO:5 or SEQ ID NO:6 (both HBA1) or SEQ ID NO:7 or SEQ ID NO:8 (both HBA2)), or an α-complex-binding portion of the 3′-UTR of an α-globin gene (also named herein as “muag”), herein SEQ ID NO:11, SEQ ID NO:12; corresponding to SEQ ID NO:1393 of patent application WO2013143700).

In another preferred embodiment, the 3′-UTR element comprises or consists of a nucleic acid sequence which is derived from a 3′-UTR of an α- or β-globin gene, preferably a vertebrate α- or β-globin gene, and preferably a mammalian β- or β-globin gene, preferably a human α- or β globin gene according to SEQ ID NO:5, 7, 9, 11 or the corresponding RNA sequences SEQ ID NO:6, 8, 10, 12. In another preferred embodiment, the 3′-UTR element comprises or consists of a nucleic acid sequence which is derived from a 3′-UTR of ALB/albumin (SEQ ID NO:13-SEQ ID NO:18) or PSMB3 (SEQ ID NO:19/20).

In this context it is also preferred that the 3′-UTR element of the mRNA sequence according to the invention comprises or consists of a corresponding RNA sequence of the nucleic acid sequence according to SEQ ID NO:11 as shown in SEQ ID NO:12, or a homolog, a fragment or variant thereof.

UTR-combination SLC7A3 (5′-UTR of mouse solute carrier family 7 (cationic amino acid transporter, y+ system), member 3)/PSMB3: In another preferred embodiment, the mRNA compound comprises a 5′-UTR element, which comprises or consists of a nucleic acid sequence which is derived from a cationic amino acid transporter 3 (solute carrier family 7 member 3, SLC7A3; preferably SEQ ID NO:77 (DNA) or SEQ ID NO:78 (RNA)) gene, wherein said 5′-UTR element comprises or consists of a DNA sequence according to SEQ ID NO:15 as disclosed in WO2019077001 or respectively a RNA sequence according to SEQ ID NO:16 as disclosed in WO2019077001. In another preferred embodiment, the mRNA compound comprises a 3′-UTR element, which comprises or consists of a nucleic acid sequence which is derived from a proteasome subunit beta type-3 (PSMB3) gene, wherein said 3′-UTR element comprises or consists of a DNA sequence according to SEQ ID NO:23 as disclosed in WO2019077001 or respectively a RNA sequence according to SEQ ID NO:24 as disclosed in WO2019077001. In further preferred embodiments, the mRNA compound comprises an UTR-combination as disclosed in WO2019077001, i.e. both a 5′-UTR element, which comprises or consists of a nucleic acid sequence which is derived from a SLC7A3 gene and a 3′-UTR element, which comprises or consists of a nucleic acid sequence which is derived from a PSMB3 gene.

UTR-combination RPL31 (5′-UTR of mouse ribosomal protein L31; preferably SEQ ID NO:75 (DNA) or SEQ ID NO:76 (RNA))/RPS9 (3-UTR of human ribosomal protein S9 (RPS9; preferably SEQ ID NO:79 (DNA) or SEQ ID NO:80 (RNA)): In another preferred embodiment, the mRNA compound comprises a 5′-UTR element, which comprises or consists of a nucleic acid sequence which is derived from a 60S ribosomal protein L31 (RPL31) gene, wherein said 5′-UTR element comprises or consists of a DNA sequence according to SEQ ID NO:13 as disclosed in WO2019077001 or respectively a RNA sequence according to SEQ ID NO:14 as disclosed in WO2019077001 or preferably SEQ ID NO:75/76. In another preferred embodiment, the mRNA compound comprises a 3′-UTR element, which comprises or consists of a nucleic acid sequence which is derived from a 40S ribosomal protein S9 (RPS9) gene, wherein said 3′-UTR element comprises or consists of a DNA sequence according to SEQ ID NO:33 as disclosed in WO2019077001 or respectively a RNA sequence according to SEQ ID NO:34 as disclosed in WO2019077001. In further preferred embodiments, the mRNA compound comprises an UTR-combination as disclosed in WO2019077001, i.e. both a 5′-UTR element, which comprises or consists of a nucleic acid sequence which is derived from a RPL31 gene and a 3′-UTR element, which comprises or consists of a nucleic acid sequence which is derived from a RPS9 gene (preferably SEQ ID NO:79/80).

In a very preferred embodiment, the 5′-UTR element of the mRNA sequence according to the invention comprises or consists of a corresponding RNA sequence of the nucleic acid sequence according to SEQ ID NO:21 or SEQ ID NO:22, i.e. HSD17B4. Also, in a very preferred embodiment, the 3′-UTR element of the mRNA sequence according to the invention comprises or consists of a corresponding RNA sequence of the nucleic acid sequence according to SEQ ID NO:19 or SEQ ID NO:20, i.e. PSMB3. In also a very preferred embodiment, the 5′-UTR element of the mRNA sequence and the 3′-UTR-element according to the invention comprises or consists of a combination of aforementioned HSD17B4 and PSMB3-UTRs.

The term “a nucleic acid sequence which is derived from the 3′-UTR of a [ . . . ] gene” preferably refers to a nucleic acid sequence which is based on the 3′-UTR sequence of a [ . . . ] gene or on a part thereof, such as on the 3′-UTR of an albumin gene, an β-globin gene, a β-globin gene, a tyrosine hydroxylase gene, a lipoxygenase gene, or a collagen alpha gene, such as a collagen alpha 1(I) gene, preferably of an albumin gene or on a part thereof. This term includes sequences corresponding to the entire 3′-UTR sequence, i.e. the full length 3′-UTR sequence of a gene, and sequences corresponding to a fragment of the 3′-UTR sequence of a gene, such as an albumin gene, α-globin gene, β-globin gene, tyrosine hydroxylase gene, lipoxygenase gene, or collagen alpha gene, such as a collagen alpha 1(I) gene, preferably of an albumin gene.

The term “a nucleic acid sequence which is derived from a variant of the 3′-UTR of a [ . . . ] gene” preferably refers to a nucleic acid sequence, which is based on a variant of the 3′-UTR sequence of a gene, such as on a variant of the 3′-UTR of an albumin gene, an α-globin gene, a β-globin gene, a tyrosine hydroxylase gene, a lipoxygenase gene, or a collagen alpha gene, such as a collagen alpha 1(I) gene, or on a part thereof as described above. This term includes sequences corresponding to the entire sequence of the variant of the 3′-UTR of a gene, i.e. the full length variant 3′-UTR sequence of a gene, and sequences corresponding to a fragment of the variant 3′-UTR sequence of a gene. A fragment in this context preferably consists of a continuous stretch of nucleotides corresponding to a continuous stretch of nucleotides in the full-length variant 3′-UTR, which represents at least 20%, preferably at least 30%, more preferably at least 40%, more preferably at least 50%, even more preferably at least 60%, even more preferably at least 70%, even more preferably at least 80%, and most preferably at least 90% of the full-length variant 3′-UTR. Such a fragment of a variant, in the sense of the present invention, is preferably a functional fragment of a variant as described herein.

According to a preferred embodiment, the mRNA compound comprising an mRNA sequence according to the invention comprises a 5′-CAP structure and/or at least one 3-untranslated region element (3′-UTR element), preferably as defined herein. More preferably, the RNA further comprises a 5′-UTR element as defined herein.

In one preferred embodiment, the mRNA compound comprises, preferably in 5′- to 3-direction:
a) a 5′-CAP structure, preferably m7GpppN, more preferably CAP1 or m7G(5′)ppp(5′)(2′OMeA)pG;
b) optionally, a 5′-UTR element,
c) at least one coding region encoding at least one antigenic peptide or protein,
d) optionally, a 3′-UTR element, preferably comprising or consisting of a nucleic acid sequence which is derived from an alpha globin gene, preferably comprising the corresponding RNA sequence of the nucleic acid sequence according to SEQ ID NO:11 as shown in SEQ ID NO:12, a homolog, a fragment or a variant thereof;
e) optionally, a poly(A) sequence, preferably comprising 64 adenosines or 100 adenosines;
f) optionally, a poly(C) sequence, preferably comprising 30 cytosines;
g) optionally, a histone stem-loop selected from SEQ ID NO:3 or 4; and/or
h) optionally, a 3-terminal sequence element selected from SEQ ID NO:41-70.

In a further preferred embodiment, the mRNA compound comprises, preferably in 5′- to 3-direction:
a) a 5′-CAP structure, preferably m7GpppN, more preferably CAP1 or m7G(5′)ppp(5′)(2′OMeA)pG;
b) optionally, a 5′-UTR element;
c) at least one coding region encoding at least one antigenic peptide or protein, preferably derived from a protein of SARS coronavirus 2 (SARS-CoV-2), nCoV-2019 coronavirus, SARS coronavirus (SARS-CoV), an Bunyavirales virus, Cytomegalovirus (CMV), Dengue viruses (DENV-1, DENV-2, DENV-3 and DENV-4), Ebola virus (EBOV), Flavivirus, Hepatitis B virus (HBV), Herpes simplex virus (HSV), Human immunodeficiency virus (HIV), Human metapneumovirus (HMPV), Human Papilloma virus (HPV), Human parainfluenza viruses (HPIV), Influenza virus, extraintestinal pathogenic E. coli (ExPEC), Lassa mammarenavirus (LASV), MERS coronavirus, Mycobacterium tuberculosis, Nipah virus, Norovirus, Rabies virus (RABV), Respiratory Syncytial virus (RSV), Rhinovirus, Rotavirus, Vaccinia virus, Yellow Fever virus (YFV), Zika virus (ZIKV), Chlamydia trachomatis (i.e. bacterium Chlamydia causing Chlamydia), or Malaria parasite (e.g. Plasmodium falciparum, Plasmodium vivax, Plasmodium malariae, or Plasmodium ovale) or a fragment or variant thereof,
d) optionally, a 3′-UTR element, preferably comprising or consisting of a nucleic acid sequence which is derived from an alpha globin gene, preferably comprising the corresponding RNA sequence of the nucleic acid sequence according to SEQ ID NO:11 as shown in SEQ ID NO:12, a homolog, a fragment or a variant thereof;
e) optionally, a poly(A) sequence, preferably comprising 64 adenosines or 100 adenosines;
f) optionally, a poly(C) sequence, preferably comprising 30 cytosines;
g) optionally, a histone stem-loop selected from SEQ ID NO:3 or 4; and/or
h) optionally, a 3-terminal sequence element selected from SEQ ID NO:41-70.

In a further preferred embodiment, the composition comprises an mRNA compound comprising at least one 5′-untranslated region element (5′-UTR element). Preferably, the at least one 5′-UTR element comprises or consists of a nucleic acid sequence, which is derived from the 5′-UTR of a TOP gene or which is derived from a fragment, homolog or variant of the 5′-UTR of a TOP gene. It is preferred that the 5′-UTR element does not comprise a TOP motif or a 5′-TOP, as defined above.

In some embodiments, the nucleic acid sequence of the 5′-UTR element, which is derived from a 5′-UTR of a TOP gene, terminates at its 3-end with a nucleotide located at position 1, 2, 3, 4, 5, 6, 7, 8, 9 or 10 upstream of the start codon (e.g. A(U/T)G) of the gene or mRNA it is derived from. Thus, the 5′-UTR element does not comprise any part of the protein coding region. Thus, preferably, the only protein coding part of the at least one mRNA sequence is provided by the coding region.

The nucleic acid sequence derived from the 5′-UTR of a TOP gene is preferably derived from a eukaryotic TOP gene, preferably a plant or animal TOP gene, more preferably a chordate TOP gene, even more preferably a vertebrate TOP gene, most preferably a mammalian TOP gene, such as a human TOP gene.

For example, the 5′-UTR element may be selected from 5′-UTR elements comprising or consisting of a nucleic acid sequence, which is derived from a nucleic acid sequence selected from the group consisting of SEQ ID NO:1-1363, SEQ ID NO:1395, SEQ ID NO:1421 and SEQ ID NO:1422 of the patent application WO2013143700, whose disclosure is incorporated herein by reference, from the homologs of SEQ ID NO:1-1363, SEQ ID NO:1395, SEQ ID NO:1421 and SEQ ID NO:1422 of the patent application WO2013143700, from a variant thereof, or preferably from a corresponding RNA sequence. The term “homologs of SEQ ID NO:1-1363, SEQ ID NO:1395, SEQ ID NO:1421 and SEQ ID NO:1422 of the patent application WO2013143700” refers to sequences of other species than Homo sapiens, which are homologous to the sequences according to SEQ ID NO:1-1363, SEQ ID NO:1395, SEQ ID NO:1421 and SEQ ID NO:1422 of the patent application WO2013143700.

In a preferred embodiment, the 5′-UTR element of the mRNA compound comprises or consists of a nucleic acid sequence, which is derived from a nucleic acid sequence extending from nucleotide position 5 (i.e. the nucleotide that is located at position 5 in the sequence) to the nucleotide position immediately 5′ to the start codon (located at the 3-end of the sequences), e.g. the nucleotide position immediately 5′ to the ATG sequence, of a nucleic acid sequence selected from SEQ ID NO:1-1363, SEQ ID NO:1395, SEQ ID NO:1421 and SEQ ID NO:1422 of the patent application WO2013143700, from the homologs of SEQ ID NO:1-1363, SEQ ID NO:1395, SEQ ID NO:1421 and SEQ ID NO:1422 of the patent application WO2013/143700 from a variant thereof, or a corresponding RNA sequence. It is particularly preferred that the 5′-UTR element is derived from a nucleic acid sequence extending from the nucleotide position immediately 3′ to the 5′-TOP to the nucleotide position immediately 5′ to the start codon (located at the 3-end of the sequences), e.g. the nucleotide position immediately 5′ to the ATG sequence, of a nucleic acid sequence selected from SEQ ID NO:1-1363, SEQ ID NO:1395, SEQ ID NO:1421 and SEQ ID NO:1422 of the patent application WO2013143700, from the homologs of SEQ ID NO:1-1363, SEQ ID NO:1395, SEQ ID NO:1421 and SEQ ID NO:1422 of the patent application WO2013143700, from a variant thereof, or a corresponding RNA sequence.

In a further preferred embodiment, the 5′-UTR element comprises or consists of a nucleic acid sequence, which is derived from a 5′-UTR of a TOP gene encoding a ribosomal protein or from a variant of a 5′-UTR of a TOP gene encoding a ribosomal protein. For example, the 5′-UTR element comprises or consists of a nucleic acid sequence, which is derived from a 5′-UTR of a nucleic acid sequence according to any of SEQ ID NO:67, 170, 193, 244, 259, 554, 650, 675, 700, 721, 913, 1016, 1063, 1120, 1138, and 1284-1360 of the patent application WO2013143700, a corresponding RNA sequence, a homolog thereof, or a variant thereof as described herein, preferably lacking the 5′-TOP motif. As described above, the sequence extending from position 5 to the nucleotide immediately 5′ to the ATG (which is located at the 3′-end of the sequences) corresponds to the 5′-UTR of said sequences.

Preferably, the 5′-UTR element comprises or consists of a nucleic acid sequence, which is derived from a 5′-UTR of a TOP gene encoding a ribosomal Large protein (RPL) or from a homolog or variant of a 5′-UTR of a TOP gene encoding a ribosomal Large protein (RPL). For example, the 5′-UTR element comprises or consists of a nucleic acid sequence, which is derived from a 5′-UTR of a nucleic acid sequence according to any of SEQ ID NO:67, 259, 1284-1318, 1344, 1346, 1348-1354, 1357, 1358, 1421 and 1422 of the patent application WO2013143700, a corresponding RNA sequence, a homolog thereof, or a variant thereof as described herein, preferably lacking the 5′-TOP motif.

In a particularly preferred embodiment, the 5′-UTR element comprises or consists of a nucleic acid sequence which is derived from the 5′-UTR of a ribosomal protein Large 32 gene, preferably from a vertebrate ribosomal protein Large 32 (L32) gene, more preferably from a mammalian ribosomal protein Large 32 (L32) gene, most preferably from a human ribosomal protein Large 32 (L32) gene, or from a variant of the 5′UTR of a ribosomal protein Large 32 gene, preferably from a vertebrate ribosomal protein Large 32 (L32) gene, more preferably from a mammalian ribosomal protein Large 32 (L32) gene, most preferably from a human ribosomal protein Large 32 (L32) gene, wherein preferably the 5′-UTR element does not comprise the 5′-TOP of said gene.

Accordingly, in a preferred embodiment, the 5′-UTR element comprises or consists of a nucleic acid sequence, which has an identity of at least about 40%, preferably of at least about 50%, preferably of at least about 60%, preferably of at least about 70%, more preferably of at least about 80%, more preferably of at least about 90%, even more preferably of at least about 95%, even more preferably of at least about 99% to the nucleic acid sequence according to SEQ ID NO:23 or SEQ ID NO:24 (5′-UTR of human ribosomal protein Large 32 lacking the 5′-terminal oligopyrimidine tract; corresponding to SEQ ID NO:1368 of the patent application WO2013143700) or preferably to a corresponding RNA sequence, or wherein the at least one 5′-UTR element comprises or consists of a fragment of a nucleic acid sequence which has an identity of at least about 40%, preferably of at least about 50%, preferably of at least about 60%, preferably of at least about 70%, more preferably of at least about 80%, more preferably of at least about 90%, even more preferably of at least about 95%, even more preferably of at least about 99% to the nucleic acid sequence according to SEQ ID NO:23 or more preferably to a corresponding RNA sequence (SEQ ID NO:24), wherein, preferably, the fragment is as described above, i.e. being a continuous stretch of nucleotides representing at least 20% etc. of the full-length 5′-UTR. Preferably, the fragment exhibits a length of at least about 20 nucleotides or more, preferably of at least about 30 nucleotides or more, more preferably of at least about 40 nucleotides or more. Preferably, the fragment is a functional fragment as described herein.

In some embodiments, the mRNA compound comprises a 5′-UTR element, which comprises or consists of a nucleic acid sequence which is derived from the 5′-UTR of a vertebrate TOP gene, such as a mammalian, e.g. a human TOP gene, selected from RPSA, RPS2, RPS3, RPS3A, RPS4, RPS5, RPS6, RPS7, RPS8, RPS9, RPS10, RPS11, RPS12, RPS13, RPS14, RPS15, RPS15A, RPS16, RPS17, RPS18, RPS19, RPS20, RPS21, RPS23, RPS24, RPS25, RPS26, RPS27, RPS27A, RPS28, RPS29, RPS30, RPL3, RPL4, RPL5, RPL6, RPL7, RPL7A, RPL8, RPL9, RPL10, RPL10A, RPL11, RPL12, RPL13, RPL13A, RPL14, RPL15, RPL17, RPL18, RPL18A, RPL19, RPL21, RPL22, RPL23, RPL23A, RPL24, RPL26, RPL27, RPL27A, RPL28, RPL29, RPL30, RPL31, RPL32, RPL34, RPL35, RPL35A, RPL36, RPL36A, RPL37, RPL37A, RPL38, RPL39, RPL40, RPL41, RPLP0, RPLP1, RPLP2, RPLP3, RPLP0, RPLP1, RPLP2, EEF1A1, EEF1B2, EEF1D, EEF1G, EEF2, EIF3E, EIF3F, EIF3H, EIF2S3, EIF3C, EIF3K, EIF3EIP, EIF4A2, PABPC1, HNRNPA1, TPT1, TUBB1, UBA52, NPM1, ATP5G2, GNB2L1, NME2, UQCRB, or from a homolog or variant thereof, wherein preferably the 5′-UTR element does not comprise a TOP motif or the 5′-TOP of said genes, and wherein optionally the 5′-UTR element starts at its 5′-end with a nucleotide located at position 1, 2, 3, 4, 5, 6, 7, 8, 9 or 10 downstream of the 5′-terminal oligopyrimidine tract (TOP) and wherein further optionally the 5′-UTR element which is derived from a 5′-UTR of a TOP gene terminates at its 3-end with a nucleotide located at position 1, 2, 3, 4, 5, 6, 7, 8, 9 or 10 upstream of the start codon (A(U/T)G) of the gene it is derived from.

In further preferred embodiments, the 5′-UTR element comprises or consists of a nucleic acid sequence, which is derived from the 5′-UTR of a ribosomal protein Large 32 gene (RPL32), a ribosomal protein Large 35 gene (RPL35), a ribosomal protein Large 21 gene (RPL21), a solute carrier family 7 (cationic amino acid transporter, y+ system), member 3, a ribosomal protein L31 (RPL31; preferably SEQ ID NO:75 (DNA) or SEQ ID NO:76 (RNA)), an ATP synthase, H+ transporting, mitochondrial F1 complex, alpha subunit 1, cardiac muscle gene (ATP5A1), an hydroxysteroid (17-beta) dehydrogenase 4 gene (HSD17B4; SEQ ID NO:21, SEQ ID NO:22), an androgen-induced 1 gene (AIG1), cytochrome c oxidase subunit VIc gene (COX6C), or a N-acylsphingosine amidohydrolase (acid ceramidase) 1 gene (ASAH1) or from a variant thereof, preferably from a vertebrate ribosomal protein Large 32 gene (RPL32), a vertebrate ribosomal protein Large 35 gene (RPL35), a vertebrate ribosomal protein Large 21 gene (RPL21), a vertebrate ATP synthase, H+ transporting, mitochondrial F1 complex, alpha subunit 1, cardiac muscle (ATP5A1) gene, a vertebrate hydroxysteroid (17-beta) dehydrogenase 4 gene (HSD17B4; SEQ ID NO:21, SEQ ID NO:22), a vertebrate androgen-induced 1 gene (AIG1), a vertebrate cytochrome c oxidase subunit VIc gene (COX6C), or a vertebrate N-acylsphingosine amidohydrolase (acid ceramidase) 1 gene (ASAH1) or from a variant thereof, more preferably from a mammalian ribosomal protein Large 32 gene (RPL32), a ribosomal protein Large 35 gene (RPL35), a ribosomal protein Large 21 gene (RPL21), a mammalian ATP synthase, H+ transporting, mitochondrial F1 complex, alpha subunit 1, cardiac muscle gene (ATP5A1), a mammalian hydroxysteroid (17-beta) dehydrogenase 4 gene (HSD17B4; SEQ ID NO:21, SEQ ID NO:22), a mammalian androgen-induced 1 gene (AIG1), a mammalian cyto-chrome c oxidase subunit VIc gene (COX6C), or a mammalian N-acylsphingosine amidohydrolase (acid ceramidase) 1 gene (ASAH1) or from a variant thereof, most preferably from a human ribosomal protein Large 32 gene (RPL32), a human ribosomal protein Large 35 gene (RPL35), a human ribosomal protein Large 21 gene (RPL21), a human ATP synthase, H+ transporting, mitochondrial F1 complex, alpha subunit 1, cardiac muscle gene (ATP5A1), a human hydroxysteroid (17-beta) dehydrogenase 4 gene (HSD17B4; SEQ ID NO:21, SEQ ID NO:22), a human androgen-induced 1 gene (AIG1), a human cytochrome c oxidase subunit VIc gene (COX6C), or a human N-acylsphingosine amidohydrolase (acid ceramidase) 1 gene (ASAH1) or from a variant thereof, wherein preferably the 5′-UTR element does not comprise the 5′-TOP of said gene.

Accordingly, in a preferred embodiment, the 5′-UTR element comprises or consists of a nucleic acid sequence, which has an identity of at least about 40%, preferably of at least about 50%, preferably of at least about 60%, preferably of at least about 70%, more preferably of at least about 80%, more preferably of at least about 90%, even more preferably of at least about 95%, even more preferably of at least about 99% to the nucleic acid sequence according to SEQ ID NO:1368, or SEQ ID NO:1412-1420 of the patent application WO2013143700, or a corresponding RNA sequence, or wherein the at least one 5′-UTR element comprises or consists of a fragment of a nucleic acid sequence which has an identity of at least about 40%, preferably of at least about 50%, preferably of at least about 60%, preferably of at least about 70%, more preferably of at least about 80%, more preferably of at least about 90%, even more preferably of at least about 95%, even more preferably of at least about 99% to the nucleic acid sequence according to SEQ ID NO:1368, or SEQ ID NO:1412-1420 of the patent application WO2013143700, wherein, preferably, the fragment is as described above, i.e. being a continuous stretch of nucleotides representing at least 20% etc. of the full-length 5′-UTR. Preferably, the fragment exhibits a length of at least about 20 nucleotides or more, preferably of at least about 30 nucleotides or more, more preferably of at least about 40 nucleotides or more. Preferably, the fragment is a functional fragment as described herein.

Preferably, the at least one 5′-UTR element and the at least one 3′-UTR element act synergistically to increase protein production from the at least one mRNA sequence as described above.

According to a preferred embodiment, the composition of the invention comprises an mRNA compound that comprises, preferably in 5′- to 3-direction:
a) a 5′-CAP structure, preferably m7GpppN, more preferably CAP1 or m7G(5′)ppp(5′)(2′OMeA)pG;
b) optionally, a 5′-UTR element which preferably comprises or consists of a nucleic acid sequence which is derived from the 5′-UTR of a TOP gene, more preferably comprising or consisting of the corresponding RNA sequence of a nucleic acid sequence according to SEQ ID NO:21, 23, 25 or respectively SEQ ID NO:22, 24 or 26, a homolog, a fragment or a variant thereof, most preferably according to SEQ ID NO:22 (HSD17B4), or a 5′-UTR element which preferably comprises or consists of a nucleic acid sequence which is derived from a solute carrier family 7 cationic amino acid transporter, y+ system), member 3 (SEQ ID NO:15 as disclosed in WO2019077001 or respectively a RNA sequence according to SEQ ID NO:16 as disclosed in WO2019077001) or a 60S ribosomal protein L31 (RPL31) gene (SEQ ID NO:13 as disclosed in WO2019077001 or respectively a RNA sequence according to SEQ ID NO:14 as disclosed in WO2019077001);
c) at least one coding region encoding at least one antigen or fragment or variant thereof, preferably derived from a protein of a SARS coronavirus 2 (SARS-CoV-2), nCoV-2019 coronavirus, SARS coronavirus (SARS-CoV), Bunyavirales virus, Cytomegalovirus (CMV), Dengue viruses (DENV-1, DENV-2, DENV-3 and DENV-4), Ebola virus (EBOV), Flavivirus, Hepatitis B virus (HBV), Herpes simplex virus (HSV), Human immunodeficiency virus (HIV), Human metapneumovirus (HMPV), Human Papilloma virus (HPV), Human parainfluenza viruses (HPIV), Influenza virus, extraintestinal pathogenic E. coli (ExPEC), Lassa mammarenavirus (LASV), MERS coronavirus, Mycobacterium tuberculosis, Nipah virus, Norovirus, Rabies virus (RABV), Respiratory Syncytial virus (RSV), Rhinovirus, Rotavirus, Vaccinia virus, Yellow Fever virus (YFV), Zika virus (ZIKV), Chlamydia trachomatis (i.e. bacterium Chlamydia causing Chlamydia), or Malaria parasite (e.g. Plasmodium falciparum, Plasmodium vivax, Plasmodium malariae, or Plasmodium ovale), or a fragment or variant thereof, preferably comprising or consisting of any one of the nucleic acid sequences as disclosed in the sequence listing having a numeric identifier <223> which starts with “derived and/or modified CDS sequence (wt)” or “derived and/or modified CDS sequence (opt1)”, “derived and/or modified CDS sequence (opt2)”, “derived and/or modified CDS sequence (opt3)”, “derived and/or modified CDS sequence (opt4)”, or “derived and/or modified CDS sequence (opt5)”, or respectively “column B” or “column C” of Tabs. 1-5 or FIGS. 20-24 or respective the sequence listing of PCT/EP2016/075843 or WO2018078053, incorporated by reference in their entirety; or an ORF comprised in SEQ ID NO:27-40 or SEQ ID NO:71-74 or of a fragment or variant of any one of these sequences; or at least one coding region encoding at least one antigenic peptide or protein preferably derived from a protein of an SARS coronavirus 2 (SARS-CoV-2), nCoV-2019 coronavirus, SARS coronavirus (SARS-CoV), Bunyavirales virus, Cytomegalovirus (CMV), Dengue viruses (DENV-1, DENV-2, DENV-3 and DENV-4), Ebola virus (EBOV), Flavivirus, Hepatitis B virus (HBV), Herpes simplex virus (HSV), Human immunodeficiency virus (HIV), Human metapneumovirus (HMPV), Human Papilloma virus (HPV), Human parainfluenza viruses (HPIV), Influenza virus, extraintestinal pathogenic E. coli (ExPEC), Lassa mammarenavirus (LASV), MERS coronavirus, Mycobacterium tuberculosis, Nipah virus, Norovirus, Rabies virus (RABV), Respiratory Syncytial virus (RSV), Rhinovirus, Rotavirus, Vaccinia virus, Yellow Fever virus (YFV), Zika virus (ZIKV), Chlamydia trachomatis (i.e. bacterium Chlamydia causing Chlamydia), or Malaria parasite (e.g. Plasmodium falciparum, Plasmodium vivax, Plasmodium malariae, or Plasmodium ovale);
d) optionally, a 3′-UTR element which preferably comprises or consists of a nucleic acid sequence which is derived from a gene providing a stable mRNA, preferably comprising or consisting of the corresponding RNA sequence of a nucleic acid sequence according to SEQ ID NO:6, 8, 10, 12, 14, 16, 18, 20, preferably according to SEQ ID NO:12 or SEQ ID NO:18 or a homolog, a fragment or a variant thereof, most preferably according to SEQ ID NO:20 (PSMB3) or a 3′-UTR element which preferably comprises or consists of a nucleic acid sequence which is derived from a 40S ribosomal protein S9 (RPS9) gene (SEQ ID NO:33 as disclosed in WO2019077001 or respectively a RNA sequence according to SEQ ID NO:34 as disclosed in WO2019077001) or SEQ ID NO:79/80;
e) optionally, a poly(A) sequence preferably comprising 64 adenosines or 100 adenosines; and
f) optionally, a poly(C) sequence, preferably comprising 30 cytosines;
g) optionally, a histone stem-loop selected from SEQ ID NO:3 or 4; and/or
h) optionally, a 3-terminal sequence element selected from SEQ ID NO:41-70.

According to one embodiment, the mRNA compound comprises an miRNA binding site. A miRNA (microRNA) is typically a small, non-coding single stranded RNA molecules of about 20 to 25 nucleotides in length which may function in gene regulation, for example, but not limited to, by mRNA degradation or translation inhibition or repression. miRNAs are typically produced from hairpin precursor RNAs (pre-miRNAs), and they may form functional complexes with proteins. Furthermore, miRNAs may bind to 3′-UTR regions of target mRNAs.

Preferably, the microRNA binding site is for a microRNA selected from the group consisting of miR-126, miR-142, miR-144, miR-146, miR-150, miR-155, miR-16, miR-21, miR-223, miR-24, miR-27, miR-26a binding site, preferably a miR-122 or miR-142 binding site, or any combination of the aforementioned miRNA binding sites

In one embodiment, the miRNA binding site is a naturally occurring miRNA binding site. In another embodiment, the miRNA binding site may be a mimetic, or a modification of a naturally-occurring miRNA binding site.

In some embodiments, a 3′-UTR comprises one or more of a polyadenylation signal, a binding site for proteins that affect a nucleic acid stability of location in a cell, or one or more miRNA or binding sites for miRNAs.

MicroRNAs (or miRNA) are 19-25 nucleotide long noncoding RNAs that bind to the 3′-UTR of nucleic acid molecules and down-regulate gene expression either by reducing nucleic acid molecule stability or by inhibiting translation. E.g., microRNAs are known to regulate RNA, and thereby protein expression, e.g. in liver (miR-122), heart (miR-Id, miR-149), endothelial cells (miR-17-92, miR-126), adipose tissue (let-7, miR-30c), kidney (miR-192, miR-194, miR-204), myeloid cells (miR-142-3p, miR-142-5p, miR-16, miR-21, miR-223, miR-24, miR-27), muscle (miR-133, miR-206, miR-208), and lung epithelial cells (let-7, miR-133, miR-126). The RNA may comprise one or more microRNA target sequences, microRNA sequences, or microRNA seeds. Such sequences may e.g. correspond to any known microRNA such as those taught in US20050261218 and US20050059005.

According to one preferred embodiment, the mRNA compound comprising an mRNA sequence according to the invention may further comprise, as defined herein:
a) a 5′-CAP structure, preferably m7GpppN, more preferably CAP1 or m7G(5′)ppp(5′)(2′OMeA)pG;
b) at least one miRNA binding site, preferably wherein the microRNA binding site is selected from the group consisting of a miR-126, miR-142, miR-144, miR-146, miR-150, miR-155, miR-16, miR-21, miR-223, miR-24, miR-27, miR-26a binding site, preferably a miR-122 or miR-142 binding site, or any combination of the aforementioned miRNA binding sites;
c) at least one 5′-UTR element;
d) at least one 3′-UTR element;
e) at least one poly(A) sequence;
f) at least one poly(C) sequence;
g) optionally, a histone stem-loop selected from SEQ ID NO:3 or 4;
h) optionally, a 3-terminal sequence element selected from SEQ ID NO:41-70;
or any combinations of these.

In other embodiments, the nucleic acid comprises a 5′-UTR which comprises or consists of a nucleic acid sequence being identical or at least 70%, 80%, 85%, 86%, 87%, 88%, 89%, 90%, 91%, 92%, 93%, 94%, 95%, 96%, 97%, 98%, or 99% identical to a sequence selected from SEQ ID NO:22848-22875 as disclosed in WO2021156267 or a fragment or a variant thereof.

In other embodiments, the nucleic acid comprises a 3′-UTR which comprises or consists of a nucleic acid sequence being identical or at least 70%, 80%, 85%, 86%, 87%, 88%, 89%, 90%, 91%, 92%, 93%, 94%, 95%, 96%, 97%, 98%, or 99% identical to a sequence selected from SEQ ID NO:22876-22891 as disclosed in WO2021156267 or a fragment or a variant thereof.

In other embodiments, the nucleic acid comprises a 5′-end which comprises or consists of a nucleic acid sequence being identical or at least 70%, 80%, 85%, 86%, 87%, 88%, 89%, 90%, 91%, 92%, 93%, 94%, 95%, 96%, 97%, 98%, or 99% identical to a single sequence selected from the group consisting of SEQ ID NO:176-177 and 22840-22844 as disclosed in WO2022137133 or a fragment or a variant thereof.

In other embodiments, the nucleic acid comprises a Kozak sequence which comprises or consists of a nucleic acid sequence being identical or at least 70%, 80%, 85%, 86%, 87%, 88%, 89%, 90%, 91%, 92%, 93%, 94%, 95%, 96%, 97%, 98%, or 99% identical to a single sequence selected from the group consisting of SEQ ID NO:180-181, and 22845-22847 as disclosed in WO2022137133 or a fragment or a variant thereof.

In other embodiments, the nucleic acid comprises a 5′-UTR which comprises or consists of a nucleic acid sequence being identical or at least 70%, 80%, 85%, 86%, 87%, 88%, 89%, 90%, 91%, 92%, 93%, 94%, 95%, 96%, 97%, 98%, or 99% identical to a single sequence selected from the group consisting of SEQ ID NO:231-252, 22848-22875, and 28522-28525 as disclosed in WO2021156267 or a fragment or a variant thereof.

In other embodiments, the nucleic acid comprises a 3′-UTR which comprises or consists of a nucleic acid sequence being identical or at least 70%, 80%, 85%, 86%, 87%, 88%, 89%, 90%, 91%, 92%, 93%, 94%, 95%, 96%, 97%, 98%, or 99% identical to a single sequence selected from the group consisting of SEQ ID NO:253-268, 22876-22911, 26996-27003, and 28526-28539 as disclosed in WO2021156267 or a fragment or a variant thereof.

In other embodiments, the nucleic acid comprises a 3-end which comprises or consists of a nucleic acid sequence being identical or at least 70%, 80%, 85%, 86%, 87%, 88%, 89%, 90%, 91%, 92%, 93%, 94%, 95%, 96%, 97%, 98%, or 99% identical to a single sequence selected from the group consisting of SEQ ID NO:182-230, and 27004-27006 as disclosed in WO2021156267 or a fragment or a variant thereof.

### Histone Stem-Loop (HSL)/Histone 3′-UTR Stem-Loop

In a further preferred embodiment, the composition comprises an mRNA compound comprising a histone stem-loop sequence/structure (HSL, hSL, histoneSL, preferably according to SEQ ID NO:3 or SEQ ID NO:4). In said embodiment, the mRNA sequence may comprise at least one (or more) histone stem loop sequence or structure.

Such histone stem-loop sequences are preferably selected from histone stem-loop sequences as disclosed in WO2012019780, the disclosure of which is incorporated herewith by reference. A histone stem-loop sequence that may be used within the present invention may preferably be derived from formulae (I) or (II) of WO2012019780. According to a further preferred embodiment the coding RNA may comprise at least one histone stem-loop sequence derived from at least one of the specific formulae (Ia) or (IIa) of the patent application WO2012019780. According to a further preferred embodiment the coding RNA may comprise at least one histone stem-loop sequence derived from a Histone stem-loop as disclosed in patent application WO2018104538 under formula (I), formula (II), formula (Ia) or on pages 49-52 under section “Histone stem-loop” and WO2018104538 SEQ ID NO:1451-1452 as disclosed in WO2018104538; WO2018104538 which is herein incorporated by reference in its entirety, also especially SEQ ID NO:1451-1452.

In particularly preferred embodiment, the RNA of the invention comprises at least one histone stem-loop sequence, wherein said histone stem-loop sequence comprises a nucleic acid sequence being identical or at least 70%, 80%, 90%, 91%, 92%, 93%, 94%, 95%, 96%, 97%, 98%, or 99% identical to SEQ ID NO:3 or 4, or fragments or variants thereof.

In other embodiments, the nucleic acid comprises a histone stem-loop which comprises or consists of a nucleic acid sequence being identical or at least 70%, 80%, 85%, 86%, 87%, 88%, 89%, 90%, 91%, 92%, 93%, 94%, 95%, 96%, 97%, 98%, or 99% identical to a sequence selected from the group consisting of SEQ ID NO:178 and 179 as disclosed in WO2022137133 or a fragment or a variant thereof.

### Signal Peptides/Signal Sequence/Leader Sequence

According to another embodiment, the composition of the invention comprises an mRNA compound which may, additionally or alternatively, encode a secretory signal peptide. Such signal peptides are sequences, which typically exhibit a length of about 15 to 30 amino acids and are preferably located at the N-terminus of the encoded peptide, without being limited thereto. Signal peptides as defined herein preferably allow the transport of the antigen as encoded by the at least one mRNA sequence into a defined cellular compartment, preferably the cell surface, the endoplasmic reticulum (ER) or the endosomal-lysosomal compartment. Examples of secretory signal peptide sequences as defined herein include, without being limited thereto, signal sequences of classical or non-classical MHC-molecules (e.g. signal sequences of MHC I and II molecules, e.g. of the MHC class I molecule HLA-A*0201), signal sequences of cytokines or immunoglobulins as defined herein, signal sequences of the invariant chain of immunoglobulins or antibodies as defined herein, signal sequences of Lamp1, Tapasin, Erp57, Calreticulin, Calnexin, and further membrane associated proteins or of proteins associated with the endoplasmic reticulum (ER) or the endosomal-lysosomal compartment. Most preferably, signal sequences of MHC class I molecule HLA-A*0201 may be used according to the present invention. For example, a signal peptide derived from HLA-A is preferably used in order to promote secretion of the encoded antigen as defined herein or a fragment or variant thereof. More preferably, an HLA-A signal peptide is fused to an encoded antigen as defined herein or to a fragment or variant thereof.

The mRNA compound to be incorporated in the composition according to the present invention may be prepared using any method known in the art, including synthetic methods such as e.g. solid phase RNA synthesis, as well as in vitro methods, such as RNA in vitro transcription reactions, particularly as described in the examples.

### Methods of Preparing Lipid Nanoparticles

A typical method of preparing the lipid nanoparticles of the present invention comprises the steps of:
(i) providing:
   a) the phospholipid phosphatidylserine;
   b) a cationic or ionizable lipid;
   c) a polymer conjugated lipid;
   d) at least one mRNA compound comprising an mRNA sequence encoding at least one antigen or fragment or variant thereof;
   e) optionally, a steroid; and
   f) optionally, at least one further phospholipid in addition to phosphatidylserine;
(ii) solubilizing the lipids provided in step (i) in an alcohol such as ethanol;
(iii) mixing the alcoholic lipid solution with an aqueous solution comprising the mRNA polynucleotide
(iv) removing the alcohol to form lipid nanoparticles encapsulating or associating with the mRNA polynucleotide; and optionally
(v) separating or purifying the lipid nanoparticles.

The alcohol may be removed by any suitable method which does not negatively affect the lipids or the forming lipid nanoparticles. In one embodiment of the invention the alcohol is removed by dialysis. In an alternative embodiment the alcohol is removed by diafiltration.

Separation and optional purification of the lipid nanoparticles might also be performed by any suitable method. Preferably the lipid nanoparticles are filtrated, more preferably the lipid nanoparticles are separated or purified by filtration through a sterile filter.

In some embodiments, the solutions are mixed in a microfluidic mixer to obtain the composition. Suitably, the microfluidic mixing conditions are chosen so as to obtain encapsulation of the pharmaceutically active compound at an encapsulation efficiency (EE) of above 80%, preferably above 90%, more preferably above 94%.

### Routes of Administration

The choice of a pharmaceutically acceptable carrier is determined, in principle, by the manner, in which the pharmaceutical composition or vaccine composition according to the invention is administered. The composition or vaccine composition of the invention can be administered, for example, systemically or locally. Routes for systemic administration in general include, for example, transdermal, oral, parenteral routes, including subcutaneous, intravenous, intramuscular, intraarterial, intradermal and intraperitoneal injections and/or intranasal administration routes. Preferably, systemic administration can be done intravenously (i.v.), subcutaneously (s.c), intradermally (i.d.) or pulmonary.

Routes for local administration in general include, for example, topical administration routes but also intradermal, transdermal, subcutaneous, or intramuscular injections or intralesional, intracranial, intrapulmonal, intracardial, intratumoral and sublingual administration or injections. Administration to the respiratory system can be performed by spray administration or inhalation may in particular be performed by aerosol administration to the lungs, bronchi, bronchioli, alveoli, or paranasal sinuses.

In further preferred embodiments, the route of administration is selected from the group consisting of extravascular administration to a subject, such as by extravascular injection, infusion or implantation; topical administration to the skin or a mucosa; inhalation such as to deliver the composition to the respiratory system; or by transdermal or percutaneous administration. In even further preferred embodiments, the composition or vaccine composition of the invention can be administered via local or locoregional injection, infusion or implantation, in particular intradermal, subcutaneous, intramuscular, intracameral, subconjunctival, suprachoroidal injection, subretinal, subtenon, retrobulbar, topical, posterior juxtascleral administration, or intrapulmonal inhalation, interstitial, locoregional, intravitreal, intratumoral, intralymphatic, intranodal, intra-articular, intrasynovial, periarticular, intraperitoneal, intra-abdominal, intracardial, intralesional, intrapericardial, intraventricular, intrapleural, perineural, intrathoracic, epidural, intradural, peridural, intrathecal, intramedullary, intracerebral, intracavernous, intracorporus cavernosum, intraprostatic, intratesticular, intracartilaginous, intraosseous, intradiscal, intraspinal, intracaudal, intrabursal, intragingival, intraovarian, intrauterine, periocular, periodontal, retrobulbar, subarachnoid, subconjunctival or suprachoroidal injection, infusion or implantation.

Moreover, topical administration to the skin or a mucosa may be performed by dermal or cutaneous, nasal, buccal, sublingual, otic or auricular, ophthalmic, conjunctival, vaginal, rectal, intracervical, endosinusial, laryngeal, oropharyngeal, ureteral, urethral administration. Even more preferred routes of administration for a vaccine are intramuscular, intradermal, intranasal and oral administration (e.g. via a tablet).

Preferably, compositions or vaccine compositions according to the present invention may be administered by an intradermal, subcutaneous, or intramuscular route, preferably by injection, which may be needle-free and/or needle injection. Compositions or vaccine compositions according to the present invention are therefore preferably formulated in liquid or solid form. The suitable amount of the vaccine or composition according to the invention to be administered can be determined by routine experiments, e.g. by using animal models. Such models include, without implying any limitation, rabbit, sheep, mouse, rat, dog and non-human primate models.

Preferred unit dose forms for injection include sterile solutions of water, physiological saline or mixtures thereof. The pH of such solutions should be adjusted to a physiologically tolerable pH, such as about 7.4. Suitable carriers for injection include hydrogels, devices for controlled or delayed release, polylactic acid and collagen matrices. Suitable pharmaceutically acceptable carriers for topical application include those which are suitable for use in lotions, creams, gels and the like. If the inventive composition or vaccine is to be administered perorally, tablets, capsules and the like are the preferred unit dose form. The pharmaceutically acceptable carriers for the preparation of unit dose forms which can be used for oral administration are well known in the prior art. The choice thereof will depend on secondary considerations such as taste, costs and storability, which are not critical for the purposes of the present invention, and can be made without difficulty by a person skilled in the art.

### Pharmaceutical Compositions and Kits

The term “treatment” or “treating” of a disease includes preventing or protecting against the disease (that is, causing the clinical symptoms not to develop); inhibiting the disease (i.e., arresting or suppressing the development of clinical symptoms; and/or relieving the disease (i.e., causing the regression of clinical symptoms). In a preferred embodiment, the term “subject” refers to a human.

The invention further relates to a pharmaceutical composition comprising at least one lipid nanoparticle according to the present invention.

In one embodiment of the invention the mRNA sequence encodes one antigenic peptide or protein. In an alternative embodiment of the invention the mRNA sequence encodes more than one antigenic peptide or protein.

In one embodiment of the invention, the pharmaceutical composition comprises a lipid nanoparticle according to the invention, wherein the lipid nanoparticle comprises more than one mRNA compounds, wherein each comprises a different mRNA sequence encoding an antigen or fragment or variant thereof.

In an alternative embodiment of the invention the pharmaceutical composition comprises a second lipid nanoparticle, wherein the mRNA compound comprised by the second lipid nanoparticle is different from the mRNA compound comprised by the first lipid nanoparticle.

In a further aspect, the present invention concerns a composition comprising mRNA comprising lipid nanoparticles wherein the mRNA comprises an mRNA sequence comprising at least one coding region encoding at least one nucleic acid encoding at least one antigen or fragment or variant thereof and a pharmaceutically acceptable carrier. The composition according to the invention is provided as a vaccine.

The composition according to the invention might also comprise suitable pharmaceutically acceptable adjuvants. In preferred embodiments the adjuvant is preferably added in order to enhance the immunostimulatory properties of the composition. In this context, an adjuvant may be understood as any compound, which is suitable to support administration and delivery of the composition according to the invention. Furthermore, such an adjuvant may, without being bound thereto, initiate or increase an immune response of the innate immune system, i.e. a non-specific immune response. In other words, when administered, the composition according to the invention typically initiates an adaptive immune response due to an antigen as defined herein or a fragment or variant thereof, which is encoded by the at least one coding sequence of the inventive mRNA contained in the composition of the present invention. Additionally, the composition according to the invention may generate an (supportive) innate immune response due to addition of an adjuvant as defined herein to the composition according to the invention.

In some embodiments, the invention provides a method of inducing an immune response in a subject, the method comprising administering to the subject the vaccine of the invention in an amount effective to produce an antigen-specific immune response in the subject. In other embodiments, the invention provides a pharmaceutical composition comprising a composition or a kit or kit of parts as described herein for use in vaccination of a subject comprising an effective dose of mRNA encoding an antigen.

Such an adjuvant may be selected from any adjuvant known to a skilled person and suitable for the present case, i.e. supporting the induction of an immune response in a mammal. Preferably, the adjuvant may be selected from the group consisting of adjuvants, without being limited thereto, as disclosed on page 160 line 3-161 line 8 in WO2018078053; WO2018078053 being incorporated herein by reference in its entirety.

Particularly preferred, an adjuvant may be selected from adjuvants, which support induction of a Th1-immune response or maturation of naïve T-cells, such as GM-CSF, IL-12, IFN-gamma, any immunostimulatory nucleic acid as defined above, preferably an immunostimulatory RNA, CpG DNA, et cetera.

In a further preferred embodiment it is also possible that the inventive composition contains besides the antigen-providing mRNA further components which are selected from the group comprising: a further immunotherapeutic agent; one or more auxiliary substances; or any further compound, which is known to be immunostimulating due to its binding affinity (as ligands) to human Toll-like receptors; and/or an adjuvant nucleic acid, preferably an immunostimulatory RNA (isRNA).

The composition of the present invention can additionally contain one or more auxiliary substances in order to increase its immunogenicity or immunostimulatory capacity, if desired. A synergistic action of the mRNA as defined herein and of an auxiliary substance, which may be optionally contained in the inventive composition, is preferably achieved thereby. Depending on the various types of auxiliary substances, various mechanisms can come into consideration in this respect. For example, compounds that permit the maturation of dendritic cells (DCs), for example lipopolysaccharides, TNF-alpha or CD40 ligand, form a first class of suitable auxiliary substances. In general, it is possible to use as auxiliary substance any agent that influences the immune system in the manner of a “danger signal” (LPS, GP96, etc.) or cytokines, such as GM-CFS, which allow an immune response to be enhanced and/or influenced in a targeted manner. Particularly preferred auxiliary substances are cytokines, such as monokines, lymphokines, interleukins or chemokines, that further promote the innate immune response, such as IL-1, IL-2, IL-3, IL-4, IL-5, IL-6, IL-7, IL-8, IL-9, IL-10, IL-12, IL-13, IL-14, IL-15, IL-16, IL-17, IL-18, IL-19, IL-20, IL-21, IL-22, IL-23, IL-24, IL-25, IL-26, IL-27, IL-28, IL-29, IL-30, IL-31, IL-32, IL-33, IFN-alpha, IFN-beta, IFN-gamma, GM-CSF, G-CSF, M-CSF, LT-beta or TNF-alpha, growth factors, such as hGH. Preferably, such immunogenicity increasing agents or compounds are provided separately (not co-formulated with the inventive vaccine or composition) and administered individually.

Suitable adjuvants may furthermore be selected from nucleic acids having the formula GₗXmGₙ, wherein: G is guanosine, uracil or an analogue of guanosine or uracil; X is guanosine, uracil, adenosine, thymidine, cytosine or an analogue of the above-mentioned nucleotides; l is an integer from 1 to 40, wherein when l=1 G is guanosine or an analogue thereof, when l>1 at least 50% of the nucleotides are guanosine or an analogue thereof; m is an integer and is at least 3; wherein when m=3 X is uracil or an analogue thereof, when m>3 at least 3 successive uracils or analogues of uracil occur; n is an integer from 1 to 40, wherein when n=1 G is guanosine or an analogue thereof, when n>1 at least 50% of the nucleotides are guanosine or an analogue thereof, or formula: (NᵤGₗXₘGₙNᵥ)ₐ, wherein: G is guanosine (guanine), uridine (uracil) or an analogue of guanosine (guanine) or uridine (uracil), preferably guanosine (guanine) or an analogue thereof; X is guanosine (guanine), uridine (uracil), adenosine (adenine), thymidine (thymine), cytidine (cytosine), or an analogue of these nucleotides (nucleosides), preferably uridine (uracil) or an analogue thereof; N is a nucleic acid sequence having a length of about 4 to 50, preferably of about 4 to 40, more preferably of about 4 to 30 or 4 to 20 nucleic acids, each N independently being selected from guanosine (guanine), uridine (uracil), adenosine (adenine), thymidine (thymine), cytidine (cytosine) or an analogue of these nucleotides (nucleosides); a is an integer from 1 to 20, preferably from 1 to 15, most preferably from 1 to 10; l is an integer from 1 to 40, wherein when l=1, G is guanosine (guanine) or an analogue thereof, when l>1, at least 50% of these nucleotides (nucleosides) are guanosine (guanine) or an analogue thereof; m is an integer and is at least 3; wherein when m=3, X is uridine (uracil) or an analogue thereof, and when m>3, at least 3 successive uridines (uracils) or analogues of uridine (uracil) occur; n is an integer from 1 to 40, wherein when n=1, G is guanosine (guanine) or an analogue thereof, when n>1, at least 50% of these nucleotides (nucleosides) are guanosine (guanine) or an analogue thereof; u, v may be independently from each other an integer from 0 to 50, preferably wherein when u=0, v>1, or when v=0, u≥1; wherein the nucleic acid molecule of formula (NᵤGₗXₘGₙNᵥ)ₐ has a length of at least 50 nucleotides, preferably of at least 100 nucleotides, more preferably of at least 150 nucleotides, even more preferably of at least 200 nucleotides and most preferably of at least 250 nucleotides. Other suitable adjuvants may furthermore be selected from nucleic acids having the formula: CₗXₘCₙ, wherein: C is cytosine, uracil or an analogue of cytosine or uracil; X is guanosine, uracil, adenosine, thymidine, cytosine or an analogue of the above-mentioned nucleotides; l is an integer from 1 to 40, wherein when l=1 C is cytosine or an analogue thereof, when l>1 at least 50% of the nucleotides are cytosine or an analogue thereof; m is an integer and is at least 3; wherein when m=3 X is uracil or an analogue thereof, when m>3 at least 3 successive uracils or analogues of uracil occur; n is an integer from 1 to 40, wherein when n=1 C is cytosine or an analogue thereof, when n>1 at least 50% of the nucleotides are cytosine or an analogue thereof.

In this context the disclosure of WO2008014979 (whole disclosure, especially the subject-matter of claim 1, claim 2, claim 3, claim 4 and claim 5) and WO2009095226 are also incorporated herein by reference in their entirety.

In embodiments, where the vaccine comprises more than one mRNA sequence (such as a plurality of RNA sequences, wherein each encodes a distinct antigen or fragment or variant thereof) encapsulated in mRNA-comprising lipid nanoparticles, the vaccine may be provided in physically separate form and may be administered by separate administration steps. The vaccine according to the invention may correspond to the (pharmaceutical) composition as described herein, especially where the mRNA sequences are provided by one single composition.

However, the inventive vaccine may also be provided physically separated. For instance, in embodiments, wherein the vaccine comprises more than one mRNA sequences/species encapsulated in mRNA comprising lipid nanoparticles as defined herein, these RNA species may be provided such that, for example, two, three, four, five or six separate compositions, which may contain at least one mRNA species/sequence each (e.g. three distinct mRNA species/sequences), each encoding distinct antigens or fragments or variants thereof, are provided, which may or may not be combined. Also, the inventive vaccine may be a combination of at least two distinct compositions, each composition comprising at least one mRNA encoding at least one antigen or fragment or variant thereof. Alternatively, the vaccine may be provided as a combination of at least one mRNA, preferably at least two, three, four, five, six or more mRNAs, each encoding one antigen or fragment or variant thereof. The vaccine may be combined to provide one single composition prior to its use or it may be used such that more than one administration is required to administer the distinct mRNA sequences/species encoding any of the antigen or fragment or variant thereof encapsulated in mRNA-comprising lipid nanoparticles as defined herein. If the vaccine contains at least one mRNA comprising lipid nanoparticles, typically comprising at least two mRNA sequences, encoding the antigen combinations defined herein, it may e.g. be administered by one single administration (combining all mRNA species/sequences) or by at least two separate administrations. Accordingly; any combination of mono-, bi- or multicistronic mRNAs encoding the at least one antigen or fragment or variant thereof or any combination of antigens as defined herein (and optionally further antigens), provided as separate entities (containing one mRNA species) or as combined entity (containing more than one mRNA species), is understood as a vaccine according to the present invention. According to a particularly preferred embodiment of the inventive vaccine, the at least one antigen, preferably a combination as defined herein of at least two, three, four, five, six or more antigens encoded by the inventive composition as a whole, is provided as an individual (monocistronic) mRNA, which is administered separately.

The entities of the vaccine may be provided in liquid and or in dry (e.g. lyophilized) form. They may contain further components, in particular further components allowing for its pharmaceutical use. The vaccine or the (pharmaceutical) composition may, e.g., additionally contain a pharmaceutically acceptable carrier and/or further auxiliary substances and additives and/or adjuvants.

The vaccine or (pharmaceutical) composition typically comprises a safe and effective amount of the mRNA compound according to the invention as defined herein, encoding an antigen or fragment or variant thereof or a combination of antigens, encapsulated within and/or associated with the lipid nanoparticles. As used herein, “safe and effective amount” means an amount of the mRNA that is sufficient to significantly induce a positive modification of cancer or a disease or disorder related to cancer. At the same time, however, a “safe and effective amount” is small enough to avoid serious side-effects, that is to say to permit a sensible relationship between advantage and risk. The determination of these limits typically lies within the scope of sensible medical judgment. In relation to the vaccine or (pharmaceutical) composition of the present invention, the expression “safe and effective amount” preferably means an amount of the mRNA (and thus of the encoded antigen) that is suitable for stimulating the adaptive immune system in such a manner that no excessive or damaging immune reactions are achieved but, preferably, also no such immune reactions below a measurable level. Such a “safe and effective amount” of the mRNA of the (pharmaceutical) composition or vaccine as defined herein may furthermore be selected in dependence of the type of mRNA, e.g. monocistronic, bi- or even multicistronic mRNA, since a bi- or even multicistronic mRNA may lead to a significantly higher expression of the encoded antigen(s) than the use of an equal amount of a monocistronic mRNA. A “safe and effective amount” of the mRNA of the (pharmaceutical) composition or vaccine as defined above will furthermore vary in connection with the particular condition to be treated and also with the age and physical condition of the patient to be treated, the severity of the condition, the duration of the treatment, the nature of the accompanying therapy, of the particular pharmaceutically acceptable carrier used, and similar factors, within the knowledge and experience of the accompanying doctor. The vaccine or composition according to the invention can be used according to the invention for human and also for veterinary medical purposes, as a pharmaceutical composition or as a vaccine.

In a preferred embodiment, the mRNA comprising lipid nanoparticle of the (pharmaceutical) composition, vaccine or kit of parts according to the invention is provided in lyophilized form. Preferably, the lyophilized mRNA comprising lipid nanoparticles are reconstituted in a suitable buffer, advantageously based on an aqueous carrier, prior to administration, e.g. Ringer-Lactate solution, Ringer solution, a phosphate buffer solution. In a preferred embodiment, the (pharmaceutical) composition, the vaccine or the kit of parts according to the invention contains at least one, two, three, four, five, six or more mRNA compounds, which may be provided as a single species of lipid nanoparticles, or separately for each LNP species, optionally in lyophilized form (optionally together with at least one further additive) and which are preferably reconstituted separately in a suitable buffer (such as Ringer-Lactate solution) prior to their use so as to allow individual administration of each of the (monocistronic) mRNAs.

The vaccine or (pharmaceutical) composition according to the invention may typically contain a pharmaceutically acceptable carrier or excipient. Examples of suitable carriers and excipients are known to those skilled in the art and include but are not limited to preserving agents, fillers, disintegrating agents, wetting agents, emulsifying agents, suspending agents, sweetening agents, flavouring agents, perfuming agents, antibacterial agents, antifungal agents, lubricating agents and dispersing agents, depending on the nature of the mode of administration and dosage forms. The term “pharmaceutical composition” in the context of this invention means a composition comprising an active agent and comprising additionally one or more pharmaceutically acceptable carriers. The composition may further contain ingredients selected from, for example, diluents, excipients, vehicles, preserving agents, fillers, disintegrating agents, wetting agents, emulsifying agents, suspending agents, sweetening agents, flavouring agents, perfuming agents, antibacterial agents, antifungal agents, lubricating agents and dispersing agents, depending on the nature of the mode of administration and dosage forms.

The expression “pharmaceutically acceptable carrier” as used herein preferably includes the liquid or non-liquid basis of the inventive vaccine. If the inventive vaccine is provided in liquid form, the carrier will be water, typically pyrogen-free water; isotonic saline or buffered (aqueous) solutions, e.g phosphate, citrate etc. buffered solutions. Particularly for injection of the inventive vaccine, water or preferably a buffer, more preferably an aqueous buffer, may be used, containing a sodium salt, preferably at least 50 mM of a sodium salt, a calcium salt, preferably at least 0.01 mM of a calcium salt, and optionally a potassium salt, preferably at least 3 mM of a potassium salt. According to a preferred embodiment, the sodium, calcium and, optionally, potassium salts may occur in the form of their halogenides, e.g. chlorides, iodides, or bromides, in the form of their hydroxides, carbonates, hydrogen carbonates, or sulfates, etc. Without being limited thereto, examples of sodium salts include e.g. NaCl, NaI, NaBr, Na₂CO₃, NaHCO₃, Na₂SO₄, examples of the optional potassium salts include e.g. KCl, KI, KBr, K₂CO₃, KHCO₃, K₂SO₄, and examples of calcium salts include e.g. CaCl₂, CaI₂, CaBr₂, CaCO₃, CaSO₄, Ca(OH)₂. Furthermore, organic anions of the aforementioned cations may be contained in the buffer. According to a more preferred embodiment, the buffer suitable for injection purposes as defined above, may contain salts selected from sodium chloride (NaCl), calcium chloride (CaCl₂) and optionally potassium chloride (KCl), wherein further anions may be present additional to the chlorides. CaCl₂ can also be replaced by another salt like KCl. Typically, the salts in the injection buffer are present in a concentration of at least 50 mM sodium chloride (NaCl), at least 3 mM potassium chloride (KCl) and at least 0.01 mM calcium chloride (CaCl₂). The injection buffer may be hypertonic, isotonic or hypotonic with reference to the specific reference medium, i.e. the buffer may have a higher, identical or lower salt content with reference to the specific reference medium, wherein preferably such concentrations of the afore mentioned salts may be used, which do not lead to damage of cells due to osmosis or other concentration effects. Reference media are e.g. in “in vivo” methods occurring liquids such as blood, lymph, cytosolic liquids, or other body liquids, or e.g. liquids, which may be used as reference media in “in vitro” methods, such as common buffers or liquids. Such common buffers or liquids are known to a skilled person.

However, one or more compatible solid or liquid fillers or diluents or encapsulating compounds may be used as well, which are suitable for administration to a person. The term “compatible” as used herein means that the excipients of the inventive vaccine are capable of being mixed with the mRNA according to the invention as defined herein, in such a manner that no interaction occurs, which would substantially reduce the pharmaceutical effectiveness of the inventive vaccine under typical use conditions. Pharmaceutically acceptable carriers, fillers and diluents must, of course, have sufficiently high purity and sufficiently low toxicity to make them suitable for administration to a person to be treated. Some examples of compounds which can be used as pharmaceutically acceptable carriers, fillers or excipients thereof are sugars, such as, for example, lactose, glucose, trehalose and sucrose; starches, such as, for example, corn starch or potato starch; dextrose; cellulose and its derivatives, such as, for example, sodium carboxymethylcellulose, ethylcellulose, cellulose acetate; powdered tragacanth; malt; gelatin; tallow; solid glidants, such as, for example, stearic acid, magnesium stearate; calcium sulfate; vegetable oils, such as, for example, groundnut oil, cottonseed oil, sesame oil, olive oil, corn oil and oil from theobroma; polyols, such as, for example, polypropylene glycol, glycerol, sorbitol, mannitol and polyethylene glycol; alginic acid.

The choice of a pharmaceutically acceptable carrier is determined, in principle, by the manner, in which the pharmaceutical composition or vaccine according to the invention is administered. The composition or vaccine can be administered, for example, systemically or locally.

Routes for systemic administration in general include, for example, transdermal, oral, parenteral routes, including subcutaneous, intravenous, intramuscular, intraarterial, intradermal and intraperitoneal injections and/or intranasal administration routes. Preferred administration routes according to the invention for the administration of vaccines are intramuscular injection, intradermal injection, or any of the herein mentioned routes of administration.

Routes for local administration in general include, for example, topical administration routes but also intradermal, transdermal, subcutaneous, or intramuscular injections or intralesional, intracranial, intrapulmonal, intracardial, and sublingual administration or injections. More preferably, composition or vaccines according to the present invention may be administered by an intradermal, subcutaneous, or intramuscular route, preferably by injection, which may be needle-free and/or needle injection, or any of the herein mentioned routes of administration.

According to preferred embodiments, the artificial nucleic acid (RNA) molecule, (pharmaceutical) composition or vaccine or kit is administered by a parenteral route, preferably via intradermal, subcutaneous, or intramuscular routes. Preferably, said artificial nucleic acid (RNA) molecule, (pharmaceutical) composition or vaccine or kit may be administered by injection, e.g. subcutaneous, intramuscular or intradermal injection, which may be needle-free and/or needle injection. Accordingly, in preferred embodiments, the medical use and/or method of treatment according to the present invention involves administration of said artificial nucleic acid (RNA) molecule, (pharmaceutical) composition or vaccine or kit by subcutaneous, intramuscular or intradermal injection, preferably by intramuscular or intradermal injection, more preferably by intradermal injection. Such injection may be carried out by using conventional needle injection or (needle-free) jet injection, preferably by using (needle-free) jet injection.

The term “jet injection”, as used herein, refers to a needle-free injection method, wherein a fluid containing at least one inventive mRNA sequence and, optionally, further suitable excipients is forced through an orifice, thus generating an ultra-fine liquid stream of high pressure that is capable of penetrating mammalian skin and, depending on the injection settings, subcutaneous tissue or muscle tissue. In principle, the liquid stream forms a hole in the skin, through which the liquid stream is pushed into the target tissue. Preferably, jet injection is used for intradermal, subcutaneous or intramuscular injection of the mRNA sequence according to the invention. In a preferred embodiment, jet injection is used for intramuscular injection of the mRNA sequence according to the invention. In a further preferred embodiment, jet injection is used for intradermal injection of the mRNA sequence according to the invention.

Compositions/vaccines are therefore preferably formulated in liquid or solid form. The suitable amount of the vaccine or composition according to the invention to be administered can be determined by routine experiments, e.g. by using animal models. Such models include, without implying any limitation, rabbit, sheep, mouse, rat, dog and non-human primate models. Preferred unit dose forms for injection include sterile solutions of water, physiological saline or mixtures thereof. The pH of such solutions should be adjusted to a physiologically tolerable pH, such as about 7.4. Suitable carriers for injection include hydrogels, devices for controlled or delayed release, polylactic acid and collagen matrices. Suitable pharmaceutically acceptable carriers for topical application include those which are suitable for use in lotions, creams, gels and the like. If the inventive composition or vaccine is to be administered perorally, tablets, capsules and the like are the preferred unit dose form. The pharmaceutically acceptable carriers for the preparation of unit dose forms which can be used for oral administration are well known in the prior art. The choice thereof will depend on secondary considerations such as taste, costs and storability, which are not critical for the purposes of the present invention, and can be made without difficulty by a person skilled in the art.

Further additives which may be included in the inventive vaccine or composition are emulsifiers, such as, for example, Tween; wetting agents, such as, for example, sodium lauryl sulfate; colouring agents; taste-imparting agents, pharmaceutical carriers; tablet-forming agents; stabilizers; antioxidants; preservatives.

The inventive vaccine or composition can also additionally contain any further compound, which is known to be immune-stimulating due to its binding affinity (as ligands) to human Toll-like receptors TLR1, TLR2, TLR3, TLR4, TLR5, TLR6, TLR7, TLR8, TLR9, TLR10, or due to its binding affinity (as ligands) to murine Toll-like receptors TLR1, TLR2, TLR3, TLR4, TLR5, TLR6, TLR7, TLR8, TLR9, TLR10, TLR11, TLR12 or TLR13.

Another class of compounds, which may be added to an inventive vaccine or composition in this context, may be CpG nucleic acids, in particular CpG-RNA or CpG-DNA. A CpG-RNA or CpG-DNA can be a single-stranded CpG-DNA (ss CpG-DNA), a double-stranded CpG-DNA (dsDNA), a single-stranded CpG-RNA (ss CpG-RNA) or a double-stranded CpG-RNA (ds CpG-RNA). The CpG nucleic acid is preferably in the form of CpG-RNA, more preferably in the form of single-stranded CpG-RNA (ss CpG-RNA). The CpG nucleic acid preferably contains at least one or more (mitogenic) cytosine/guanine dinucleotide sequence(s) (CpG motif(s)). According to a first preferred alternative, at least one CpG motif contained in these sequences, that is to say the C (cytosine) and the G (guanine) of the CpG motif, is unmethylated. All further cytosines or guanines optionally contained in these sequences can be either methylated or unmethylated. According to a further preferred alternative, however, the C (cytosine) and the G (guanine) of the CpG motif can also be present in methylated form.

According to another aspect of the present invention, the present invention also provides a kit, in particular a kit of parts, comprising the mRNA compound comprising mRNA sequence as defined herein In a further embodiment the kit comprises a lipid nanoparticle as defined above or the (pharmaceutical) composition comprising a lipid nanoparticle as defined above, and/or the vaccine according to the invention, optionally a liquid vehicle for solubilizing and optionally technical instructions with information on the administration and dosage of the mRNA comprising lipid nanoparticles, the composition and/or the vaccine. The technical instructions may contain information about administration and dosage of the mRNA comprising lipid nanoparticles, the composition and/or the vaccine. Such kits, preferably kits of parts, may be applied e.g. for any of the above mentioned applications or uses, preferably for the use of the lipid nanoparticle according to the invention (for the preparation of an inventive medicament, preferably a vaccine) for the treatment or prophylaxis of SARS coronavirus 2 (SARS-CoV-2), nCoV-2019 coronavirus, SARS coronavirus (SARS-CoV), Bunyavirales virus, Cytomegalovirus (CMV), Dengue viruses (DENV-1, DENV-2, DENV-3 and DENV-4), Ebola virus (EBOV), Flavivirus, Hepatitis B virus (HBV), Herpes simplex virus (HSV), Human immunodeficiency virus (HIV), Human metapneumovirus (HMPV), Human Papilloma virus (HPV), Human parainfluenza viruses (HPIV), Influenza virus, extraintestinal pathogenic E. coli (ExPEC), Lassa mammarenavirus (LASV), MERS coronavirus, Mycobacterium tuberculosis, Nipah virus, Norovirus, Rabies virus (RABV), Respiratory Syncytial virus (RSV), Rhinovirus, Rotavirus, Vaccinia virus, Yellow Fever virus (YFV), Zika virus (ZIKV), Chlamydia trachomatis (i.e. bacterium Chlamydia causing Chlamydia), or Malaria parasite (e.g. Plasmodium falciparum, Plasmodium vivax, Plasmodium malariae, or Plasmodium ovale) infections or diseases or disorders related thereto.

The kits may also be applied for the use of the lipid nanoparticle, the composition or the vaccine as defined herein (for the preparation of an inventive vaccine) for the treatment or prophylaxis of SARS coronavirus 2 (SARS-CoV-2), nCoV-2019 coronavirus, SARS coronavirus (SARS-CoV), Bunyavirales virus, Cytomegalovirus (CMV), Dengue viruses (DENV-1, DENV-2, DENV-3 and DENV-4), Ebola virus (EBOV), Flavivirus, Hepatitis B virus (HBV), Herpes simplex virus (HSV), Human immunodeficiency virus (HIV), Human metapneumovirus (HMPV), Human Papilloma virus (HPV), Human parainfluenza viruses (HPIV), Influenza virus, extraintestinal pathogenic E. coli (ExPEC), Lassa mammarenavirus (LASV), MERS coronavirus, Mycobacterium tuberculosis, Nipah virus, Norovirus, Rabies virus (RABV), Respiratory Syncytial virus (RSV), Rhinovirus, Rotavirus, Vaccinia virus, Yellow Fever virus (YFV), Zika virus (ZIKV), Chlamydia trachomatis (i.e. bacterium Chlamydia causing Chlamydia), or Malaria parasite (e.g. Plasmodium falciparum, Plasmodium vivax, Plasmodium malariae, or Plasmodium ovale) infections or diseases or disorders related thereto, wherein the lipid nanoparticle, the composition and/or the vaccine may be capable of inducing or enhancing an immune response in a mammal as defined above.

Such kits may further be applied for the use of the lipid nanoparticle, the composition or the vaccine as defined herein (for the preparation of an inventive vaccine) for modulating, preferably for eliciting, e.g. to induce or enhance, an immune response in a mammal as defined above, and preferably for supporting treatment or prophylaxis of SARS coronavirus 2 (SARS-CoV-2), nCoV-2019 coronavirus, SARS coronavirus (SARS-CoV), Bunyavirales virus, Cytomegalovirus (CMV), Dengue viruses (DENV-1, DENV-2, DENV-3 and DENV-4), Ebola virus (EBOV), Flavivirus, Hepatitis B virus (HBV), Herpes simplex virus (HSV), Human immunodeficiency virus (HIV), Human metapneumovirus (HMPV), Human Papilloma virus (HPV), Human parainfluenza viruses (HPIV), Influenza virus, extraintestinal pathogenic E. coli (ExPEC), Lassa mammarenavirus (LASV), MERS coronavirus, Mycobacterium tuberculosis, Nipah virus, Norovirus, Rabies virus (RABV), Respiratory Syncytial virus (RSV), Rhinovirus, Rotavirus, Vaccinia virus, Yellow Fever virus (YFV), Zika virus (ZIKV), Chlamydia trachomatis (i.e. bacterium Chlamydia causing Chlamydia), or Malaria parasite (e.g. Plasmodium falciparum, Plasmodium vivax, Plasmodium malariae, or Plasmodium ovale) infections or diseases or disorders related thereto.

Kits of parts, as a special form of kits, may contain one or more identical or different compositions and/or one or more identical or different vaccines as described herein in different parts of the kit. Kits of parts may also contain an (e.g. one) composition, an (e.g. one) vaccine and/or the mRNA comprising lipid nanoparticles according to the invention in different parts of the kit, e.g. each part of the kit containing an mRNA comprising lipid nanoparticles as defined herein, preferably encoding a distinct antigen. Preferably, the kit or the kit of parts contains as a part a vehicle for solubilizing the mRNA according to the invention, the vehicle optionally being Ringer-lactate solution. Any of the above kits may be used in a treatment or prophylaxis as defined above.

In another embodiment of this aspect, the kit according to the present invention may additionally contain at least one adjuvant. In a further embodiment, the kit according to the present invention may additionally contain at least one further pharmaceutically active component, preferably a therapeutic compound suitable for treatment and/or prophylaxis of cancer or a related disorder. Moreover, in another embodiment, the kit may additionally contain parts and/or devices necessary or suitable for the administration of the composition or the vaccine according to the invention, including needles, applicators, patches, injection-devices.

### Antagonists of RNA Sensing Pattern Recognition Receptors:

In preferred embodiments, in particular in embodiments where the nucleic acid of the composition is an RNA, the pharmaceutical composition may comprise at least one antagonist of at least one RNA sensing pattern recognition receptor.

In preferred embodiments in that context, the pharmaceutical composition comprises at least one antagonist of at least one RNA sensing pattern recognition receptor selected from a Toll-like receptor, preferably a TLR7 antagonist and/or a TLR8 antagonist.

Suitable antagonist of at least one RNA sensing pattern recognition receptor are disclosed in published PCT patent application WO2021028439, the full disclosure herewith incorporated by reference. In particular, the disclosure relating to suitable antagonist of at least one RNA sensing pattern recognition receptors as defined in any one of the claims 1 to 94 of WO2021028439 are incorporated by reference.

In preferred embodiments, the at least one antagonist of at least one RNA sensing pattern recognition receptor is a single stranded oligonucleotide that comprises or consists of a nucleic acid sequence being identical or at least 90%, 91%, 92%, 93%, 94%, 95%, 96%, 97%, 98%, 99%, or 100% identical to a nucleic acid sequence selected from the group consisting of SEQ ID NOs: 85-212 of WO2021028439, or fragments of any of these sequences. A particularly preferred antagonist in that context is 5′-GAG CGmG CCA-3′ (SEQ ID NO: 85 of WO2021028439), or a fragment or variant thereof.

In preferred embodiments, the molar ratio of the at least one antagonist of at least one RNA sensing pattern recognition receptor to the at least one RNA suitably ranges from about 20:1 to about 80:1.

In preferred embodiments, the weight to weight ratio of the at least one antagonist of at least one RNA sensing pattern recognition receptor to the at least one RNA suitably ranges from about 1:2 to about 1:10.

In embodiments, the at least one antagonist of at least one RNA sensing pattern recognition receptor and the at least one RNA encoding are separately formulated in the lipid-based carriers as defined herein or co-formulated in the lipid-based carriers as defined herein.

### Uses of Compositions

The composition according to the invention is useful as a medicament, as will be clear from the description of the active ingredient that may be incorporated within the composition and delivered to a subject, such as a human subject, by means of the composition and/or of the lipid nanoparticles contained therein. As such, a further aspect of the invention is the use of the composition as described above as a medicament. Such use may also be expressed as the use of the composition for the manufacture of a medicament. According to a related aspect, the invention provides a method of treatment, the method comprising a step of administering the composition to a subject, such as a human subject in need thereof, the composition. According to a related aspect, the invention provides a method of treating, the method comprising administration of the composition to a subject, such as a human subject in need thereof, the composition.

In a preferred embodiment, the composition of the invention is used as a medicament, wherein the medicament is a vaccine.

In another preferred embodiment, the composition of the invention is used as a medicament, wherein the medicament is for or suitable for the prevention, prophylaxis, treatment and/or amelioration of a disease selected from infectious diseases including viral, bacterial or protozoological infectious diseases, cancer or tumor diseases, liver diseases, autoimmune diseases and allergies.

In another preferred embodiment, the composition of the invention is used as a medicament, wherein the medicament is for or suitable for the prevention, prophylaxis, treatment and/or amelioration of an infectious disease including viral, bacterial or protozoological infectious diseases, wherein the medicament is a vaccine.

In another embodiment, the vaccine of the invention comprises a composition or a kit or kit of parts as described herein for prevention, prophylaxis, treatment and/or amelioration of a disease selected from infectious diseases including viral, bacterial or protozoological infectious diseases, cancer or tumor diseases.

In yet another aspect of the invention, a method of treating, a method of treatment or prophylaxis of infectious diseases; cancer or tumor diseases, disorders or conditions; liver diseases selected from the group consisting of liver fibrosis, liver cirrhosis and liver cancer; allergies; or autoimmune disease; disorder or condition is provided comprising the steps:
a) providing the mRNA, the composition, the vaccine, the kit or kit of parts as described herein; and
b) applying or administering the mRNA, the composition, the vaccine or the kit or kit of parts to a tissue or an organism.

In another embodiment, a method is provided, wherein the composition, the vaccine or the kit or kit of parts is administered to the tissue or to the organism by intravenous, intramuscular, subcutaneous or intradermal injection.

In yet a further embodiment, a method of inducing an immune response in a subject, the method comprising administering to the subject the vaccine of the invention in an amount effective to produce an antigen-specific immune response in the subject is provided.

In a further embodiment, a pharmaceutical composition comprising a composition or a kit or kit of parts as described herein for use or suitable for use in vaccination of a subject comprising an effective dose of mRNA encoding a virus antigen is provided.

In another preferred embodiment, use of a pharmaceutical composition comprising a composition or a kit or kit of parts as described herein for (i) inducing an immune response or for (ii) inducing CD8+ T cells responses is provided.

In a specific embodiment, a method for preventing, ameliorating or treating a disease or condition in a subject in need comprising administering to the subject a composition or a kit or kit of parts as described herein is provided.

Further, a method is provided, wherein administration of the composition results in an antigen specific antibody response, preferably wherein the antigen specific antibody response is measured by the presence of antigen-specific antibodies in serum.

In one of the preferred embodiments, the medicament is a SARS coronavirus 2 (SARS-CoV-2), nCoV-2019 coronavirus, SARS coronavirus (SARS-CoV), Bunyavirales virus, Cytomegalovirus (CMV), Dengue viruses (DENV-1, DENV-2, DENV-3 and DENV-4), Ebola virus (EBOV), Flavivirus, Hepatitis B virus (HBV), Herpes simplex virus (HSV), Human immunodeficiency virus (HIV), Human metapneumovirus (HMPV), Human Papilloma virus (HPV), Human parainfluenza viruses (HPIV), Influenza virus, extraintestinal pathogenic E. coli (ExPEC), Lassa mammarenavirus (LASV), MERS coronavirus, Mycobacterium tuberculosis, Nipah virus, Norovirus, Rabies virus (RABV), Respiratory Syncytial virus (RSV), Rhinovirus, Rotavirus, Vaccinia virus, Yellow Fever virus (YFV), Zika virus (ZIKV), Chlamydia trachomatis (i.e. bacterium Chlamydia causing Chlamydia), or Malaria parasite (e.g. Plasmodium falciparum, Plasmodium vivax, Plasmodium malariae, or Plasmodium ovale) vaccine.

In an alternative embodiment the present invention relates to the use of the pharmaceutical composition or the mRNA comprising lipid in the manufacture of a medicament. In particular said medicament is for therapeutically or prophylactically raising an immune response of a subject in need thereof.

In particular the medicament is for the treatment of a subject, preferably a vertebrate. In a preferred embodiment the subject is a mammal, preferably selected from the group comprising goat, cattle, swine, dog, cat, donkey, monkey, ape, a rodent such as a mouse, hamster, rabbit and, particularly, human.

With respect to the administration of the composition to a subject, in particular to a human subject, any suitable route may be used. In one embodiment, the composition is adapted for administration by injection or infusion. As used herein, the expression “adapted for” means that the composition is formulated and processed such as to be suitable for the respective route of administration.

The present invention furthermore comprises the use of the mRNA comprising lipid nanoparticles, the (pharmaceutical) composition or the vaccine according to the invention as defined herein for modulating, preferably for inducing or enhancing, an immune response in a mammal as defined herein, more preferably for preventing and/or treating SARS coronavirus 2 (SARS-CoV-2), nCoV-2019 coronavirus, SARS coronavirus (SARS-CoV), Bunyavirales virus, Cytomegalovirus (CMV), Dengue viruses (DENV-1, DENV-2, DENV-3 and DENV-4), Ebola virus (EBOV), Flavivirus, Hepatitis B virus (HBV), Herpes simplex virus (HSV), Human immunodeficiency virus (HIV), Human metapneumovirus (HMPV), Human Papilloma virus (HPV), Human parainfluenza viruses (HPIV), Influenza virus, extraintestinal pathogenic E. coli (ExPEC), Lassa mammarenavirus (LASV), MERS coronavirus, Mycobacterium tuberculosis, Nipah virus, Norovirus, Rabies virus (RABV), Respiratory Syncytial virus (RSV), Rhinovirus, Rotavirus, Vaccinia virus, Yellow Fever virus (YFV), Zika virus (ZIKV), Chlamydia trachomatis (i.e. bacterium Chlamydia causing Chlamydia), or Malaria parasite (e.g. Plasmodium falciparum, Plasmodium vivax, Plasmodium malariae, or Plasmodium ovale) infections, or of diseases or disorders related thereto.

In this context, support of the treatment or prophylaxis of SARS coronavirus 2 (SARS-CoV-2), nCoV-2019 coronavirus, SARS coronavirus (SARS-CoV), Bunyavirales virus, Cytomegalovirus (CMV), Dengue viruses (DENV-1, DENV-2, DENV-3 and DENV-4), Ebola virus (EBOV), Flavivirus, Hepatitis B virus (HBV), Herpes simplex virus (HSV), Human immunodeficiency virus (HIV), Human metapneumovirus (HMPV), Human Papilloma virus (HPV), Human parainfluenza viruses (HPIV), Influenza virus, extraintestinal pathogenic E. coli (ExPEC), Lassa mammarenavirus (LASV), MERS coronavirus, Mycobacterium tuberculosis, Nipah virus, Norovirus, Rabies virus (RABV), Respiratory Syncytial virus (RSV), Rhinovirus, Rotavirus, Vaccinia virus, Yellow Fever virus (YFV), Zika virus (ZIKV), Chlamydia trachomatis (i.e. bacterium Chlamydia causing Chlamydia), or Malaria parasite (e.g. Plasmodium falciparum, Plasmodium vivax, Plasmodium malariae, or Plasmodium ovale) infections may be any combination of a conventional SARS coronavirus 2 (SARS-CoV-2), nCoV-2019 coronavirus, SARS coronavirus (SARS-CoV), Bunyavirales virus, Cytomegalovirus (CMV), Dengue viruses (DENV-1, DENV-2, DENV-3 and DENV-4), Ebola virus (EBOV), Flavivirus, Hepatitis B virus (HBV), Herpes simplex virus (HSV), Human immunodeficiency virus (HIV), Human metapneumovirus (HMPV), Human Papilloma virus (HPV), Human parainfluenza viruses (HPIV), Influenza virus, extraintestinal pathogenic E. coli (ExPEC), Lassa mammarenavirus (LASV), MERS coronavirus, Mycobacterium tuberculosis, Nipah virus, Norovirus, Rabies virus (RABV), Respiratory Syncytial virus (RSV), Rhinovirus, Rotavirus, Vaccinia virus, Yellow Fever virus (YFV), Zika virus (ZIKV), Chlamydia trachomatis (i.e. bacterium Chlamydia causing Chlamydia), or Malaria parasite (e.g. Plasmodium falciparum, Plasmodium vivax, Plasmodium malariae, or Plasmodium ovale) therapy method such as therapy with antivirals such as neuraminidase inhibitors (e.g. oseltamivir and zanamivir) and M2 protein inhibitors (e.g. adamantane derivatives), and a therapy using the RNA or the pharmaceutical composition as defined herein.

Support of the treatment or prophylaxis of SARS coronavirus 2 (SARS-CoV-2), nCoV-2019 coronavirus, SARS coronavirus (SARS-CoV), Bunyavirales virus, Cytomegalovirus (CMV), Dengue viruses (DENV-1, DENV-2, DENV-3 and DENV-4), Ebola virus (EBOV), Flavivirus, Hepatitis B virus (HBV), Herpes simplex virus (HSV), Human immunodeficiency virus (HIV), Human metapneumovirus (HMPV), Human Papilloma virus (HPV), Human parainfluenza viruses (HPIV), Influenza virus, extraintestinal pathogenic E. coli (ExPEC), Lassa mammarenavirus (LASV), MERS coronavirus, Mycobacterium tuberculosis, Nipah virus, Norovirus, Rabies virus (RABV), Respiratory Syncytial virus (RSV), Rhinovirus, Rotavirus, Vaccinia virus, Yellow Fever virus (YFV), Zika virus (ZIKV), Chlamydia trachomatis (i.e. bacterium Chlamydia causing Chlamydia), or Malaria parasite (e.g. Plasmodium falciparum, Plasmodium vivax, Plasmodium malariae, or Plasmodium ovale) infections may be also envisaged in any of the other embodiments defined herein. Accordingly, any use of the mRNA comprising lipid nanoparticles, the (pharmaceutical) composition or the vaccine according to the invention in co-therapy with any other approach, preferably one or more of the above therapeutic approaches, in particular in combination with antivirals is within the scope of the present invention.

For administration, preferably any of the administration routes may be used as defined herein. In particular, an administration route is used, which is suitable for treating or preventing an SARS coronavirus 2 (SARS-CoV-2), nCoV-2019 coronavirus, SARS coronavirus (SARS-CoV), Bunyavirales virus, Cytomegalovirus (CMV), Dengue viruses (DENV-1, DENV-2, DENV-3 and DENV-4), Ebola virus (EBOV), Flavivirus, Hepatitis B virus (HBV), Herpes simplex virus (HSV), Human immunodeficiency virus (HIV), Human metapneumovirus (HMPV), Human Papilloma virus (HPV), Human parainfluenza viruses (HPIV), Influenza virus, extraintestinal pathogenic E. coli (ExPEC), Lassa mammarenavirus (LASV), MERS coronavirus, Mycobacterium tuberculosis, Nipah virus, Norovirus, Rabies virus (RABV), Respiratory Syncytial virus (RSV), Rhinovirus, Rotavirus, Vaccinia virus, Yellow Fever virus (YFV), Zika virus (ZIKV), Chlamydia trachomatis (i.e. bacterium Chlamydia causing Chlamydia), or Malaria parasite (e.g. Plasmodium falciparum, Plasmodium vivax, Plasmodium malariae, or Plasmodium ovale) infection as defined herein or diseases or disorders related thereto, by inducing or enhancing an adaptive immune response on the basis of an antigen encoded by the mRNA comprising lipid nanoparticles according to the invention.

Administration of the composition and/or the vaccine according to the invention may then occur prior, concurrent and/or subsequent to administering another composition and/or vaccine as defined herein, which may—in addition contain another mRNA comprising lipid nanoparticle or combination of mRNA comprising lipid nanoparticles encoding a different antigen or combination of antigens, wherein each antigen encoded by the mRNA sequence according to the invention is preferably suitable for the treatment or prophylaxis of SARS coronavirus 2 (SARS-CoV-2), nCoV-2019 coronavirus, SARS coronavirus (SARS-CoV), Bunyavirales virus, Cytomegalovirus (CMV), Dengue viruses (DENV-1, DENV-2, DENV-3 and DENV-4), Ebola virus (EBOV), Flavivirus, Hepatitis B virus (HBV), Herpes simplex virus (HSV), Human immunodeficiency virus (HIV), Human metapneumovirus (HMPV), Human Papilloma virus (HPV), Human parainfluenza viruses (HPIV), Influenza virus, extraintestinal pathogenic E. coli (ExPEC), Lassa mammarenavirus (LASV), MERS coronavirus, Mycobacterium tuberculosis, Nipah virus, Norovirus, Rabies virus (RABV), Respiratory Syncytial virus (RSV), Rhinovirus, Rotavirus, Vaccinia virus, Yellow Fever virus (YFV), Zika virus (ZIKV), Chlamydia trachomatis (i.e. bacterium Chlamydia causing Chlamydia), or Malaria parasite (e.g. Plasmodium falciparum, Plasmodium vivax, Plasmodium malariae, or Plasmodium ovale) infections and diseases or disorders related thereto.

In this context, a treatment as defined herein may also comprise the modulation of a disease associated to SARS coronavirus 2 (SARS-CoV-2), nCoV-2019 coronavirus, SARS coronavirus (SARS-CoV), Bunyavirales virus, Cytomegalovirus (CMV), Dengue viruses (DENV-1, DENV-2, DENV-3 and DENV-4), Ebola virus (EBOV), Flavivirus, Hepatitis B virus (HBV), Herpes simplex virus (HSV), Human immunodeficiency virus (HIV), Human metapneumovirus (HMPV), Human Papilloma virus (HPV), Human parainfluenza viruses (HPIV), Influenza virus, extraintestinal pathogenic E. coli (ExPEC), Lassa mammarenavirus (LASV), MERS coronavirus, Mycobacterium tuberculosis, Nipah virus, Norovirus, Rabies virus (RABV), Respiratory Syncytial virus (RSV), Rhinovirus, Rotavirus, Vaccinia virus, Yellow Fever virus (YFV), Zika virus (ZIKV), Chlamydia trachomatis (i.e. bacterium Chlamydia causing Chlamydia), or Malaria parasite (e.g. Plasmodium falciparum, Plasmodium vivax, Plasmodium malariae, or Plasmodium ovale) infection and of diseases or disorders related thereto.

According to a preferred embodiment of this aspect of the invention, the (pharmaceutical) composition or the vaccine according to the invention is administered by injection. Any suitable injection technique known in the art may be employed. Preferably, the inventive composition is administered by injection, preferably by needle-less injection, for example by jet-injection.

In one embodiment, the inventive composition comprises at least one, two, three, four, five, six, seven, eight, nine, ten, eleven, twelve or more mRNAs as defined herein, each of which is preferably injected separately, preferably by needle-less injection. Alternatively, the inventive composition comprises at least one, two, three, four, five, six, seven, eight, nine, ten, eleven, twelve or more mRNAs, wherein the at least one, two, three, four, five, six, seven, eight, nine, ten, eleven, twelve or more mRNAs are administered, preferably by injection as defined herein, as a mixture.

In a further aspect the invention relates to a method of immunization of a subject against an antigen or a combination of antigens.

The immunization protocol for the immunization of a subject against an antigen or a combination of at least two, three, four, five, six, seven, eight, nine, ten, eleven, twelve or more antigens as defined herein typically comprises a series of single doses or dosages of the (pharmaceutical) composition or the vaccine according to the invention. A single dosage, as used herein, refers to the initial/first dose, a second dose or any further doses, respectively, which are preferably administered in order to “boost” the immune reaction. In this context, each single dosage preferably comprises the administration of the same antigen or the same combination of antigens as defined herein, wherein the interval between the administration of two single dosages can vary from at least one day, preferably 2, 3, 4, 5, 6 or 7 days, to at least one week, preferably 2, 3, 4, 5, 6, 7 or 8 weeks. The intervals between single dosages may be constant or vary over the course of the immunization protocol, e.g. the intervals may be shorter in the beginning and longer towards the end of the protocol. Depending on the total number of single dosages and the interval between single dosages, the immunization protocol may extend over a period of time, which preferably lasts at least one week, more preferably several weeks (e.g. 2, 3, 4, 5, 6, 7, 8, 9, 10, 11 or 12 weeks), even more preferably several months (e.g. 3, 4, 5, 6, 7, 8, 9, 10, 11, 12, 18 or 24 months). Each single dosage preferably encompasses the administration of an antigen, preferably of a combination of at least two, three, four, five, six, seven, eight, nine, ten, eleven, twelve or more antigens as defined herein and may therefore involve at least one, preferably 1, 2, 3, 4, 5, 6, 7, 8, 9, 10, 11 or 12 injections. In some cases, the composition or the vaccine according to the invention is administered as a single dosage typically in one injection. In the case, where the vaccine according to the invention comprises separate mRNA formulations encoding distinct antigens as defined herein, the minimum number of injections carried out during the administration of a single dosage corresponds to the number of separate components of the vaccine. In certain embodiments, the administration of a single dosage may encompass more than one injection for each component of the vaccine (e.g. a specific mRNA formulation comprising an mRNA encoding, for instance, one antigenic peptide or protein as defined herein). For example, parts of the total volume of an individual component of the vaccine may be injected into different body parts, thus involving more than one injection. In a more specific example, a single dosage of a vaccine comprising four separate mRNA formulations, each of which is administered in two different body parts, comprises eight injections. Typically, a single dosage comprises all injections required to administer all components of the vaccine, wherein a single component may be involve more than one injection as outlined above. In the case, where the administration of a single dosage of the vaccine according to the invention encompasses more than one injection, the injection are carried out essentially simultaneously or concurrently, i.e. typically in a time-staggered fashion within the time-frame that is required for the practitioner to carry out the single injection steps, one after the other. The administration of a single dosage therefore preferably extends over a time period of several minutes, e.g. 2, 3, 4, 5, 10, 15, 30 or 60 minutes.

Administration of the mRNA comprising lipid nanoparticles as defined herein, the (pharmaceutical) composition or the vaccine according to the invention may be carried out in a time staggered treatment. A time staggered treatment may be e.g. administration of the mRNA comprising lipid nanoparticles, the composition or the vaccine prior, concurrent and/or subsequent to a conventional therapy of SARS coronavirus 2 (SARS-CoV-2), nCoV-2019 coronavirus, SARS coronavirus (SARS-CoV), Bunyavirales virus, Cytomegalovirus (CMV), Dengue viruses (DENV-1, DENV-2, DENV-3 and DENV-4), Ebola virus (EBOV), Flavivirus, Hepatitis B virus (HBV), Herpes simplex virus (HSV), Human immunodeficiency virus (HIV), Human metapneumovirus (HMPV), Human Papilloma virus (HPV), Human parainfluenza viruses (HPIV), Influenza virus, extraintestinal pathogenic E. coli (ExPEC), Lassa mammarenavirus (LASV), MERS coronavirus, Mycobacterium tuberculosis, Nipah virus, Norovirus, Rabies virus (RABV), Respiratory Syncytial virus (RSV), Rhinovirus, Rotavirus, Vaccinia virus, Yellow Fever virus (YFV), Zika virus (ZIKV), Chlamydia trachomatis (i.e. bacterium Chlamydia causing Chlamydia), or Malaria parasite (e.g. Plasmodium falciparum, Plasmodium vivax, Plasmodium malariae, or Plasmodium ovale) infections or diseases or disorders related thereto, e.g. by administration of the mRNA comprising lipid nanoparticles, the composition or the vaccine prior, concurrent and/or subsequent to a therapy or an administration of a therapeutic suitable for the treatment or prophylaxis of SARS coronavirus 2 (SARS-CoV-2), nCoV-2019 coronavirus, SARS coronavirus (SARS-CoV), Bunyavirales virus, Cytomegalovirus (CMV), Dengue viruses (DENV-1, DENV-2, DENV-3 and DENV-4), Ebola virus (EBOV), Flavivirus, Hepatitis B virus (HBV), Herpes simplex virus (HSV), Human immunodeficiency virus (HIV), Human metapneumovirus (HMPV), Human Papilloma virus (HPV), Human parainfluenza viruses (HPIV), Influenza virus, extraintestinal pathogenic E. coli (ExPEC), Lassa mammarenavirus (LASV), MERS coronavirus, Mycobacterium tuberculosis, Nipah virus, Norovirus, Rabies virus (RABV), Respiratory Syncytial virus (RSV), Rhinovirus, Rotavirus, Vaccinia virus, Yellow Fever virus (YFV), Zika virus (ZIKV), Chlamydia trachomatis (i.e. bacterium Chlamydia causing Chlamydia), or Malaria parasite (e.g. Plasmodium falciparum, Plasmodium vivax, Plasmodium malariae, or Plasmodium ovale) infections or diseases or disorders related thereto. Such time staggered treatment may be carried out using e.g. a kit, preferably a kit of parts as defined herein.

Time staggered treatment may additionally or alternatively also comprise an administration of the mRNA comprising lipid nanoparticles as defined herein, the (pharmaceutical) composition or the vaccine according to the invention in a form, wherein the mRNA encoding an antigenic peptide or protein as defined herein or a fragment or variant thereof, preferably forming part of the composition or the vaccine, is administered parallel, prior or subsequent to another mRNA comprising lipid nanoparticles as defined above, preferably forming part of the same inventive composition or vaccine. Preferably, the administration (of all mRNA comprising lipid nanoparticles) occurs within an hour, more preferably within 30 minutes, even more preferably within 15, 10, 5, 4, 3, or 2 minutes or even within 1 minute. Such time staggered treatment may be carried out using e.g. a kit, preferably a kit of parts as defined herein.

In a preferred embodiment, the pharmaceutical composition or the vaccine of the present invention is administered repeatedly, wherein each administration preferably comprises individual administration of the at least one mRNA comprising lipid nanoparticles of the inventive composition or vaccine. At each time point of administration, the at least one mRNA may be administered more than once (e.g. 2 or 3 times). In a particularly preferred embodiment of the invention, at least two, three, four, five, six or more mRNA sequences (each encoding a distinct one of the antigens as defined herein) encapsulated or associated with mRNA comprising lipid nanoparticles as defined above, wherein the mRNA sequences are part of mRNA compounds of the same or different lipid nanoparticles, are administered at each time point, wherein each mRNA is administered twice by injection, distributed over the four limbs.

In another preferred embodiment, the use of a pharmaceutical composition comprising a composition of the invention or a kit or kit of parts of the invention for (i) inducing an immune response, for (ii) inducing an antigen specific T-cell response or preferably for (iii) inducing CD8+ T cells responses is provided. Said method for (i) inducing an immune response, for (ii) inducing an antigen specific T-cell response or preferably for (iii) inducing CD8+ T cells responses in a subject; comprises administering to a subject in need thereof at least once an effective amount of a composition as described herein comprises an mRNA encoding at least one immunogenic peptide or polypeptide as also described herein. In another embodiment, the use of a pharmaceutical composition comprising a composition of the invention or a kit or kit of parts of the invention for (i) inducing an immune response, for (ii) inducing an antigen specific T-cell response or preferably for (iii) inducing CD8+ T cells responses is provided when compared to a reference (lipid nanoparticle) formulation or composition. Said reference (lipid nanoparticle) formulation or composition in a preferred embodiment does not comprise DPhyPE and/or a polymer conjugated lipid according to formula (I).

In embodiments, the nucleic acid as comprised in a composition of the invention is used for delivering said nucleic acid as defined herein is provided in an amount of about 100 ng to about 500 μg, in an amount of about 1 μg to about 200 μg, in an amount of about 1 μg to about 100 μg, in an amount of about 5 μg to about 100 μg, preferably in an amount of about 10 μg to about 50 μg, specifically, in an amount of about 1 μg, 2 μg, 3 μg, 4 μg, 5 μg, 10 μg, 15 μg, 20 μg, 25 μg, 30 μg, 35 μg, 40 μg, 45 μg, 50 μg, 55 μg, 60 μg, 65 μg, 70 μg, 75 μg, 80 μg, 85 μg, 90 μg, 95 μg or 100 μg.

In one embodiment, the immunization protocol for the treatment or prophylaxis of a subject against coronavirus, preferably SARS-CoV-2 coronavirus comprises one single doses of the composition or the vaccine, wherein the composition of the invention, comprising the inventive lipid excipient(s), is used for delivering said nucleic acid.

In some embodiments, the effective amount is a dose of 1 μg, 2 μg, 3 μg, 4 μg, 5 μg, 6 μg, 7 μg, 8 μg, 9 μg, 10 μg, 11 μg, 12 μg, 13 μg, 14 μg, 15 μg, 16 μg, 20 μg, 30 μg, 40 μg, 50 μg, 75 μg, 100 μg or 200 μg administered to the subject in one vaccination, wherein the composition of the invention is used for delivering said nucleic acid. In preferred embodiments, the immunization protocol for the treatment or prophylaxis of a coronavirus, preferably a SARS-CoV-2 coronavirus infection comprises a series of single doses or dosages, preferably a total of two doses, of the composition or the vaccine, wherein the composition of the invention, comprising the inventive lipid excipient(s), is used for delivering said nucleic acid. A single dosage, as used herein, refers to the initial/first dose, a second dose or any further doses, respectively, which are preferably administered in order to “boost” the immune reaction, wherein the composition of the invention, comprising the inventive lipid excipient(s), is used for delivering said nucleic acid.

In preferred embodiments, the vaccine/composition immunizes the subject against a coronavirus, preferably against a SARS-CoV-2 coronavirus infection (upon administration as defined herein) for at least 1 year, preferably at least 2 years, wherein for immunization the composition of the invention, comprising the inventive lipid excipient(s), is used for delivering said nucleic acid.

### Standard Therapy

The subject receiving the vaccine comprising RNAs of the invention may be a patient suffering from a tumor or cancer disease as described herein and who received or receives chemotherapy (e.g. first-line or second-line chemotherapy), radiotherapy, chemoradiotherapy/chemoradiation (combination of chemotherapy and radiotherapy), kinase inhibitors, antibody therapy and/or checkpoint modulators (e.g. CTLA4 inhibitors, PD1 pathway inhibitors), or a patient, who has achieved partial response or stable disease after having received one or more of the treatments specified above. More preferably, the subject is a patient suffering from a tumor or cancer disease as described herein and who received or receives a compound conventionally used in any of these diseases as described herein, more preferably a patient who receives or received a checkpoint modulator.

Compounds which preferably are used in standard therapies and which can be applied in combination with the pharmaceutical compositions or vaccines comprising RNAs of the invention include but are not limited to those disclosed on pages 56-58 in WO2018078053; WO2018078053 being incorporated herein by reference in its entirety.

### Tumor Indications

As used herein, the terms “tumor”, “cancer” or “cancer disease” refer to a malignant disease, which is preferably selected from, but not limited to, the group of malignant diseases disclosed on pages 58-59 in WO2018078053; WO2018078053 being incorporated herein by reference in its entirety.

### EXEMPLARY EMBODIMENTS

In the following, different embodiments of the invention are disclosed. It is intended herein, that each and every embodiment can be combined with each other, i.e. embodiment 1 may be combined with e.g. embodiment 4 or 14 or 24, or sub-embodiments like e.g. “embodiment 1.1”. Also, several sets of different embodiments of the invention are disclosed within. It is also intended herein, that each and every embodiment stemming from a different set of embodiments can be combined with each other, i.e. embodiment 1 from the First Set of Embodiments may be combined with e.g. embodiment 5 or embodiment 25 from the Second Set of Embodiments. Also back references to, e.g. “embodiment 1” are intended to comprise also a back-reference to, e.g. sub-embodiment 1.1, 1.2, et cetera.

### FIRST SET OF EMBODIMENTS

Embodiment 1. A vaccine composition comprising
a) at least one nucleic acid encoding at least one antigen or fragment or variant thereof; and
b) a carrier composition, wherein the carrier composition comprises the phospholipid phosphatidylserine.

Embodiment 1.1 A vaccine composition comprising
a) at least one nucleic acid encoding at least one antigen or fragment or variant thereof; and
b) a carrier composition, wherein the carrier composition comprises the phospholipid phosphatidylserine, wherein the amount of the phosphatidylserine is not more than 9 mol %, preferably not more than 5 mol %, of the total molar amount of all lipidic excipients in the carrier composition.

Embodiment 2. The vaccine composition according to embodiment 1, wherein the at least one nucleic acid is not a tolerogenic nucleic acid; and/or wherein the at least one nucleic acid does not encode a tolerogenic polypeptide; and/or wherein the vaccine composition does not comprise an antigen or fragment or variant thereof; and/or wherein the vaccine composition comprises the at least one nucleic acid as the sole payload; and/or wherein the vaccine composition is not a tolerogenic composition.

Embodiment 3. The vaccine composition according to embodiment 1 or 2, wherein the carrier composition at least partly encapsulates the at least one nucleic acid.

Embodiment 4. The vaccine composition according to any one of embodiments 1 to 3, wherein the carrier composition encapsulates the at least one nucleic acid.

Embodiment 5. The vaccine composition according to any one of embodiments 1 to 4, wherein the carrier composition comprises an inner surface and an outer surface facing the outside, wherein the phosphatidylserine is located at the outer surface of the carrier composition.

Embodiment 6. The vaccine composition according to embodiment 5, wherein the hydrophilic head group of the phosphatidylserine is located at the outer surface of the carrier composition.

Embodiment 7. The vaccine composition according to any one of embodiments 1 to 6, wherein the hydrophilic head group of the phosphatidylserine comprised in the carrier composition is accessible from the outside of the carrier composition.

Embodiment 8. The vaccine composition according to any one of embodiments 1 to 7, wherein the phosphatidylserine is selected from the group consisting of DPhyPS, WT-PS, 16:0—PS, 14:0—PS, 10:0—PS, 6:0—PS, 18:1—PS DOPS, 18:1-Lyso PS and 18:0-Lyso PS.

Embodiment 9. The vaccine composition according to any one of embodiments 1 to 8, wherein the carrier composition is a lipid nanoparticle composition.

Embodiment 10. The vaccine composition according to embodiment 9, wherein the lipid nanoparticle composition further comprises
(i) a cationic or ionizable lipid; and/or
(ii) a steroid; and/or
(iii) a further phospholipid in addition to phosphatidylserine, preferably DPhyPE; and/or
(iv) a polymer conjugated lipid.

Embodiment 10.1 The vaccine composition according to embodiment 9, wherein the lipid nanoparticle composition further comprises
(i) a cationic or ionizable lipid; and/or
(ii) a steroid; and/or
(iii) a second phospholipid in addition to phosphatidylserine, preferably DPhyPE; and/or
(iv) PMOZ 4.

Embodiment 11. The vaccine composition according to embodiment 9 or 10, wherein the lipid nanoparticle composition further comprises
(i) a cationic or ionizable lipid;
(ii) a steroid;
(iii) a further phospholipid in addition to phosphatidylserine, preferably DPhyPE; and
(iv) a polymer conjugated lipid.

Embodiment 11.1 The vaccine composition according to embodiment 9 or 10, wherein the lipid nanoparticle composition further comprises
(i) a cationic or ionizable lipid;
(ii) a steroid;
(iii) a second phospholipid in addition to phosphatidylserine, preferably DPhyPE; and
(iv) PMOZ 4.

Embodiment 12. The vaccine composition according to embodiment 10 or 11, wherein the cationic or ionizable lipid carries a net positive charge at physiological pH, preferably wherein the cationic or ionizable lipid comprises a tertiary nitrogen group or quaternary nitrogen group, more preferably wherein the cationic or ionizable lipid is selected from the group consisting of HEXA1, HEXA2 and THIOETHER with the structures shown in FIGS. 1A, 1B and 1C, respectively.

Embodiment 13. The vaccine composition according to any one of embodiments 10 to 12, wherein the steroid is selected from the group consisting of cholesterol, cholesteryl hemisuccinate (CHEMS) and a derivate thereof, preferably wherein the steroid is cholesterol.

Embodiment 14. The vaccine composition according to any one of embodiments 10 to 13, wherein the further phospholipid is selected from the group consisting of 1,2-diphytanoyl-sn-glycero-3-phosphoethanolamine (DPhyPE; 1,2-di-(3,7,11,15-tetramethylhexadecanoyl)-sn-glycero-3-phosphoethanolamine), 1,2-diphytanoyl-sn-glycero-3-phosphocholine (DPhyPC), 1,2-dioleoyl-sn-glycero-3-phosphocholine (DOPC; dioleoylphosphatidylcholine), 1,2-Dipalmitoyl-sn-glycero-3-phosphocholine (DPPC; dipalmitoylphosphatidylcholine), 1,2-dioleoyl-sn-glycero-3-phosphoethanolamine (DOPE), phosphatidylethanolamines, distearoylphosphatidylcholines, dioleoyl-phosphatidylethanolamine (DOPEA), 1,2-distearoyl-sn-glycero-3-phosphoethanolamine (DSPE), palmitoyloleoylphosphatidylcholine (POPC), palmitoyloleoyl-phosphatidylethanolamine (POPE), 1,2-Dipalmitoyl-sn-glycero-3-phosphoethanolamine (DPPE), dioleoyl-phosphatidylethanolamine 4-(N-maleimidomethyl)-cyclohexane-1-carboxylate (DOPE-mal), 1,2-Dimyristoyl-sn-glycero-3-phosphoethanolamine (DMPE), 1,2-Dilinoleoyl-sn-glycero-3-phosphoethanolamine (DLoPE), distearoyl-phosphatidylethanolamine (DSPE), 1-Palmitoyl-2-oleoyl-sn-glycero-3-phosphoethanolamine (POPE), 1,2-Dilauroyl-sn-glycero-3-phosphoethanolamine (DLPE), 16-O-monomethylphosphoethanolamine, 16-O-dimethyl phosphatidylethanolamine, 1,2-Dierucoyl-sn-glycero-3-phosphoethanolamine (DEPE), 18-1-trans phosphatidylethanolamine, 1-stearoyl-2-oleoylphosphatidyethanolamine (SOPE), 1,2-Disqualeoyl-sn-glycero-3-phosphoethanolamine (DSQPE), 1,2-dielaidoyl-sn-glycero-3-phosphoethanolamine (transDOPE), 1-Stearoyl-2-linoleoyl-sn-glycero-3-phosphoethanolamine (SLPE), 1-tridecanoyl-sn-glycero-3-phospho-L-serine (sodium salt), 1-oleoyl-2-hydroxy-sn-glycero-3-phospho-L-serine (sodium salt), 1-palmitoyl-2-oleoyl-sn-glycero-3-phospho-L-serine (sodium salt) (POPS), 1-1-stearoyl-2-oleoyl-sn-glycero-3-phospho-L-serine (sodium salt), 1,2-dioleoyl-sn-glycero-3-phospho-L-serine (sodium salt) (DOPS), 1,2-distearoyl-sn-glycero-3-phospho-L-serine (sodium salt), 1,2-diphytanoyl-sn-glycero-3-phospho-L-serine (sodium salt), 1-O-hexadecanyl-2-O-(9Z-octadecenyl)-sn-glycero-3-phosphoethanolamine, 1,2-distearoyl-sn-glycero-3-phosphatidylcholine or 1,2-distearoyl-sn-glycero-3-phosphocholine (DSPC), 1,2-di-O-phytanyl-sn-glycero-3-phosphoethanolamine, 1-palmitoyl-2-cholesterylhemisuccinoyl-sn-glycero-3-phosphocholine (PChemsPC), 1,2-dicholesterylhemisuccinoyl-sn-glycero-3-phosphocholine (DChemsPC), 2-((2,3-bis(oleoyloxy)propyl)dimethylammonio)ethyl hydrogen phosphate (DOCP), 2-((2,3-bis(oleoyloxy)propyl)dimtheylammonio)ethyl ethyl phosphate (DOCPe), and 1-O-octadecyl-2-O-methyl-sn-glycero-3-phosphocholine (Edelfosine), preferably wherein the further phospholipid is DPhyPE; and wherein the phospholipid, preferably DPhyPE, is optionally present in combination with a phospholipid having at least two alkyl chains, wherein each alkyl chain independently has a length of preferably C6, C7, C8, C9, or C10, more preferably a length of C6, C7, or C8, most preferably a length of C7, further most preferably a phospholipid selected from the group consisting of DHPC (1,2-diheptanoyl-sn-glycero-3-phosphocholine), 05:0 PC (1,2-dipentanoyl-sn-glycero-3-phosphocholine), 04:0 PC (1,2-dibutyryl-sn-glycero-3-phosphocholine), 06:0 PC (1,2-dihexanoyl-sn-glycero-3-phosphocholine), 08:0 PC (1,2-dioctanoyl-sn-glycero-3-phosphocholine), and 09:0 PC (1,2-dinonanoyl-sn-glycero-3-phosphocholine), with DHPC being most preferred as the optionally present phospholipid having at least two alkyl chains.

Embodiment 14.1 The vaccine composition according to any one of embodiments 10 to 13 comprising a third phospholipid next to phosphatidylserine and 1,2-diphytanoyl-sn-glycero-3-phosphoethanolamine (DPhyPE), wherein the third phospholipid is DHPC (1,2-diheptanoyl-sn-glycero-3-phosphocholine).

Embodiment 15. The vaccine composition according to any one of embodiments 10 to 14, wherein the polymer conjugated lipid is a pegylated lipid or a PMOZ-lipid.

Embodiment 16. The vaccine composition according to any one of embodiments 11 to 15, wherein the composition comprises excipients in a ratio selected from the group consisting of
(a-i) the cationic lipid at an amount of 30-70 mol %; the steroid at an amount of 20-50 mol %; the phospholipid at an amount of 5-25 mol %; and the polymer conjugated lipid at an amount of 0.5-5 mol %;
(a-ii) the cationic lipid at an amount of 40-60 mol %; the steroid at an amount of 20-40 mol %; the phospholipid at an amount of 10-20 mol %; and the polymer conjugated lipid at an amount of 1-2 mol %;
(a-iii) the cationic lipid of embodiment 12 at an amount of 30-70 mol %; the steroid of embodiment 13 at an amount of 20-50 mol %; the phospholipid phosphatidylserine and the phospholipid of embodiment 14 at an amount of 5-25 mol %; and the polymer conjugated lipid of embodiment 15 at an amount of 0.5-5 mol %; and
(a-iv) the cationic lipid of embodiment 12 at an amount of 40-60 mol %; the steroid of embodiment 13 at an amount of 20-40 mol %; the phospholipid phosphatidylserine and the phospholipid of embodiment 14 at an amount of 10-20 mol %; and the polymer conjugated lipid of embodiment 15 at an amount of 1-2 mol %; preferably the composition comprising excipients in a ratio selected from the group consisting of
(b-i) the cationic lipid at an amount of 59 mol %; the steroid at an amount of 29.3 mol %; the phospholipid at an amount of 10 mol %; and the polymer conjugated lipid at an amount of 1.7 mol %;
(b-li) the cationic lipid at an amount of 58 mol %; the steroid at an amount of 29.3 mol %; the phospholipid at an amount of 11 mol %; and the polymer conjugated lipid at an amount of 1.7 mol %;
(b-iii) the cationic lipid at an amount of 49 mol %; the steroid at an amount of 29.3 mol %; the phospholipid at an amount of 20 mol %; and the polymer conjugated lipid at an amount of 1.7 mol %;
(b-iv) the cationic lipid of embodiment 12 at an amount of 59 mol %; the steroid of embodiment 13 at an amount of 29.3 mol %; the phospholipid phosphatidylserine and the phospholipid of embodiment 14 at an amount of 10 mol %; and the polymer conjugated lipid of embodiment 15 at an amount of 1.7 mol %;
(b-v) the cationic lipid of embodiment 12 at an amount of 58 mol %; the steroid of embodiment 13 at an amount of 29.3 mol %; the phospholipid phosphatidylserine and the phospholipid of embodiment 14 at an amount of 11 mol %; and the polymer conjugated lipid of embodiment 15 at an amount of 1.7 mol %; and
(b-vi) the cationic lipid of embodiment 12 at an amount of 49 mol %; the steroid of embodiment 13 at an amount of 29.3 mol %; the phospholipid phosphatidylserine and the phospholipid of embodiment 14 at an amount of 20 mol %; and the polymer conjugated lipid of embodiment 15 at an amount of 1.7 mol %; more preferably the composition comprising excipients in a ratio selected from the group consisting of
(c-i) the cationic lipid of embodiment 12 at an amount of 59 mol %; the steroid of embodiment 13 at an amount of 29.3 mol %; the phospholipid phosphatidylserine at an amount of 5 mol % and DPhyPE at an amount of 5 mol %; and the polymer conjugated lipid of embodiment 15 at an amount of 1.7 mol %;
(c-ii) the cationic lipid of embodiment 12 at an amount of 59 mol %; the steroid of embodiment 13 at an amount of 29.3 mol %; the phospholipid phosphatidylserine at an amount of 2 mol % and DPhyPE at an amount of 8 mol %; and the polymer conjugated lipid of embodiment 15 at an amount of 1.7 mol %;
(c-iii) the cationic lipid of embodiment 12 at an amount of 58 mol %; the steroid of embodiment 13 at an amount of 29.3 mol %; the phospholipid phosphatidylserine at an amount of 5 mol %, DPhyPE at an amount of 5 mol % and DHPC at an amount of 1 mol %; and the polymer conjugated lipid of embodiment 15 at an amount of 1.7 mol %;
(c-iv) the cationic lipid of embodiment 12 at an amount of 49 mol %; the steroid of embodiment 13 at an amount of 29.3 mol %; the phospholipid phosphatidylserine at an amount of 5 mol %, DPhyPE at an amount of 5 mol % and DHPC at an amount of 10 mol %; and the polymer conjugated lipid of embodiment 15 at an amount of 1.7 mol %.

Embodiment 17. The vaccine composition according to any one of the preceding embodiments, wherein the at least one nucleic acid is DNA or RNA.

Embodiment 18. The vaccine composition according to embodiment 17, wherein the at least one nucleic acid is RNA, preferably mRNA comprising a coding sequence encoding the at least one antigen or fragment or variant thereof and optionally a coding sequence encoding at least one self-amplifying enzyme.

Embodiment 19. The vaccine composition according to embodiment 18, wherein the lipid nanoparticles comprise the mRNA
(i) at an amount such as to achieve an N/P ratio in the range of 10 to 20, preferably about 2 to about 15, more preferably about 3 to about 10, even more preferably about 4 to about 9, most preferably about 6; or
(ii) at an amount such as to achieve an N/P ratio in the range of about 5 to about 20, more preferably about 10 to about 18, even more preferably about 12 to about 16, most preferably about 14; and/or
(iii) at an amount such as to achieve a lipid: mRNA weight ratio in the range of about 20 to about 60, preferably from about 3 to about 15, about 5 to about 13, about 4 to about 8 or from about 7 to about 11; and/or
wherein the lipid nanoparticles have a mean hydrodynamic diameter as determined by dynamic laser scattering from about 50 nm to about 300 nm, or from about 60 nm to about 250 nm, or from about 60 nm to about 200 nm, or from about 70 nm to 200 nm, or from about 75 nm to about 160 nm, or from about 90 nm to about 140 nm, or from about 100 nm to about 140 nm; and/or
wherein the lipid nanoparticles exhibit a zeta potential in the range of −50 mV to +50 mV.

Embodiment 20. The vaccine composition according to embodiment 18 or 19, wherein the mRNA is a mono-, bi-, or multicistronic mRNA.

Embodiment 21. The vaccine composition according to any one of embodiments 18 to 20, wherein the mRNA comprises at least one chemical modification.

Embodiment 22. The vaccine composition according to embodiment 21, wherein the chemical modification is selected from the group consisting of base modifications, sugar modifications, backbone modifications and lipid modifications, preferably wherein the chemical modification is a base modification, more preferably wherein the base modification preferably is selected from the group consisting of pseudouridine (psi or Lp), N1-methylpseudouracil (N1MPU, N1Mpsi or N1MLp), 1-ethylpseudouracil, 2-thiouracil (s2U), 4-thiouracil, 5-methylcytosine, 5-methyluracil, 5-methoxyuracil, and any combination thereof.

Embodiment 23. The vaccine composition according to any one of embodiments 18 to 22, wherein the coding sequence exhibits a sequence modification.

Embodiment 24. The vaccine composition according to embodiment 23, wherein the sequence modification is selected from a G/C content modification, a codon modification, a codon optimization or a C-optimization of the sequence; preferably wherein, compared with the coding sequence of the corresponding wild-type mRNA, the
a) G/C content of the coding sequence is increased;
b) C content of the coding sequence is increased;
c) codon usage in the coding sequence is adapted to the human codon usage; and/or
d) codon adaptation index (CAI) is increased or maximised in the coding sequence.

Embodiment 25. The vaccine composition according to any one of embodiments 18 to 24, wherein the mRNA further comprises
a) a 5′-CAP structure, preferably m7GpppN, more preferably CAP1 or m7G(5′)ppp(5′)(2′OMeA)pG;
b) at least one miRNA binding site sequence, preferably wherein the microRNA binding site is for a microRNA selected from the group consisting of a miR-126, miR-142, miR-144, miR-146, miR-150, miR-155, miR-16, miR-21, miR-223, miR-24, miR-27, miR-26a binding site, preferably a miR-122 or miR-142 binding site, or any combination of the aforementioned miRNA binding sites thereof;
c) at least one 5′-UTR element;
d) at least one 3′-UTR element;
e) at least one poly(A) sequence;
f) at least one poly(C) sequence;
g) optionally, a histone stem-loop selected from SEQ ID NO:3 or 4;
h) optionally, a 3-terminal sequence element selected from SEQ ID NO:41-70;
or any combinations of these.

Embodiment 26. The vaccine composition according to any one of embodiments 18 to 25, wherein the mRNA comprises a 5′-CAP structure, preferably m7G, CAP0, CAP1, CAP2, a modified CAP0 or a modified CAP1 structure.

Embodiment 27. The vaccine composition according to embodiment 25, wherein the at least one coding RNA comprises at least one heterologous 5′-UTR and/or at least one heterologous 3′-UTR, preferably wherein the at least one heterologous 5′-UTR comprises a nucleic acid sequence derived from a 5′-UTR of a gene selected from HSD17B4, RPL32, ASAH1, ATP5A1, MP68, NDUFA4, NOSIP, RPL31, SLC7A3, TUBB4B and UBQLN2, or from a homolog, a fragment or variant of any one of these genes; and/or preferably wherein the at least one heterologous 3′-UTR comprises a nucleic acid sequence derived from a 3′-UTR of a gene selected from PSMB3, ALB/albumin, alpha-globin, CASP1 (preferably SEQ ID NO:81 (DNA) or SEQ ID NO:82 (RNA)), COX6B1 (preferably SEQ ID NO:83 (DNA) or SEQ ID NO:84 (RNA)), GNAS (preferably SEQ ID NO:85 (DNA) or SEQ ID NO:86 (RNA)), NDUFA1 (preferably SEQ ID NO:87 (DNA) or SEQ ID NO:88 (RNA)) and RPS9 (preferably SEQ ID NO:79 (DNA) or SEQ ID NO:80 (RNA)), or from a homolog, a fragment or a variant of any one of these genes.

Embodiment 28. The vaccine composition according to embodiment 27, wherein the at least one coding RNA comprises a (i) HSD17B4 5′-UTR and a PSMB3 3′-UTR or (ii) a RPL32 5′-UTR and an ALB/albumin 3′-UTR, preferably a mutated alpha-globin 3′-UTR (SEQ ID NO:11, 12), more preferably a HSD17B4 5′-UTR (SEQ ID NO:21, 22) and a PSMB3 3′-UTR (SEQ ID NO:19, 20).

Embodiment 29. The vaccine composition according to any one of embodiments 18 to 24, wherein the mRNA comprises the following elements in the 5′ to 3′ direction:
a) a 5′-CAP structure, preferably selected from the group consisting of m7G(5′), m7G(5′)ppp(5′)(2′OMeA) and m7G(5′)ppp(5′)(2′OMeG);
b) a 5′-UTR element comprising a nucleic acid sequence derived from the 5′-UTR of a TOP gene, said nucleic acid sequence preferably comprising an RNA sequence that corresponds to the nucleic acid sequence according to SEQ ID NO:22, SEQ ID NO:24, SEQ ID NO:26 or preferably SEQ ID NO:77/78 (SLC7A3) or SEQ ID NO:75/76 (RPL31), or a homolog, a fragment or a variant thereof; most preferably according to SEQ ID NO:22 (HSD17B4);
c) the at least one coding sequence;
d) a 3′-UTR element comprising a nucleic acid sequence derived from an α-globin gene, said nucleic acid sequence preferably comprising an RNA sequence that corresponds to the nucleic acid sequence according to SEQ ID NO:6, 8 or SEQ ID NO:10, 12, 14, 16, 18, or preferably SEQ ID NO:20, or a homolog, a fragment or a variant thereof; and/or a 3′-UTR element comprising a nucleic acid sequence derived from an albumin gene, said nucleic acid sequence preferably comprising an RNA sequence that corresponds to the nucleic acid sequence according to SEQ ID NO:18 (ALB/albumin) or preferably SEQ ID NO:79/80 (RPS9), or a homolog, a fragment or a variant thereof; most preferably according to SEQ ID NO:20 (PSMB3);
e) optionally, at least one poly(A) sequence, preferably consisting of 10 to 200, 10 to 100, 40 to 80, or 50 to 70 adenosine nucleotides, more preferably at least 70 adenosine nucleotides, even more preferably about 100 adenosine nucleotides;
f) optionally, at least one poly(C) sequence, preferably consisting of 10 to 200, 10 to 100, 20 to 70, 20 to 60 or 10 to 40 cytosine nucleotides; and
g) optionally, at least one histone stem-loop, preferably comprising the RNA sequence according to SEQ ID NO:4.

Embodiment 30. The vaccine composition according to any one of the preceding embodiments, wherein the antigen is derived from a pathogenic antigen, a tumour antigen, an allergenic antigen or an autoimmune self-antigen.

Embodiment 31. The vaccine composition according to embodiment 30, wherein the pathogenic antigen is selected from the group consisting of a bacterial antigen, a viral antigen, a fungal antigen and a protozoal antigen.

Embodiment 32. The vaccine composition according to embodiment 30 or 31, wherein the pathogenic antigen
(i) is derived from a SARS coronavirus 2 (SARS-CoV-2), nCoV-2019 coronavirus, SARS coronavirus (SARS-CoV), Bunyavirales virus, Cytomegalovirus (CMV), Dengue viruses (DENV-1, DENV-2, DENV-3 and DENV-4), Ebola virus (EBOV), Flavivirus, Hepatitis B virus (HBV), Herpes simplex virus (HSV), Human immunodeficiency virus (HIV), Human metapneumovirus (HMPV), Human Papilloma virus (HPV), Human parainfluenza viruses (HPIV), Influenza virus, extraintestinal pathogenic E. coli (ExPEC), Lassa mammarenavirus (LASV), MERS coronavirus, Mycobacterium tuberculosis, Nipah virus, Norovirus, Rabies virus (RABV), Respiratory Syncytial virus (RSV), Rhinovirus, Rotavirus, Vaccinia virus, Yellow Fever virus (YFV), Zika virus (ZIKV), Chlamydia trachomatis (i.e. bacterium Chlamydia causing Chlamydia), or Malaria parasite (e.g. Plasmodium falciparum, Plasmodium vivax, Plasmodium malariae, or Plasmodium ovale); and/or
(ii) is derived from a structural protein, an accessory protein, or a replicase protein from a SARS coronavirus 2 (SARS-CoV-2), nCoV-2019 coronavirus, SARS coronavirus (SARS-CoV), or an immunogenic fragment or immunogenic variant of any of these; and/or
(iii) is derived from a spike protein (S), an envelope protein (E), a membrane protein (M) or a nucleocapsid protein (N) from a SARS coronavirus 2 (SARS-CoV-2), nCoV-2019 coronavirus, SARS coronavirus (SARS-CoV), or an immunogenic fragment or immunogenic variant of any of these, preferably wherein the spike protein (S) comprises or consists of spike protein fragment S1 or spike protein fragment S2, more preferably spike protein fragment S1, or an immunogenic fragment or immunogenic variant thereof; and/or
(iv) is derived from a pre-fusion stabilized spike protein (S) (S_stab) from a SARS coronavirus 2 (SARS-CoV-2), nCoV-2019 coronavirus, SARS coronavirus (SARS-CoV) comprising at least one pre-fusion stabilizing mutation.

Embodiment 33. A pharmaceutical composition comprising the vaccine composition according to any one of embodiments 30 to 32 and a pharmaceutically acceptable carrier, diluent or excipient, preferably wherein the pharmaceutical composition is a sterile solid composition for reconstitution with a sterile liquid carrier, and wherein the composition further comprises one or more inactive ingredients selected from pH-modifying agents, bulking agents, stabilizers, non-ionic surfactants and antioxidants, and wherein the sterile liquid carrier is an aqueous carrier.

Embodiment 34. The vaccine composition according to any one of embodiments 30 to 32 or the pharmaceutical composition according to embodiment 33 for use in the treatment or prophylaxis of infectious diseases; cancer or tumor diseases, disorders or conditions; liver diseases selected from the group consisting of liver fibrosis, liver cirrhosis and liver cancer; allergies; or autoimmune disease, disorder or condition; in a subject.

Embodiment 35. The vaccine composition according to embodiment 32 or a pharmaceutical composition comprising the vaccine composition according to embodiment 32 for use in the treatment or prophylaxis of infectious diseases including viral, bacterial or protozoological infectious diseases in a subject.

Embodiment 36. The vaccine composition and the pharmaceutical composition for use according to embodiment 34 or 35, wherein the vaccine composition or pharmaceutical composition is administered via local or locoregional injection, infusion or implantation, in particular intradermal, subcutaneous, intramuscular, intracameral, subconjunctival, suprachoroidal injection, subretinal, subtenon, retrobulbar, topical, posterior juxtascleral administration, or intrapulmonal inhalation, interstitial, locoregional, intravitreal, intratumoral, intralymphatic, intranodal, intra-articular, intrasynovial, periarticular, intraperitoneal, intra-abdominal, intracardial, intralesional, intrapericardial, intraventricular, intrapleural, perineural, intrathoracic, epidural, intradural, peridural, intrathecal, intramedullary, intracerebral, intracavernous, intracorporus cavernosum, intraprostatic, intratesticular, intracartilaginous, intraosseous, intradiscal, intraspinal, intracaudal, intrabursal, intragingival, intraovarian, intrauterine, periocular, periodontal, retrobulbar, subarachnoid, subconjunctival, suprachoroidal injection, infusion, implantation, nasal, buccal, sublingual, otic or auricular, ophthalmic, conjunctival, vaginal, rectal, intracervical, endosinusial, laryngeal, oropharyngeal, ureteral, urethral administration, more preferably said lipid nanoparticle is administered intramuscularly, intravenously, intradermally, subcutaneously, intratumorally, intranasally, or by inhalation to a subject, preferably via local or locoregional injection or infusion to a subject.

Embodiment 37. A kit or kit of parts, comprising the vaccine composition according to any one of embodiments 30 to 32 or the pharmaceutical composition according to embodiment 33, optionally comprising a liquid vehicle for solubilizing, and, optionally, technical instructions providing information on administration and dosage of the components.

Embodiment 38. A method of treatment or prophylaxis of infectious diseases; cancer or tumor diseases, disorders or conditions; liver diseases selected from the group consisting of liver fibrosis, liver cirrhosis and liver cancer; allergies; or autoimmune disease, disorder or condition; in a subject comprising the steps:
a) providing the vaccine composition of any one of embodiments 30 to 32 or the pharmaceutical composition according to embodiment 33 or the kit or kit of parts according to embodiment 37; and
b) applying or administering the vaccine composition or the pharmaceutical composition or the kit or kit of parts to a tissue or an organism of the subject.

Embodiment 39. A method of inducing an immune response in a subject, the method comprising administering to the subject the vaccine composition of any one of embodiments 1 to 32 or the pharmaceutical composition of embodiment 33 in an amount effective to produce an antigen-specific immune response in the subject.

Embodiment 40. A method of targeting a vaccine composition comprising a) at least one nucleic acid, preferably mRNA, encoding at least one antigen or fragment or variant thereof; and b) a carrier composition, preferably a lipid nanoparticle composition, to antigen-presenting cells including dendritic cells and macrophages, and/or to the spleen, the method comprising administering to the subject the vaccine composition of any one of embodiments 1 to 32 or the pharmaceutical composition of embodiment 33.

Embodiment 41. Use of a vaccine composition of any one of embodiments 1 to 32 or the pharmaceutical composition according to embodiment 33 or the kit or kit of parts according to embodiment 37 for (i) inducing an immune response, for (ii) inducing an antigen specific T-cell response, preferably for (iii) inducing CD8+ T cells responses, and/or for (iv) targeting the vaccine composition or the pharmaceutical composition to antigen-presenting cells, including dendritic cells and macrophages, and/or to the spleen, in a subject.

Embodiment 42. Use of phosphatidylserine in a vaccine of any one of the above embodiments or in a carrier composition of any one of the above embodiments comprising a) at least one nucleic acid, preferably mRNA, encoding at least one antigen or fragment or variant thereof; and b) a carrier composition, preferably a lipid nanoparticle composition, for targeting the vaccine composition to antigen-presenting cells, including dendritic cells and macrophages, and/or to the spleen, in a subject.

Embodiment 42.1 Use of phosphatidylserine in the carrier composition of any of the above embodiments of a vaccine composition comprising a) at least one nucleic acid, preferably mRNA, encoding at least one antigen or fragment or variant thereof; and b) a carrier composition, preferably a lipid nanoparticle composition, for targeting the vaccine composition to antigen-presenting cells, including dendritic cells and macrophages, and/or to the spleen, in a subject.

Embodiment 42.2 Use of phosphatidylserine in a carrier composition of any one of the above embodiments comprising a) at least one nucleic acid, preferably mRNA, encoding at least one antigen or fragment or variant thereof; and b) a carrier composition, preferably a lipid nanoparticle composition, for targeting the vaccine composition to antigen-presenting cells, in a subject.

Embodiment 42.3 Use of phosphatidylserine in a carrier composition of any one of the above embodiments comprising a) at least one nucleic acid, preferably mRNA, encoding at least one antigen or fragment or variant thereof; and b) a carrier composition, preferably a lipid nanoparticle composition, for targeting the vaccine composition to the spleen, in a subject.

Embodiment 43. The vaccine composition or the pharmaceutical composition for use according to embodiment 34 or 35, the method according to embodiment 38, 39 or 40, or the use according to embodiment 41 or 42, wherein the subject is a mammalian subject, preferably a human subject.

Embodiment 44. Any polymer conjugated lipid of the previous embodiments, wherein said polymer conjugated lipid does not comprise a polyethylene glycol-(PEG)-moiety or residue; and/or does not comprise a sulphur group (—S—); and/or a terminating nucleophile.

Embodiment 45. Any polymer conjugated lipid of the previous embodiments, wherein said polymer conjugated lipid does not comprise a polyethylene glycol-(PEG)-moiety or residue.

Embodiment 46. Any polymer conjugated lipid of the previous embodiments, wherein said polymer conjugated lipid does not comprise a sulphur group (—S—).

Embodiment 47. Any polymer conjugated lipid of the previous embodiments, wherein said polymer conjugated lipid does not comprise a terminating nucleophile.

Embodiment 48. Any polymer conjugated lipid of the previous embodiments, wherein said polymer conjugated lipid does not comprise a sulphur group (—S—); and a terminating nucleophile.

Embodiment 49. A vaccine composition or carrier composition comprising
a) at least one nucleic acid encoding at least one antigen or fragment or variant thereof; and
b) a carrier composition, wherein the carrier composition comprises the phospholipid 1,2-diheptanoyl-sn-glycero-3-phosphocholine (DHPC).

Embodiment 50. Use of DHPC in the carrier composition of a vaccine composition comprising a) at least one nucleic acid, preferably mRNA, encoding at least one antigen or fragment or variant thereof; and b) a vaccine or carrier composition, preferably a lipid nanoparticle composition, for targeting the vaccine composition to antigen-presenting cells, including dendritic cells and macrophages, and/or to the spleen, in a subject.

Embodiment 51. A vaccine composition or the pharmaceutical composition comprising DHPC for use according to embodiment 34 or 35, the method according to embodiment 38, 39 or 40, or the use according to embodiment 41 or 42, wherein the subject is a mammalian subject, preferably a human subject.

### SECOND SET OF EMBODIMENTS

Embodiment 1. A polymer conjugated lipid according to formula (I):

[P]-[linker]-[L] formula (I)

or a pharmaceutically acceptable salt, prodrug, tautomer or stereoisomer thereof, wherein
[P] is a heteropolymer moiety or homopolymer moiety, preferably a homopolymer moiety, comprising at least one polyoxazoline (POZ) monomer unit

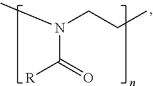

wherein R is C1-9 alkyl or C2-9 alkenyl, preferably C1 or C2 alkyl, and n has a mean value ranging from about 45 to about 55, preferably n is about 50 or wherein n is selected such that the [P] moiety has an average molecular weight of about 4.4 kDa, or most preferably about 4.3 kDa
[linker] is an optional linker group, and
[L] is a lipid moiety.
Embodiment 2. The polymer conjugated lipid of embodiment 1, wherein [P] is a heteropolymer moiety or homopolymer moiety comprising multiple monomer units selected from the group consisting of

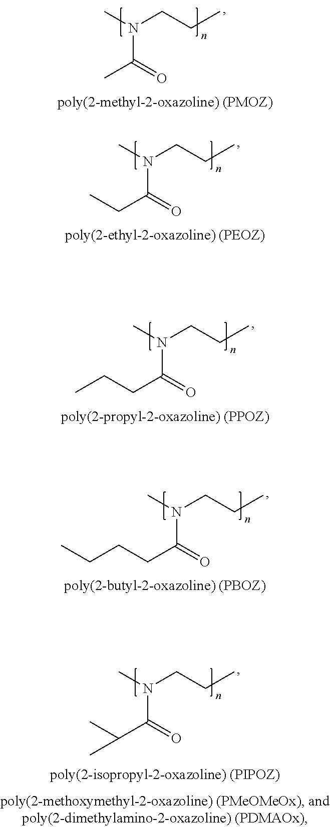

preferably wherein [P] is a homopolymer moiety comprising multiple PMOZ or PEOZ monomer units, more preferably wherein [P] comprises or preferably consists of multiple PMOZ monomer units,
wherein
(i) n has a mean value ranging from about 45 to about 55, preferably n is about 50 or wherein
(ii) n is selected such that the [P] moiety has an average molecular weight of about 3 kDa to about 6 kDa, preferably an average molecular weight of about 4.2 kDa to about 4.4 kDa, or most preferably about 4.3 kDa.
Embodiment 3. The polymer conjugated lipid of any one of embodiment 1 to embodiment 2, wherein the polymer conjugated lipid is selected from the group consisting of a POZ-monoacylglycerol conjugate, POZ-diacylglycerol conjugate, a POZ-dialkyloxypropyl conjugate, a POZ-steroid or POZ-sterol conjugate, a POZ-phospholipid conjugate, a POZ-ceramide conjugate, and a mixture thereof.
Embodiment 4. The polymer conjugated lipid of any one of embodiment 1 to embodiment 3, wherein
   (i) the lipid moiety [L] comprises at least one straight or branched, saturated or unsaturated alkyl chain containing from 6 to 30 carbon atoms, preferably wherein the lipid moiety [L] comprises at least one straight or branched saturated alkyl chain, wherein the alkyl chain is optionally interrupted by one or more biodegradable group(s) and/or optionally comprises one terminal biodegradable group, wherein the biodegradable group is selected from the group consisting of but not limited to a pH-sensitive moiety, an alkyl or alkenyl moiety (C₁₋₉ alkyl or C₂₋₉ alkenyl), a zwitterionic linker, non-ester containing linker moieties and ester-containing linker moieties (—C(O)O— or —OC(O)—), amido (—C(O)NH—), disulfide (—S—S—), carbonyl (—C(O)—), ether (—O—), thioether (—S—), oxime (e.g., —C(H)═N—O— or —O—N═C(H)—), carbamate (—NHC(O)O—), urea (—NHC(O)NH—), succinyl (—(O)CCH₂CH₂C(O)—), succinamidyl (—NHC(O)CH₂CH₂C(O)NH—), (—NHC(O)CH₂CH₂C(O)—), —C(R⁵)═N—, —N═C(R⁵)—, —C(R⁵)═N—O—, —O—N═C(R⁵)—, —O—C(O)O—, —C(O)N(R⁵), —N(R⁵)C(O)—, —C(S)(NR⁵)—, (NR⁵)C(S)—, —N(R⁵)C(O)N(R⁵)—, —C(O)S—, —SC(O)—, —C(S)O—, —OC(S)—, —OSi(R⁵)₂O—, —C(O)(CR³R⁴)C(O)O—, or —OC(O)(CR³R⁴)C(O)—, carbonate (—OC(O)O—), nitrogen (N), succinoyl, succinate, phosphate esters (—O—(O)POH—O—), cyclic compound, heterocyclic compound, piperidine, pyrazine, pyridine, piperazine, and sulfonate esters, as well as combinations thereof, wherein R³, R⁴ and R⁵ are, independently H or alkyl (e.g. C₁-C₄ alkyl), or
   (ii) the lipid moiety [L] comprises ditetradecylamin, preferably wherein the linker group [linker] is (—NHC(O)CH₂CH₂C(O)—).
Embodiment 5. The polymer conjugated lipid of any one of embodiment 1 to embodiment 4, wherein the lipid moiety [L] comprises at least one, preferably two, straight or branched, saturated or unsaturated alkyl chain comprising 6, 7, 8, 9, 10, 11, 12, 13, 14, 15, 16, 17, 18, 19, 20, 21, 22, 23, 24, 25, 26, 27, 28, 29, or 30 carbon atoms, preferably in the range of 10 to 20 carbon atoms, more preferably in the range of 12 to 18 carbon atoms, even more preferably 14, 16 or 18 carbon atoms, even more preferably 16 or 18 carbon atoms, most preferably 14 carbon atoms, wherein all selections are independent of one another.
Embodiment 6. The polymer conjugated lipid of any one of embodiment 1 to embodiment 5, wherein the linker group [linker] is selected from the group consisting of but not limited to a pH-sensitive moiety, a peptide or amid bond (—CO—NH—), an alkyl or alkenyl moiety (C₁₋₉ alkyl or C₂₋₉ alkenyl), a zwitterionic linker, non-ester containing linker moieties and ester-containing linker moieties (—C(O)O— or —OC(O)—), amido (—C(O)NH—), disulfide (—S—S—), carbonyl (—C(O)—), ether (—O—), thioether (—S—), oxime (e.g., —C(H)═N—O— or —O—N═C(H)—), carbamate (—NHC(O)O—), urea (—NHC(O)NH—), succinyl (—(O)CCH₂CH₂C(O)—), succinamidyl (—NHC(O)CH₂CH₂C(O)NH—), (—NHC(O)CH₂CH₂C(O)—), (—NHC(O)CH₂CH₂C(O)O—), —C(R⁵)═N—, —N═C(R⁵)—, —C(R⁵)═N—O—, —O—N═C(R⁵)—, —O—C(O)O—, —C(O)N(R⁵), —N(R⁵)C(O)—, —C(S)(NR⁵)—, (NR⁵)C(S)—, —N(R⁵)C(O)N(R⁵)—, —C(O)S—, —SC(O)—, —C(S)O—, —OC(S)—, —OSi(R⁵)₂O—, —C(O)(CR³R⁴)C(O)O—, or —OC(O)(CR³R⁴)C(O)—, carbonate (—OC(O)O—), nitrogen (N), succinoyl, succinate, phosphate esters (—O—(O)POH—O—), and sulfonate esters, as well as combinations thereof, wherein R³, R⁴ and R⁵ are, independently H or alkyl (e.g. C₁-C₄ alkyl), preferably wherein the linker group [linker] is selected from the group consisting of (—NHC(O)CH2CH2C(O)—), a peptide bond or amid bond (—CO—NH—), (—NHC(O)CH2CH2C(O)O—), and —NH—CH2-.
Embodiment 7. The polymer conjugated lipid of any one of embodiment 1 to embodiment 6, wherein the linker group [linker] comprises an amide linker moiety, preferably an ester linker moiety, or wherein the linker group [linker] comprises

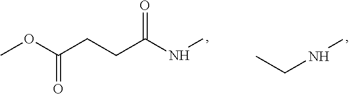

succinate, a peptide or amid bond (—CO—NH—), an amine, or a secondary amine, most preferably wherein the linker group [linker] comprises (—NHC(O)CH₂CH₂C(O)—).
Embodiment 8. The polymer conjugated lipid of any one of embodiment 1 to embodiment 7, wherein the polymer conjugated lipid is selected from the group consisting of “PMOZ 1”, “PMOZ 2”, “PMOZ 3”, “PMOZ 4” and “PMOZ 5”, most preferably the polymer conjugated lipid is “PMOZ 4”;
   whereby n has a mean value ranging from 2 to 200, preferably from 20 to 100, more preferably from 24 to 26, even more preferably about 100, or further even more preferably from 45 to 50, most preferably 50 or wherein n is selected such that the [P] moiety has an average molecular weight of about 3 kDa to about 6 kDa, preferably has an average molecular weight of about 4.2 kDa to about 4.4 kDa, or most preferably about 4.3 kDa;
   most preferably wherein the polymer conjugated lipid of any one of embodiment 1 to embodiment 7 is “PMOZ 4” with n having a mean value from 45 to 50, most preferably 50.
Embodiment 9. A lipid nanoparticle comprising a homopolymer moiety comprising at least one polyoxazoline (POZ) monomer unit

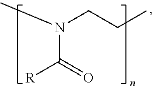

wherein R is C₁-C₉ alkyl or C₂-C₉ alkenyl, preferably C₁ or C₂ alkyl, and n has a mean value ranging from about 45 to about 55, preferably n is about 50 or wherein n is selected such that the [P] moiety has an average molecular weight of about 4.2 kDa to about 4.4 kDa, or most preferably about 4.3 kDa, preferably, wherein the homopolymer moiety comprising multiple monomer units comprises poly(2-methyl-2-oxazoline) (PMOZ), poly(2-ethyl-2-oxazoline) (PEOZ), poly(2-propyl-2-oxazoline) (PPOZ), poly(2-butyl-2-oxazoline) (PBOZ), poly(2-isopropyl-2-oxazoline) (PIPOZ), poly(2-methoxymethyl-2-oxazoline) (PMeOMeOx), or poly(2-dimethylamino-2-oxazoline) (PDMAOx), more preferably the polymer conjugated lipid according to any one of embodiment 1 to embodiment 8.
Embodiment 10. The lipid nanoparticle of embodiment 9, wherein the lipid nanoparticle further comprises a cationic or ionizable lipid.
Embodiment 11. The lipid nanoparticle of embodiment 9 to embodiment 10, wherein the lipid nanoparticles
   (i) do not comprise a polyethylene glycol-(PEG)-lipid conjugate or a conjugate of PEG and a lipid-like material, and preferably do not comprise PEG; and/or
   (ii) do not comprise a polymer conjugated lipid according to any one of embodiment 1 to embodiment 8 comprising a sulphur group (—S—), a terminating nucleophile, and/or being covalently coupled to a biologically active ingredient being a nucleic acid compound selected from the group consisting of RNA, an artificial mRNA, chemically modified or unmodified messenger RNA (mRNA) comprising at least one coding sequence, self-replicating RNA, circular RNA, viral RNA, and replicon RNA.
Embodiment 12. The lipid nanoparticle of any one of embodiment 9 to embodiment 11, wherein the cationic or ionizable lipid preferably carries a net positive charge at physiological pH, more preferably wherein the cationic or ionizable lipid comprises a tertiary nitrogen group or quaternary nitrogen group.
Embodiment 13. The lipid nanoparticle of any one of embodiment 9 to embodiment 12, wherein the lipid nanoparticle further comprises a phospholipid, wherein preferably the phospholipid is a zwitterionic compound selected from, but not limited to the group of 1,2-diphytanoyl-sn-glycero-3-phosphoethanolamine (DPhyPE; 1,2-di-(3,7,11,15-tetramethylhexadecanoyl)-sn-glycero-3-phosphoethanolamine), 1,2-diphytanoyl-sn-glycero-3-phosphocholine (DPhyPC), 1,2-dioleoyl-sn-glycero-3-phosphocholine (DOPC; dioleoylphosphatidylcholine), 1,2-Dipalmitoyl-sn-glycero-3-phosphocholine (DPPC; dipalmitoylphosphatidylcholine), 1,2-dioleoyl-sn-glycero-3-phosphoethanolamine (DOPE), phosphatidylethanolamines, distearoylphosphatidylcholines, dioleoyl-phosphatidylethanolamine (DOPEA), 1,2-distearoyl-sn-glycero-3-phosphoethanolamine (DSPE), palmitoyloleoylphosphatidylcholine (POPC), palmitoyloleoyl-phosphatidylethanolamine (POPE), 1,2-Dipalmitoyl-sn-glycero-3-phosphoethanolamine (DPPE), dioleoyl-phosphatidylethanolamine 4-(N-maleimidomethyl)-cyclohexane-1-carboxylate (DOPE-mal), 1,2-Dimyristoyl-sn-glycero-3-phosphoethanolamine (DMPE), 1,2-Dilinoleoyl-sn-glycero-3-phosphoethanolamine (DLoPE), distearoyl-phosphatidylethanolamine (DSPE), 1-Palmitoyl-2-oleoyl-sn-glycero-3-phosphoethanolamine (POPE), 1,2-Dilauroyl-sn-glycero-3-phosphoethanolamine (DLPE), 16-O-monomethylphosphoethanolamine, 16-O-dimethyl phosphatidylethanolamine, 1,2-Dierucoyl-sn-glycero-3-phosphoethanolamine (DEPE), 18-1-trans phosphatidylethanolamine, 1-stearoyl-2-oleoylphosphatidyethanolamine (SOPE), 1,2-Disqualeoyl-sn-glycero-3-phosphoethanolamine (DSQPE), 1,2-dielaidoyl-sn-glycero-3-phosphoethanolamine (transDOPE), 1-Stearoyl-2-linoleoyl-sn-glycero-3-phosphoethanolamine (SLPE), 1-tridecanoyl-sn-glycero-3-phospho-L-serine (sodium salt), 1-oleoyl-2-hydroxy-sn-glycero-3-phospho-L-serine (sodium salt), 1-palmitoyl-2-oleoyl-sn-glycero-3-phospho-L-serine (sodium salt) (POPS), 1-1-stearoyl-2-oleoyl-sn-glycero-3-phospho-L-serine (sodium salt), 1,2-dioleoyl-sn-glycero-3-phospho-L-serine (sodium salt) (DOPS), 1,2-distearoyl-sn-glycero-3-phospho-L-serine (sodium salt), 1,2-diphytanoyl-sn-glycero-3-phospho-L-serine (sodium salt), 1-O-hexadecanyl-2-O-(9Z-octadecenyl)-sn-glycero-3-phosphoethanolamine, 1,2-distearoyl-sn-glycero-3-phosphatidylcholine or 1,2-distearoyl-sn-glycero-3-phosphocholine (DSPC), 1,2-di-O-phytanyl-sn-glycero-3-phosphoethanolamine, 1-palmitoyl-2-cholesterylhemisuccinoyl-sn-glycero-3-phosphocholine (PChemsPC), 1,2-dicholesterylhemisuccinoyl-sn-glycero-3-phosphocholine (DChemsPC), 2-((2,3-bis(oleoyloxy)propyl)dimethylammonio)ethyl hydrogen phosphate (DOCP), 2-((2,3-bis(oleoyloxy)propyl)dimtheylammonio)ethyl ethyl phosphate (DOCPe), and 1-O-octadecyl-2-O-methyl-sn-glycero-3-phosphocholine (Edelfosine), preferably wherein the phospholipid is DSPC or DPhyPE, most preferably DPhyPE.
Embodiment 14. The lipid nanoparticle of any one of embodiment 9 to embodiment 13, wherein the lipid nanoparticle further comprises a sterol or steroid, preferably selected from the group consisting of cholesterol, cholesteryl hemisuccinate (CHEMS) and a derivate thereof, preferably wherein the lipid nanoparticle further comprises cholesterol.
Embodiment 15. The lipid nanoparticle of any one of embodiment 9 to embodiment 14, wherein preferably the lipid nanoparticle comprises
   (i) an amount of 1, 2, 3, 4, 5, 6, 7, 8, 9, or 10 mol % of the polymer conjugated lipid of any one of embodiment 1 to embodiment 8;
   (ii) preferably an amount of 5 mol % of the polymer conjugated lipid of any one of embodiment 1 to embodiment 8,
   (iii) more preferably an amount of 2.5 mol % of the polymer conjugated lipid of any one of embodiment 1 to embodiment 8;
   based upon a mol-percentage of the composition of 100% of all lipid components or excipients.
Embodiment 16. The lipid nanoparticle of any one of embodiment 9 to embodiment 15, wherein the polymer conjugated lipid is a PMOZ-lipid according to any one of embodiment 1 to embodiment 8.
Embodiment 17. The lipid nanoparticle of any one of embodiment 9 to embodiment 16, wherein the lipid nanoparticle comprises excipients selected from ratios selected from the group consisting of
   (i) 59 mol % cationic or ionizable lipid, preferably one of the ionizable lipid structures of C1 to C24, more preferably the ionizable lipid structure C24 or formula III-3 ((4-hydroxybutyl)azanediyl)bis (hexane-6,1-diyl)bis(2-hexyldecanoate)), 29.3 mol % cholesterol, 10 mol % neutral lipid and 1.7 mol % of the polymer conjugated lipid of any one of embodiment 1 to embodiment 8;
   (ii) 59 mol % cationic or ionizable lipid, preferably one of the ionizable lipid structures of C1 to C24, more preferably the ionizable lipid structure C24 or formula III-3 ((4-hydroxybutyl)azanediyl)bis (hexane-6,1-diyl)bis(2-hexyldecanoate)), 28.5 mol % cholesterol, 10 mol % neutral lipid and 2.5 mol % of the polymer conjugated lipid of any one of embodiment 1 to embodiment 8;
   (iii) 59 mol % cationic or ionizable lipid, preferably one of the ionizable lipid structures of C1 to C24, more preferably the ionizable lipid structure C24 or formula III-3 ((4-hydroxybutyl)azanediyl)bis (hexane-6,1-diyl)bis(2-hexyldecanoate)), 28.3 mol % cholesterol, 10 mol % DSPC or DPhyPE, preferably DPhyPE, 1 mol % DHPC, and 2.5 mol % of the polymer conjugated lipid of any one of embodiment 1 to embodiment 8;
   (iv) 49 mol % cationic or ionizable lipid, preferably one of the ionizable lipid structures of C1 to C24, more preferably the ionizable lipid structure C24 or formula III-3 ((4-hydroxybutyl)azanediyl)bis (hexane-6,1-diyl)bis(2-hexyldecanoate)), 29.3 mol % cholesterol, 10 mol % DSPC or DPhyPE, preferably DPhyPE, 10 mol % DHPC, and 2.5 mol % of the polymer conjugated lipid of any one of embodiment 1 to embodiment 8;
   (v) 47.4 mol % cationic or ionizable lipid, preferably one of the ionizable lipid structures of C1 to C27, more preferably the ionizable lipid structure C24 or formula III-3 (((4-hydroxybutyl)azanediyl)bis(hexane-6,1-diyl)bis(2-hexyldecanoate)), 40.9 mol % cholesterol, 10 mol % DSPC or DPhyPE, preferably DPhyPE, and 1.7 mol % of the polymer conjugated lipid of any one of embodiment 1 to embodiment 8;
   (vi) 47.4 mol % formula III-3 (((4-hydroxybutyl)azanediyl)bis(hexane-6,1-diyl)bis(2-hexyldecanoate)), 40.1 mol % cholesterol, 10 mol % DSPC and 2.5 mol % of the polymer conjugated lipid of any one of embodiment 1 to embodiment 8;
   (vii) 47.4 mol % formula III-3 (((4-hydroxybutyl)azanediyl)bis(hexane-6,1-diyl)bis(2-hexyldecanoate)), 40.9 mol % cholesterol, 10 mol % DSPC, and 1.7 mol % of the polymer conjugated lipid of any one of embodiment 1 to embodiment 8;
   (viii) 47.4 mol % formula III-3 (((4-hydroxybutyl)azanediyl)bis(hexane-6,1-diyl)bis(2-hexyldecanoate)), 40.1 mol % cholesterol, 10 mol % DSPC and 2.5 mol % 2-[(PMOZ)]n-N,N-ditetradecylacetamide];
   (ix) 47.4 mol % formula III-3 (((4-hydroxybutyl)azanediyl)bis(hexane-6,1-diyl)bis(2-hexyldecanoate)), 40.9 mol % cholesterol, 10 mol % DSPC and 1.7 mol % 2-[(PMOZ)]n-N,N-ditetradecylacetamide]; and most preferably
   (x) 59 mol % C24, 28.5 mol % cholesterol, 10 mol % DPhyPE and 2.5 mol % “PMOZ 4”,
   wherein n of the polymer-conjugated lipid has a mean value ranging from about 45 to about 55, preferably n is about 50 or wherein n is selected such that the polymer moiety has an average molecular weight of about 4.2 kDa to about 4.4 kDa, or most preferably about 4.3 kDa.
Embodiment 18. The lipid nanoparticle of any one of embodiment 9 to embodiment 17, wherein the polymer conjugated lipid of embodiment 1 to embodiment 8 inhibits aggregation of the lipid nanoparticles.
Embodiment 19. The lipid nanoparticle of any one of embodiment 9 to embodiment 18, further comprising a biologically active ingredient.
Embodiment 20. The lipid nanoparticle of embodiment 19, wherein the biologically active ingredient is a nucleic acid compound selected from the group consisting of RNA, an artificial mRNA, chemically modified or unmodified messenger RNA (mRNA) comprising at least one coding sequence, self-replicating RNA, circular RNA, viral RNA, and replicon RNA; or any combination thereof, more preferably wherein the biologically active ingredient is chemically modified mRNA or chemically unmodified mRNA.
Embodiment 21. The lipid nanoparticle of any one of embodiment 9 to embodiment 20, wherein the mRNA is associated with the lipid nanoparticle, preferably wherein the mRNA is encapsulated in the lipid nanoparticle.
Embodiment 22. The lipid nanoparticle of any one of embodiment 9 to embodiment 21, wherein the lipid nanoparticles comprise the mRNA at an amount such as to achieve an N/P ratio in the range of about 5 to about 20, more preferably about 10 to about 18, even more preferably about 12 to about 16, most preferably about 14.
Embodiment 23. The lipid nanoparticle of any one of embodiment 9 to embodiment 22, wherein the lipid nanoparticle is a sterile solid composition for reconstitution with a sterile liquid carrier, and wherein the lipid nanoparticle further comprises one or more inactive ingredients selected from pH-modifying agents, bulking agents, stabilizers, non-ionic surfactants and antioxidants, and wherein the sterile liquid carrier is an aqueous carrier.
Embodiment 24. The lipid nanoparticle of any one of embodiment 9 to embodiment 23, wherein the lipid nanoparticle is a sterile liquid composition, and wherein the lipid nanoparticles have a mean hydrodynamic diameter as determined by dynamic laser scattering from about 50 nm to about 300 nm, or from about 60 nm to about 250 nm, or from about 60 nm to about 200 nm, or from about 70 to 200 nm, or from about 75 nm to about 160, or from about 85 nm to about 140 nm, or from about 90 nm to about 130 nm, or from about 50 nm to about 120 nm.
Embodiment 25. The lipid nanoparticle of any one of embodiment 9 to embodiment 24, wherein the lipid nanoparticles exhibit a zeta potential in the range of −50 mV to +50 mV, preferably in the range of −25 mV to +25 mV, more preferably in the range of −10 mV to +10 mV, most preferably in the range of −5 mV to +5 mV.
Embodiment 26. The lipid nanoparticle of any one of embodiment 9 to embodiment 25, wherein the mRNA compound is a mono-, bi-, or multicistronic mRNA.
Embodiment 27. The lipid nanoparticle of any one embodiment 9 to embodiment 26, wherein the mRNA compound comprises at least one chemical modification.
Embodiment 28. The lipid nanoparticle of embodiment 27, wherein the chemical modification is selected from the group consisting of base modifications, sugar modifications, backbone modifications and lipid modifications, preferably wherein the chemical modification is a base modification, more preferably wherein the base modification preferably is selected from the group consisting of pseudouridine (psi or Lp), N1-methylpseudouridine (N1 MPU, N1Mpsi or N1MLp), 1-ethylpseudouracil, 2-thiouracil (s2U), 4-thiouracil, 5-methylcytosine, 5-methyluracil, 5-methoxyuracil, and any combination thereof.
Embodiment 29. The lipid nanoparticle of any one of embodiment 9 to embodiment 28, wherein the mRNA compound comprises a coding region encoding a peptide or protein, wherein the coding region exhibits a sequence modification.
Embodiment 30. The lipid nanoparticle of embodiment 29, wherein the sequence modification is selected from a G/C content modification, a codon modification, a codon optimization or a C-optimization of the sequence; preferably wherein, compared with the coding region of the corresponding wild-type mRNA, the
   G/C content of the coding region is increased;
   C content of the coding region is increased;
   codon usage in the coding region is adapted to the human codon usage; and/or
   codon adaptation index (CAI) is increased or maximized in the coding region.
Embodiment 31. The lipid nanoparticle of any one of embodiment 9 to embodiment 30, wherein the mRNA compound further comprises
   a) a 5′-CAP structure, preferably m7GpppN, more preferably CAP1 or m7G(5′)ppp(5′)(2′OMeA)pG;
   b) optionally at least one miRNA sequence, preferably wherein the microRNA binding site is for a microRNA selected from the group consisting of miR-126, miR-142, miR-144, miR-146, miR-150, miR-155, miR-16, miR-21, miR-223, miR-24, miR-27, miR-26a, or any combination thereof;
   c) at least one 5′-UTR element;
   d) a coding sequence;
   e) at least one 3′-UTR element;
   f) at least one poly(A) sequence;
   g) at least one poly(C) sequence;
   or any combinations of these.
Embodiment 32. The lipid nanoparticle of any one of embodiment 9 to embodiment 31, wherein the least one coding RNA comprises a 5′-CAP structure, preferably m7G, CAP0, CAP1, CAP2, a modified CAP0 or a modified CAP1 structure.
Embodiment 33. The lipid nanoparticle of any one of embodiment 9 to embodiment 32, wherein the at least one coding RNA comprises at least one heterologous 5′-UTR and/or at least one heterologous 3′-UTR, preferably wherein the at least one heterologous 5′-UTR comprises a nucleic acid sequence derived from a 5′-UTR of a gene selected from HSD17B4, RPL32, ASAH1, ATP5A1, MP68, NDUFA4, NOSIP, RPL31, SLC7A3, TUBB4B and UBQLN2, or from a homolog, a fragment or variant of any one of these genes; and/or preferably wherein the at least one heterologous 3′-UTR comprises a nucleic acid sequence derived from a 3′-UTR of a gene selected from PSMB3, ALB7, alpha-globin, CASP1, COX6B1, GNAS, NDUFA1 and RPS9, or from a homolog, a fragment or a variant of any one of these genes.
Embodiment 34. The lipid nanoparticle of any one of embodiment 9 to embodiment 33, wherein the at least one coding RNA comprises a (i) HSD17B4 5′-UTR and a PSMB3 3′-UTR or (ii) a RPL32 5′-UTR and an ALB7 3′-UTR, preferably a mutated alpha-globin 3′-UTR (SEQ ID NO:11/12), more preferably a HSD17B4 5′-UTR (SEQ ID NO:21/22) and a PSMB3 3′-UTR (SEQ ID NO:19/20).
Embodiment 35. The lipid nanoparticle of any one of embodiment 9 to embodiment 34, comprising the following elements in the 5′ to 3′ direction:
   a) a 5′-CAP structure, preferably selected from the group consisting of m7G(5′), m7G(5′)ppp(5′)(2′OMeA)pG and m7G(5′)ppp(5′)(2′OMeG)pG;
   b) a 5′-UTR element comprising a nucleic acid sequence derived from the 5′-UTR of a TOP gene, said nucleic acid sequence preferably comprising an RNA sequence that corresponds to the nucleic acid sequence according to SEQ ID NO:22, 24, 26, or a homolog, a fragment or a variant thereof, most preferably according to SEQ ID NO:22 (HSD17B4);
   c) at least one coding sequence;
   d) a 3′-UTR element comprising a nucleic acid sequence derived from an α-globin gene, said nucleic acid sequence preferably comprising an RNA sequence that corresponds to the nucleic acid sequence according to SEQ ID NO:6, 8, 10, 12, 14, 16, 18, 20, or a homolog, a fragment or a variant thereof; and/or a 3′-UTR element comprising a nucleic acid sequence derived from an albumin gene, said nucleic acid sequence preferably comprising an RNA sequence that corresponds to the nucleic acid sequence according to SEQ ID NO:18, or a homolog, a fragment or a variant thereof, most preferably according to SEQ ID NO:20 (PSMB3);
   e) optionally, at least one poly(A) sequence, preferably consisting of 10 to 200, 10 to 100, 40 to 80, or 50 to 70 adenosine nucleotides;
   f) optionally, at least one poly(C) sequence, preferably consisting of 10 to 200, 10 to 100, 20 to 70, 20 to 60 or 10 to 40 cytosine nucleotides; and
   g) optionally, at least one histone stem-loop, preferably comprising the RNA sequence according to SEQ ID NO:4.
Embodiment 36. The lipid nanoparticle of any one of embodiment 9 to embodiment 35, wherein the biologically active ingredient is
   (a) an mRNA comprising at least one coding sequence encoding a peptide or protein, or a fragment or variant thereof, wherein the peptide or protein is an antigen, wherein the antigen preferably is derived from pathogenic antigens, tumor antigens, allergenic antigens or autoimmune self-antigens, or a fragment or variant thereof; or
   (b) an mRNA comprising at least one coding sequence encoding a therapeutic protein, or a fragment or variant thereof, wherein the therapeutic protein is selected from the group consisting of
   (i) therapeutic proteins for use in enzyme replacement therapy for the treatment of metabolic, endocrine or amino acid disorders or for use in replacing an absent, deficient or mutated protein;
   (ii) therapeutic proteins for use in the treatment of blood disorders, diseases of the circulatory system, diseases of the respiratory system, infectious diseases or immune deficiencies;
   (iii) therapeutic proteins for use in the treatment of cancer or tumor diseases;
   (iv) therapeutic proteins for use in hormone replacement therapy;
   (v) therapeutic proteins for use in reprogramming somatic cells into pluri- or omnipotent stem cells;
   (vi) therapeutic proteins for use as adjuvant or immunostimulation;
   (vii) therapeutic proteins being a therapeutic antibody;
   (viii) therapeutic proteins being a gene editing agent; and
   (ix) therapeutic proteins for use in treating or preventing a liver disease selected from the group consisting of liver fibrosis, liver cirrhosis and liver cancer.
Embodiment 37. The lipid nanoparticle of embodiment 36 subitem (a), wherein the at least one coding sequence encoding a pathogenic antigen is selected from the group consisting of a bacterial, viral, fungal and protozoal antigen.
Embodiment 38. The lipid nanoparticle of embodiment 37, wherein the at least one coding sequence encoding a pathogenic antigen
   (i) is derived from a SARS coronavirus 2 (SARS-CoV-2), nCoV-2019 coronavirus, SARS coronavirus (SARS-CoV), Bunyavirales virus, Cytomegalovirus (CMV), Dengue viruses (DENV-1, DENV-2, DENV-3 and DENV-4), Ebola virus, Epstein-Barr virus (EBV), Flavivirus, Hepatitis B virus (HBV), Herpes simplex virus (HSV), Human immunodeficiency virus (HIV), Human metapneumovirus (HMPV), Human Papilloma virus (HPV), Human parainfluenza viruses (HPIV), Influenza virus, extraintestinal pathogenic E. coli (ExPEC), Lassa mammarenavirus (LASV), MERS coronavirus, Mycobacterium tuberculosis, Nipah virus, Norovirus, Rabies virus, Respiratory Syncytial virus (RSV), Rhinovirus, Rota virus, Vaccinia virus, Yellow Fever virus (YFV), Zika virus (ZIKV), Chlamydia trachomatis (i.e. bacterium Chlamydia causing Chlamydia), or Malaria parasite (e.g. Plasmodium falciparum, Plasmodium vivax, Plasmodium malariae, or Plasmodium ovale); and/or
   (ii) is derived from a structural protein, an accessory protein, or a replicase protein from a SARS coronavirus 2 (SARS-CoV-2), nCoV-2019 coronavirus, SARS coronavirus (SARS-CoV), or an immunogenic fragment or immunogenic variant of any of these; and/or
   (iii) is derived from a spike protein (S), an envelope protein (E), a membrane protein (M) or a nucleocapsid protein (N) from a SARS coronavirus 2 (SARS-CoV-2), nCoV-2019 coronavirus, SARS coronavirus (SARS-CoV), or an immunogenic fragment or immunogenic variant of any of these, preferably wherein the spike protein (S) comprises or consists of spike protein fragment S1 or spike protein fragment S2, more preferably spike protein fragment S1, or an immunogenic fragment or immunogenic variant thereof (e.g. receptor binding domain (RBD), critical neutralisation domain (CND)); and/or
   (iv) is derived from a pre-fusion stabilized spike protein (S) (S_stab) from a SARS coronavirus 2 (SARS-CoV-2), nCoV-2019 coronavirus, SARS coronavirus (SARS-CoV) comprising at least one pre-fusion stabilizing mutation.
Embodiment 39. The lipid nanoparticle of any one of embodiment 9 to embodiment 38 for use
   (i) in the treatment or prophylaxis of infectious diseases; cancer or tumor diseases, disorders or conditions; liver diseases selected from the group consisting of liver fibrosis, liver cirrhosis and liver cancer; allergies; or autoimmune disease; disorder or condition; and/or
   (ii) for use in enzyme replacement therapy for the treatment of metabolic or endocrine disorders or for use in replacing an absent, deficient or mutated protein.
Embodiment 40. The lipid nanoparticle of any one of embodiment 9 to embodiment 39 for use in the treatment or prophylaxis of infectious diseases.
Embodiment 41. The lipid nanoparticle of embodiment 9 or embodiment 40 comprising at least one coding RNA, wherein said at least one coding RNA comprises at least one coding sequence encoding at least one peptide or protein for use in treatment or prevention of a disease, disorder or condition, wherein said lipid nanoparticle is administered via local or locoregional injection, infusion or implantation, in particular intradermal, subcutaneous, intramuscular, intracameral, subconjunctival, suprachoroidal injection, subretinal, subtenon, retrobulbar, topical, posterior juxtascleral administration, or intrapulmonal inhalation, interstitial, locoregional, intravitreal, intratumoral, intralymphatic, intranodal, intra-articular, intrasynovial, periarticular, intraperitoneal, intra-abdominal, intracardial, intralesional, intrapericardial, intraventricular, intrapleural, perineural, intrathoracic, epidural, intradural, peridural, intrathecal, intramedullary, intracerebral, intracavernous, intracorporus cavernosum, intraprostatic, intratesticular, intracartilaginous, intraosseous, intradiscal, intraspinal, intracaudal, intrabursal, intragingival, intraovarian, intrauterine, intraocular, periocular, periodontal, retrobulbar, subarachnoid, subconjunctival, suprachoroidal injection, infusion, implantation, nasal, buccal, sublingual, otic or auricular, ophthalmic, conjunctival, vaginal, rectal, intracervical, endosinusial, laryngeal, oropharyngeal, ureteral, urethral administration, more preferably said lipid nanoparticle is administered intramuscularly, intravenously, intradermally, subcutaneously, intratumorally, intranasally, or by inhalation, most preferably intramuscularly, to a subject in need thereof.
Embodiment 42. A kit or kit of parts, comprising any one of the lipid nanoparticle of embodiment 9 to embodiment 41, optionally comprising a liquid vehicle for solubilizing, and, optionally, technical instructions providing information on administration and dosage of the components.
Embodiment 43. The lipid nanoparticle of any one of embodiment 9 to embodiment 41 or the kit or kit of parts of embodiment 42 for use in in vivo drug delivery, preferably for use in delivering a nucleic acid, preferably a mRNA.
Embodiment 44. The lipid nanoparticle of any one of embodiment 9 to embodiment 41 or the kit or kit of parts of embodiment 43 for use as a medicament.
Embodiment 45. The lipid nanoparticle for use as a medicament according to embodiment 44, wherein the medicament is for the prevention, prophylaxis, treatment and/or amelioration of a disease selected from infectious diseases including viral, bacterial or protozoological infectious diseases, cancer or tumor diseases, liver diseases, autoimmune diseases, allergies, monogenetic diseases including hereditary diseases, genetic diseases in general, diseases which have a genetic inherited background and which are typically caused by a defined gene defect and are inherited according to Mendel's laws; cardiovascular diseases, neuronal diseases, diseases of the respiratory system, diseases of the digestive system, diseases of the skin, musculoskeletal disorders, disorders of the connective tissue, neoplasms, immune deficiencies, endocrine, nutritional and metabolic diseases, eye diseases, ear diseases and diseases associated with a peptide or protein deficiency.
Embodiment 46. The lipid nanoparticle for use as a medicament according to embodiment 44 or embodiment 45, wherein the medicament is a vaccine composition.
Embodiment 47. A vaccine composition comprising a lipid nanoparticle of any one of embodiment 9 to embodiment 46 or a kit or kit of parts of embodiment 42 for use as a medicament, and/or for prevention, prophylaxis, treatment and/or amelioration of a disease selected from infectious diseases including viral, bacterial or protozoological infectious diseases, cancer or tumor diseases.
Embodiment 48. A method of treatment or prophylaxis of infectious diseases; cancer or tumor diseases, disorders or conditions; liver diseases selected from the group consisting of liver fibrosis, liver cirrhosis and liver cancer; allergies; or autoimmune disease; disorder or condition comprising the steps:
   a) providing a lipid nanoparticle of any one of embodiment 9 to embodiment 45, comprising a homopolymer moiety comprising at least one polyoxazoline (POZ) monomer, preferably the polymer conjugated lipid according to any one of embodiment 1 to embodiment 8, the vaccine composition of embodiment 47, or the kit or kit of parts of embodiment 42; and
   b) applying or administering the mRNA, the lipid nanoparticle, the vaccine composition or the kit or kit of parts to a tissue or an organism.
Embodiment 49. A method for delivering mRNA encoding an antigen or a therapeutic peptide or protein to a subject, the method comprising administering to a subject a lipid nanoparticle of any one of embodiments 1 to 33, wherein the mRNA encodes an antigen or a therapeutic peptide or protein, and wherein delivering the mRNA to the subject is beneficial in treating or preventing a disease or disorder, preferably wherein the subject is a mammal, more preferably wherein the subject is a human.
Embodiment 50. The method according to any one of embodiments embodiment 48 to embodiment 49, wherein the mRNA, the lipid nanoparticle of any one of embodiment 9 to embodiment 48, the vaccine composition of embodiment 47 or the kit or kit of parts of embodiment 42 is administered to the tissue or to the organism by intravenous, intramuscular, subcutaneous, intradermal or intratumoral injection or any administration route as disclosed in any preceding embodiment.
Embodiment 51. A method of inducing an immune response in a subject, the method comprising administering to the subject the vaccine composition of embodiment 47 in an amount effective to produce an antigen-specific immune response in the subject.
Embodiment 52. A pharmaceutical composition comprising a lipid nanoparticle of any one of embodiment 9 to embodiment 48 or a kit or kit of parts of embodiment 42 or the vaccine composition of embodiment 47 for use in vaccination of a subject comprising an effective dose of mRNA encoding a virus antigen.
Embodiment 53. Use of a pharmaceutical composition according to embodiment 52 or a kit or kit of parts according to embodiment 42 for (i) inducing an immune response, for (ii) inducing an antigen specific T-cell response or preferably for (iii) inducing CD8+ T cells responses.
Embodiment 54. Use of the pharmaceutical composition of embodiment 52 for the prophylaxis of an infectious disease or in the manufacture of a medicament for the prophylaxis of an infectious disease, wherein said medicament preferably is a vaccine composition.
Embodiment 55. A method for preventing, ameliorating or treating a disease or condition in a subject in need comprising administering to the subject a lipid nanoparticle of any one of embodiment 9 to embodiment 48, a pharmaceutical composition of embodiment 52 or a kit or kit of parts of embodiment 42.
Embodiment 56. The method of any one of the preceding method embodiments, wherein administration of the lipid nanoparticle results in expression of the antigen encoded by mRNA in the lymphocytes of the subject.
Embodiment 57. A method of treating or preventing a disorder of any one of embodiments 36, 39, 41, 45, 48, or 49, wherein the disorder is an infection with coronavirus, or a disorder related to such an infection.
Embodiment 58. A method of treating or preventing a disorder of any one of embodiments 36, 39, 41, 45, 48, or 49, wherein the subject in need is a mammalian subject, preferably a human subject.
Embodiment 59. The method of any one of the preceding method embodiments, wherein the administration of the lipid nanoparticle results in an antigen specific antibody response, preferably wherein the antigen specific antibody response is measured by the presence of antigen-specific antibodies in serum.
Embodiment 60. The lipid nanoparticle of any one of embodiment 9 to embodiment 16, wherein the lipid nanoparticle comprises excipients selected from ratios selected from the group consisting of
   (i) 59 mol % cationic or ionizable lipid, preferably one of the ionizable lipid structures of C1 to C24, more preferably the ionizable lipid structure C24 or formula III-3 ((4-hydroxybutyl)azanediyl)bis (hexane-6,1-diyl)bis(2-hexyldecanoate)), 29.3 mol % cholesterol, 10 mol % neutral lipid and 1.7 mol % of the polymer conjugated lipid of any one of embodiment 1 to embodiment 8;
   (ii) 59 mol % cationic or ionizable lipid, preferably one of the ionizable lipid structures of C1 to C24, more preferably the ionizable lipid structure C24 or formula III-3 ((4-hydroxybutyl)azanediyl)bis (hexane-6,1-diyl)bis(2-hexyldecanoate)), 28.5 mol % cholesterol, 10 mol % neutral lipid and 2.5 mol % of the polymer conjugated lipid of any one of embodiment 1 to embodiment 8;
   (iii) 59 mol % cationic or ionizable lipid, preferably one of the ionizable lipid structures of C1 to C24, more preferably the ionizable lipid structure C24 or formula III-3 ((4-hydroxybutyl)azanediyl)bis (hexane-6,1-diyl)bis(2-hexyldecanoate)), 28.3 mol % cholesterol, 10 mol % DSPC or DPhyPE, preferably DPhyPE, 1 mol % DHPC, and 2.5 mol % of the polymer conjugated lipid of any one of embodiment 1 to embodiment 8;
   (iv) 49 mol % cationic or ionizable lipid, preferably one of the ionizable lipid structures of C1 to C24, more preferably the ionizable lipid structure C24 or formula III-3 ((4-hydroxybutyl)azanediyl)bis (hexane-6,1-diyl)bis(2-hexyldecanoate)), 29.3 mol % cholesterol, 10 mol % DSPC or DPhyPE, preferably DPhyPE, 10 mol % DHPC, and 2.5 mol % of the polymer conjugated lipid of anyone of embodiment 1 to embodiment 8;
   (vi) 47.4 mol % cationic or ionizable lipid, preferably one of the ionizable lipid structures of C1 to C27, more preferably the ionizable lipid structure C24 or formula III-3 (((4-hydroxybutyl)azanediyl)bis(hexane-6,1-diyl)bis(2-hexyldecanoate)), 40.9 mol % cholesterol, 10 mol % DSPC or DPhyPE, preferably DPhyPE, and 1.7 mol % of the polymer conjugated lipid of any one of embodiment 1 to embodiment 8;
   (vi) 47.4 mol % cationic or ionizable lipid, preferably one of the ionizable lipid structures of C1 to C27, more preferably the ionizable lipid structure C24 or formula III-3 (((4-hydroxybutyl)azanediyl)bis(hexane-6,1-diyl)bis(2-hexyldecanoate)), 40.1 mol % cholesterol, 10 mol % DSPC and 2.5 mol % of the polymer conjugated lipid of any one of embodiment 1 to embodiment 8;
   (vii) 47.4 mol % formula III-3 (((4-hydroxybutyl)azanediyl)bis(hexane-6,1-diyl)bis(2-hexyldecanoate)), 40.9 mol % cholesterol, 10 mol % DSPC, and 1.7 mol % of the polymer conjugated lipid of any one of embodiment 1 to embodiment 8;
   (viii) 47.4 mol % formula III-3 (((4-hydroxybutyl)azanediyl)bis(hexane-6,1-diyl)bis(2-hexyldecanoate)), 40.1 mol % cholesterol, 10 mol % DSPC and 2.5 mol % 2-[(PMOZ)]n-N,N-ditetradecylacetamide];
   (ix) 47.4 mol % formula III-3 (((4-hydroxybutyl)azanediyl)bis(hexane-6,1-diyl)bis(2-hexyldecanoate)), 40.9 mol % cholesterol, 10 mol % DSPC and 1.7 mol % 2-[(PMOZ)]n-N,N-ditetradecylacetamide]; and most preferably
   (x) 59 mol % C24, 28.5 mol % cholesterol, 10 mol % DPhyPE and 2.5 mol % “PMOZ 4”, wherein n of the polymer-conjugated lipid has a mean value ranging from about 45 to about 55, preferably n is about 50 or wherein n is selected such that the polymer moiety has an average molecular weight of about 4.2 kDa to about 4.4 kDa, or most preferably about 4.3 kDa.
Embodiment 61. The lipid nanoparticle of any one of embodiment 9 to embodiment 16 or embodiment 60, wherein the lipid nanoparticle comprises a neutral lipid or phospholipid having at least one alkyl chain with a length of C₅, C₆, C₇, C₈, C₉, C₁₀, C₁₁, C₁₂, C₁₃ or C₁₄, preferably with a length of C₆, C₇, C₈, C₉, or C₁₀, more preferably with a length of C₆, C₇, C₈, most preferably with a length of C₇, or further most preferably wherein the lipid nanoparticle comprises a combination of two neutral lipids wherein the combination comprises a neutral lipid or phospholipid having at least two alkyl chains, whereby each alkyl chain independently has a length of preferably C₆, C₇, C₈, C₉, or C₁₀, more preferably with a length of C₆, C₇, C₈, most preferably with a length of C₇, further most preferably a phospholipid selected from the group consisting of 05:0 PC (1,2-dipentanoyl-sn-glycero-3-phosphocholine), 04:0 PC (1,2-dibutyryl-sn-glycero-3-phosphocholine), 06:0 PC (DHPC, 1,2-dihexanoyl-sn-glycero-3-phosphocholine), 07:0 PC (DHPC, 1,2-diheptanoyl-sn-glycero-3-phosphocholine), 08:0 PC (1,2-dioctanoyl-sn-glycero-3-phosphocholine), and 09:0 PC (1,2-dinonanoyl-sn-glycero-3-phosphocholine), preferably 07:0 PC (DHPC, 1,2-diheptanoyl-sn-glycero-3-phosphocholine).
Embodiment 62. The lipid nanoparticle of any one of embodiment 9 to embodiment 16 or embodiment 60 to embodiment 61, wherein the lipid nanoparticles comprise a neutral lipid or phospholipid having at least two alkyl chains, whereby each alkyl chain independently has a length of C₅, C₆, C₇, C₈, C₉, C₁₀, C₁₁, C₁₂, C₁₃ or C₁₄, preferably with a length of C₆, C₇, C₈, C₉, or C₁₀, more preferably with a length of C₆, C₇, C₈, most preferably with a length of C₇, or further most preferably wherein the lipid nanoparticle comprises a combination of two neutral lipids wherein the combination comprises a neutral lipid or phospholipid having at least two alkyl chains, whereby each alkyl chain independently has a length of preferably C₆, C₇, C₈, C₉, or C₁₀, more preferably with a length of C₆, C₇, C₈, most preferably with a length of C₇, further most preferably a phospholipid selected from the group consisting of 05:0 PC (1,2-dipentanoyl-sn-glycero-3-phosphocholine), 04:0 PC (1,2-dibutyryl-sn-glycero-3-phosphocholine), 06:0 PC (DHPC, 1,2-dihexanoyl-sn-glycero-3-phosphocholine), 07:0 PC (DHPC, 1,2-diheptanoyl-sn-glycero-3-phosphocholine), 08:0 PC (1,2-dioctanoyl-sn-glycero-3-phosphocholine), and 09:0 PC (1,2-dinonanoyl-sn-glycero-3-phosphocholine), preferably 07:0 PC (DHPC, 1,2-diheptanoyl-sn-glycero-3-phosphocholine).
Embodiment 63. A lipid nanoparticle comprising a polymer conjugated lipid according to any one of embodiment 1 to embodiment 8, wherein the lipid nanoparticle has a lower PDI and/or lower size upon (i) freezing and thawing or (ii) freeze-drying (lypophilizing) and reconstitution, as compared to a control lipid nanoparticle comprising a PEG-lipid instead said polymer conjugated lipid according to any one of embodiment 1 to embodiment 8.
Embodiment 64. A method of making a frozen lipid nanoparticle of any one of embodiment 9 to embodiment 16 or embodiment 60 to embodiment 62 or a lipid nanoparticle comprising a polymer conjugated lipid according to any one of embodiment 1 to embodiment 8, wherein the lipid nanoparticle upon thawing has a lower PDI and/or lower size as compared to a control lipid nanoparticle comprising a PEG-lipid instead of a polymer conjugated lipid according to any one of embodiment 1 to embodiment 8.
Embodiment 65. A method of making a lyophilized lipid nanoparticle of any one of embodiment 9 to embodiment 16 or embodiment 60 to embodiment 62 or a lipid nanoparticle comprising a polymer conjugated lipid according to any one of embodiment 1 to embodiment 8, wherein the lipid nanoparticle upon reconstitution has a lower PDI and/or lower size as compared to a control lipid nanoparticle comprising a PEG-lipid instead of a polymer conjugated lipid according to any one of embodiment 1 to embodiment 8.
Embodiment 66. An improved lyophilization process for the preparation of lyophilized lipid nanoparticles of any one of embodiment 9 to embodiment 16 or embodiment 60 to embodiment 62, said process comprising the step of using a polymer conjugated lipid according to any one of embodiment 1 to embodiment 8 as excipient instead of a PEG-lipid, wherein the lipid nanoparticle upon reconstitution has a lower PDI and/or lower size as compared to a control lipid nanoparticle comprising a PEG-lipid instead of a polymer conjugated lipid according to any one of embodiment 1 to embodiment 8.
Embodiment 67. A vaccine composition, comprising a lipid nanoparticle of any one of embodiment 9 to embodiment 16 or embodiment 60 to embodiment 62 or a polymer conjugated lipid according to any one of embodiment 1 to embodiment 8.
Embodiment 68. A vaccine composition or a lipid nanoparticle of any one of the preceding embodiments comprising a polymer-conjugated lipid according to any of the preceding embodiments, wherein the formulation has an increase in LNP mean size of about 30%, 29%, 28%, 27%, 26%, 25%, 24%, 23%, 22%, 21%, 20%, 19%, 18%, 17%, 16%, 15%, 14%, 13%, 12%, 11%, 10%, 9%, 8%, 7%, 6%, 5%, 4%, 3%, 2%, 1%, or less after one or more freeze/thaw cycles as compared to that prior to freeze/thaw cycles.
Embodiment 69. A vaccine composition or a lipid nanoparticle of any one of the preceding embodiments comprising a polymer-conjugated lipid according to any of the preceding embodiments, wherein the formulation has an increase in LNP mean size of about 10%, 9%, 8%, 7%, 6%, 5%, 4%, 3%, 2%, 1%, or less after one or more freeze/thaw cycles as compared to that prior to freeze/thaw cycles.
Embodiment 70. A vaccine composition or a lipid nanoparticle of any one of the preceding embodiments comprising a polymer-conjugated lipid according to any of the preceding embodiments, wherein the formulation has an increase in LNP mean size of about 30%, 29%, 28%, 27%, 26%, 25%, 24%, 23%, 22%, 21%, 20%, 19%, 18%, 17%, 16%, 15%, 14%, 13%, 12%, 11%, 10%, 9%, 8%, 7%, 6%, 5%, 4%, 3%, 2%, 1%, or less after lyophilization as compared to that prior to lyophilization.
Embodiment 71. A vaccine composition or a lipid nanoparticle of any one of the preceding embodiments comprising a polymer-conjugated lipid according to any of the preceding embodiments, wherein the formulation has an increase in LNP mean size of about 10%, 9%, 8%, 7%, 6%, 5%, 4%, 3%, 2%, 1%, or less after lyophilization as compared to that prior to lyophilization.
Embodiment 72. A vaccine composition or a lipid nanoparticle of any one of the preceding embodiments comprising a polymer-conjugated lipid according to any of the preceding embodiments, wherein the formulation has an increase in LNP mean size of about 30%, 29%, 28%, 27%, 26%, 25%, 24%, 23%, 22%, 21%, 20%, 19%, 18%, 17%, 16%, 15%, 14%, 13%, 12%, 11%, 10%, 9%, 8%, 7%, 6%, 5%, 4%, 3%, 2%, 1%, or less after dilution as compared to that prior to dilution.
Embodiment 73. A vaccine composition or a lipid nanoparticle of any one of the preceding embodiments comprising a polymer-conjugated lipid according to any of the preceding embodiments, wherein the formulation has an increase in LNP mean size of about 10%, 9%, 8%, 7%, 6%, 5%, 4%, 3%, 2%, 1%, or less after dilution as compared to that prior to dilution.
Embodiment 74. A vaccine composition or a lipid nanoparticle of any one of the preceding embodiments comprising a polymer-conjugated lipid according to any of the preceding embodiments, wherein the encapsulation efficiency of the formulation is substantially the same after storage at about 4° C. or lower for at least one month.
Embodiment 75. A vaccine composition or a lipid nanoparticle of any one of the preceding embodiments comprising a polymer-conjugated lipid according to any of the preceding embodiments, wherein the LNP mean size of the LNPs is substantially the same after storage at about 4° C. or lower for at least one month.

### EXAMPLES

In the following section, particular examples illustrating various embodiments and aspects of the invention are presented. The present invention, however, is not limited in scope by the exemplified embodiments, which are intended as illustrations of single aspects of the invention only, and methods which are functionally equivalent are within the scope of the invention. Indeed, various modifications of the invention in addition to those described herein will become readily apparent to those skilled in the art from the foregoing description, accompanying figures and the examples below. All such modifications fall within the scope of the claims as disclosed herein.

### Example 1: Generation of RNA Constructs

The following RNA constructs were used in the experiments described in Examples 3 to 7: i) RNA encoding circumsporozoite protein (CSP mRNA); ii) RNA encoding Rabies virus glycoprotein (RABV-G mRNA); iii) RNA encoding Photinus pyralis luciferase (PpLuc mRNA); and iv) RNA encoding tyrosine-related protein 2 (Trp2) or respectively dopachrome tautomerase.

To prepare the RNA constructs, DNA sequences encoding the desired proteins as listed above (i.e. CSP, RABV-G, PpLuc and Trp2) were prepared and used for subsequent RNA in vitro transcription, wherein the DNA sequences were prepared by optionally modifying the wild type CDS sequences by introducing a GC optimized CDS. Sequences were introduced into a plasmid vector comprising the motifs described below (UTR sequences, a stretch of adenosines, optionally a histone stem-loop structure, and a stretch of 30 cytosines). The obtained plasmid DNA was transformed and propagated in bacteria using common protocols and plasmid DNA was extracted, purified, enzymatically linearized using a restriction enzyme and used for DNA dependent RNA in vitro transcription using T7 RNA polymerase in the presence of a nucleotide mixture (ATP/GTP/CTP/UTP) and cap analogue (e.g., m7GpppG or m7G(5′)ppp(5′)(2′OMeA)pG or m7G(5′)ppp(5′)(2′OMeG)pG)) under suitable buffer conditions. The obtained RNA was purified using RP-HPLC (PureMessenger®; according to WO2008077592) and used for further experimentation. The obtained mRNA was enzymatically polyadenylated using a commercial polyadenylation kit.

The details of the RNA constructs that were used in all working examples are as follows:
Ad i): Pf-CSP mRNA comprised a HSD17B4 5′-UTR, a GC-optimized Plasmodium falciparum circumsporozoite protein ORF, a PSMB3 3′-UTR, 64× adenosine (polyA-tail); 5 nucleotides, 30× cytosine (polyC-tail), a histone stem-loop sequence and 5 additional nucleotides at the 3-terminal end (SEQ ID NO:38);
Ad ii): RABV-G mRNA comprised a mCap, a GC-optimized Rabies virus glycoprotein ORF, a muag 3′-UTR; 64× adenosine (polyA-tail); 5 nucleotides, 30× cytosine (polyC-tail), a histone stem-loop sequence and 5 additional nucleotides at the 3-terminal end (SEQ ID NO:32);
Ad iii): PpLuc mRNA comprised a CleanCap® AG cap analog, a HSD17B4 5′-UTR, a GC-optimized Photinus pyralis luciferase ORF (firefly luciferase), a PSMB3 3′-UTR, a polyA tail having 100× adenosines, and a histone stem-loop sequence (SEQ ID NO:71);
Ad iv) Trp2 mRNA comprised a RPL32 5′-UTR, an ALB7 3′-UTR, 64× adenosine (polyA-tail); 5 nucleotides, 30× cytosine (polyC-tail), a histone stem-loop sequence and 5 additional nucleotides at the 3-terminal end and is detailed in SEQ ID NO:29.

### Example 2: Synthesis of Lipids and Preparation of LNPs

The present example provides methods and information to obtain cationic lipids used herein as well as methods of generating LNPs of the invention.

### Example 2.1: Synthesis of Lipids

The following three lipids were synthesized according to general protocols of ChiroBlock GmbH (Bitterfeld-Wolfen, Germany). Three lipids as shown in Table Ex-1 and FIG. 1 were synthesized, see Examples 7 to 9.

TABLE Ex-1
Overview of the synthesized lipids

| Lipid name | Ref. in | Compound name*** | Ester |
|---|---|---|---|
| used herein | FIG. 1 | [Tail-Linker-Head] | structure |
| HEXA1 | FIG. 1A | HEXA-C5DE-PipSS | Diester |
| HEXA2 | FIG. 1B | HEXA-C5DE-PipC3SS | Diester |
| THIOETHER | FIG. 1C | VitE-C4DE-Pip-Thioether | Diester |

***The name of the lipids was derived as follows: the basic name consists of three parts: “Tail-Linker-Head”. The tail is “HEXA” which refers to a hexyldecanoic acid (Hexyl-1-decanol) or “VitE” which refers to vitamin E. The linker is “C5DE” or “C4DE”, which refers to a C5 or C4 group comprising a diester linker (C5 = 5 Carbon atoms of the linker with two esters; C4 = 4 Carbon atoms of the linker with two esters). The reference to the head “PipSS” indicates piperidine residues connected via a disulfide bridge, “PipC3SS” relates to a structure with 3 carbon atoms between the piperidine ring and the disulfide bridge, and “Pip-Thioether” indicates piperidine residues connected via a thioether bridge.

“PMOZ 2” as used herein throughout the examples section has the structure of:

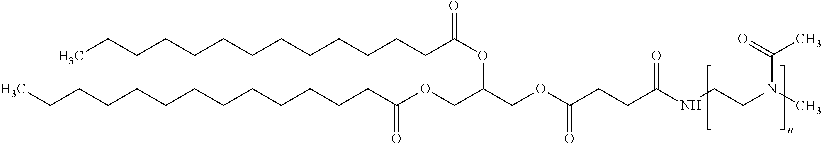

[“PMOZ 2” ] with n=50 i.e. having 50 monomer repeats.

“PMOZ 4” as used herein throughout in the examples section has the structure of:

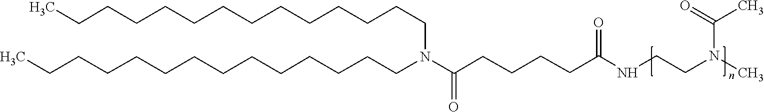

[“PMOZ 4” ] with n=50 i.e. having 50 monomer repeats.

Summarized, “PMOZ 2” was synthesized as follows:

### Step 1 (Synthesis of PMOZ-Polymer):

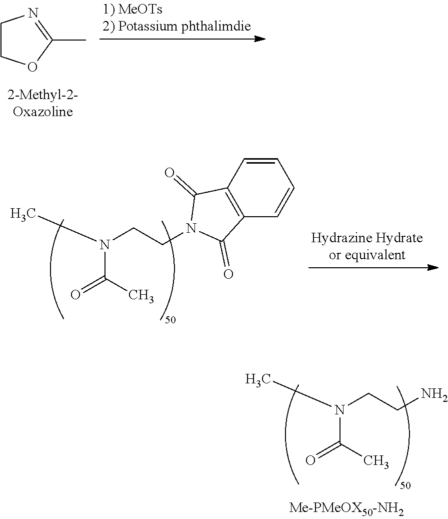

Thereafter, 1.0 g of DMG (1) was reacted with succinic anhydride (2) to afford 848 mg (71%) of ester (3). 344 mg of this material was reacted with 1.92 g of PMOz polymer (4) from step 1:

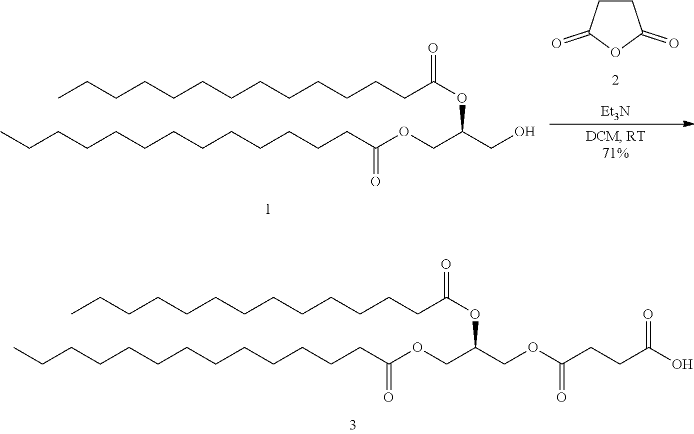 [SMILES: "Et3N" ; CCCCCCCCCCCCCC(=O)OC[C@H](CO)OC(=O)CCCCCCCCCCCCC ; O=C1CCC(=O)O1 ; CCCCCCCCCCCCCC(=O)OC[C@H](COC(=O)CCC(=O)O)OC(=O)CCCCCCCCCCCCC]

### Step 2:

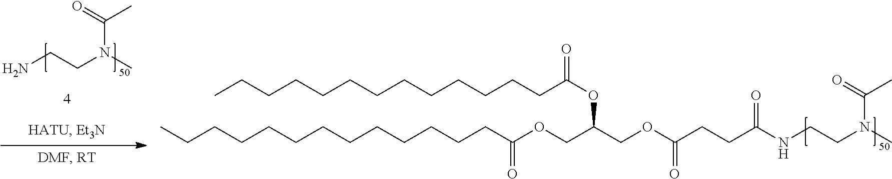

Summarized, “PMOZ 4” was synthesized as follows:

### Step 1 (Synthesis of PMOZ-Polymer):

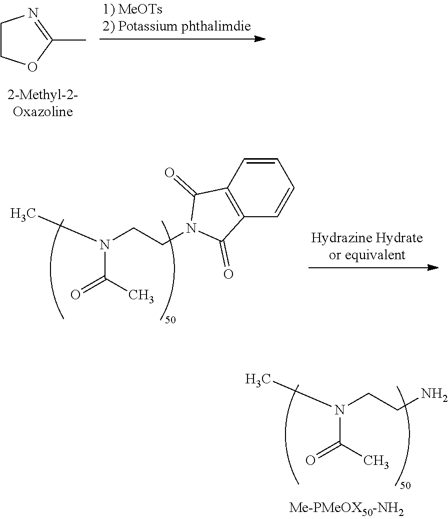

Thereafter, 0.5 g of di(tetradecyl)amine (1) was reacted with succinic anhydride (2) to afford 563 mg (90%) of amide (3). 312 mg of this material was reacted with 2.10 g of PMOz polymer (4) from step 1.

### Step 2:

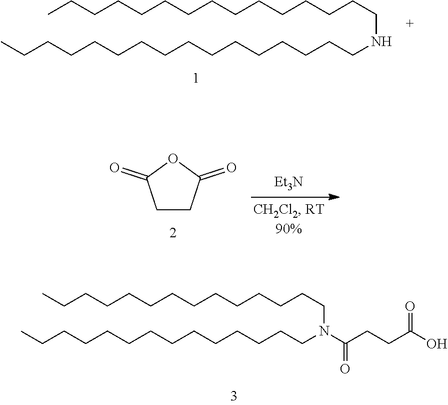 [SMILES: "Et3N" ; CCCCCCCCCCCCCCCCNCCCCCCCCCCCCCCC ; O=C1CCC(=O)O1 ; CCCCCCCCCCCCCCN(CCCCCCCCCCCCCC)C(=O)CCC(=O)O]

### Step 3:

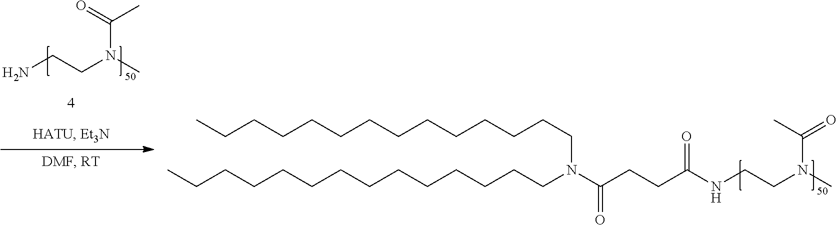

For all lipids, purity and structural identity of the lipids were confirmed by nuclear magnetic resonance spectroscopy (H-NMR, 500.13 MHz) and mass spectrometry (electrospray ionization-ESI or atmospheric pressure chemical ionization-APCI, via direct injection).

### Example 2.2: Preparation of LNPs Using the NanoAssembr™ Microfluidic System

The LNPs were prepared using the NanoAssembr™ microfluidic system (Precision NanoSystems Inc., Vancouver, BC) according to standard protocols which enables controlled, bottom-up, molecular self-assembly of nanoparticles via custom-engineered microfluidic mixing chips that enable millisecond mixing of nanoparticle components at a nanolitre scale.

In the present examples, the cationic lipid (either i) HEXA1, ii) HEXA2 or iii) THIOETHER) was used together with cholesterol, at least one neutral lipid or phospholipid, and a polymer conjugated lipid for preparation of lipid nanoparticle compositions. In more detail, cholesterol (Avanti Polar Lipids; Alabaster, AL), the neutral lipid/phospholipid i) DPhyPE (Avanti Polar Lipids; Alabaster, AL) and/or ii) DPhyPS (Avanti Polar Lipids; Alabaster, AL) and/or iii) WT-PS or 18:0-18:1 PS (Avanti Polar Lipids; Alabaster, AL) and/or iv) (07:0) PC (DHPC) (Avanti Polar Lipids; Alabaster, AL) with the structures shown below, or v) DSPC (Avanti Polar Lipids; Alabaster, AL in LNP-C) were used. Still further, either i) DMG-PEG2000 (NOF Corporation, Tokyo, Japan) or ii) DSG-PEG2000 (NOF Corporation, Tokyo, Japan) was used as polymer conjugated lipid.

The structures of DPhyPS is:

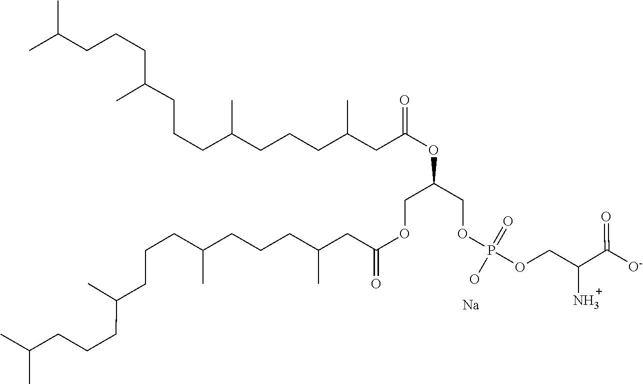

The structure of WT-PS (18:0-18:1 PS) is:

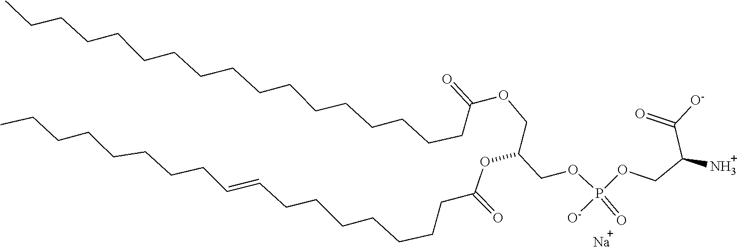 [SMILES: CCCCCCCC/C=C/CCCCCCCC(=O)O[C@H](COC(=O)CCCCCCCCCCCCCCCCC)COP(=O)([O-])OC[C@H]([NH3+])C(=O)[O-] ; [Na+]]

The structure of (07:0) PC (DHPC), i.e. 1,2-diheptanoyl-sn-glycero-3-phosphocholine, is:

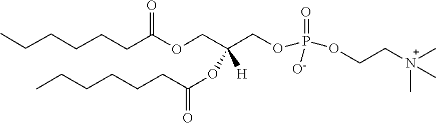 [SMILES: CCCCCCC(=O)OC[C@H](COP(=O)([O-])OCC[N+](C)(C)C)OC(=O)CCCCCC]

In the context of the working examples and also the disclosure of the invention, if only “DMG-PEG” or “DMGmPEG” is indicated, reference is made herein to DMG-PEG2000. In the context of the working examples and also the disclosure of the invention, if only “DSG-PEG” or “DSGmPEG” is indicated, reference is made herein to DSG-PEG2000:

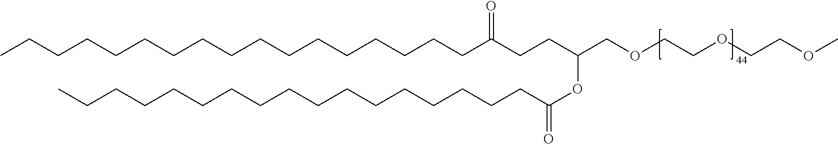

In the context of the working examples and also the disclosure of the invention, if only “PMOZ” is indicated, reference is made herein to an exemplary PMOZ-lipid according to FIG. 4.

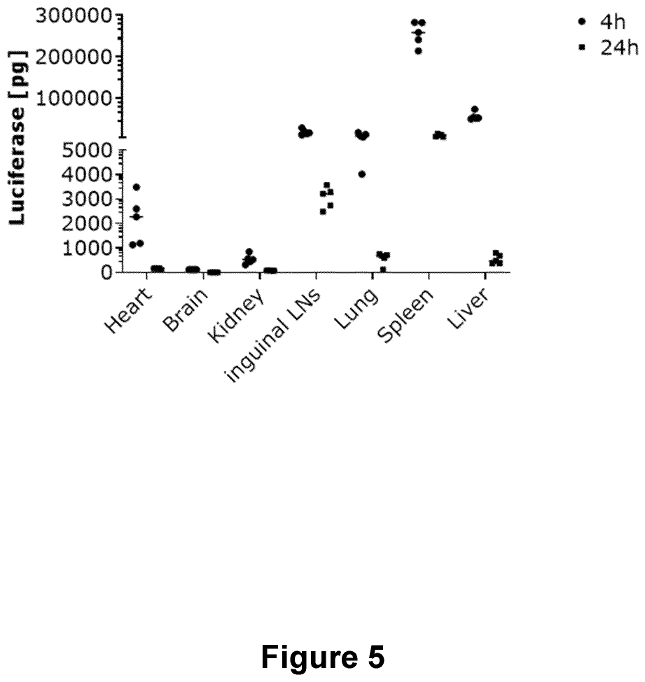

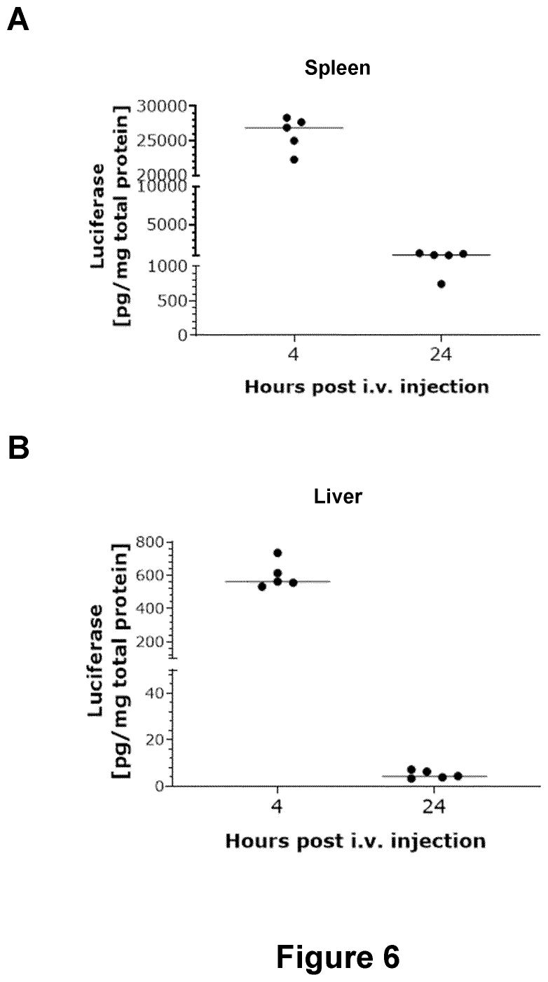

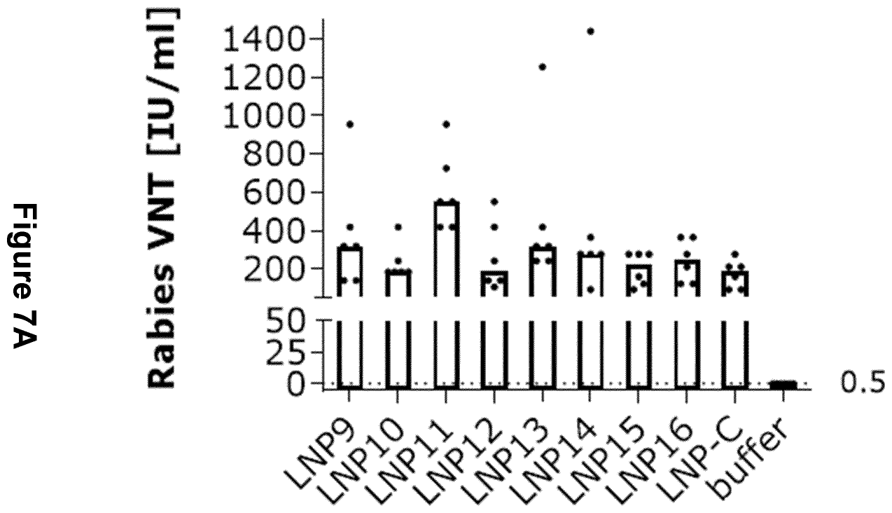

Stock solutions of the lipids were prepared by solubilizing the lipids in alcoholic solution (see details below) according to standard procedures. LNPs were prepared by mixing appropriate volumes of lipid stock solutions in alcoholic solution (100% ethanol buffer) with an aqueous phase containing appropriate amounts of mRNA as indicated herein below. The lipid stock solutions for the cationic lipid, cholesterol, the phospholipid (except for DPhyPS and WT-PS) and the polymer conjugated lipid were 20-30 mg/ml in EtOH; the THIOETHER lipid was dissolved 30 mg/ml in t-butanol. The stock solution for DPhyPS was 10 mg/ml in EtOH: t-butanol (1:1) for experimental procedures leading to results as shown in FIGS. 1-3 and 10 mg/ml in t-butanol for experimental procedures leading to results as shown in FIGS. 5-7. The solution for WT-PS was a “premix solution” of WT-PS hydrated with premixed lipids in EtOH. In order to arrive at this premix solution, WT-PS dissolved in Cl₃CH was used as stock solution, the Cl₃CH was removed, and the WT-PS dry film was hydrated with the premixed lipids in EtOH prior to the actual mixing step described in the following. The solution for (07:0) PC (DHPC) was also a “premix solution” of (07:0) PC (DHPC) hydrated with premixed lipids in EtOH. In order to arrive at this premix solution, (07:0) PC (DHPC) dissolved in Cl₃CH was used as stock solution, the Cl₃CH was removed, and the (07:0) PC (DHPC) dry film was hydrated with the premixed lipids in EtOH prior to the actual mixing step described in the following.

The mRNA was diluted to 0.16 mg/ml in 50 mM acetate buffer, pH 4, optionally comprising (07:0) PC (DHPC). Syringes were inserted into inlet parts of the NanoAssemblr™ (Precision NanoSystems Inc., Vancouver, BC) and used to mix the ethanolic lipid solution (optionally comprising the WT-PS premix) with the mRNA aqueous solution at a ratio of about 1:3 (vol/vol) with total flow rates from about 14 ml/min to about 18 ml/min.

The ethanol was then removed and the external buffer replaced with PBS/Sucrose buffer by dialysis (Slide-A-Lyzer™ Dialysis Cassettes, ThermoFisher). Finally, the lipid nanoparticles were filtered through a 0.2 μm pore sterile filter. Lipid nanoparticle particle diameter size was from about 90 nm to about 140 nm as determined by quasi-elastic light scattering using a Malvern Zetasizer Nano (Malvern Instruments Ltd.; Malvern, UK) and the final LNPs were present in 10 mM PBS, pH 7.4 comprising 9% Sucrose.

The LNP formulations of Tables Ex-2 to Ex-5 were prepared:

TABLE Ex-2

| Name of LNP | | mol-percentages |
|---|---|---|
| formulation/ | Excipients [cationic lipid:steroid:phospholipid | for excipients |
| composition No. | (optionally three as indicated):polymer conjugated lipid] | [mol %] |
| LNP1 | HEXA1:Chol:DPhyPE + DPhyPS:DMG-PEG2000 | 59:29.3:5 + 5:1.7 |
| LNP2 | HEXA1:Chol:DPhyPE + WT-PS:DMG-PEG2000 | 59:29.3:8 + 2:1.7 |
| LNP3 (GN02) | HEXA1:Chol:DPhyPE:DMG-PEG2000 | 59:29.3:10:1.7 |

TABLE Ex-3
[for prophetic Example 4]:

| Name of LNP | | mol-percentages |
|---|---|---|
| formulation/ | Excipients [cationic lipid:steroid:phospholipid(s) | for excipients |
| composition No. | (optionally three as indicated):polymer conjugated lipid] | [mol %] |
| LNP4 | HEXA1:Chol:DPhyPE + DPhyPS:PMOZ-lipid* | 59:29.3:5 + 5:1.7 |
| LNP5 | HEXA1:Chol:DPhyPE + WT-PS:PMOZ-lipid* | 59:29.3:8 + 2:1.7 |
| LNP6 | HEXA1:Chol:DPhyPE + WT-PS + (07:0) PC (DHPC):PMOZ- | 49:29.3:8 + 2 + 10:1.7 |
| | lipid* | |
| LNP7 (GN02-PMOZ) | HEXA1:Chol:DPhyPE:PMOZ-lipid* | 59:29.3:10:1.7 |

*an exemplary PMOZ-lipid is shown in FIG. 4, preferably the PMOZ lipid used is “PMOZ 4” having n = 50

TABLE Ex-4

| Name of LNP | | mol-percentages |
|---|---|---|
| formulation/ | Excipients [cationic lipid:steroid:phospholipid | for excipients |
| composition No. | (optionally three as indicated):polymer conjugated lipid] | [mol %] |
| LNP8 | HEXA2:Chol:DPhyPE + DPhyPS:DSG-PEG2000 | 59:29.3:5 + 5:1.7 |
| LNP-C | CL*:Chol:DSPC:PL** | 47.4:40.9:10:1.7 |

*The term “CL” indicates a state of the art cationic lipid
**The term “PL” indicates a state of the art polymer conjugated lipid comprising mPEG2000

TABLE Ex-5

| Name of LNP | | |
|---|---|---|
| formulation/ | Excipients [cationic lipid as disclosed in | mol-percentages |
| composition | Table Ex-24:steroid:neutral lipid(s) | for excipients |
| designation | (optionally three as indicated):polymer conjugated lipid] | [mol %] |
| LNP9 | THIOETHER:Chol:DPhyPE + DPhyPS:DMG-PEG2000 | 59:29.3:5 + 5:1.7 |
| LNP10 | THIOETHER:Chol:DPhyPE + (07:0) PC (DHPC):DMG-PEG2000 | 58:29.3:10 + 1:1.7 |
| LNP11 | THIOETHER:Chol:DPhyPE + DPhyPS + (07:0) PC (DHPC):DMG- | 58:29.3:5 + 5 + 1:1.7 |
| | PEG2000 | |
| LNP12 | THIOETHER:Chol:DPhyPE + (07:0) PC (DHPC):DMG-PEG2000 | 49:29.3:10 + 10:1.7 |
| LNP13 | THIOETHER:Chol:DPhyPE + DPhyPS + (07:0) PC (DHPC):DMG- | 49:29.3:5 + 5 + 10:1.7 |
| | PEG2000 | |
| LNP14 | THIOETHER:Chol:DPhyPE + DPhyPS + (07:0) PC (DHPC):DMG- | 39:39.3:5 + 5 + 10:1.7 |
| | PEG2000 | |
| LNP15 | THIOETHER:Chol:DPhyPE:DMG-PEG2000 | 59:29.3:10:1.7 |
| LNP16 | THIOETHER:Chol:DPhyPE:DMG-PEG2000 | 49:39.3:10:1.7 |

In the above LNPs, the N/P (lipid to mRNA mol ratio) preferably was about 17.5 and the total eipid/mRNA mass ratio preferably was about 40.

### Example 3: Effect of the Presence of PS for In Viva Malaria Vaccination

Pf-CSP mRNA was formulated in LNPs1 to 3 of Table Ex-2 as described above in Example 2.2.

The resulting LNP formulations were applied to female Balb/c mice on days 0 and 21 intramuscularly (im.; musculus tibialis) with doses of mRNA, formulations, and control groups as shown in Table Ex-6. A negative control group received buffer only. The mice were terminated at day 35. Serum samples were taken at days 1, 21 and 35, and the spleens were isolated at day 35.

TABLE Ex-6
Vaccination scheme of Example 3

| | No. of | | | | | | |
|---|---|---|---|---|---|---|---|
| Group | mice | RNA Treatment | Formulation | mRNA | Dose | Route | Volume |
| 1 | 6 | mRNA encoding Pf-CSP | LNP1 | Pf-CSP | 1 μg | i.m. | 1 × 25 μl |
| 2 | 6 | mRNA encoding Pf-CSP | LNP2 | Pf-CSP | 1 μg | i.m. | 1 × 25 μl |
| 3 | 6 | mRNA encoding Pf-CSP | LNP3 (GN02) | Pf-CSP | 1 μg | i.m. | 1 × 25 μl |
| 4 | 6 | None | Buffer (PBS) | none | n.a. | i.m. | 1 × 25 μl |

### Determination of Specific Humoral Immune Responses by ELISA:

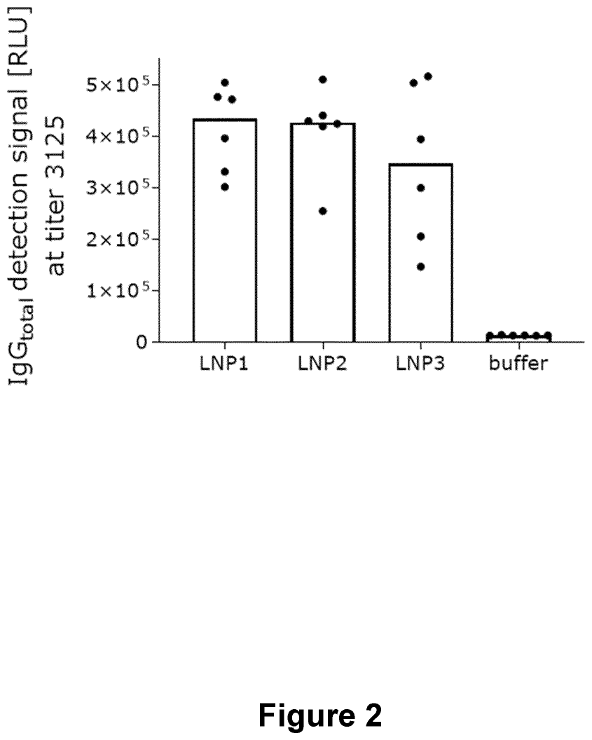

ELISA was performed using malaria [NANP]₇ peptide for coating (1 μg/ml in 100% DMSO). Coated plates were incubated using respective serum dilutions, and binding of specific antibodies to the respective malaria [NANP]₇ peptide were detected using biotinylated isotype specific anti-mouse antibodies followed by streptavidin-HRP (horseradish peroxidase) with Amplex™ Red Reagent as substrate. Endpoint titers of antibodies (total IgG) directed against the malaria [NANP]₇ peptide were measured by ELISA on day 35 post prime (14 days post 1^{st} boost). Results are shown in FIG. 2 (IgG total detection signal at titer 3125).

### Intracellular Cytokine Staining (ICS):

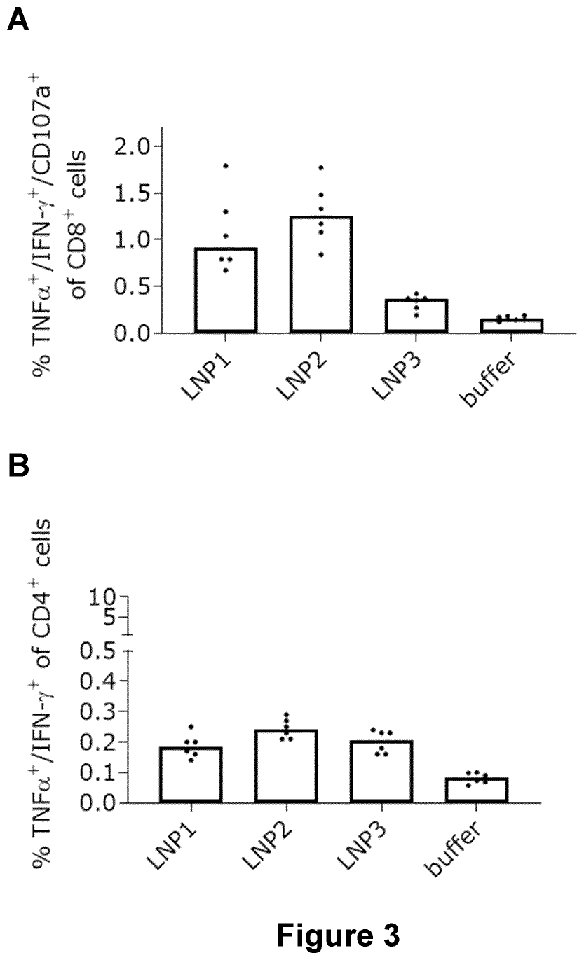

Splenocytes from vaccinated mice were isolated on day 35 according to a standard protocol known in the art. Briefly, isolated spleens were grinded through a cell strainer and washed in PBS/1% FBS followed by red blood cell lysis. After an extensive washing step with PBS/1% FBS, splenocytes were seeded into 96-well plates (2×10⁶ cells per well). Cells were stimulated with a mixture of CSP peptides (0.5 μg/ml) in the presence of 2.5 μg/ml of an anti-CD28 antibody (BD Biosciences) and a protein transport inhibitor for 6 h at 37° C. After stimulation, cells were washed and stained for intracellular cytokines using the Cytofix/Cytoperm™ reagent (BD Biosciences) according to the manufacturer's instructions. Antibodies against the following molecules were used for staining: Thy1.2-FITC (1:100), CD8-PE-Cy7 (1:200), CD107a-PE-Cy7 (1:100), TNF-PE (1:100), IFNγ-APC (1:100) (eBioscience), CD4-BD Horizon V450 (1:200) (BD Biosciences) and incubated with Fcγ-block diluted 1:100. Aqua Dye was used to distinguish live/dead cells (Invitrogen). Cells were acquired using a BD FACS Canto II flow cytometer (Becton Dickinson). Flow cytometry data was analyzed using FlowJo software (Tree Star, Inc.). Results are shown in FIG. 3.

### Results:

As shown in FIG. 2, the LNPs comprising PS (either in the form of DPhyPS [LNP1] or WT-PS [LNP2]) induced a stronger humoral immune responses in mice than the LNP not comprising PS [LNP3]. As expected, all three LNP-formulations induced a humoral immune response compared to the buffer as control.

As shown in FIG. 3, the LNPs comprising PS (either in the form of DPhyPS [LNP1] or WT-PS [LNP2]) induced a stronger cellular immune response when looking at the CD8+ T-cell responses in mice than the LNP not comprising PS [LNP3] (see FIG. 3A). When it comes to the CD4+ T-cell responses, the LNPs comprising WT-PS [LNP2] induced a stronger cellular immune response in mice than the LNP not comprising PS [LNP3], whereas the response to the LNP comprising DPhyPS [LNP1] was comparable to that of the LNP not comprising PS [LNP3](see FIG. 3B). As CD8+ T cells are a major protective immune mechanism against intracellular infections caused by Malaria parasites, an effective Malaria vaccine should induce strong CD8+ T cells responses. The strongest responses were obtained when using LNPs comprising PS as vaccines.

### Example 4 [Prophetic]: Effect of the Presence of PS for In Vivo Rabies Vaccination

RABV-G mRNA is formulated in LNP4 to LNP7 of Table Ex-3 as described above in Example 2.2.

The LNP formulations are applied to 7 weeks old female Balb/C mice on days 0 and 21 intramuscularly (i.m.; musculus tibialis) with doses of mRNA, formulations, and control groups as shown in Table Ex-7. A negative control group receives buffer only. The mice are terminated at day 28. Serum samples are taken at days 1, 21 and 28, and the spleens are isolated at day 28.

TABLE Ex-7
Vaccination scheme of Example 4

| | No. of | | | | | | |
|---|---|---|---|---|---|---|---|
| Group | mice | RNA Treatment | Formulation | mRNA | Dose | Route | Volume |
| 1 | 8 | mRNA encoding RABV-G | LNP4 | RABV-G | 1 μg | i.m. | 1 × 25 μl |
| 2 | 8 | mRNA encoding RABV-G | LNP5 | RABV-G | 1 μg | i.m. | 1 × 25 μl |
| 3 | 8 | mRNA encoding RABV-G | LNP6 | RABV-G | 1 μg | i.m. | 1 × 25 μl |
| 4 | 8 | mRNA encoding RABV-G | LNP7 | RABV-G | 1 μg | i.m. | 1 × 25 μl |
| 5 | 8 | None | Buffer (PBS) | none | n.a. | i.m. | 1 × 25 μl |

For determining the levels of antibody against the Rabies virus in serum, a classical virus neutralization test is performed (Fluorescent Antibody Virus Neutralization (FAVN) assay). Thus, 28 days after the first mRNA administration, mice are sacrificed and blood samples are collected for further analysis, i.e. for Virus neutralizing antibodies (VNA) analysis via FAVN assay. For said immunogenicity assays, the VNT is measured, i.e. anti-Rabies virus neutralizing titers (VNTs) in serum are analyzed by the Eurovir® Hygiene-Labor GmbH, Germany, using the FAVN assay and the Standard Challenge Virus CVS-11 according to WHO protocol.

### Example 5: Effect of the Presence of PS on Organ-Targeting

PpLuc mRNA was formulated into LNP8 and LNP-C of Table Ex-4 as described above in Example 2.2.

The LNP8 and the LNP-C formulations were applied to female Balb/c mice on day 0 intravenously (i.v.) with doses of mRNA as shown in Table Ex-8. Termination was carried out at the time point indicated below (i.e. either at 4 h or 24 h after application), and the organs were collected. Furthermore, serum samples were taken 14 days before the application and at the time point indicated below (i.e. either at 4 h or 24 h after application).

TABLE Ex-8
Setup of Example 5

| | No. of | | | Time | | | | |
|---|---|---|---|---|---|---|---|---|
| Group | mice | RNA | Formulation | point | | Dose | Route | Volume |
| 1 | 5 | mRNA encoding PpLuc | LNP8 | 4 | h | 20 μg | i.v. | 100 μl |
| 2 | 5 | mRNA encoding PpLuc | LNP8 | 24 | h | 20 μg | i.v. | 100 μl |
| 3 | 6 | mRNA encoding PpLuc | LNP-C | 4 | h | 20 μg | i.v. | 100 μl |

The PpLuc expression was determined by Luciferase assay in the tissue lysates prepared according to standard methods after the organ collection. The following organs were collected and analyzed: i) heart; ii) brain; iii) kidney; iv) lymph-nodes; v) lung; vi) spleen; and vii) liver. For LNP-C, the spleen and the liver were prepared and analyzed.

Furthermore, the levels of cytokines and chemokines in the serum were determined by the LEGENDplex CBA-assay according to the manufacturer's instructions.

### Results:

FIG. 5 shows the results of LNP8 comprising DPhyPS (i.e. group 1 and group 2 of Table Ex-8). It can be derived from FIG. 5 that the LNPs comprising PS, which were administered systemically by i.v. application, were mostly targeted to the spleen. Thus, the luciferase expression rates at 4 h is clearly highest in the spleen, followed by liver, lung and lymph nodes (which are at about the same level), followed by heart, kidney and brain, wherein there is almost no luciferase expression in the latter two organs. At 24 h, there are no to weak signals in heart, brain, kidney, lung and liver. While luciferase expression can be detected to some extent in the lymph nodes, it is clearly still at the highest level in the spleen at 24 h. The results are shown as pg of total luciferase protein/organ.

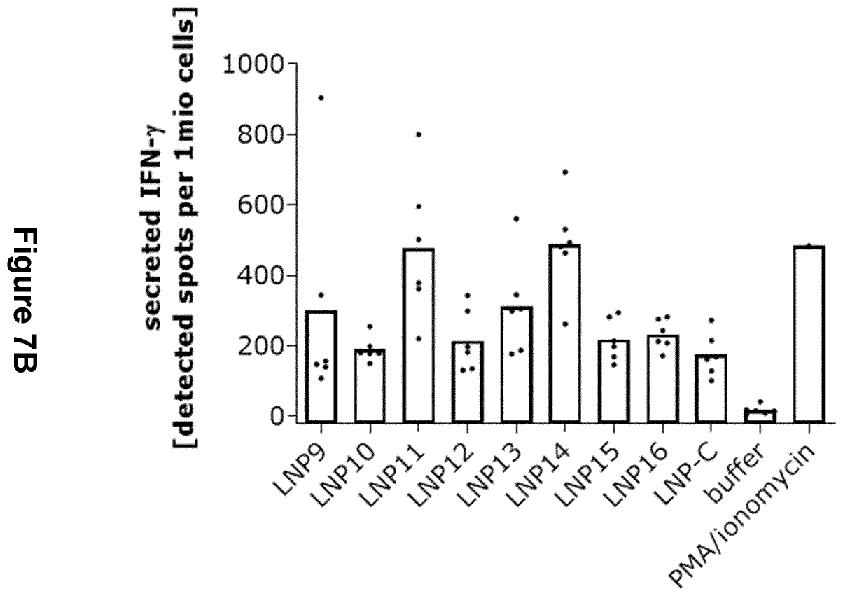

FIG. 6 also shows the results of LNP8 (groups 1 and 2 of Table Ex-8) but this time only for spleen and liver and as pg/mg of total protein. Thus, unlike in the results shown in FIG. 5, where the pg of total luciferase protein are depicted per organ, the results were normalized in that the pg of total luciferase detected in the spleen and the liver were set in relation to the mg of total protein determined in the spleen and the liver, respectively. It is noted that the y-axes of the two graphs shown in FIG. 6 differ in order to account for the fact that the expression in the spleen is much higher than in the liver. The results confirm that the LNPs comprising PS are mainly targeted to the spleen, where an expression of luciferase is not only detected after 4 h but still after 24 h.

Table Ex-9 shows the results for groups 1 and 3 of Table Ex-8, where the organs spleen and liver were analyzed after 4 h for expression of luciferase, wherein a PS-comprising LNP-formulation was used in group 1 vs. a standard-LNP not comprising PS in group 3. It is evident that the PS-comprising LNP-formulation is routed to (“targeting”) the spleen, whereas for the standard-LNP-formulation predominantly expression is detected in liver.

TABLE Ex-9
luciferase expression in the spleen and liver

| | | Expression in | Expression in | Ratio |
|---|---|---|---|---|
| | | spleen after 4 h | liver after 4 h | spleen/ |
| | LNP- | (pg/mg total | (pg/mg total | liver |
| Group | formulations | protein), median | protein), median | after 4 h |
| 1 | LNP8 with PpLuc | 26898 | 562 | 48 |
| 3 | LNP-C with PpLuc | 98671 | 652108 | 0.2 |

Finally, Table Ex-10 shows that the PS-comprising LNP-formulation resulted in a different cytokine profile compared to the control LNP-formulation not comprising PS.

TABLE Ex-10
levels of cytokines and chemokines in serum

| Serum | LNP8 with PpLuc | LNP-C with PpLuc |
|---|---|---|
| IL-6 | +++ | − |
| TNF-α | + | −/+ |
| IL-12p70 | ++ | − |
| IL-1β | +/− | + |
| KC (CXCL1) | +++ | +++ |
| MCP-1 (CCL2) | ++++ | +++ |
| CXCL10 (IP-10) | ++++ | ++++ |
| RANTES (CCL5) | ++ | − |
| GM-CSF | − | − |
| IL-10 | − | − |
| IFN-α | +++ | − |
| IFN-β | +++ | − |
| IFN-γ | +++ | − |

++++ = 6000-20000 pg/ml
+++ = 1000-5000 pg/ml
++ = 400-1000 pg/ml
+ = 50-300 pg/ml
− = under LLOQ/no or very small changes over baseline or buffer/high variability

### Example 6: Effect of the Presence of PS for In Vivo Rabies Vaccination—(i) VNTs and (ii) ELIspot Measurements

RABV-G mRNA was formulated in LNP9 to LNP16 of Table Ex-5 and in LNP-C of Table Ex-4 as described above in Example 2.2.

The LNP formulations were applied to 7 weeks old female Balb/C mice on days 0 and 21 intramuscularly (i.m.; musculus tibialis) with doses of mRNA, formulations, and control groups as shown in Table Ex-11. As indicated, the i.m. immunizations were carried out with 1 μg RABV-G-mRNA formulated as shown in Tables Ex-5/4 and Ex-11. A negative control group received buffer only. The mice were terminated at day 28.

For conducting (i) VNT and (ii) ELISpot analyses, serum samples were taken at days 21 and 28, and the spleens were isolated at day 28. Splenocytes from vaccinated mice were isolated according to a standard protocol known in the art and as described above and then cryo-conserved under standard conditions until further experimentation was started.

TABLE Ex-11
Vaccination scheme of Example 6

| | No. of | | | | | | |
|---|---|---|---|---|---|---|---|
| Group | mice | RNA Treatment | Formulation | mRNA | Dose | Route | Volume |
| 1 | 6 | mRNA encoding RABV-G | LNP9 | RABV-G | 1 μg | i.m. | 1 × 25 μl |
| 2 | 6 | mRNA encoding RABV-G | LNP10 | RABV-G | 1 μg | i.m. | 1 × 25 μl |
| 3 | 6 | mRNA encoding RABV-G | LNP11 | RABV-G | 1 μg | i.m. | 1 × 25 μl |
| 4 | 6 | mRNA encoding RABV-G | LNP12 | RABV-G | 1 μg | i.m. | 1 × 25 μl |
| 5 | 6 | mRNA encoding RABV-G | LNP13 | RABV-G | 1 μg | i.m. | 1 × 25 μl |
| 6 | 6 | mRNA encoding RABV-G | LNP14 | RABV-G | 1 μg | i.m. | 1 × 25 μl |
| 7 | 6 | mRNA encoding RABV-G | LNP15 | RABV-G | 1 μg | i.m. | 1 × 25 μl |
| 8 | 6 | mRNA encoding RABV-G | LNP16 | RABV-G | 1 μg | i.m. | 1 × 25 μl |
| 9 | 6 | mRNA encoding RABV-G | LNP-C | RABV-G | 1 μg | i.m. | 1 × 25 μl |
| 10 | 6 | None | Buffer (PBS) | none | n.a. | i.m. | 1 × 25 μl |

### (i) VNTs Analysis

### Anti-Rabies Virus Neutralizing Titers (VNTs) Analysis Post Prime (d21 Post Prime) and 7 Days Post Boost (d28 Post Prime):

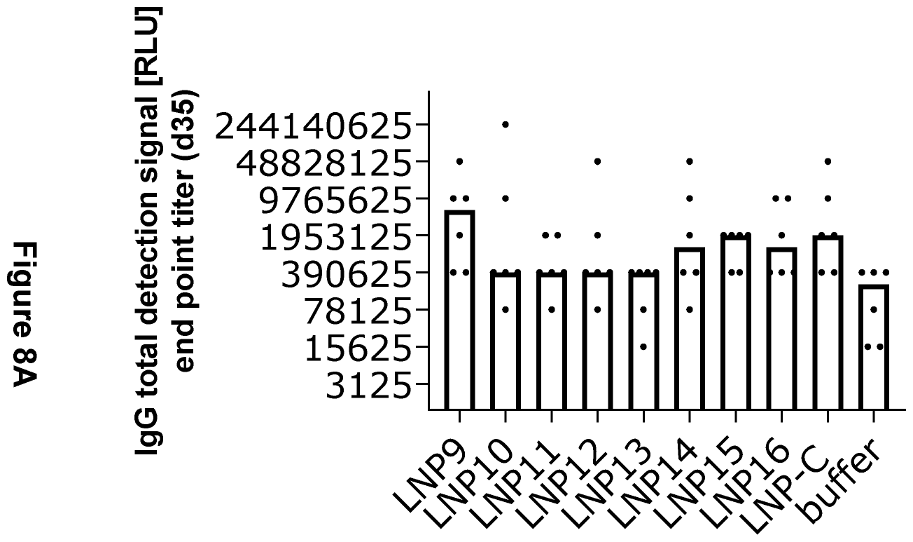

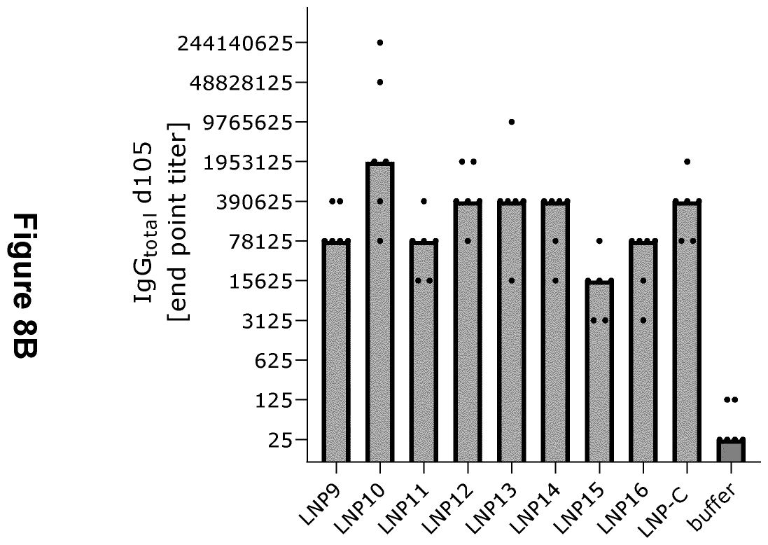

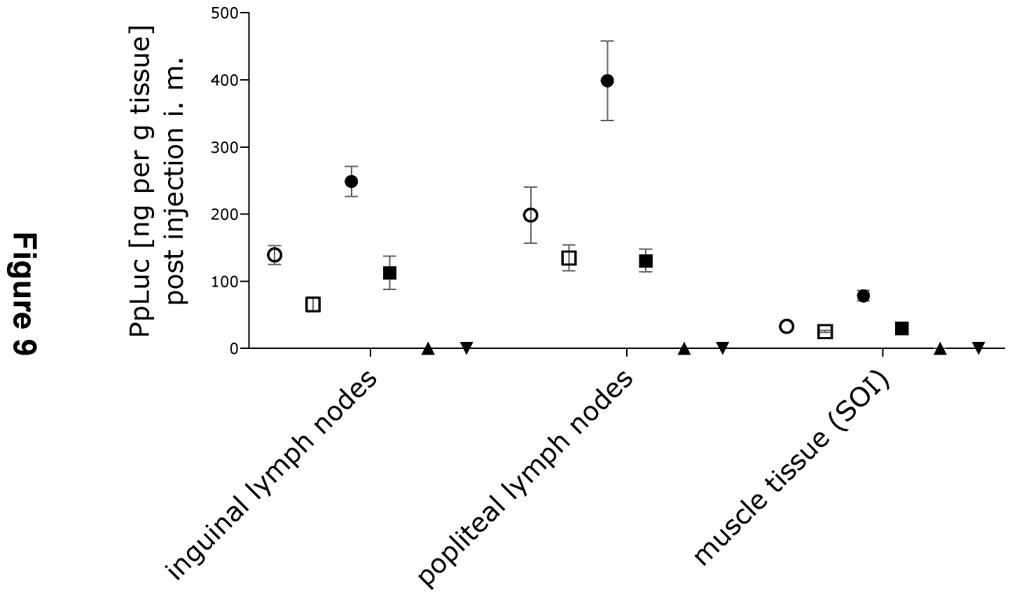

For determining the levels of antibody against the Rabies virus in serum, a classical virus neutralization test was performed (Fluorescent Antibody Virus Neutralization (FAVN) assay). Thus, 21 days after the first mRNA administration, blook samples were collected for further analysis, and 28 days after the first mRNA administration, mice were sacrificed and blood samples were collected for further analysis, i.e. for Virus neutralizing antibodies (VNA) analysis via FAVN assay. For said immunogenicity assays, the VNT was measured, i.e. anti-Rabies virus neutralizing titers (VNTs) in serum were analyzed by the Eurovir® Hygiene-Labor GmbH, Germany, using the FAVN assay and the Standard Challenge Virus CVS-11 according to WHO protocol. Results for the VNTs analysis at day 28 are shown in FIG. 7A (results for the VNTs analysis at day 21 not shown).

### Results of Anti-Rabies Virus Neutralizing Titers (VNTs) Analysis Post Prime (d21 Post Prime) and 7 Days Post Boost (d28 Post Prime):

As apparent from FIG. 7A (day 28, 7 days after boost), significant increased VNT levels can be seen after booster vaccination, whereas the described additives seemed to be responsible for a significant efficacy increase. As expected, the buffer control did not result in a protective titer. Titres above the protective titre were, however, already achieved after prime (i.e. without the booster vaccination), namely at day 21 post prime (VNT analysis at day 21, data not shown).

### (ii) ELISpot Analysis

Detection of an RABV-G Antigen Specific Cellular Immune Response by ELISpot Analysis Through Spleen Cell Re-Stimulation with a RABV-G Peptide Library 7 Days Post Boost (d28 Post Prime):

Cryo-conserved splenocytes which were isolated as described above from mice immunized with different RABV-G-encoding mRNA formulations LNP9 to LNP16 were thawed and re-stimulated with a Rabies virus G protein peptide library (RABV-G peptide cocktail comprising 129 RABV-G peptides). Consequently, RABV-G-specific cell activation was analysed by an ELISpot assay. For detection of IFN-gamma, a 96 well ELISpot plate (Nitrocellulose Multiscreen HTS, Millipore) was incubated overnight with 100 μl coating buffer (15 mM Na₂CO₃, 15 mM NaHCO₃, 0.02% Na-Azide, pH 9.6) comprising antibody against IFN-gamma (Biotinylated Rat anti-mouse IFN-gamma antibody, BD Pharmingen, Heidelberg, Germany). The next day, 5×10⁵ splenocyte cells/well were added and re-stimulated with a RABV-G peptide mix (Rabies peptide library PepMix, JPT Peptide Technologies GmbH, Berlin, Germany) of 129 peptides (final concentration of 5 μg/peptide—said peptide mix comprised the amino acid sequence of the Rabies G protein from Pasteur vaccine strain of Rabies virus according to SEQ ID NO:1 of WO2015024665 displayed as 15 amino acid peptides with an overlap of 11 amino acids between adjacent peptides). Afterwards the cells were incubated for 24 h at 37° C. The next day the plates were washed twice with PBS, once with water and once with PBS/0.05% Tween-20 and afterwards incubated with a biotin-coupled secondary antibody (BD Pharmingen) for 11-24 h at 4° C. Then the plates were washed with PBS/0.05% Tween-20 and incubated for 2 h with alkaline phosphatase coupled to streptavidin in blocking buffer. After washing with PBS/0.05% Tween-20 the substrate (5-Bromo-4-Cloro-3-Indolyl Phosphate/Nitro Blue Tetrazolium Liquid Substrate System from Sigma Aldrich, Taufkirchen, Germany) was added to the plate and the conversion of the substrate could be detected visually. The reaction was then stopped by washing the plates with water. The dried plates were then read out by an ELISPOT plate reader (ELISpot plate reader BA-R-0564). For visualization of the spot levels the numbers were corrected by background subtraction.

As negative control during the vaccination experiment, mice were treated with buffer; as positive control during the ELISpot assay, Phorbol-12-myristat-13-acetat (PMA)/ionomycin was used to stimulate cytokine production through activation of lymphocytes.

Results of the Detection of an RABV-G Antigen Specific Cellular Immune Response by ELISpot Analysis Through Spleen Cell Re-Stimulation with a RABV-G Peptide Library 7 Days Post Boost (d28 Post Prime):

As apparent from FIG. 7B, the addition of Phosphatidylserine to the respective LNP formulations significantly increased the number of detected IFN-gamma spots indicative of induction of RABV-G protein specific T cells including cytotoxic T cell (CTL).

### Example 7: Effect of the Presence of PS for In Vivo Malaria Vaccination in a Long-Term Experiment

Pf-CSP mRNA was formulated in LNPs9 to 16 of Table Ex-5 and in LNP-C of Table Ex-4 as described above in Example 2.2.

The LNP formulations were applied to 7 weeks old female Balb/C mice on days 0, 21 and 98 intramuscularly (i.m.; musculus tibialis) with doses of mRNA, formulations, and control groups as shown in Table Ex-12. A negative control group received buffer only. The mice were terminated at day 105. Serum samples were taken at days 1, 21, 35, 98, 105, and the spleens were isolated at day 105.

TABLE Ex-12
Vaccination scheme of Example 7

| | No. of | | | | | | |
|---|---|---|---|---|---|---|---|
| Group | mice | RNA Treatment | Formulation | mRNA | Dose | Route | Volume |
| 1 | 6 | mRNA encoding Pf-CSP | LNP9 | Pf-CSP | 1 μg | i.m. | 1 × 25 μl |
| 2 | 6 | mRNA encoding Pf-CSP | LNP10 | Pf-CSP | 1 μg | i.m. | 1 × 25 μl |
| 3 | 6 | mRNA encoding Pf-CSP | LNP11 | Pf-CSP | 1 μg | i.m. | 1 × 25 μl |
| 4 | 6 | mRNA encoding Pf-CSP | LNP12 | Pf-CSP | 1 μg | i.m. | 1 × 25 μl |
| 5 | 6 | mRNA encoding Pf-CSP | LNP13 | Pf-CSP | 1 μg | i.m. | 1 × 25 μl |
| 6 | 6 | mRNA encoding Pf-CSP | LNP14 | Pf-CSP | 1 μg | i.m. | 1 × 25 μl |
| 7 | 6 | mRNA encoding Pf-CSP | LNP15 | Pf-CSP | 1 μg | i.m. | 1 × 25 μl |
| 8 | 6 | mRNA encoding Pf-CSP | LNP16 | Pf-CSP | 1 μg | i.m. | 1 × 25 μl |
| 9 | 6 | mRNA encoding Pf-CSP | LNP-C | Pf-CSP | 1 μg | i.m. | 1 × 25 μl |
| 10 | 6 | None | Buffer (PBS) | none | n.a. | i.m. | 1 × 25 μl |

Determination of specific humoral immune responses by ELISA and Intracellular cytokine staining was performed analog to Example 3.

### Results:

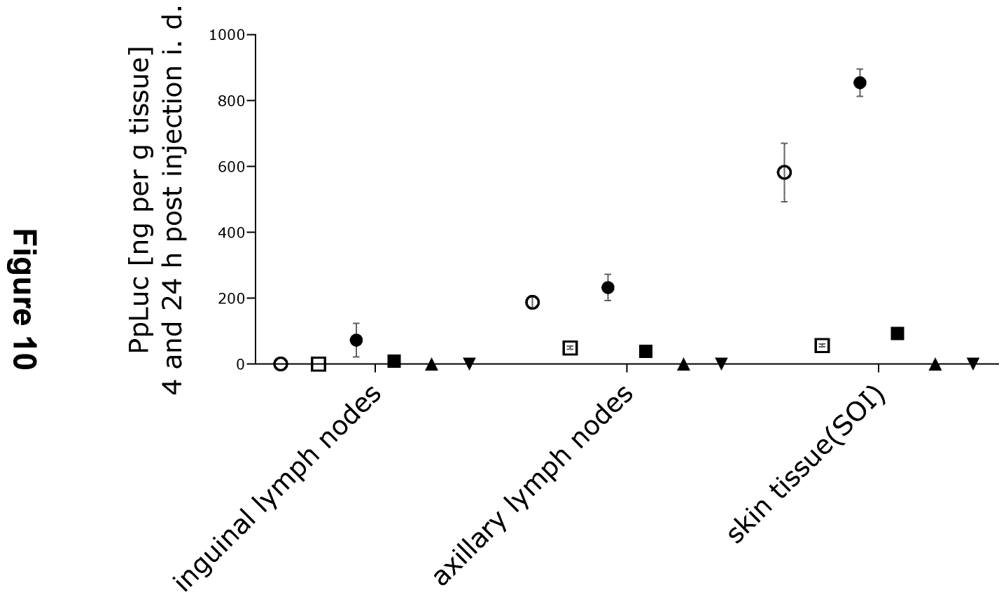

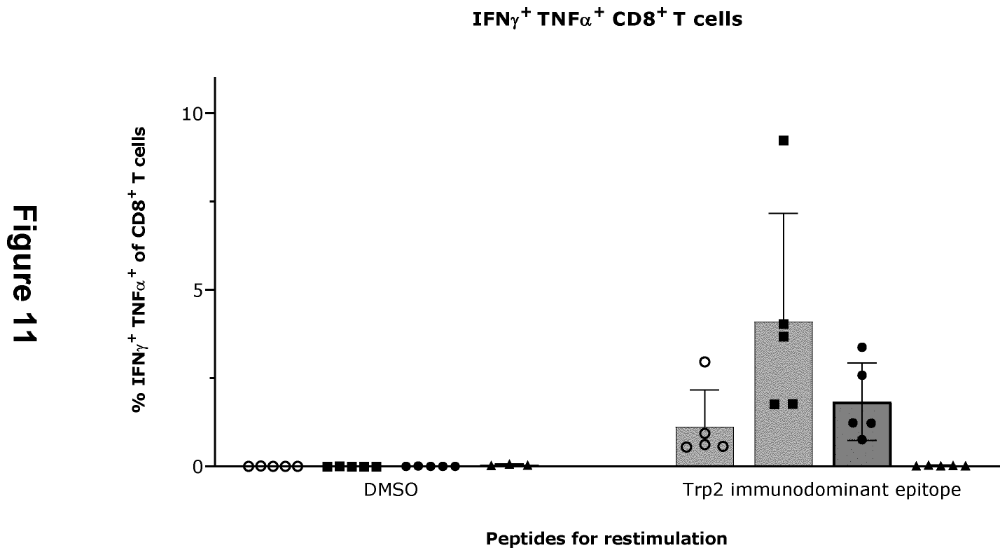

FIG. 8A shows that i.m. immunizations with 1 μg Pf-CSP-mRNA with LNPs comprising PS induced a humoral immune response in mice in comparison to LNPs not comprising PS at day 35. FIG. 8B shows that i.m. immunizations with 1 μg Pf-CSP-mRNA with LNPs comprising PS induced a very strong humoral immune response in mice in comparison to LNPs not comprising PS at day 105.

### Example 8: Synthesis of HEXA1

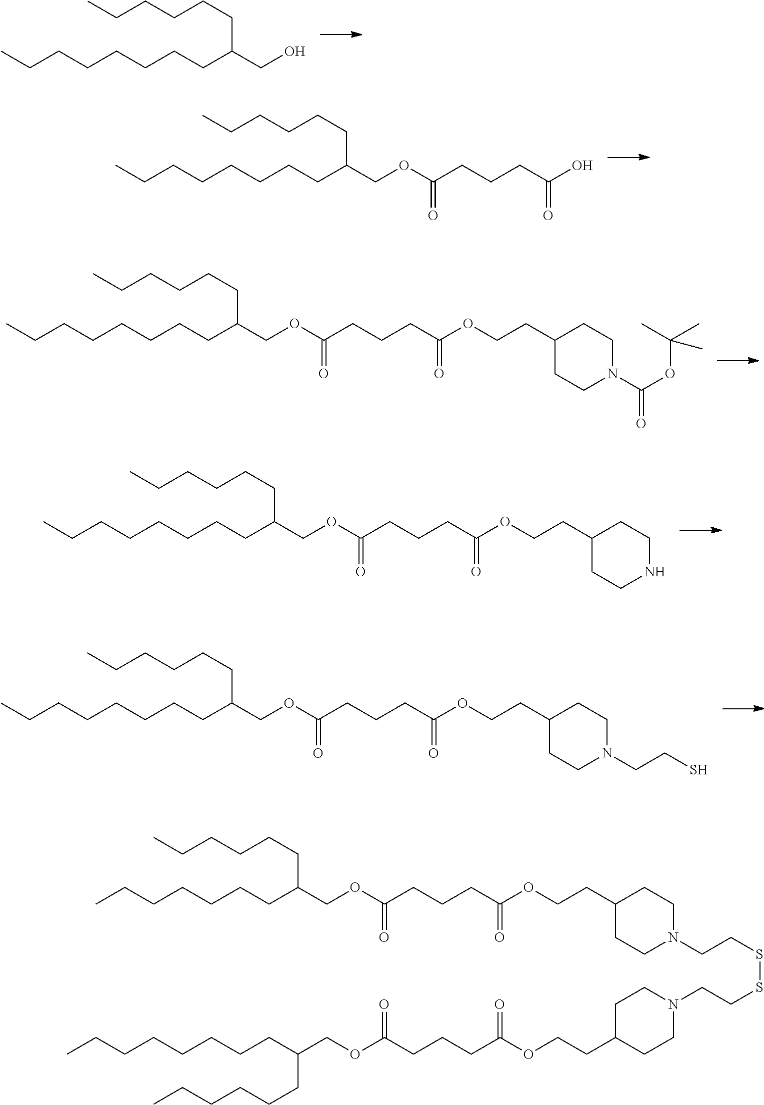

### Example 8.1

### Synthesis of

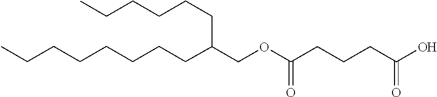 [SMILES: CCCCCCCCC(CCCCCC)COC(=O)CCCC(=O)O]

### 5-(2-hexyldecoxy)-5-oxo-pentanoic acid

To a solution of 2-Hexyl-1-decanol (150 g) and glutaric anhydride (74.13 g) 1000 ml of dry dichloromethane dimethylaminopyridine (90.71 g) was added and the reaction mixture was stirred for 65 hours under nitrogen at room temperature. The white precipitate that had formed was filtered off and discharged. The filtrate was concentrated in vacuum and mixed with 200 ml of petrol ether for 40 minutes resulting in a white suspension. The precipitate was filtered off and the filtrate concentrated. The crude was partitioned between 300 ml 1N hydrochloric acid and 500 ml of ethyl acetate. The organic phase was separated, washed with 500 ml of water and dried over anhydrous sodium sulphate. The sodium sulphate was filtered off and the solvent evaporated in vacuum. The crude residue was purified by flash chromatography on silica eluting with a gradient dichloromethane→dichloromethane:methanol 90:10. Fractions containing the product are combined and concentrated to give the pure target compound as a yellow oil (123.9 g, 56.1% yield).

### Example 8.2

### Synthesis of

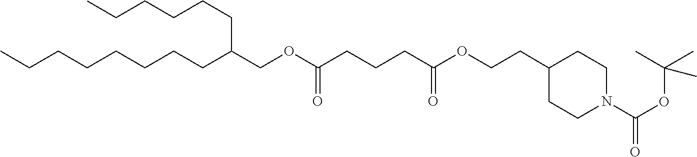 [SMILES: CCCCCCCCC(CCCCCC)COC(=O)CCCC(=O)OCCC1CCN(C(=O)OC(C)(C)C)CC1]

### O1-[2-(1-tert-butoxycarbonyl-4-piperidyl)ethyl] O5-(2-hexyldecyl) pentanedioate

The product from Example 8.1 (52.6 g) and tert-Butyl 4-(2-hydroxyethyl)piperidine-1-carboxylate (37.2 g) are dissolved in 600 ml of dichloromethane at room temperature giving a clear yellow solution. N,N′-Dicyclohexylcarbodiimid (33.48 g) was added and the reaction mixture was stirred at room temperature for 22 hours. More N,N′-Dicyclohexylcarbodiimid (15.2 g) was added and the mixture stirred at room temperature for another 42 hours. The white precipitate that has formed was filtered off and washed with a small volume of petrol ether. The combined filtrates are concentrated in vacuum and the residue purified by flash chromatography on silica with a solvent gradient from pure petrol ether to petrol ether:ethylacetate 90:10. The pure fractions of the product are combined and concentrated to give the target compound as an oil (32.8 g, 39.2% yield).

### Example 8.3

### Synthesis of

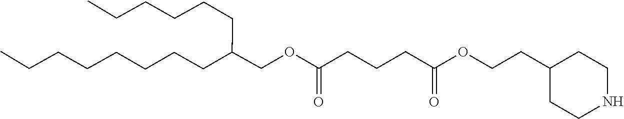 [SMILES: CCCCCCCCC(CCCCCC)COC(=O)CCCC(=O)OCCC1CCNCC1]

### O5-(2-hexyldecyl) O1-[2-(4-piperidyl)ethyl] pentanedioate

The product from Example 8.2 (32.8 g) was dissolved in 1000 ml of dichloromethane at room temperature. The solution was cooled in an ice bath and trifluoroacetic acid (35.6 ml) was added slowly at −0° C. The mixture was allowed to warm up to room temperature and stirred overnight. The mixture was washed with saturated sodium hydrogen carbonate solution and the aqueous phase was back-extracted with dichloromethane. The combined organic solutions are washed with brine, dried over anhydrous sodium sulphate, filtered and concentrated to give the target compound as a yellow oil (27.15 g, quantitative yield). The product was used without further purification in the next step.

### Example 8.4

### Synthesis of

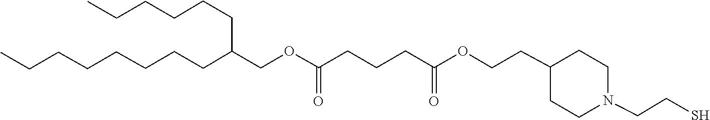 [SMILES: CCCCCCCCC(CCCCCC)COC(=O)CCCC(=O)OCCC1CCN(CCS)CC1]

### O5-(2-hexyldecyl) O1-[2-[1-(2-sulfanylethyl)-4-piperidyl]ethyl] pentanedioate

The crude product from Example 8.3 (1.37 g) was dissolved in 10 ml of dry toluene. N,N-diisopropylethylamine (0.533 ml) was added at room temperature resulting in a clear solution. The mixture was transferred to a pressure vial and 0.7 ml of ethylene sulphide was added. The vial was sealed and heated in an oil bath at 65° C. overnight. After cooling to room temperature, the complex reaction mixture was concentrated and used as obtained in the subsequent step.

### Example 8.5

### Synthesis of

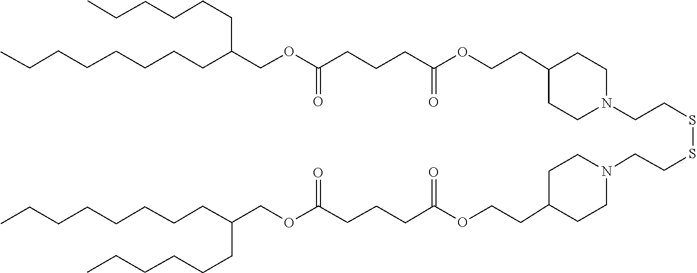 [SMILES: CCCCCCCCC(CCCCCC)COC(=O)CCCC(=O)OCCC1CCN(CCSSCCN2CCC(CCOC(=O)CCCC(=O)OCC(CCCCCC)CCCCCCCC)CC2)CC1]

### O1-[2-[1-[2-[2-[4-[2-[5-(2-hexyldecoxy)-5-oxo-pentanoyl]oxyethyl]-1-piperidyl]ethyldisulfanyl]ethyl]-4-piperidyl]ethyl] O5-(2-hexyldecyl) pentanedioate

The crude product mixture from Example 8.4 was dissolved in 15 ml acetonitrile. A solution of iodine in acetonitrile:water 9:1 was added drop wise at room temperature while stirring until a brown colour remains. The reaction mixture was concentrated and taken up in ethylacetate. This solution was washed subsequently with sodium hydrogen carbonate solution and sodium thiosulphate solution. The organic phase was dried over anhydrous sodium sulphate, filtered and concentrated in vacuum. The target compound was isolated by flash chromatography on silica, eluting with a gradient chloroform->chloroform:methanol 80:20. The respective fractions are combined and the solvents are evaporated to provide the pure target compound as a yellow oil (562 mg, 22% yield over two steps).

1H-NMR (500 MHz, CDCl3): 4.11 ppm (4H), 3.98 ppm (4H), 3.15-2.5 ppm (12H), 2.37 (8H), 2.17-1.84 ppm (8H), 1.81-1.5 ppm (10H), 1.49-1.08 ppm (54H), 0.88 (12H)

### Example 9: Synthesis of HEXA2

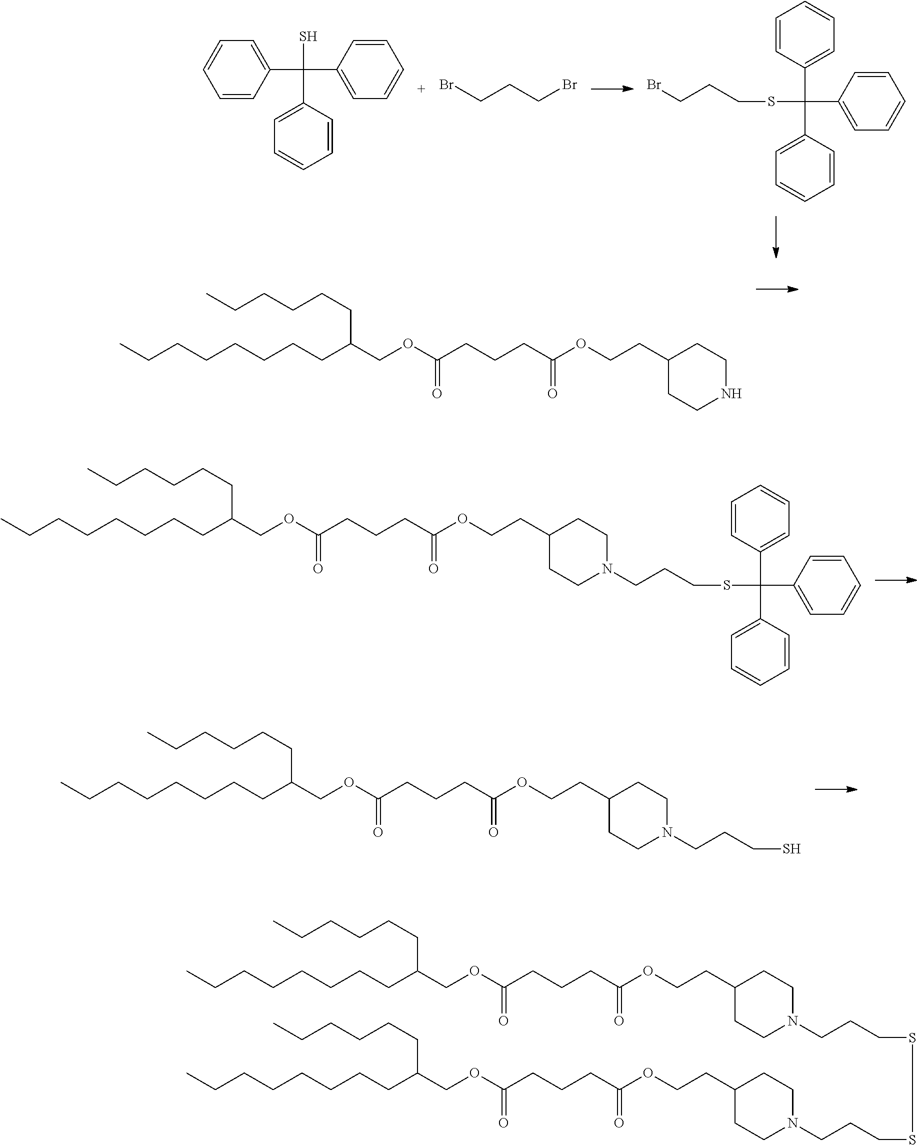

### Example 9.1

### Synthesis of

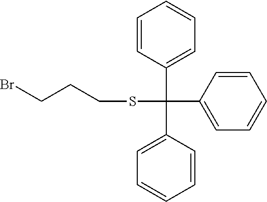 [SMILES: BrCCCSC(c1ccccc1)(c1ccccc1)c1ccccc1]

### [3-bromopropylsulfanyl(diphenyl)methyl]benzene

To a solution of triphenylmethanethiole (12 g) and 1,3-dibromopropane (22 ml) in 225 ml of dry tetrahydrofurane was added potassium carbonate (6.6 g). The reaction mixture was stirred at 50° C. for two days and at 60° C. for another 4 days under nitrogen. The insoluble salts are filtered off and the filtrate was concentrated in vacuum. The solution of the residue in 150 ml of dichloromethane was washed with 500 ml water and dried over anhydrous sodium sulphate, filtered and concentrated. The residue was purified by flash chromatography on silica eluting with petrol ether:dichloromethane 4:1. Relevant fractions are combined and the solvents evaporated in vacuum to give the pure target compound as a solid (4.2 g, 24.3% yield).

### Example 9.2

### Synthesis of

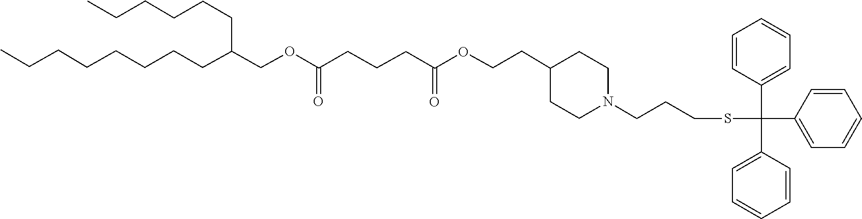 [SMILES: CCCCCCCCC(CCCCCC)COC(=O)CCCC(=O)OCCC1CCN(CCCSC(c2ccccc2)(c2ccccc2)c2ccccc2)CC1]

### O5-(2-hexyldecyl) O1-[2-[1-(3-tritylsulfanylpropyl)-4-piperidyl]ethyl] pentanedioate

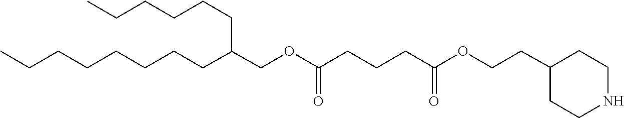 [SMILES: CCCCCCCCC(CCCCCC)COC(=O)CCCC(=O)OCCC1CCNCC1]

(4g) and the product from Example 9.1 (3.4 g) was mixed with 85 ml acetonitrile at room temperature to give a suspension. Dimethyl formamide (2 ml) was added and the mixture was stirred at 55° C. for 16 hours and additionally 24 hours at 65° C. Potassium carbonate (600 mg) was added and the mixture stirred for another 3 hours. The reaction mixture was filtered, concentrated and purified by flash chromatography (silica, dichloromethane:methanol 90:10 as eluent) to provide the essentially pure target compound (3.55 g, 52.9% yield) containing some residual dimethyl formamide.

### Example 9.3

### Synthesis of

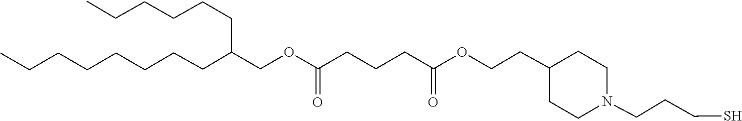 [SMILES: CCCCCCCCC(CCCCCC)COC(=O)CCCC(=O)OCCC1CCN(CCCS)CC1]

### O5-(2-hexyldecyl) O1-[2-[1-(3-sulfanylpropyl)-4-piperidyl]ethyl] pentanedioate

The product from Example 9.2 (1 g) was dissolved in 5 ml of dry dichloromethane and the solution was cooled to 0° C. in an ice bath. Subsequently trifluoro acetic acid (1.965 ml) and triethyl silane (0.206 ml) was added to give a slightly brownish solution. The reaction mixture was stirred at 0° C. for 30 minutes, diluted with 50 ml dichloromethane and washed with 100 ml of saturated sodium hydrogen carbonate solution. The organic phase was separated, dried over anhydrous sodium sulphate and concentrated. The residue was purified by flash chromatography on silica eluting with a gradient dichloromethane->dichloromethane:methanol 95:5 to give the target compound (0.47 g, 68% yield).

### Example 9.4

### Synthesis of

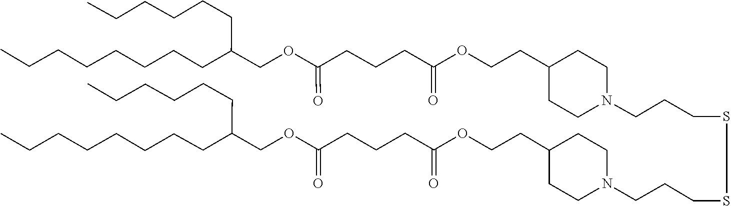 [SMILES: CCCCCCCCC(CCCCCC)COC(=O)CCCC(=O)OCCC1CCN(CCCSSCCCN2CCC(CCOC(=O)CCCC(=O)OCC(CCCCCC)CCCCCCCC)CC2)CC1]

### O1-[2-[1-[3-[3-[4-[2-[5-(2-hexyldecoxy)-5-oxo-pentanoyl]oxyethyl]-1-piperidyl]propyldisulfanyl]propyl]-4-piperidyl]ethyl] O5-(2-hexyldecyl) pentanedioate

The product from Example 9.3 (0.47 g) was dissolved in 10 ml acetonitrile. A solution of iodine in acetonitrile:water 9:1 was added drop wise at room temperature until a brown colour remains. The reaction mixture was stirred for 58 hours at room temperature, concentrated and purified by flash chromatography (silica, dichloromethane 4 dichloromethane:methanol 95:5) to give the target compound as the hydroiodic acid salt (240 mg, 45.8% yield).

1H-NMR (500 MHz, CDCl3): 10.01 ppm (2H), 4.15 ppm (4H), 3.99 ppm (4H), 3.83 ppm (4H), 3.28 ppm (4H), 2.94-2.80 ppm (8H), 2.53-2.44 ppm (4H), 2.43-2.36 ppm (8H), 2.21-2.09 ppm (4H), 2.04-1.91 ppm (8H), 1.84-1.67 ppm (6H), 1.36-1.2 ppm (50H), 0.90 ppm (12H)

### Example 10: Synthesis of THIOETHER

For the synthesis of THIOETHER (Mercachem B. V., Nijmegen, The Netherlands), coupling of dicarboxylic acid derivative 3 and 4-piperidineethanol gave bisamide 4 in 86%. Subsequent reduction with LiAlH₄ afforded 5 in 24% after purification by flash chromatography. Final coupling with commercially available Vitamin E derivative 6 gave THIOETHER in 52%, which was purified by extraction between heptane and MeCN, followed by flash chromatography.

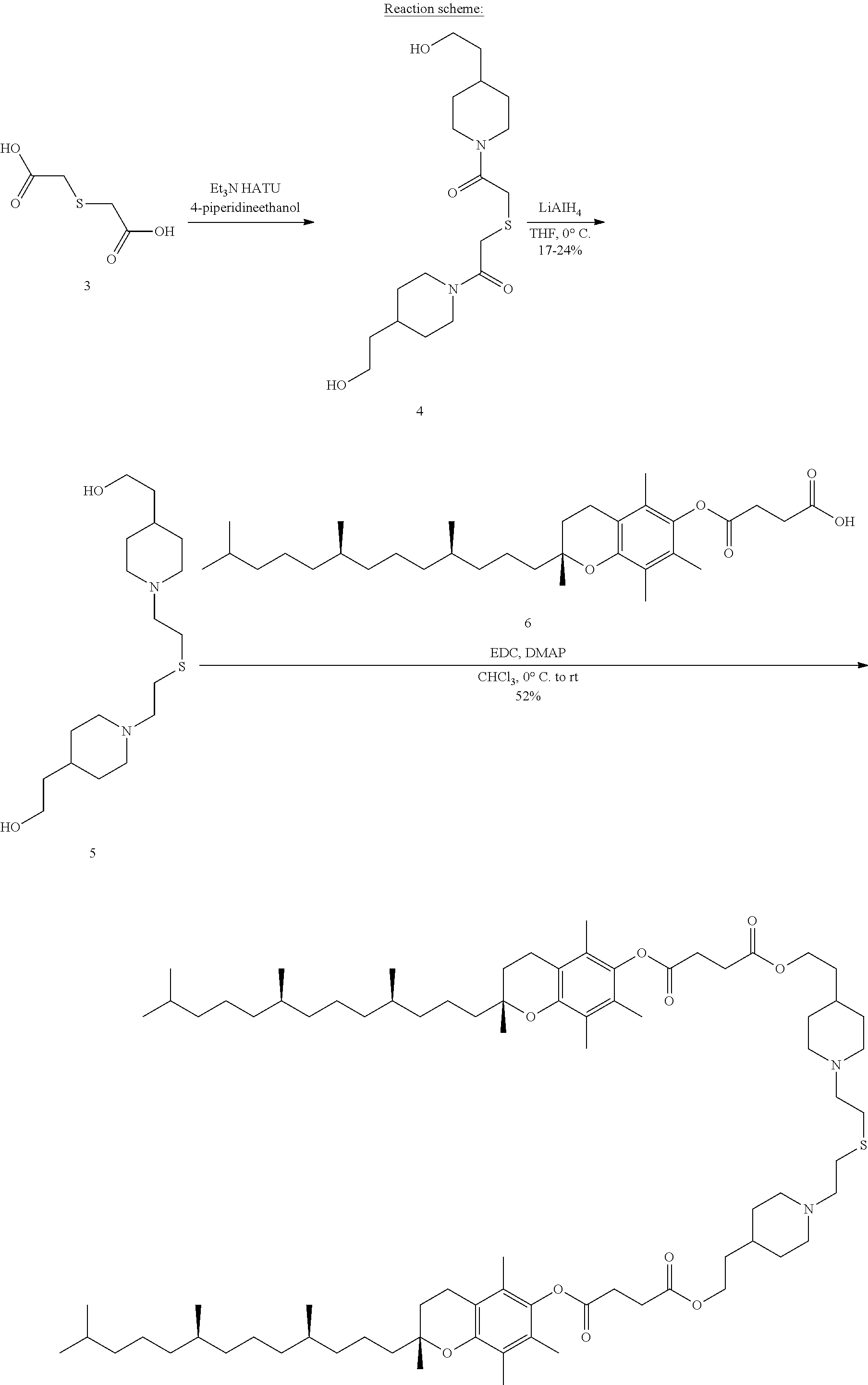

### Example 11: Mouse Immunization with Tyrosinase-Related Protein 2 (Trp2) Encoding mRNA Formulated in LNPs Comprising PS [Prophetic]

Trp2 encoding mRNA (SEQ ID NO:29) is formulated into the LNPs as indicated in Table Ex-7, in accordance with the formulations of Table Ex-2 or Table Ex-4 as described above.

The resulting LNP formulations are applied to female Balb/c mice on days 0 and 21 intramuscularly (i.m.; musculus tibialis) with doses of mRNA, formulations, and control groups as shown in Table Ex-7. A negative control group receives buffer only. The mice are terminated at day 35. Serum samples are taken at days 1, 21 and 35, and the spleens are isolated at day 35.

TABLE Ex-7
Vaccination scheme of Example 11

| | No. of | | | | | | |
|---|---|---|---|---|---|---|---|
| Group | mice | RNA Treatment | Formulation | mRNA | Dose | Route | Volume |
| 1 | 6 | mRNA encoding Trp2 | LNP9 | Trp2 | 1 μg | i.m. | 1 × 25 μl |
| 2 | 6 | mRNA encoding Trp2 | LNP10 | Trp2 | 1 μg | i.m. | 1 × 25 μl |
| 3 | 6 | mRNA encoding Trp2 | LNP11 | Trp2 | 1 μg | i.m. | 1 × 25 μl |
| 4 | 6 | mRNA encoding Trp2 | LNP12 | Trp2 | 1 μg | i.m. | 1 × 25 μl |
| 5 | 6 | mRNA encoding Trp2 | LNP13 | Trp2 | 1 μg | i.m. | 1 × 25 μl |
| 6 | 6 | mRNA encoding Trp2 | LNP14 | Trp2 | 1 μg | i.m. | 1 × 25 μl |
| 7 | 6 | mRNA encoding Trp2 | LNP15 | Trp2 | 1 μg | i.m. | 1 × 25 μl |
| 8 | 6 | mRNA encoding Trp2 | LNP16 | Trp2 | 1 μg | i.m. | 1 × 25 μl |
| 9 | 6 | mRNA encoding Trp2 | LNP-C | Trp2 | 1 μg | i.m. | 1 × 25 μl |
| 10 | 6 | None | Buffer (PBS) | none | n.a. | i.m. | 1 × 25 μl |

### Determination of Specific Humoral Immune Responses by ELISA:

ELISA is performed according to standard techniques. Coated plates are incubated using respective serum dilutions, and binding of specific antibodies to the respective peptide are detected using biotinylated isotype specific anti-mouse antibodies followed by streptavidin-HRP (horseradish peroxidase) with Amplex™ Red Reagent as substrate. Endpoint titers of antibodies (total IgG) directed against the respective peptide are measured by ELISA on day 35 post prime (14 days post 1^{st} boost).

### Example 12: Organ Distribution of PpLuc after Intramuscular LNP Injection

20 μg doses of PpLuc mRNA were formulated according to Table Ex-13 according to previous working examples. LNPs were then applied to female Balb/c mice on day 0 intramuscularly (i.m.; musculus tibialis). Termination was carried out at the time point indicated below (i.e. either at 4 h or 24 h after application), and organs were collected. Furthermore, serum samples were taken 14 days before the application and at the time point indicated below (i.e. either at 4 h or 24 h after application).

TABLE Ex-13
Formulation setup of Example 12

| | FIG. 9 | | | | | | |
|---|---|---|---|---|---|---|---|
| Group/ | legend/ | Time | | Ionizable | | Neutral | Polymer conjugated |
| Order | group | point | | lipid | Cholesterol | lipid(s) | lipid |
| 1 | open | 4 | h | 49 mol % | 38.5 mol % | 10 mol % DPhyPE | 2.5 mol % “PMOZ 4” |
| | circles | | | THIOETHER | | | (with with 50 |
| | | | | | | | monomer repeats |
| | | | | | | | (n = 50)) |
| 2 | open | 24 | h | 49 mol % | 38.5 mol % | 10 mol % DPhyPE | 2.5 mol % “PMOZ 4” |
| | squares | | | THIOETHER | | | (with with 50 |
| | | | | | | | monomer repeats |
| | | | | | | | (n = 50)) |
| 3 | filled | 4 | h | 59 mol % | 28.5 mol % | 5 mol % DPhyPE, | 2.5 mol % “PMOZ 4” |
| | circles | | | THIOETHER | | 5 mol % DPhyPS | (with with 50 |
| | | | | | | | monomer repeats |
| | | | | | | | (n = 50)) |
| 4 | filled | 24 | h | 59 mol % | 28.5 mol % | 5 mol % DPhyPE, | 2.5 mol % “PMOZ 4” |
| | squares | | | THIOETHER | | 5 mol % DPhyPS | (with with 50 |
| | | | | | | | monomer repeats |
| | | | | | | | (n = 50)) |

| | | | | |
|---|---|---|---|---|
| 5 | triangle | 4 | h | PBS buffer |
| | up | | | |
| 6 | triangle | 24 | h | PBS buffer |
| | down | | | |

PpLuc expression was determined by Luciferase assay in the tissue lysates prepared according to standard methods after the organ collection.

Results: it is apparent from FIG. 9 that LNPs comprising DPhyPS showed higher PpLuc concentrations at both time points (4 h and 24 h; filled symbols) as when compared to LNPs not comprising DPhyPS (4 h and 24 h; open symbols). This is the case for inguinal lymph nodes, popliteal lymph nodes and muscle (i.e. the site of injection (Sol)).

### Example 13: Organ Distribution of PpLuc after Intradermal LNP Injection

20 μg doses of PpLuc mRNA were formulated according to Table Ex-14 according to previous working examples. LNPs were then applied to female Balb/c mice on day 0 intradermally (i.d.). Termination was carried out at the time point indicated below (i.e. either at 4 h or 24 h after application), and organs were collected. Furthermore, serum samples were taken 14 days before the application and at the time point indicated below (i.e. either at 4 h or 24 h after application).

### Table Ex-14: Formulation Setup of Example 13

TABLE EX-14
Formulation setup of Example 13

| | FIG. 10 | | | | | |
|---|---|---|---|---|---|---|
| Group/ | legend/ | Time | Ionizable | | | Polymer conjugated |
| Order | group | point | lipid | Cholesterol | Neutral lipid(s) | lipid |
| 1 | open | 4 h | 49 mol % | 38.5 mol % | 10 mol % DPhyPE | 2.5 mol % “PMOZ 4” |
| | circles | | THIOETHER | | | (with with 50 |
| | | | | | | monomer repeats |
| | | | | | | (n = 50)) |
| 2 | open | 24 h | 49 mol % | 38.5 mol % | 10 mol % DPhyPE | 2.5 mol % “PMOZ 4” |
| | squares | | THIOETHER | | | (with with 50 |
| | | | | | | monomer repeats |
| | | | | | | (n = 50)) |
| 3 | filled circles | 4 h | 59 mol % | 28.5 mol % | 5 mol % DPhyPE, | 2.5 mol % “PMOZ 4” |
| | | | THIOETHER | | 5 mol % DPhyPS | (with with 50 |
| | | | | | | monomer repeats |
| | | | | | | (n = 50)) |
| 4 | filled | 24 h | 59 mol % | 28.5 mol % | 5 mol % DPhyPE, | 2.5 mol % “PMOZ 4” |
| | squares | | THIOETHER | | 5 mol % DPhyPS | (with with 50 |
| | | | | | | monomer repeats |
| | | | | | | (n = 50)) |

| | | | |
|---|---|---|---|
| 5 | triangle up | 4 h | PBS buffer |
| 6 | triangle down | 24 h | PBS buffer |

Results: it is apparent from FIG. 10 that LNPs comprising DPhyPS showed higher PpLuc concentrations at both time points (4 h and 24 h; filled symbols) as when compared to LNPs not comprising DPhyPS (4 h and 24 h; open symbols). This is the case for inguinal lymph nodes, axillary lymph nodes and skin (i.e. the site of injection (Sol)).

### Example 14: Mouse Immunization with Trp2 Formulated in LNPs Comprising PS or DHPC

For evaluating the effect of the addition of PS or DHPC on LNPs in a further cancer therapy setting, Trp2 encoding mRNA (SEQ ID NO:29) was formulated into the LNPs as indicated in Table Ex-15 and Table Ex-16. The resulting LNP formulations were applied to female C57BL/6 mice on days 0, day 7 and day 21 intramuscularly (i.m.; musculus tibialis) with doses of mRNA, formulations, and control groups as shown in Table Ex-15. A negative control group received PBS buffer only. Blood samples were collected 8 h after the first vaccination and 18 h after the second vaccination. Mice were terminated at day 21, spleens were collected for analyses in ICS and serum was collected for analysis in an antibody ELISA.

Subsequently it was assayed for the T cell response (CD8; (IFNy/TNFα producing CD8 T cells and IFNy TNFα producing CD8 T cells), i.e. spleen samples were re-stimulated with a Trp2 immunodominant epitope, i.e. Tyrosinase-related protein 2 (TRP2, 180-188) peptide according to SEQ ID NO:1 as disclosed in WO2020176984. As DMSO group served as control; assays were performed as described before and as known in the art.

Endpoint titers of antibodies (IgG2) directed against Trp2 were measured via antibody ELISA according to standard techniques. I.e. ELISA plates were coated with DCT (the Trp2 approved HGNC symbol is dopachrome tautomerase (DCT)); coated plates were incubated using respective serum dilutions, and binding of specific antibodies to the respective peptide were detected using biotinylated isotype specific anti-mouse detection antibodies followed by streptavidin-HRP (horseradish peroxidase) with Amplex™ Red Reagent as substrate.

TABLE Ex-15
Vaccination scheme of Example 14

| | Indicated in | No. of | | | | | |
|---|---|---|---|---|---|---|---|
| Group | FIG. 11 as | mice | RNA Treatment | Formulation | Dose | Route | Volume |
| 1 | open | 5 | mRNA encoding Trp2 | LNP9 | 1 μg | i.m. | 1 × 25 μl |
| | circles | | (SEQ ID NO: 29) | | | | |
| 2 | filled | 5 | mRNA encoding Trp2 | LNP10 | 1 μg | i.m. | 1 × 25 μl |
| | squares | | (SEQ ID NO: 29) | | | | |
| 3 | filled | 5 | mRNA encoding Trp2 | LNP10 | 1 μg | i.m. | 1 × 25 μl |
| | circles | | (SEQ ID NO: 29) | | | | |
| 4 | filled | 5 | None | Buffer (PBS) | n.a. | i.m. | 1 × 25 μl |
| | triangles | | | | | | |

TABLE Ex-16
LNP formulation for groups 1-3 of Table Ex-15 of Example 14

| | Lipids (mol %) | | | | | |
|---|---|---|---|---|---|---|
| | ionizable lipid - steroid - neutral lipid - polymer lipid - additive | | | | | |
| | | | | “PMOZ 4” | | |
| | | | | (with with 50 | | |
| | | | | monomer repeats | | N:P |
| Group | THIOETHER | Chol | DPhyPE | (n = 50)) | Additive | ratio |
| 1 | 49 | 38.5 | 10 | 2.5 | — | 14 |
| 2 | 49 | 38.5 | 8 | 2.5 | 2 | 14 |
| | | | | | Phosphatidylserine | |
| 3 | 49 | 28.5 | 10 | 2.5 | 10 | 14 |
| | | | | | DHPC | |

Results: after peptide restimulation with a Trp2 immunodominant epitope, it was clearly apparent from FIG. 11 that LNPs comprising DPhyPS (filled squares) and LNPs comprising DHPC (filled circles) showed a significantly higher CD8T cell response as when compared to LNPs not comprising DPhyPS or DHPC (open circles). Furthermore, it was clearly apparent from FIG. 12 that LNPs comprising DPhyPS (filled squares) and LNPs comprising DHPC (filled circles) showed showed significantly higher IgG2a endpoint titers as when compared to LNPs not comprising DPhyPS or DHPC (open circles).

1. A vaccine composition comprising
a) at least one nucleic acid encoding at least one antigen or fragment or variant thereof; and
b) a carrier composition, wherein the carrier composition comprises the phospholipid phosphatidylserine, wherein the amount of the phosphatidylserine is not more than 9 mol %, preferably not more than 5 mol %, of the total molar amount of all lipidic excipients in the carrier composition.

2. The vaccine composition according to claim 1, wherein the at least one nucleic acid is not a tolerogenic nucleic acid; and/or wherein the at least one nucleic acid does not encode a tolerogenic polypeptide; and/or wherein the vaccine composition does not comprise an antigen or fragment or variant thereof; and/or wherein the vaccine composition comprises the at least one nucleic acid as the sole payload; and/or wherein the vaccine composition is not a tolerogenic composition.

3. The vaccine composition according to claim 1 or 2, wherein the carrier composition at least partly encapsulates the at least one nucleic acid.

4. The vaccine composition according to any one of claims 1 to 3, wherein the carrier composition encapsulates the at least one nucleic acid.

5. The vaccine composition according to any one of claims 1 to 4, wherein the carrier composition comprises an inner surface and an outer surface facing the outside, wherein the phosphatidylserine is located at the outer surface of the carrier composition.

6. The vaccine composition according to claim 5, wherein the hydrophilic head group of the phosphatidylserine is located at the outer surface of the carrier composition.

7. The vaccine composition according to any one of claims 1 to 6, wherein the hydrophilic head group of the phosphatidylserine comprised in the carrier composition is accessible from the outside of the carrier composition.

8. The vaccine composition according to any one of claims 1 to 7, wherein the phosphatidylserine is selected from the group consisting of DPhyPS, WT-PS, 16:0—PS, 14:0—PS, 10:0—PS, 6:0—PS, 18:1—PS DOPS, 18:1-Lyso PS and 18:0-Lyso PS.

9. The vaccine composition according to any one of claims 1 to 8, wherein the carrier composition is a lipid nanoparticle composition.

10. The vaccine composition according to claim 9, wherein the lipid nanoparticle composition further comprises
(i) a cationic or ionizable lipid; and/or
(ii) a steroid; and/or
(iii) a further phospholipid in addition to phosphatidylserine, preferably DPhyPE; and/or
(iv) a polymer conjugated lipid.

11. The vaccine composition according to claim 9 or 10, wherein the lipid nanoparticle composition further comprises
(i) a cationic or ionizable lipid;
(ii) a steroid;
(iii) a further phospholipid in addition to phosphatidylserine, preferably DPhyPE; and
(iv) a polymer conjugated lipid.

12. The vaccine composition according to claim 10 or 11, wherein the cationic or ionizable lipid carries a net positive charge at physiological pH, preferably wherein the cationic or ionizable lipid comprises a tertiary nitrogen group or quaternary nitrogen group, more preferably wherein the cationic or ionizable lipid is selected from the group consisting of HEXA1, HEXA2 and THIOETHER with the structures shown in FIGS. 1A, 1B and 1C, respectively.

13. The vaccine composition according to any one of claims 10 to 12, wherein the steroid is selected from the group consisting of cholesterol, cholesteryl hemisuccinate (CHEMS) and a derivate thereof, preferably wherein the steroid is cholesterol.

14. The vaccine composition according to any one of claims 10 to 13, wherein the further phospholipid is selected from the group consisting of 1,2-diphytanoyl-sn-glycero-3-phosphoethanolamine (DPhyPE; 1,2-di-(3,7,11,15-tetramethylhexadecanoyl)-sn-glycero-3-phosphoethanolamine), 1,2-diphytanoyl-sn-glycero-3-phosphocholine (DPhyPC), 1,2-dioleoyl-sn-glycero-3-phosphocholine (DOPC; dioleoylphosphatidylcholine), 1,2-Dipalmitoyl-sn-glycero-3-phosphocholine (DPPC; dipalmitoylphosphatidylcholine), 1,2-dioleoyl-sn-glycero-3-phosphoethanolamine (DOPE), phosphatidylethanolamines, distearoylphosphatidylcholines, dioleoyl-phosphatidylethanolamine (DOPEA), 1,2-distearoyl-sn-glycero-3-phosphoethanolamine (DSPE), palmitoyloleoylphosphatidylcholine (POPC), palmitoyloleoyl-phosphatidylethanolamine (POPE), 1,2-Dipalmitoyl-sn-glycero-3-phosphoethanolamine (DPPE), dioleoyl-phosphatidylethanolamine 4-(N-maleimidomethyl)-cyclohexane-1-carboxylate (DOPE-mal), 1,2-Dimyristoyl-sn-glycero-3-phosphoethanolamine (DMPE), 1,2-Dilinoleoyl-sn-glycero-3-phosphoethanolamine (DLoPE), distearoyl-phosphatidylethanolamine (DSPE), 1-Palmitoyl-2-oleoyl-sn-glycero-3-phosphoethanolamine (POPE), 1,2-Dilauroyl-sn-glycero-3-phosphoethanolamine (DLPE), 16-O-monomethylphosphoethanolamine, 16-O-dimethyl phosphatidylethanolamine, 1,2-Dierucoyl-sn-glycero-3-phosphoethanolamine (DEPE), 18-1-trans phosphatidylethanolamine, 1-stearoyl-2-oleoylphosphatidyethanolamine (SOPE), 1,2-Disqualeoyl-sn-glycero-3-phosphoethanolamine (DSQPE), 1,2-dielaidoyl-sn-glycero-3-phosphoethanolamine (transDOPE), 1-Stearoyl-2-linoleoyl-sn-glycero-3-phosphoethanolamine (SLPE), 1-tridecanoyl-sn-glycero-3-phospho-L-serine (sodium salt), 1-oleoyl-2-hydroxy-sn-glycero-3-phospho-L-serine (sodium salt), 1-palmitoyl-2-oleoyl-sn-glycero-3-phospho-L-serine (sodium salt) (POPS), 1-1-stearoyl-2-oleoyl-sn-glycero-3-phospho-L-serine (sodium salt), 1,2-dioleoyl-sn-glycero-3-phospho-L-serine (sodium salt) (DOPS), 1,2-distearoyl-sn-glycero-3-phospho-L-serine (sodium salt), 1,2-diphytanoyl-sn-glycero-3-phospho-L-serine (sodium salt), 1-O-hexadecanyl-2-O-(9Z-octadecenyl)-sn-glycero-3-phosphoethanolamine, 1,2-distearoyl-sn-glycero-3-phosphatidylcholine or 1,2-distearoyl-sn-glycero-3-phosphocholine (DSPC), 1,2-di-O-phytanyl-sn-glycero-3-phosphoethanolamine, 1-palmitoyl-2-cholesterylhemisuccinoyl-sn-glycero-3-phosphocholine (PChemsPC), 1,2-dicholesterylhemisuccinoyl-sn-glycero-3-phosphocholine (DChemsPC), 2-((2,3-bis(oleoyloxy)propyl)dimethylammonio)ethyl hydrogen phosphate (DOCP), 2-((2,3-bis(oleoyloxy)propyl)dimtheylammonio)ethyl ethyl phosphate (DOCPe), and 1-O-octadecyl-2-O-methyl-sn-glycero-3-phosphocholine (Edelfosine),
preferably wherein the further phospholipid is DPhyPE; and wherein the phospholipid, preferably DPhyPE, is optionally present in combination with a phospholipid having at least two alkyl chains, wherein each alkyl chain independently has a length of preferably C6, C7, C8, C9, or C10, more preferably a length of C6, C7, or C8, most preferably a length of C7, further most preferably a phospholipid selected from the group consisting of DHPC (1,2-diheptanoyl-sn-glycero-3-phosphocholine), 05:0 PC (1,2-dipentanoyl-sn-glycero-3-phosphocholine), 04:0 PC (1,2-dibutyryl-sn-glycero-3-phosphocholine), 06:0 PC (1,2-dihexanoyl-sn-glycero-3-phosphocholine), 08:0 PC (1,2-dioctanoyl-sn-glycero-3-phosphocholine), and 09:0 PC (1,2-dinonanoyl-sn-glycero-3-phosphocholine), with DHPC being most preferred as the optionally present phospholipid having at least two alkyl chains.

15. The vaccine composition according to any one of claims 10 to 14, wherein the polymer conjugated lipid is a pegylated lipid or a PMOZ-lipid.

16. The vaccine composition according to any one of claims 11 to 15, wherein the composition comprises excipients in a ratio selected from the group consisting of
(a-i) the cationic lipid at an amount of 30-70 mol %; the steroid at an amount of 20-50 mol %; the phospholipid at an amount of 5-25 mol %; and the polymer conjugated lipid at an amount of 0.5-5 mol %;
(a-ii) the cationic lipid at an amount of 40-60 mol %; the steroid at an amount of 20-40 mol %; the phospholipid at an amount of 10-20 mol %; and the polymer conjugated lipid at an amount of 1-2 mol %;
(a-iii) the cationic lipid of claim 12 at an amount of 30-70 mol %; the steroid of claim 13 at an amount of 20-50 mol %; the phospholipid phosphatidylserine and the phospholipid of claim 14 at an amount of 5-25 mol %; and the polymer conjugated lipid of claim 15 at an amount of 0.5-5 mol %; and
(a-iv) the cationic lipid of claim 12 at an amount of 40-60 mol %; the steroid of claim 13 at an amount of 20-40 mol %; the phospholipid phosphatidylserine and the phospholipid of claim 14 at an amount of 10-20 mol %; and the polymer conjugated lipid of claim 15 at an amount of 1-2 mol %;
preferably the composition comprising excipients in a ratio selected from the group consisting of
(b-i) the cationic lipid at an amount of 59 mol %; the steroid at an amount of 29.3 mol %; the phospholipid at an amount of 10 mol %; and the polymer conjugated lipid at an amount of 1.7 mol %;
(b-li) the cationic lipid at an amount of 58 mol %; the steroid at an amount of 29.3 mol %; the phospholipid at an amount of 11 mol %; and the polymer conjugated lipid at an amount of 1.7 mol %;
(b-iii) the cationic lipid at an amount of 49 mol %; the steroid at an amount of 29.3 mol %; the phospholipid at an amount of 20 mol %; and the polymer conjugated lipid at an amount of 1.7 mol %;
(b-iv) the cationic lipid of claim 12 at an amount of 59 mol %; the steroid of claim 13 at an amount of 29.3 mol %; the phospholipid phosphatidylserine and the phospholipid of claim 14 at an amount of 10 mol %; and the polymer conjugated lipid of claim 15 at an amount of 1.7 mol %;
(b-v) the cationic lipid of claim 12 at an amount of 58 mol %; the steroid of claim 13 at an amount of 29.3 mol %; the phospholipid phosphatidylserine and the phospholipid of claim 14 at an amount of 11 mol %; and the polymer conjugated lipid of claim 15 at an amount of 1.7 mol %; and
(b-vi) the cationic lipid of claim 12 at an amount of 49 mol %; the steroid of claim 13 at an amount of 29.3 mol %; the phospholipid phosphatidylserine and the phospholipid of claim 14 at an amount of 20 mol %; and the polymer conjugated lipid of claim 15 at an amount of 1.7 mol %;
more preferably the composition comprising excipients in a ratio selected from the group consisting of
(c-i) the cationic lipid of claim 12 at an amount of 59 mol %; the steroid of claim 13 at an amount of 29.3 mol %; the phospholipid phosphatidylserine at an amount of 5 mol % and DPhyPE at an amount of 5 mol %; and the polymer conjugated lipid of claim 15 at an amount of 1.7 mol %;
(c-ii) the cationic lipid of claim 12 at an amount of 59 mol %; the steroid of claim 13 at an amount of 29.3 mol %; the phospholipid phosphatidylserine at an amount of 2 mol % and DPhyPE at an amount of 8 mol %; and the polymer conjugated lipid of claim 15 at an amount of 1.7 mol %;
(c-iii) the cationic lipid of claim 12 at an amount of 58 mol %; the steroid of claim 13 at an amount of 29.3 mol %; the phospholipid phosphatidylserine at an amount of 5 mol %, DPhyPE at an amount of 5 mol % and DHPC at an amount of 1 mol %; and the polymer conjugated lipid of claim 15 at an amount of 1.7 mol %;
(c-iv) the cationic lipid of claim 12 at an amount of 49 mol %; the steroid of claim 13 at an amount of 29.3 mol %; the phospholipid phosphatidylserine at an amount of 5 mol %, DPhyPE at an amount of 5 mol % and DHPC at an amount of 10 mol %; and the polymer conjugated lipid of claim 15 at an amount of 1.7 mol %.

17. The vaccine composition according to any one of the preceding claims, wherein the at least one nucleic acid is DNA or RNA.

18. The vaccine composition according to claim 17, wherein the at least one nucleic acid is RNA, preferably mRNA comprising a coding sequence encoding the at least one antigen or fragment or variant thereof and optionally a coding sequence encoding at least one self-amplifying enzyme.

19. The vaccine composition according to claim 18, wherein the lipid nanoparticles comprise the mRNA
(i) at an amount such as to achieve an N/P ratio in the range of 10 to 20, preferably about 2 to about 15, more preferably about 3 to about 10, even more preferably about 4 to about 9, most preferably about 6; or
(ii) at an amount such as to achieve an N/P ratio in the range of about 5 to about 20, more preferably about 10 to about 18, even more preferably about 12 to about 16, most preferably about 14; and/or
(iii) at an amount such as to achieve a lipid: mRNA weight ratio in the range of about 20 to about 60, preferably from about 3 to about 15, about 5 to about 13, about 4 to about 8 or from about 7 to about 11; and/or
wherein the lipid nanoparticles have a mean hydrodynamic diameter as determined by dynamic laser scattering from about 50 nm to about 300 nm, or from about 60 nm to about 250 nm, or from about 60 nm to about 200 nm, or from about 70 nm to 200 nm, or from about 75 nm to about 160 nm, or from about 90 nm to about 140 nm, or from about 100 nm to about 140 nm; and/or
wherein the lipid nanoparticles exhibit a zeta potential in the range of −50 mV to +50 mV, preferably in the range of −25 mV to +25 mV, more preferably in the range of −10 mV to +10 mV, most preferably in the range of −5 mV to +5 mV.

20. The vaccine composition according to claim 18 or 19, wherein the mRNA is a mono-, bi-, or multicistronic mRNA.

21. The vaccine composition according to any one of claims 18 to 20, wherein the mRNA comprises at least one chemical modification.

22. The vaccine composition according to claim 21, wherein the chemical modification is selected from the group consisting of base modifications, sugar modifications, backbone modifications and lipid modifications, preferably wherein the chemical modification is a base modification, more preferably wherein the base modification preferably is selected from the group consisting of pseudouridine (psi or ψ), N1-methylpseudouracil (N1MPU, N1Mpsi or N1Mψ), 1-ethylpseudouracil, 2-thiouracil (s2U), 4-thiouracil, 5-methylcytosine, 5-methyluracil, 5-methoxyuracil, and any combination thereof.

23. The vaccine composition according to any one of claims 18 to 22, wherein the coding sequence exhibits a sequence modification.

24. The vaccine composition according to claim 23, wherein the sequence modification is selected from a G/C content modification, a codon modification, a codon optimization or a C-optimization of the sequence; preferably wherein, compared with the coding sequence of the corresponding wild-type mRNA, the
a) G/C content of the coding sequence is increased;
b) C content of the coding sequence is increased;
c) codon usage in the coding sequence is adapted to the human codon usage; and/or
d) codon adaptation index (CAI) is increased or maximised in the coding sequence.

25. The vaccine composition according to any one of claims 18 to 24, wherein the mRNA further comprises
a) a 5′-CAP structure, preferably m7GpppN, more preferably CAP1 or m7G(5′)ppp(5′)(2′OMeA)pG;
b) at least one miRNA binding site sequence, preferably wherein the microRNA binding site is for a microRNA selected from the group consisting of a miR-126, miR-142, miR-144, miR-146, miR-150, miR-155, miR-16, miR-21, miR-223, miR-24, miR-27, miR-26a binding site, preferably a miR-122 or miR-142 binding site, or any combination of the aforementioned miRNA binding sites thereof;
c) at least one 5′-UTR element;
d) at least one 3′-UTR element;
e) at least one poly(A) sequence;
f) at least one poly(C) sequence;
g) optionally, a histone stem-loop selected from SEQ ID NO:3 or 4;
h) optionally, a 3-terminal sequence element selected from SEQ ID NO:41-70;
or any combinations of these.

26. The vaccine composition according to any one of claims 18 to 25, wherein the mRNA comprises a 5′-CAP structure, preferably m7G, CAP0, CAP1, CAP2, a modified CAP0 or a modified CAP1 structure.

27. The vaccine composition according to claim 25, wherein the at least one coding RNA comprises at least one heterologous 5′-UTR and/or at least one heterologous 3′-UTR, preferably wherein the at least one heterologous 5′-UTR comprises a nucleic acid sequence derived from a 5′-UTR of a gene selected from HSD17B4, RPL32, ASAH1, ATP5A1, MP68, NDUFA4, NOSIP, RPL31, SLC7A3, TUBB4B and UBQLN2, or from a homolog, a fragment or variant of any one of these genes; and/or preferably wherein the at least one heterologous 3′-UTR comprises a nucleic acid sequence derived from a 3′-UTR of a gene selected from PSMB3, ALB/albumin, alpha-globin, CASP1 (preferably SEQ ID NO:81 (DNA) or SEQ ID NO:82 (RNA)), COX6B1 (preferably SEQ ID NO:83 (DNA) or SEQ ID NO:84 (RNA)), GNAS (preferably SEQ ID NO:85 (DNA) or SEQ ID NO:86 (RNA)), NDUFA1 (preferably SEQ ID NO:87 (DNA) or SEQ ID NO:88 (RNA)) and RPS9 (preferably SEQ ID NO:79 (DNA) or SEQ ID NO:80 (RNA)), or from a homolog, a fragment or a variant of any one of these genes.

28. The vaccine composition according to claim 27, wherein the at least one coding RNA comprises a (i) HSD17B4 5′-UTR and a PSMB3 3′-UTR or (ii) a RPL32 5′-UTR and an ALB/albumin 3′-UTR, preferably a mutated alpha-globin 3′-UTR (SEQ ID NO:11, 12), more preferably a HSD17B4 5′-UTR (SEQ ID NO:21, 22) and a PSMB3 3′-UTR (SEQ ID NO:19, 20).

29. The vaccine composition according to any one of claims 18 to 24, wherein the mRNA comprises the following elements in the 5′ to 3′ direction:
a) a 5′-CAP structure, preferably selected from the group consisting of m7G(5′), m7G(5′)ppp(5′)(2′OMeA) and m7G(5′)ppp(5′)(2′OMeG);
b) a 5′-UTR element comprising a nucleic acid sequence derived from the 5′-UTR of a TOP gene, said nucleic acid sequence preferably comprising an RNA sequence that corresponds to the nucleic acid sequence according to SEQ ID NO:22, SEQ ID NO:24, SEQ ID NO:26 or preferably SEQ ID NO:77/78 (SLC7A3) or SEQ ID NO:75/76 (RPL31), or a homolog, a fragment or a variant thereof; most preferably according to SEQ ID NO:22 (HSD17B4);
c) the at least one coding sequence;
d) a 3′-UTR element comprising a nucleic acid sequence derived from an α-globin gene,
said nucleic acid sequence preferably comprising an RNA sequence that corresponds to the nucleic acid sequence according to SEQ ID NO:6, 8 or SEQ ID NO:10, 12, 14, 16, 18, or preferably SEQ ID NO:20, or a homolog, a fragment or a variant thereof; and/or a 3′-UTR element comprising a nucleic acid sequence derived from an albumin gene, said nucleic acid sequence preferably comprising an RNA sequence that corresponds to the nucleic acid sequence according to SEQ ID NO:18 (ALB/albumin) or preferably SEQ ID NO:79/80 (RPS9), or a homolog, a fragment or a variant thereof; most preferably according to SEQ ID NO:20 (PSMB3);
e) optionally, at least one poly(A) sequence, preferably consisting of 10 to 200, 10 to 100, 40 to 80, or 50 to 70 adenosine nucleotides, more preferably at least 70 adenosine nucleotides, even more preferably about 100 adenosine nucleotides;
f) optionally, at least one poly(C) sequence, preferably consisting of 10 to 200, 10 to 100, 20 to 70, 20 to 60 or 10 to 40 cytosine nucleotides; and
g) optionally, at least one histone stem-loop, preferably comprising the RNA sequence according to SEQ ID NO:4.

30. The vaccine composition according to any one of the preceding claims, wherein the antigen is derived from a pathogenic antigen, a tumour antigen, an allergenic antigen or an autoimmune self-antigen.

31. The vaccine composition according to claim 30, wherein the pathogenic antigen is selected from the group consisting of a bacterial antigen, a viral antigen, a fungal antigen and a protozoal antigen.

32. The vaccine composition according to claim 30 or 31, wherein the pathogenic antigen
(i) is derived from a SARS coronavirus 2 (SARS-CoV-2), nCoV-2019 coronavirus, SARS coronavirus (SARS-CoV), Bunyavirales virus, Cytomegalovirus (CMV), Dengue viruses (DENV-1, DENV-2, DENV-3 and DENV-4), Ebola virus (EBOV), Flavivirus, Hepatitis B virus (HBV), Herpes simplex virus (HSV), Human immunodeficiency virus (HIV), Human metapneumovirus (HMPV), Human Papilloma virus (HPV), Human parainfluenza viruses (HPIV), Influenza virus, extraintestinal pathogenic E. coli (ExPEC), Lassa mammarenavirus (LASV), MERS coronavirus, Mycobacterium tuberculosis, Nipah virus, Norovirus, Rabies virus (RABV), Respiratory Syncytial virus (RSV), Rhinovirus, Rotavirus, Vaccinia virus, Yellow Fever virus (YFV), Zika virus (ZIKV), Chlamydia trachomatis (i.e. bacterium Chlamydia causing Chlamydia), or Malaria parasite (e.g. Plasmodium falciparum, Plasmodium vivax, Plasmodium malariae, or Plasmodium ovale); and/or
(ii) is derived from a structural protein, an accessory protein, or a replicase protein from a SARS coronavirus 2 (SARS-CoV-2), nCoV-2019 coronavirus, SARS coronavirus (SARS-CoV), or an immunogenic fragment or immunogenic variant of any of these; and/or
(iii) is derived from a spike protein (S), an envelope protein (E), a membrane protein (M) or a nucleocapsid protein (N) from a SARS coronavirus 2 (SARS-CoV-2), nCoV-2019 coronavirus, SARS coronavirus (SARS-CoV), or an immunogenic fragment or immunogenic variant of any of these, preferably wherein the spike protein (S) comprises or consists of spike protein fragment S1 or spike protein fragment S2, more preferably spike protein fragment S1, or an immunogenic fragment or immunogenic variant thereof; and/or
(iv) is derived from a pre-fusion stabilized spike protein (S) (S_stab) from a SARS coronavirus 2 (SARS-CoV-2), nCoV-2019 coronavirus, SARS coronavirus (SARS-CoV) comprising at least one pre-fusion stabilizing mutation.

33. A pharmaceutical composition comprising the vaccine composition according to any one of claims 30 to 32 and a pharmaceutically acceptable carrier, diluent or excipient, preferably wherein the pharmaceutical composition is a sterile solid composition for reconstitution with a sterile liquid carrier, and wherein the composition further comprises one or more inactive ingredients selected from pH-modifying agents, bulking agents, stabilizers, non-ionic surfactants and antioxidants, and wherein the sterile liquid carrier is an aqueous carrier.

34. The vaccine composition according to any one of claims 30 to 32 or the pharmaceutical composition according to claim 33 for use in the treatment or prophylaxis of infectious diseases; cancer or tumor diseases, disorders or conditions; liver diseases selected from the group consisting of liver fibrosis, liver cirrhosis and liver cancer; allergies; or autoimmune disease, disorder or condition; in a subject.

35. The vaccine composition according to claim 32 or a pharmaceutical composition comprising the vaccine composition according to claim 32 for use in the treatment or prophylaxis of infectious diseases including viral, bacterial or protozoological infectious diseases in a subject.

36. The vaccine composition and the pharmaceutical composition for use according to claim 34 or 35, wherein the vaccine composition or pharmaceutical composition is administered via local or locoregional injection, infusion or implantation, in particular intradermal, subcutaneous, intramuscular, intracameral, subconjunctival, suprachoroidal injection, subretinal, subtenon, retrobulbar, topical, posterior juxtascleral administration, or intrapulmonal inhalation, interstitial, locoregional, intravitreal, intratumoral, intralymphatic, intranodal, intra-articular, intrasynovial, periarticular, intraperitoneal, intra-abdominal, intracardial, intralesional, intrapericardial, intraventricular, intrapleural, perineural, intrathoracic, epidural, intradural, peridural, intrathecal, intramedullary, intracerebral, intracavernous, intracorporus cavernosum, intraprostatic, intratesticular, intracartilaginous, intraosseous, intradiscal, intraspinal, intracaudal, intrabursal, intragingival, intraovarian, intrauterine, periocular, periodontal, retrobulbar, subarachnoid, subconjunctival, suprachoroidal injection, infusion, implantation, nasal, buccal, sublingual, otic or auricular, ophthalmic, conjunctival, vaginal, rectal, intracervical, endosinusial, laryngeal, oropharyngeal, ureteral, urethral administration, more preferably said lipid nanoparticle is administered intramuscularly, intravenously, intradermally, subcutaneously, intratumorally, intranasally, or by inhalation to a subject, preferably via local or locoregional injection or infusion to a subject.

37. A kit or kit of parts, comprising the vaccine composition according to any one of claims 30 to 32 or the pharmaceutical composition according to claim 33, optionally comprising a liquid vehicle for solubilizing, and, optionally, technical instructions providing information on administration and dosage of the components.

38. A method of treatment or prophylaxis of infectious diseases; cancer or tumor diseases, disorders or conditions; liver diseases selected from the group consisting of liver fibrosis, liver cirrhosis and liver cancer; allergies; or autoimmune disease, disorder or condition; in a subject comprising the steps:
a) providing the vaccine composition of any one of claims 30 to 32 or the pharmaceutical composition according to claim 33 or the kit or kit of parts according to claim 37; and
b) applying or administering the vaccine composition or the pharmaceutical composition or the kit or kit of parts to a tissue or an organism of the subject.

39. A method of inducing an immune response in a subject, the method comprising administering to the subject the vaccine composition of any one of claims 1 to 32 or the pharmaceutical composition of claim 33 in an amount effective to produce an antigen-specific immune response in the subject.

40. A method of targeting a vaccine composition comprising a) at least one nucleic acid, preferably mRNA, encoding at least one antigen or fragment or variant thereof; and b) a carrier composition, preferably a lipid nanoparticle composition, to antigen-presenting cells including dendritic cells and macrophages, and/or to the spleen, the method comprising administering to the subject the vaccine composition of any one of claims 1 to 32 or the pharmaceutical composition of claim 33.

41. Use of a vaccine composition of any one of claims 1 to 32 or the pharmaceutical composition according to claim 33 or the kit or kit of parts according to claim 37 for (i) inducing an immune response, for (ii) inducing an antigen specific T-cell response, preferably for (iii) inducing CD8+ T cells responses, and/or for (iv) targeting the vaccine composition or the pharmaceutical composition to antigen-presenting cells, including dendritic cells and macrophages, and/or to the spleen, in a subject.

42. Use of phosphatidylserine in a vaccine of any one of the above claims or in a carrier composition of any one of the above claims comprising a) at least one nucleic acid, preferably mRNA, encoding at least one antigen or fragment or variant thereof; and b) a carrier composition, preferably a lipid nanoparticle composition, for targeting the vaccine composition to antigen-presenting cells, including dendritic cells and macrophages, and/or to the spleen, in a subject.

43. The vaccine composition or the pharmaceutical composition for use according to claim 34 or 35, the method according to claim 38, 39 or 40, or the use according to claim 41 or 42, wherein the subject is a mammalian subject, preferably a human subject.

44. A vaccine composition or carrier composition comprising
a) at least one nucleic acid encoding at least one antigen or fragment or variant thereof; and
b) a carrier composition, wherein the carrier composition comprises the phospholipid 1,2-diheptanoyl-sn-glycero-3-phosphocholine (DHPC).

45. Use of DHPC in the carrier composition of a vaccine composition comprising a) at least one nucleic acid, preferably mRNA, encoding at least one antigen or fragment or variant thereof; and b) a vaccine or carrier composition, preferably a lipid nanoparticle composition, for targeting the vaccine composition to antigen-presenting cells, including dendritic cells and macrophages, and/or to the spleen, in a subject.

46. A vaccine composition or the pharmaceutical composition comprising DHPC for use according to claim 34 or 35, the method according to claim 38, 39 or 40, or the use according to claim 41 or 42, wherein the subject is a mammalian subject, preferably a human subject.


SEQUENCE LISTING (88 sequences)

SEQ ID NO: 1 - DNA, 13 bp, synthetic construct
  source 1..13: mol_type = other DNA; note = Kozak sequence
gccgccaccatgg

SEQ ID NO: 2 - RNA, 13 nt, synthetic construct
  source 1..13: mol_type = other RNA; note = Kozak sequence
gccgccaccatgg

SEQ ID NO: 3 - DNA, 24 bp, Homo sapiens
  source 1..24: mol_type = genomic DNA; note = histone stem-loop
caaaggctcttttcagagccacca

SEQ ID NO: 4 - RNA, 24 nt, Homo sapiens
  source 1..24: mol_type = mRNA; note = histone stem-loop
caaaggctcttttcagagccacca

SEQ ID NO: 5 - DNA, 110 bp, Homo sapiens
  source 1..110: mol_type = genomic DNA; note = HBA1 3'-UTR
gctggagcctcggtggccatgcttcttgccccttgggcctccccccagcccctcctccccttcctgcacccgtacccccgtggtctttgaataaagtctgagtgggcggc

SEQ ID NO: 6 - RNA, 110 nt, Homo sapiens
  source 1..110: mol_type = mRNA; note = HBA1 3'-UTR
gctggagcctcggtggccatgcttcttgccccttgggcctccccccagcccctcctccccttcctgcacccgtacccccgtggtctttgaataaagtctgagtgggcggc

SEQ ID NO: 7 - DNA, 108 bp, Homo sapiens
  source 1..108: mol_type = genomic DNA; note = HBA2 3'-UTR
gctggagcctcggtagccgttcctcctgcccgctgggcctcccaacgggccctcctcccctccttgcaccggcccttcctggtctttgaataaagtctgagtgggcag

SEQ ID NO: 8 - RNA, 108 nt, Homo sapiens
  source 1..108: mol_type = mRNA; note = HBA2 3'-UTR
gctggagcctcggtagccgttcctcctgcccgctgggcctcccaacgggccctcctcccctccttgcaccggcccttcctggtctttgaataaagtctgagtgggcag

SEQ ID NO: 9 - DNA, 132 bp, Homo sapiens
  source 1..132: mol_type = genomic DNA; note = HBB 3'-UTR
gctcgctttcttgctgtccaatttctattaaaggttcctttgttccctaagtccaactactaaactgggggatattatgaagggccttgagcatctggattctgcctaataaaaaacatttattttcattgc

SEQ ID NO: 10 - RNA, 132 nt, Homo sapiens
  source 1..132: mol_type = mRNA; note = HBB 3'-UTR
gctcgctttcttgctgtccaatttctattaaaggttcctttgttccctaagtccaactactaaactgggggatattatgaagggccttgagcatctggattctgcctaataaaaaacatttattttcattgc

SEQ ID NO: 11 - DNA, 44 bp, Homo sapiens
  source 1..44: mol_type = genomic DNA; note = muag 3'-UTR
gcccgatgggcctcccaacgggccctcctcccctccttgcaccg

SEQ ID NO: 12 - RNA, 44 nt, Homo sapiens
  source 1..44: mol_type = mRNA; note = muag 3'-UTR
gcccgatgggcctcccaacgggccctcctcccctccttgcaccg

SEQ ID NO: 13 - DNA, 186 bp, Homo sapiens
  source 1..186: mol_type = genomic DNA; note = albumin 3'-UTR
catcacatttaaaagcatctcagcctaccatgagaataagagaaagaaaatgaagatcaaaagcttattcatctgtttttctttttcgttggtgtaaagccaacaccctgtctaaaaaacataaatttctttaatcattttgcctcttttctctgtgcttcaattaataaaaaatggaaagaatct

SEQ ID NO: 14 - RNA, 186 nt, Homo sapiens
  source 1..186: mol_type = mRNA; note = albumin 3'-UTR
catcacatttaaaagcatctcagcctaccatgagaataagagaaagaaaatgaagatcaaaagcttattcatctgtttttctttttcgttggtgtaaagccaacaccctgtctaaaaaacataaatttctttaatcattttgcctcttttctctgtgcttcaattaataaaaaatggaaagaatct

SEQ ID NO: 15 - DNA, 186 bp, Homo sapiens
  source 1..186: mol_type = genomic DNA; note = albumin7 3'-UTR
catcacatttaaaagcatctcagcctaccatgagaataagagaaagaaaatgaagatcaatagcttattcatctctttttctttttcgttggtgtaaagccaacaccctgtctaaaaaacataaatttctttaatcattttgcctcttttctctgtgcttcaattaataaaaaatggaaagaacct

SEQ ID NO: 16 - RNA, 186 nt, Homo sapiens
  source 1..186: mol_type = mRNA; note = albumin7 3'-UTR
catcacatttaaaagcatctcagcctaccatgagaataagagaaagaaaatgaagatcaatagcttattcatctctttttctttttcgttggtgtaaagccaacaccctgtctaaaaaacataaatttctttaatcattttgcctcttttctctgtgcttcaattaataaaaaatggaaagaacct

SEQ ID NO: 17 - DNA, 187 bp, Homo sapiens
  source 1..187: mol_type = genomic DNA; note = ALB7 3'-UTR
gcatcacatttaaaagcatctcagcctaccatgagaataagagaaagaaaatgaagatcaatagcttattcatctctttttctttttcgttggtgtaaagccaacaccctgtctaaaaaacataaatttctttaatcattttgcctcttttctctgtgcttcaattaataaaaaatggaaagaacct

SEQ ID NO: 18 - RNA, 187 nt, Homo sapiens
  source 1..187: mol_type = mRNA; note = ALB7 3'-UTR
gcatcacatttaaaagcatctcagcctaccatgagaataagagaaagaaaatgaagatcaatagcttattcatctctttttctttttcgttggtgtaaagccaacaccctgtctaaaaaacataaatttctttaatcattttgcctcttttctctgtgcttcaattaataaaaaatggaaagaacct

SEQ ID NO: 19 - DNA, 57 bp, Homo sapiens
  source 1..57: mol_type = genomic DNA; note = PSMB3 3'-UTR
ccctgttcccagagcccacttttttttctttttttgaaataaaatagcctgtctttc

SEQ ID NO: 20 - RNA, 57 nt, Homo sapiens
  source 1..57: mol_type = mRNA; note = PSMB3 3'-UTR
ccctgttcccagagcccacttttttttctttttttgaaataaaatagcctgtctttc

SEQ ID NO: 21 - DNA, 62 bp, Homo sapiens
  source 1..62: mol_type = genomic DNA; note = HSD17B4 5'-UTR
gtcccgcagtcggcgtccagcggctctgcttgttcgtgtgtgtgtcgttgcaggccttattc

SEQ ID NO: 22 - RNA, 62 nt, Homo sapiens
  source 1..62: mol_type = mRNA; note = HSD17B4 5'-UTR
gtcccgcagtcggcgtccagcggctctgcttgttcgtgtgtgtgtcgttgcaggccttattc

SEQ ID NO: 23 - DNA, 42 bp, Homo sapiens
  source 1..42: mol_type = genomic DNA; note = RPL32 (32L4) 5'-UTR
ggcgctgcctacggaggtggcagccatctccttctcggcatc

SEQ ID NO: 24 - RNA, 42 nt, Homo sapiens
  source 1..42: mol_type = mRNA; note = RPL32 (32L4) 5'-UTR
ggcgctgcctacggaggtggcagccatctccttctcggcatc

SEQ ID NO: 25 - DNA, 75 bp, Homo sapiens
  source 1..75: mol_type = genomic DNA; note = ATP5A1 5'-UTR
gcggctcggccattttgtcccagtcagtccggaggctgcggctgcagaagtaccgcctgcggagtaactgcaaag

SEQ ID NO: 26 - RNA, 75 nt, Homo sapiens
  source 1..75: mol_type = mRNA; note = ATP5A1 5'-UTR
gcggctcggccattttgtcccagtcagtccggaggctgcggctgcagaagtaccgcctgcggagtaactgcaaag

SEQ ID NO: 27 - RNA, 2035 nt, synthetic construct
  source 1..2035: mol_type = other RNA; note = PpLuc mRNA R2244
ggggcgctgcctacggaggtggcagccatctccttctcggcatcaagcttgaggatggaggacgccaagaacatcaagaagggcccggcgcccttctacccgctggaggacgggaccgccggcgagcagctccacaaggccatgaagcggtacgccctggtgccgggcacgatcgccttcaccgacgcccacatcgaggtcgacatcacctacgcggagtacttcgagatgagcgtgcgcctggccgaggccatgaagcggtacggcctgaacaccaaccaccggatcgtggtgtgctcggagaacagcctgcagttcttcatgccggtgctgggcgccctcttcatcggcgtggccgtcgccccggcgaacgacatctacaacgagcgggagctgctgaacagcatggggatcagccagccgaccgtggtgttcgtgagcaagaagggcctgcagaagatcctgaacgtgcagaagaagctgcccatcatccagaagatcatcatcatggacagcaagaccgactaccagggcttccagtcgatgtacacgttcgtgaccagccacctcccgccgggcttcaacgagtacgacttcgtcccggagagcttcgaccgggacaagaccatcgccctgatcatgaacagcagcggcagcaccggcctgccgaagggggtggccctgccgcaccggaccgcctgcgtgcgcttctcgcacgcccgggaccccatcttcggcaaccagatcatcccggacaccgccatcctgagcgtggtgccgttccaccacggcttcggcatgttcacgaccctgggctacctcatctgcggcttccgggtggtcctgatgtaccggttcgaggaggagctgttcctgcggagcctgcaggactacaagatccagagcgcgctgctcgtgccgaccctgttcagcttcttcgccaagagcaccctgatcgacaagtacgacctgtcgaacctgcacgagatcgccagcgggggcgccccgctgagcaaggaggtgggcgaggccgtggccaagcggttccacctcccgggcatccgccagggctacggcctgaccgagaccacgagcgcgatcctgatcacccccgagggggacgacaagccgggcgccgtgggcaaggtggtcccgttcttcgaggccaaggtggtggacctggacaccggcaagaccctgggcgtgaaccagcggggcgagctgtgcgtgcgggggccgatgatcatgagcggctacgtgaacaacccggaggccaccaacgccctcatcgacaaggacggctggctgcacagcggcgacatcgcctactgggacgaggacgagcacttcttcatcgtcgaccggctgaagtcgctgatcaagtacaagggctaccaggtggcgccggccgagctggagagcatcctgctccagcaccccaacatcttcgacgccggcgtggccgggctgccggacgacgacgccggcgagctgccggccgcggtggtggtgctggagcacggcaagaccatgacggagaaggagatcgtcgactacgtggccagccaggtgaccaccgccaagaagctgcggggcggcgtggtgttcgtggacgaggtcccgaagggcctgaccgggaagctcgacgcccggaagatccgcgagatcctgatcaaggccaagaagggcggcaagatcgccgtgtaagactagtgcatcacatttaaaagcatctcagcctaccatgagaataagagaaagaaaatgaagatcaatagcttattcatctctttttctttttcgttggtgtaaagccaacaccctgtctaaaaaacataaatttctttaatcattttgcctcttttctctgtgcttcaattaataaaaaatggaaagaacctagatctaaaaaaaaaaaaaaaaaaaaaaaaaaaaaaaaaaaaaaaaaaaaaaaaaaaaaaaaaaaaaaaatgcatcccccccccccccccccccccccccccccccaaaggctcttttcagagccaccagaatt

SEQ ID NO: 28 - RNA, 858 nt, synthetic construct
  source 1..858: mol_type = other RNA; note = HsEPO mRNA R7339
gggagagtcccgcagtcggcgtccagcggctctgcttgttcgtgtgtgtgtcgttgcaggccttattcaagcttaccatgggcgtgcacgagtgccccgcctggctgtggctcctgctgagcctcctgtccctgccgctcgggctgcccgtcctgggcgcccccccgcggctcatctgcgacagccgcgtgctggagcggtacctgctcgaggcgaaggaggccgagaacatcaccaccgggtgcgccgagcactgctccctgaacgagaacatcacggtgccggacaccaaggtcaacttctacgcctggaagcgcatggaggtgggccagcaggccgtggaggtctggcaggggctggcgctcctgagcgaggccgtgctgcggggccaggccctcctggtgaactccagccagccctgggagcccctgcagctccacgtcgacaaggccgtgtccggcctgcgcagcctgaccaccctcctgcgggcgctgggggcccagaaggaggccatctcccccccggacgccgccagcgcggccccgctccgcacgatcaccgccgacaccttccggaagctgttccgcgtgtactccaacttcctgcggggcaagctcaagctgtacaccggggaggcctgccgcacgggcgaccggtgaggactagtccctgttcccagagcccacttttttttctttttttgaaataaaatagcctgtctttcagatctaaaaaaaaaaaaaaaaaaaaaaaaaaaaaaaaaaaaaaaaaaaaaaaaaaaaaaaaaaaaaaaatgcatcccccccccccccccccccccccccccccccaaaggctcttttcagagccaccagaatt

SEQ ID NO: 29 - RNA, 1975 nt, synthetic construct
  source 1..1975: mol_type = other RNA; note = MmDCT mRNA R8094
agggcgctgcctacggaggtggcagccatctccttctcggcatcaagcttaccatgggcctggtggggtggggcctcctgctggggtgcctcggctgcgggatcctgctgcgggcccgcgcccagttcccccgggtctgcatgaccctcgacggcgtgctgaacaaggagtgctgcccgcccctgggccccgaggcgaccaacatctgcgggttcctcgagggccgcgggcagtgcgccgaggtgcagacggacacccggccctggagcggcccgtacatcctgcgcaaccaggacgaccgggagcagtggccccgcaagttcttcaaccggacctgcaagtgcaccgggaacttcgccggctacaactgcggcgggtgcaagttcggctggacggggcccgactgcaaccgcaagaagcccgccatcctgcggcgcaacatccactccctcaccgcccaggagcgggagcagttcctgggcgcgctggacctcgccaagaagagcatccacccggactacgtcatcaccacccagcactggctgggcctgctcgggcccaacggcacgcagccccagatcgccaactgctccgtgtacgacttcttcgtgtggctgcactactacagcgtccgcgacaccctgctcgggcccggccggccgtacaaggccatcgacttctcccaccaggggcccgcgttcgtgacctggcaccgctaccacctgctgtggctcgagcgggagctgcagcgcctgaccggcaacgagagcttcgccctcccctactggaacttcgccacgggcaagaacgagtgcgacgtgtgcaccgacgagctgctgggggccgcccggcaggacgaccccaccctgatctcccgcaacagccggttctccacctgggagatcgtctgcgacagcctcgacgactacaaccgccgggtgacgctgtgcaacggcacctacgaggggctgctccggcgcaacaaggtgggccggaacaacgagaagctgccgaccctgaagaacgtccaggactgcctctccctgcagaagttcgacagcccccccttcttccagaactccaccttcagcttccgcaacgcgctggaggggttcgacaaggccgacggcacgctcgactcccaggtgatgaacctgcacaacctggcccacagcttcctcaacggcaccaacgccctgccccactccgccgcgaacgacccggtgttcgtcgtgctgcacagcttcaccgacgccatcttcgacgagtggctcaagcggaacaacccctccaccgacgcctggccccaggagctggcccccatcgggcacaaccgcatgtacaacatggtgccgttcttcccgcccgtcacgaacgaggagctgttcctgaccgccgagcagctcggctacaactacgcggtggacctgagcgaggaggaggcccccgtgtggagcaccaccctgtccgtcgtgatcgggatcctcggcgccttcgtgctgctgctcgggctgctggccttcctccagtaccggcgcctgcggaagggctacgccccgctgatggagacgggcctcagctccaagcgctacaccgaggaggcggccaagttcgtggccgcctggaccctgaaggcggccgcctgaggactagtgcatcacatttaaaagcatctcagcctaccatgagaataagagaaagaaaatgaagatcaatagcttattcatctctttttctttttcgttggtgtaaagccaacaccctgtctaaaaaacataaatttctttaatcattttgcctcttttctctgtgcttcaattaataaaaaatggaaagaacctagatctaaaaaaaaaaaaaaaaaaaaaaaaaaaaaaaaaaaaaaaaaaaaaaaaaaaaaaaaaaaaaaaatgcatcccccccccccccccccccccccccccccccaaaggctcttttcagagccaccagaatt

SEQ ID NO: 30 - RNA, 1707 nt, synthetic construct
  source 1..1707: mol_type = other RNA; note = anti-Rabies Antibody HC mRNA R6266
gggagagtcccgcagtcggcgtccagcggctctgcttgttcgtgtgtgtgtcgttgcaggccttattcaagcttaccatggactggacctggcgcttcctgttcgtggtcgccgccgcgaccggcgtgcagagccaggtgcagctcgtccagtccggggccgaggtgaagaagcccggcagctccgtgaaggtcagctgcaaggcctccgggggcacgttcaaccggtacaccgtgaactgggtgcgccaggccccggggcagggcctggagtggatgggcgggatcatccccatcttcggcaccgccaactacgcgcagcggttccaggggcgcctgaccatcacggccgacgagagcacctccaccgcctacatggagctcagctccctgcggagcgacgacaccgccgtctacttctgcgcgcgcgagaacctggacaactccggcacgtactactacttcagcgggtggttcgacccctggggccagggcaccctcgtgaccgtgtccagcgcctccaccaaggggcccagcgtcttcccgctggccccctccagcaagtccacgagcggcgggaccgccgccctgggctgcctcgtgaaggactacttccccgagcccgtgaccgtcagctggaactccggcgcgctgaccagcggggtgcacacgttcccggccgtgctgcagtccagcggcctctactccctgagctccgtcgtgaccgtgcccagctccagcctggggacccagacctacatctgcaacgtcaaccacaagccctccaacacgaaggtggacaagcgggtggagcccaagagctgcgacaagacccacacctgcccgccctgccccgccccggagctcctgggcgggccgtccgtcttcctgttccctcccaagcccaaggacaccctcatgatcagccgcacgccggaggtgacctgcgtcgtggtggacgtgtcccacgaggaccccgaggtgaagttcaactggtacgtggacggcgtcgaggtgcacaacgccaagaccaagccccgggaggagcagtacaacagcacctaccgcgtggtctccgtgctgacggtgctgcaccaggactggctcaacggcaaggagtacaagtgcaaggtcagcaacaaggccctgcccgcgccgatcgagaagaccatctccaaggccaaggggcagccccgggagccccaggtgtacaccctgcccccgagccgcgaggagatgaccaagaaccaggtgagcctcacgtgcctggtcaagggcttctacccctccgacatcgccgtggagtgggagagcaacgggcagcccgagaacaactacaagaccaccccgcccgtgctggactccgacggcagcttcttcctctactccaagctgaccgtcgacaagagccggtggcagcaggggaacgtgttctcctgcagcgtgatgcacgaggccctgcacaaccactacacgcagaagtccctcagcctgtcccccggcaagtgaggactagtccctgttcccagagcccacttttttttctttttttgaaataaaatagcctgtctttcagatctaaaaaaaaaaaaaaaaaaaaaaaaaaaaaaaaaaaaaaaaaaaaaaaaaaaaaaaaaaaaaaaatgcatcccccccccccccccccccccccccccccccaaaggctcttttcagagccaccagaatt

SEQ ID NO: 31 - RNA, 1005 nt, synthetic construct
  source 1..1005: mol_type = other RNA; note = anti-Rabies Antibody LC mRNA R6267
gggagagtcccgcagtcggcgtccagcggctctgcttgttcgtgtgtgtgtcgttgcaggccttattcaagcttaccatgagcgtgcccaccatggcctgggccctgctcctgctgtccctcctgacccagggcacggggagctgggcgcagtccgccctgacccagccgcgcagcgtctccggcagccccgggcagtccgtgaccatcagctgcaccggcacgtccagcgacatcgggggctacaacttcgtgtcctggtaccagcagcaccccggcaaggcccccaagctcatgatctacgacgccaccaagcggccgagcggggtccccgaccgcttctccggcagcaagtccgggaacaccgccagcctgaccatctccggcctgcaggcggaggacgaggccgactactactgctgcagctacgccggggactacacgcccggcgtggtgttcggcgggggcaccaagctcaccgtcctggggcagccgaaggccgcgccgtccgtgaccctgttccctcccagcagcgaggagctccaggccaacaaggccacgctggtgtgcctgatctccgacttctaccccggcgccgtcaccgtggcctggaaggcggacagctccccggtgaaggccggcgtcgagaccaccacgcccagcaagcagtccaacaacaagtacgccgccagctcctacctcagcctgacccccgagcagtggaagtcccaccggagctactcctgccaggtgacccacgaggggagcaccgtggagaagacggtcgcccccaccgagtgctcctgaggactagtccctgttcccagagcccacttttttttctttttttgaaataaaatagcctgtctttcagatctaaaaaaaaaaaaaaaaaaaaaaaaaaaaaaaaaaaaaaaaaaaaaaaaaaaaaaaaaaaaaaaatgcatcccccccccccccccccccccccccccccccaaaggctcttttcagagccaccagaatt

SEQ ID NO: 32 - RNA, 1792 nt, synthetic construct
  source 1..1792: mol_type = other RNA; note = RABV-G mRNA R1803, R7621
gggagaaagcttaccatggtgccccaggccctgctcttcgtcccgctgctggtgttccccctctgcttcggcaagttccccatctacaccatccccgacaagctggggccgtggagccccatcgacatccaccacctgtcctgccccaacaacctcgtggtcgaggacgagggctgcaccaacctgagcgggttctcctacatggagctgaaggtgggctacatcagcgccatcaagatgaacgggttcacgtgcaccggcgtggtcaccgaggcggagacctacacgaacttcgtgggctacgtgaccaccaccttcaagcggaagcacttccgccccacgccggacgcctgccgggccgcctacaactggaagatggccggggacccccgctacgaggagtccctccacaacccctaccccgactaccactggctgcggaccgtcaagaccaccaaggagagcctggtgatcatctccccgagcgtggcggacctcgacccctacgaccgctccctgcacagccgggtcttccccggcgggaactgctccggcgtggccgtgagctccacgtactgcagcaccaaccacgactacaccatctggatgcccgagaacccgcgcctggggatgtcctgcgacatcttcaccaacagccggggcaagcgcgcctccaagggcagcgagacgtgcgggttcgtcgacgagcggggcctctacaagtccctgaagggggcctgcaagctgaagctctgcggcgtgctgggcctgcgcctcatggacgggacctgggtggcgatgcagaccagcaacgagaccaagtggtgcccccccggccagctggtcaacctgcacgacttccggagcgacgagatcgagcacctcgtggtggaggagctggtcaagaagcgcgaggagtgcctggacgccctcgagtccatcatgacgaccaagagcgtgtccttccggcgcctgagccacctgcggaagctcgtgcccgggttcggcaaggcctacaccatcttcaacaagaccctgatggaggccgacgcccactacaagtccgtccgcacgtggaacgagatcatcccgagcaaggggtgcctgcgggtgggcggccgctgccacccccacgtcaacggggtgttcttcaacggcatcatcctcgggcccgacggcaacgtgctgatccccgagatgcagtccagcctgctccagcagcacatggagctgctggtctccagcgtgatcccgctcatgcaccccctggcggacccctccaccgtgttcaagaacggggacgaggccgaggacttcgtcgaggtgcacctgcccgacgtgcacgagcggatcagcggcgtcgacctcggcctgccgaactgggggaagtacgtgctgctctccgccggcgccctgaccgccctgatgctgatcatcttcctcatgacctgctggcgccgggtgaaccggagcgagcccacgcagcacaacctgcgcgggaccggccgggaggtctccgtgaccccgcagagcgggaagatcatctccagctgggagtcctacaagagcggcggcgagaccgggctgtgaggactagttataagactgactagcccgatgggcctcccaacgggccctcctcccctccttgcaccgagattaataaaaaaaaaaaaaaaaaaaaaaaaaaaaaaaaaaaaaaaaaaaaaaaaaaaaaaaaaaaaaaaatgcatcccccccccccccccccccccccccccccccaaaggctcttttcagagccaccagaatt

SEQ ID NO: 33 - RNA, 1870 nt, synthetic construct
  source 1..1870: mol_type = other RNA; note = PpLuc mRNA R6598
aggagaaagcttaccatggaggacgccaagaacatcaagaagggcccggcgcccttctacccgctggaggacgggaccgccggcgagcagctccacaaggccatgaagcggtacgccctggtgccgggcacgatcgccttcaccgacgcccacatcgaggtcgacatcacctacgcggagtacttcgagatgagcgtgcgcctggccgaggccatgaagcggtacggcctgaacaccaaccaccggatcgtggtgtgctcggagaacagcctgcagttcttcatgccggtgctgggcgccctcttcatcggcgtggccgtcgccccggcgaacgacatctacaacgagcgggagctgctgaacagcatggggatcagccagccgaccgtggtgttcgtgagcaagaagggcctgcagaagatcctgaacgtgcagaagaagctgcccatcatccagaagatcatcatcatggacagcaagaccgactaccagggcttccagtcgatgtacacgttcgtgaccagccacctcccgccgggcttcaacgagtacgacttcgtcccggagagcttcgaccgggacaagaccatcgccctgatcatgaacagcagcggcagcaccggcctgccgaagggggtggccctgccgcaccggaccgcctgcgtgcgcttctcgcacgcccgggaccccatcttcggcaaccagatcatcccggacaccgccatcctgagcgtggtgccgttccaccacggcttcggcatgttcacgaccctgggctacctcatctgcggcttccgggtggtcctgatgtaccggttcgaggaggagctgttcctgcggagcctgcaggactacaagatccagagcgcgctgctcgtgccgaccctgttcagcttcttcgccaagagcaccctgatcgacaagtacgacctgtcgaacctgcacgagatcgccagcgggggcgccccgctgagcaaggaggtgggcgaggccgtggccaagcggttccacctcccgggcatccgccagggctacggcctgaccgagaccacgagcgcgatcctgatcacccccgagggggacgacaagccgggcgccgtgggcaaggtggtcccgttcttcgaggccaaggtggtggacctggacaccggcaagaccctgggcgtgaaccagcggggcgagctgtgcgtgcgggggccgatgatcatgagcggctacgtgaacaacccggaggccaccaacgccctcatcgacaaggacggctggctgcacagcggcgacatcgcctactgggacgaggacgagcacttcttcatcgtcgaccggctgaagtcgctgatcaagtacaagggctaccaggtggcgccggccgagctggagagcatcctgctccagcaccccaacatcttcgacgccggcgtggccgggctgccggacgacgacgccggcgagctgccggccgcggtggtggtgctggagcacggcaagaccatgacggagaaggagatcgtcgactacgtggccagccaggtgaccaccgccaagaagctgcggggcggcgtggtgttcgtggacgaggtcccgaagggcctgaccgggaagctcgacgcccggaagatccgcgagatcctgatcaaggccaagaagggcggcaagatcgccgtgtgaggactagttataagactgactagcccgatgggcctcccaacgggccctcctcccctccttgcaccgagattaataaaaaaaaaaaaaaaaaaaaaaaaaaaaaaaaaaaaaaaaaaaaaaaaaaaaaaaaaaaaaaaatgcatcccccccccccccccccccccccccccccccaaaggctcttttcagagccaccagaatt

SEQ ID NO: 34 - RNA, 727 nt, synthetic construct
  source 1..727: mol_type = other RNA; note = Monoepitope construct MmPmel(14-43) mRNA R8118
aggagaaagcttaccatggcctgcctgggcttccagcgccacaaggcccagctcaacctggcgacccggacctggccctgcacgctcctgttcttcctgctgttcatcccggtgttctgcggcgggggtggaagcggcgggggtggaagtctgagcgccctcctggccgtgggcgcgctggaggggccccgcaaccaggactggctcggcgtcccgcggcagctggtgaccaagaccggaggtggcgggtctggaggtggcgggtcagccaagttcgtggccgcgtggaccctgaaggccgccgccggtggcggagggtcgggtggcggagggtccttcctgctctggatcctggccgccgtgagctccggcctgttcttctacagcttcctcctgaccgcggtctccctgagcaagatgctcaagaagcgctcccccctgaccacgggggtgtacgtgaagatgccgcccaccgagcccgagtgcgagaagcagttccagccctacttcatcccgatcaactgaggactagttataagactgactagcccgatgggcctcccaacgggccctcctcccctccttgcaccgagattaataaaaaaaaaaaaaaaaaaaaaaaaaaaaaaaaaaaaaaaaaaaaaaaaaaaaaaaaaaaaaaaatgcatcccccccccccccccccccccccccccccccaaaggctcttttcagagccaccagaatt

SEQ ID NO: 35 - RNA, 727 nt, synthetic construct
  source 1..727: mol_type = other RNA; note = Monoepitope construct MmTrp1(455-473) mRNA R7858
aggagaaagcttaccatggcctgcctgggcttccagcgccacaaggcccagctcaacctggcgacccggacctggccctgcacgctcctgttcttcctgctgttcatcccggtgttctgcggcgggggtggaagcggcgggggtggaagtcccccggtgaccaacaccgagatgttcgtcacggcccccgacaacctgggctacatgtacgaggtgcagtggcccgggcaggagttcggaggtggcgggtctggaggtggcgggtcagccaagttcgtggccgcgtggaccctgaaggccgccgccggtggcggagggtcgggtggcggagggtccttcctgctctggatcctggccgccgtgagctccggcctgttcttctacagcttcctcctgaccgcggtctccctgagcaagatgctcaagaagcgctcccccctgaccacgggggtgtacgtgaagatgccgcccaccgagcccgagtgcgagaagcagttccagccctacttcatcccgatcaactgaggactagttataagactgactagcccgatgggcctcccaacgggccctcctcccctccttgcaccgagattaataaaaaaaaaaaaaaaaaaaaaaaaaaaaaaaaaaaaaaaaaaaaaaaaaaaaaaaaaaaaaaaatgcatcccccccccccccccccccccccccccccccaaaggctcttttcagagccaccagaatt

SEQ ID NO: 36 - RNA, 727 nt, synthetic construct
  source 1..727: mol_type = other RNA; note = Monoepitope construct MmObsl1(1750-1778) mRNA R7860
aggagaaagcttaccatggcctgcctgggcttccagcgccacaaggcccagctcaacctggcgacccggacctggccctgcacgctcctgttcttcctgctgttcatcccggtgttctgcggcgggggtggaagcggcgggggtggaagtatgcgcgagggcgtggagctgtgccccgggaacaagtacgagatgcggcgccacggcaccacccacagcctcgtcatccacgacgtgggaggtggcgggtctggaggtggcgggtcagccaagttcgtggccgcgtggaccctgaaggccgccgccggtggcggagggtcgggtggcggagggtccttcctgctctggatcctggccgccgtgagctccggcctgttcttctacagcttcctcctgaccgcggtctccctgagcaagatgctcaagaagcgctcccccctgaccacgggggtgtacgtgaagatgccgcccaccgagcccgagtgcgagaagcagttccagccctacttcatcccgatcaactgaggactagttataagactgactagcccgatgggcctcccaacgggccctcctcccctccttgcaccgagattaataaaaaaaaaaaaaaaaaaaaaaaaaaaaaaaaaaaaaaaaaaaaaaaaaaaaaaaaaaaaaaaatgcatcccccccccccccccccccccccccccccccaaaggctcttttcagagccaccagaatt

SEQ ID NO: 37 - RNA, 1918 nt, synthetic construct
  source 1..1918: mol_type = other RNA; note = H3N2-HA mRNA R4936
gggagaaagcttaccatgaagaccatcatcgccctgagctacatcctctgcctggtgttcgcccagaagatccccggcaacgacaactccaccgcgacgctgtgcctcgggcaccacgccgtcccgaacggcaccatcgtgaagaccatcaccaacgaccgcatcgaggtgacgaacgccaccgagctggtccagaacagctccatcggggagatctgcgacagcccccaccagatcctggacggcgagaactgcaccctcatcgacgccctgctgggggacccccagtgcgacggcttccagaacaagaagtgggacctcttcgtggagcggtccaaggcctacagcaactgctacccctacgacgtgccggactacgcgtccctgcgcagcctggtcgcctccagcggcaccctcgagttcaacaacgagtccttcaactggacgggggtgacccagaacggcaccagctccgcctgcatccggcgcagctccagctccttcttcagccggctgaactggctgacccacctcaactacaagtaccccgccctgaacgtgacgatgcccaacaacgagcagttcgacaagctgtacatctggggcgtccaccaccccgggaccgacaaggaccagatcttcccgtacgcgcagtccagcgggcgcatcatcgtgagcaccaagcggtcccagcaggccgtgatccccaacatcggcagccgcccccggatccgcgacatcccgtcccggatcagcatctactggaccatcgtcaagccgggcgacatcctcctgatcaactccacggggaacctgatcgccccgcgcggctacttcaagctccggagcgggaagtccagcatcatgcgctccgacgcccccatcggcaagtgcaagagcgagtgcatcacccccaacggctccatcccgaacgacaagcccttccagaacgtgaaccggatcacctacggggcctgcccccgctacgtgaagcacagcaccctgaagctggcgacgggcatgcggaacgtccccgagaagcagacccgcgggatcttcggcgccatcgccgggttcatcgagaacggctgggagggcatggtggacgggtggtacggcttccggcaccagaactccgaggggcgcggccaggccgccgacctcaagagcacccaggcggccatcgaccagatcaacgggaagctgaaccggctgatcggcaagaccaacgagaagttccaccagatcgagaaggagttctccgaggtggagggccgcatccaggacctcgagaagtacgtcgaggacacgaagatcgacctgtggagctacaacgccgagctgctcgtggccctggagaaccagcacaccatcgacctgaccgactccgagatgaacaagctcttcgagaagaccaagaagcagctgcgcgagaacgccgaggacatggggaacggctgcttcaagatctaccacaagtgcgacaacgcgtgcatcgggagcatccggaacggcacgtacgaccacaacgtgtaccgcgacgaggccctgaacaaccggttccagatcaagggggtcgagctcaagtccggctacaaggactggatcctgtggatcagcttcgccatcagctgcttcctgctctgcgtggccctgctgggcttcatcatgtgggcctgccagaaggggaacatccgctgcaacatctgcatctgaggactagttataagactgactagcccgatgggcctcccaacgggccctcctcccctccttgcaccgagattaataaaaaaaaaaaaaaaaaaaaaaaaaaaaaaaaaaaaaaaaaaaaaaaaaaaaaaaaaaaaaaaatgcatcccccccccccccccccccccccccccccccaaaggctcttttcagagccaccagaatt

SEQ ID NO: 38 - RNA, 1470 nt, synthetic construct
  source 1..1470: mol_type = other RNA; note = PfCSP mRNA R7111
gggagagtcccgcagtcggcgtccagcggctctgcttgttcgtgtgtgtgtcgttgcaggccttattcaagcttaccatgatgcgcaagctggccatcctcagcgtgtccagcttcctgttcgtcgaggccctgttccaggagtaccagtgctacggctccagctccaacacccgggtgctcaacgagctgaactacgacaacgcggggaccaacctgtacaacgagctcgagatgaactactacggcaagcaggagaactggtacagcctgaagaagaactcccgcagcctgggggagaacgacgacggcaacaacgaggacaacgagaagctccggaagcccaagcacaagaagctgaagcagccggccgacgggaaccccgaccccaacgccaaccccaacgtggacccgaacgccaaccccaacgtcgaccccaacgccaaccccaacgtggacccgaacgcgaaccccaacgccaaccccaacgccaaccccaacgccaacccgaacgcgaaccccaacgccaaccccaacgccaaccccaacgccaacccgaacgccaaccccaacgcgaaccccaacgccaaccccaacgccaacccgaacgccaaccccaacgccaaccccaacgcgaaccccaacgccaacccgaacgccaaccccaacgtggaccccaacgccaaccccaacgccaacccgaacgcgaaccccaacgccaaccccaacgccaacccgaacgccaaccccaacgccaaccccaacgcgaaccccaacgccaacccgaacgccaaccccaacgccaaccccaacgcgaaccccaacgccaacccgaacgccaaccccaacgccaaccccaacgccaaccccaacgcgaacccgaacgccaaccccaacaagaacaaccagggcaacggccaggggcacaacatgcccaacgaccccaaccgcaacgtcgacgagaacgccaacgccaactccgccgtgaagaacaacaacaacgaggagccgagcgacaagcacatcaaggagtacctgaacaagatccagaactccctcagcacggagtggtccccctgcagcgtgacctgcggcaacgggatccaggtccggatcaagcccggctccgcgaacaagcccaaggacgagctggactacgccaacgacatcgagaagaagatctgcaagatggagaagtgcagctccgtgttcaacgtggtcaacagctccatcgggctgatcatggtgctcagcttcctgttcctgaactgaggactagtccctgttcccagagcccacttttttttctttttttgaaataaaatagcctgtctttcagatctaaaaaaaaaaaaaaaaaaaaaaaaaaaaaaaaaaaaaaaaaaaaaaaaaaaaaaaaaaaaaaaatgcatcccccccccccccccccccccccccccccccaaaggctcttttcagagccaccagaatt

SEQ ID NO: 39 - RNA, 847 nt, synthetic construct
  source 1..847: mol_type = other RNA; note = HsFGF21 mRNA R7970
aggagagtcccgcagtcggcgtccagcggctctgcttgttcgtgtgtgtgtcgttgcaggccttattcaagcttaccatggactcggacgagaccgggttcgagcactcaggactgtgggtttctgtgctggctggtcttctgctgggagcctgccaggcacaccccatccctgactccagtcctctcctgcaattcgggggccaagtccggcagcggtacctctacacagatgatgcccagcagacagaagcccacctggagatcagggaggatgggacggtggggggcgctgctgaccagagccccgaaagtctcctgcagctgaaagccttgaagccgggagttattcaaatcttgggagtcaagacatccaggttcctgtgccagcggccagatggggccctgtatggatcgctccactttgaccctgaggcctgcagcttccgggagcggcttcttgaggacggatacaatgtttaccagtccgaagcccacggcctcccgctgcacctcccagggaacaagtccccacaccgggaccctgcaccccgaggaccagctcgcttcctgccactaccaggcctgccccccgcactcccggagccacccggaatcctggccccccagccccccgatgtgggctcctcggaccctctgagcatggtgggaggttcccagggccgaagccccagctacgagtcctgaggactagtccctgttcccagagcccacttttttttctttttttgaaataaaatagcctgtctttcagatctaaaaaaaaaaaaaaaaaaaaaaaaaaaaaaaaaaaaaaaaaaaaaaaaaaaaaaaaaaaaaaaagaatt

SEQ ID NO: 40 - RNA, 1470 nt, synthetic construct
  source 1..1470: mol_type = other RNA; note = PfCSP mRNA R8143
aggagagtcccgcagtcggcgtccagcggctctgcttgttcgtgtgtgtgtcgttgcaggccttattcaagcttaccatgatgcgcaagctggccatcctcagcgtgtccagcttcctgttcgtcgaggccctgttccaggagtaccagtgctacggctccagctccaacacccgggtgctcaacgagctgaactacgacaacgcggggaccaacctgtacaacgagctcgagatgaactactacggcaagcaggagaactggtacagcctgaagaagaactcccgcagcctgggggagaacgacgacggcaacaacgaggacaacgagaagctccggaagcccaagcacaagaagctgaagcagccggccgacgggaaccccgaccccaacgccaaccccaacgtggacccgaacgccaaccccaacgtcgaccccaacgccaaccccaacgtggacccgaacgcgaaccccaacgccaaccccaacgccaaccccaacgccaacccgaacgcgaaccccaacgccaaccccaacgccaaccccaacgccaacccgaacgccaaccccaacgcgaaccccaacgccaaccccaacgccaacccgaacgccaaccccaacgccaaccccaacgcgaaccccaacgccaacccgaacgccaaccccaacgtggaccccaacgccaaccccaacgccaacccgaacgcgaaccccaacgccaaccccaacgccaacccgaacgccaaccccaacgccaaccccaacgcgaaccccaacgccaacccgaacgccaaccccaacgccaaccccaacgcgaaccccaacgccaacccgaacgccaaccccaacgccaaccccaacgccaaccccaacgcgaacccgaacgccaaccccaacaagaacaaccagggcaacggccaggggcacaacatgcccaacgaccccaaccgcaacgtcgacgagaacgccaacgccaactccgccgtgaagaacaacaacaacgaggagccgagcgacaagcacatcaaggagtacctgaacaagatccagaactccctcagcacggagtggtccccctgcagcgtgacctgcggcaacgggatccaggtccggatcaagcccggctccgcgaacaagcccaaggacgagctggactacgccaacgacatcgagaagaagatctgcaagatggagaagtgcagctccgtgttcaacgtggtcaacagctccatcgggctgatcatggtgctcagcttcctgttcctgaactgaggactagtccctgttcccagagcccacttttttttctttttttgaaataaaatagcctgtctttcagatctaaaaaaaaaaaaaaaaaaaaaaaaaaaaaaaaaaaaaaaaaaaaaaaaaaaaaaaaaaaaaaaatgcatcccccccccccccccccccccccccccccccaaaggctcttttcagagccaccagaatt

SEQ ID NO: 41 - RNA, 128 nt, synthetic construct
  source 1..128: mol_type = other RNA; note = mRNA 3'-end A64-N5-C30-histoneSL-N5
aaaaaaaaaaaaaaaaaaaaaaaaaaaaaaaaaaaaaaaaaaaaaaaaaaaaaaaaaaaaaaaatgcatcccccccccccccccccccccccccccccccaaaggctcttttcagagccaccagaatt

SEQ ID NO: 42 - RNA, 129 nt, synthetic construct
  source 1..129: mol_type = other RNA; note = mRNA 3'-end histoneSL-A100-N5
caaaggctcttttcagagccaccaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaagaatt

SEQ ID NO: 43 - RNA, 93 nt, synthetic construct
  source 1..93: mol_type = other RNA; note = mRNA 3'-end histoneSL-A64-N5
caaaggctcttttcagagccaccaaaaaaaaaaaaaaaaaaaaaaaaaaaaaaaaaaaaaaaaaaaaaaaaaaaaaaaaaaaaaaaaagaatt

SEQ ID NO: 44 - RNA, 105 nt, synthetic construct
  source 1..105: mol_type = other RNA; note = mRNA 3'-end A100-N5
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaagaatt

SEQ ID NO: 45 - RNA, 69 nt, synthetic construct
  source 1..69: mol_type = other RNA; note = mRNA 3'-end A64-N5
aaaaaaaaaaaaaaaaaaaaaaaaaaaaaaaaaaaaaaaaaaaaaaaaaaaaaaaaaaaaaaaagaatt

SEQ ID NO: 46 - RNA, 124 nt, synthetic construct
  source 1..124: mol_type = other RNA; note = mRNA 3'-end histoneSL-A100
caaaggctcttttcagagccaccaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 47 - RNA, 88 nt, synthetic construct
  source 1..88: mol_type = other RNA; note = mRNA 3'-end histoneSL-A64
caaaggctcttttcagagccaccaaaaaaaaaaaaaaaaaaaaaaaaaaaaaaaaaaaaaaaaaaaaaaaaaaaaaaaaaaaaaaaaa

SEQ ID NO: 48 - RNA, 100 nt, synthetic construct
  source 1..100: mol_type = other RNA; note = mRNA 3'-end A100
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 49 - RNA, 64 nt, synthetic construct
  source 1..64: mol_type = other RNA; note = mRNA 3'-end A64
aaaaaaaaaaaaaaaaaaaaaaaaaaaaaaaaaaaaaaaaaaaaaaaaaaaaaaaaaaaaaaaa

SEQ ID NO: 50 - RNA, 93 nt, synthetic construct
  source 1..93: mol_type = other RNA; note = mRNA 3'-end A64-histoneSL-N5
aaaaaaaaaaaaaaaaaaaaaaaaaaaaaaaaaaaaaaaaaaaaaaaaaaaaaaaaaaaaaaaacaaaggctcttttcagagccaccagaatt

SEQ ID NO: 51 - RNA, 134 nt, synthetic construct
  source 1..134: mol_type = other RNA; note = mRNA 3'-end N6-A64-N5-C30-histoneSL-N5
attaataaaaaaaaaaaaaaaaaaaaaaaaaaaaaaaaaaaaaaaaaaaaaaaaaaaaaaaaaaaaaaaatgcatcccccccccccccccccccccccccccccccaaaggctcttttcagagccaccagaatt

SEQ ID NO: 52 - RNA, 134 nt, synthetic construct
  source 1..134: mol_type = other RNA; note = mRNA 3'-end N6-A64-N5-C30-histoneSL-N5
agatctaaaaaaaaaaaaaaaaaaaaaaaaaaaaaaaaaaaaaaaaaaaaaaaaaaaaaaaaaaaaaaaatgcatcccccccccccccccccccccccccccccccaaaggctcttttcagagccaccagaatt

SEQ ID NO: 53 - RNA, 135 nt, synthetic construct
  source 1..135: mol_type = other RNA; note = mRNA 3'-end N6-histoneSL-A100-N5
attaatcaaaggctcttttcagagccaccaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaagaatt

SEQ ID NO: 54 - RNA, 135 nt, synthetic construct
  source 1..135: mol_type = other RNA; note = mRNA 3'-end N6-histoneSL-A100-N5
agatctcaaaggctcttttcagagccaccaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaagaatt

SEQ ID NO: 55 - RNA, 99 nt, synthetic construct
  source 1..99: mol_type = other RNA; note = mRNA 3'-end N6-histoneSL-A64-N5
attaatcaaaggctcttttcagagccaccaaaaaaaaaaaaaaaaaaaaaaaaaaaaaaaaaaaaaaaaaaaaaaaaaaaaaaaaaaaaaaaaagaatt

SEQ ID NO: 56 - RNA, 99 nt, synthetic construct
  source 1..99: mol_type = other RNA; note = mRNA 3'-end N6-histoneSL-A64-N5
agatctcaaaggctcttttcagagccaccaaaaaaaaaaaaaaaaaaaaaaaaaaaaaaaaaaaaaaaaaaaaaaaaaaaaaaaaaaaaaaaaagaatt

SEQ ID NO: 57 - RNA, 111 nt, synthetic construct
  source 1..111: mol_type = other RNA; note = mRNA 3'-end N6-A100-N5
attaataaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaagaatt

SEQ ID NO: 58 - RNA, 111 nt, synthetic construct
  source 1..111: mol_type = other RNA; note = mRNA 3'-end N6-A100-N5
agatctaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaagaatt

SEQ ID NO: 59 - RNA, 75 nt, synthetic construct
  source 1..75: mol_type = other RNA; note = mRNA 3'-end N6-A64-N5
attaataaaaaaaaaaaaaaaaaaaaaaaaaaaaaaaaaaaaaaaaaaaaaaaaaaaaaaaaaaaaaaaagaatt

SEQ ID NO: 60 - RNA, 75 nt, synthetic construct
  source 1..75: mol_type = other RNA; note = mRNA 3'-end N6-A64-N5
agatctaaaaaaaaaaaaaaaaaaaaaaaaaaaaaaaaaaaaaaaaaaaaaaaaaaaaaaaaaaaaaaaagaatt

SEQ ID NO: 61 - RNA, 130 nt, synthetic construct
  source 1..130: mol_type = other RNA; note = mRNA 3'-end N6-histoneSL-A100
attaatcaaaggctcttttcagagccaccaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 62 - RNA, 130 nt, synthetic construct
  source 1..130: mol_type = other RNA; note = mRNA 3'-end N6-histoneSL-A100
agatctcaaaggctcttttcagagccaccaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 63 - RNA, 94 nt, synthetic construct
  source 1..94: mol_type = other RNA; note = mRNA 3'-end N6-histoneSL-A64
attaatcaaaggctcttttcagagccaccaaaaaaaaaaaaaaaaaaaaaaaaaaaaaaaaaaaaaaaaaaaaaaaaaaaaaaaaaaaaaaaaa

SEQ ID NO: 64 - RNA, 94 nt, synthetic construct
  source 1..94: mol_type = other RNA; note = mRNA 3'-end N6-histoneSL-A64
agatctcaaaggctcttttcagagccaccaaaaaaaaaaaaaaaaaaaaaaaaaaaaaaaaaaaaaaaaaaaaaaaaaaaaaaaaaaaaaaaaa

SEQ ID NO: 65 - RNA, 106 nt, synthetic construct
  source 1..106: mol_type = other RNA; note = mRNA 3'-end N6-A100
attaataaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 66 - RNA, 106 nt, synthetic construct
  source 1..106: mol_type = other RNA; note = mRNA 3'-end N6-A100
agatctaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 67 - RNA, 70 nt, synthetic construct
  source 1..70: mol_type = other RNA; note = mRNA 3'-end N6-A64
attaataaaaaaaaaaaaaaaaaaaaaaaaaaaaaaaaaaaaaaaaaaaaaaaaaaaaaaaaaaaaaaaa

SEQ ID NO: 68 - RNA, 70 nt, synthetic construct
  source 1..70: mol_type = other RNA; note = mRNA 3'-end N6-A64
agatctaaaaaaaaaaaaaaaaaaaaaaaaaaaaaaaaaaaaaaaaaaaaaaaaaaaaaaaaaaaaaaaa

SEQ ID NO: 69 - RNA, 99 nt, synthetic construct
  source 1..99: mol_type = other RNA; note = mRNA 3'-end N6-A64-histoneSL-N5
attaataaaaaaaaaaaaaaaaaaaaaaaaaaaaaaaaaaaaaaaaaaaaaaaaaaaaaaaaaaaaaaaacaaaggctcttttcagagccaccagaatt

SEQ ID NO: 70 - RNA, 99 nt, synthetic construct
  source 1..99: mol_type = other RNA; note = mRNA 3'-end N6-A64-histoneSL-N5
agatctaaaaaaaaaaaaaaaaaaaaaaaaaaaaaaaaaaaaaaaaaaaaaaaaaaaaaaaaaaaaaaaacaaaggctcttttcagagccaccagaatt

SEQ ID NO: 71 - RNA, 1925 nt, synthetic construct
  source 1..1925: mol_type = other RNA; note = PpLuc mRNA R8730
aggagagtcccgcagtcggcgtccagcggctctgcttgttcgtgtgtgtgtcgttgcaggccttattcaagcttaccatggaggacgccaagaacatcaagaagggcccggcgcccttctacccgctggaggacgggaccgccggcgagcagctccacaaggccatgaagcggtacgccctggtgccgggcacgatcgccttcaccgacgcccacatcgaggtcgacatcacctacgcggagtacttcgagatgagcgtgcgcctggccgaggccatgaagcggtacggcctgaacaccaaccaccggatcgtggtgtgctcggagaacagcctgcagttcttcatgccggtgctgggcgccctcttcatcggcgtggccgtcgccccggcgaacgacatctacaacgagcgggagctgctgaacagcatggggatcagccagccgaccgtggtgttcgtgagcaagaagggcctgcagaagatcctgaacgtgcagaagaagctgcccatcatccagaagatcatcatcatggacagcaagaccgactaccagggcttccagtcgatgtacacgttcgtgaccagccacctcccgccgggcttcaacgagtacgacttcgtcccggagagcttcgaccgggacaagaccatcgccctgatcatgaacagcagcggcagcaccggcctgccgaagggggtggccctgccgcaccggaccgcctgcgtgcgcttctcgcacgcccgggaccccatcttcggcaaccagatcatcccggacaccgccatcctgagcgtggtgccgttccaccacggcttcggcatgttcacgaccctgggctacctcatctgcggcttccgggtggtcctgatgtaccggttcgaggaggagctgttcctgcggagcctgcaggactacaagatccagagcgcgctgctcgtgccgaccctgttcagcttcttcgccaagagcaccctgatcgacaagtacgacctgtcgaacctgcacgagatcgccagcgggggcgccccgctgagcaaggaggtgggcgaggccgtggccaagcggttccacctcccgggcatccgccagggctacggcctgaccgagaccacgagcgcgatcctgatcacccccgagggggacgacaagccgggcgccgtgggcaaggtggtcccgttcttcgaggccaaggtggtggacctggacaccggcaagaccctgggcgtgaaccagcggggcgagctgtgcgtgcgggggccgatgatcatgagcggctacgtgaacaacccggaggccaccaacgccctcatcgacaaggacggctggctgcacagcggcgacatcgcctactgggacgaggacgagcacttcttcatcgtcgaccggctgaagtcgctgatcaagtacaagggctaccaggtggcgccggccgagctggagagcatcctgctccagcaccccaacatcttcgacgccggcgtggccgggctgccggacgacgacgccggcgagctgccggccgcggtggtggtgctggagcacggcaagaccatgacggagaaggagatcgtcgactacgtggccagccaggtgaccaccgccaagaagctgcggggcggcgtggtgttcgtggacgaggtcccgaagggcctgaccgggaagctcgacgcccggaagatccgcgagatcctgatcaaggccaagaagggcggcaagatcgccgtgtgaggactagtccctgttcccagagcccacttttttttctttttttgaaataaaatagcctgtctttcagatctcaaaggctcttttcagagccaccaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 72 - RNA, 1631 nt, synthetic construct
  source 1..1631: mol_type = other RNA; note = HsPAH mRNA R8844
aggagagtcccgcagtcggcgtccagcggctctgcttgttcgtgtgtgtgtcgttgcaggccttattcaagcttaccatgagcaccgccgtgctggagaaccccggcctcgggcgcaagctgtccgacttcggccaggagaccagctacatcgaggacaactgcaaccagaacggggccatctccctgatcttcagcctcaaggaggaggtcggcgcgctggccaaggtgctgcggctgttcgaggagaacgacgtgaacctgacgcacatcgagtcccgcccgagccggctgaagaaggacgagtacgagttcttcacccacctcgacaagcgctccctgcccgccctgaccaacatcatcaagatcctccggcacgacatcggggccaccgtccacgagctgagccgcgacaagaagaaggacacggtgccctggttcccccggaccatccaggagctggaccgcttcgccaaccagatcctctcctacggcgcggagctggacgccgaccacccgggcttcaaggaccccgtgtaccgggcccgccggaagcagttcgccgacatcgcgtacaactaccgccacgggcagcccatcccccgggtcgagtacatggaggaggagaagaagacctggggcaccgtgttcaagacgctcaagagcctctacaagacccacgcctgctacgagtacaaccacatcttcccgctgctggagaagtactgcgggttccacgaggacaacatcccccagctcgaggacgtgtcccagttcctgcagacctgcaccggcttccgcctgcggcccgtcgccgggctcctgagctcccgcgacttcctgggcggcctcgccttccgggtgttccactgcacgcagtacatccgccacgggagcaagcccatgtacaccccggagcccgacatctgccacgagctgctgggccacgtgcccctcttctccgaccggagcttcgcccagttctcccaggagatcgggctggcgagcctgggcgcccccgacgagtacatcgagaagctcgccaccatctactggttcaccgtcgagttcggcctgtgcaagcagggggactccatcaaggcctacggcgccgggctgctcagcagcttcggcgagctgcagtactgcctgtccgagaagccgaagctcctgcccctggagctcgagaagacggcgatccagaactacaccgtgaccgagttccagcccctgtactacgtggccgagagcttcaacgacgccaaggagaaggtccgcaacttcgccgccaccatcccccggccgttctccgtgcgctacgacccctacacgcagcgcatcgaggtgctggacaacacccagcagctcaagatcctggcggacagcatcaactccgagatcgggatcctgtgcagcgccctgcagaagatcaagtgaggactagtccctgttcccagagcccacttttttttctttttttgaaataaaatagcctgtctttcagatctcaaaggctcttttcagagccaccaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 73 - RNA, 1538 nt, synthetic construct
  source 1..1538: mol_type = other RNA; note = HsGLA mRNA R8037
aggagagtcccgcagtcggcgtccagcggctctgcttgttcgtgtgtgtgtcgttgcaggccttattcaagcttaccatgcagctgcgcaaccccgagctccacctgggctgcgccctggccctccggttcctggcgctggtgagctgggacatcccgggggcccgcgccctcgacaacggcctggcccggacccccaccatggggtggctgcactgggagcgcttcatgtgcaacctcgactgccaggaggagcccgactcctgcatcagcgagaagctgttcatggagatggccgagctgatggtctccgagggctggaaggacgcggggtacgagtacctctgcatcgacgactgctggatggcgccccagcgggacagcgagggccgcctgcaggccgacccgcagcggttcccccacggcatccgccagctggccaactacgtgcactccaaggggctcaagctgggcatctacgccgacgtggggaacaagacgtgcgccggcttccccgggagcttcggctactacgacatcgacgcccagaccttcgccgactggggcgtcgacctgctcaagttcgacgggtgctactgcgactccctggagaacctggccgacggctacaagcacatgagcctcgcgctgaaccggaccgggcgctccatcgtgtacagctgcgagtggcccctgtacatgtggccgttccagaagcccaactacaccgagatccggcagtactgcaaccactggcgcaacttcgccgacatcgacgactcctggaagtccatcaagtccatcctcgactggacgagcttcaaccaggagcggatcgtggacgtcgccggccccggcgggtggaacgaccccgacatgctggtgatcggcaacttcgggctgtcctggaaccagcaggtcacccagatggccctctgggccatcatggcggccccgctgttcatgagcaacgacctgcgccacatcagcccccaggccaaggccctcctgcaggacaaggacgtgatcgccatcaaccaggaccccctgggcaagcaggggtaccagctccggcagggcgacaacttcgaggtgtgggagcgccccctgagcggcctggcgtgggccgtggccatgatcaaccggcaggagatcgggggcccgcgctcctacaccatcgccgtcgccagcctcgggaagggcgtggcgtgcaaccccgcctgcttcatcacccagctgctgcccgtgaagcggaagctcgggttctacgagtggacgagccgcctgcggtcccacatcaaccccaccggcaccgtcctgctccagctggagaacaccatgcagatgagcctgaaggacctcctgtgaggactagtccctgttcccagagcccacttttttttctttttttgaaataaaatagcctgtctttcagatctaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 74 - RNA, 878 nt, synthetic construct
  source 1..878: mol_type = other RNA; note = HsFGF21 mRNA R9659
aggagagtcccgcagtcggcgtccagcggctctgcttgttcgtgtgtgtgtcgttgcaggccttattcaagcttaccatggactcggacgagaccgggttcgagcactcaggactgtgggtttctgtgctggctggtcttctgctgggagcctgccaggcacaccccatccctgactccagtcctctcctgcaattcgggggccaagtccggcagcggtacctctacacagatgatgcccagcagacagaagcccacctggagatcagggaggatgggacggtggggggcgctgctgaccagagccccgaaagtctcctgcagctgaaagccttgaagccgggagttattcaaatcttgggagtcaagacatccaggttcctgtgccagcggccagatggggccctgtatggatcgctccactttgaccctgaggcctgcagcttccgggagcggcttcttgaggacggatacaatgtttaccagtccgaagcccacggcctcccgctgcacctcccagggaacaagtccccacaccgggaccctgcaccccgaggaccagctcgcttcctgccactaccaggcctgccccccgcactcccggagccacccggaatcctggccccccagccccccgatgtgggctcctcggaccctctgagcatggtgggaggttcccagggccgaagccccagctacgagtcctgaggactagtccctgttcccagagcccacttttttttctttttttgaaataaaatagcctgtctttcagatctaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 75 - DNA, 84 bp, Mus musculus
  source 1..84: mol_type = genomic DNA; note = Rpl31 5'-UTR
cccgtgacccggaagttgtacggctacgcgactttccctcccacaaaccctcgcgcccttcctttcctacttgggcccggcaga

SEQ ID NO: 76 - RNA, 84 nt, Mus musculus
  source 1..84: mol_type = mRNA; note = Rpl31 5'-UTR
cccgtgacccggaagttgtacggctacgcgactttccctcccacaaaccctcgcgcccttcctttcctacttgggcccggcaga

SEQ ID NO: 77 - DNA, 159 bp, Mus musculus
  source 1..159: mol_type = genomic DNA; note = Slc7a3 5'-UTR
gggcgcttggcttgcaaggaccctgagctgcggcattgaagcacacccaacccaactcgactgaagtcagcctcactgaaccggatctgagaatcttctctctctgggcttgccagggctctccgaacctagctagcatcctcttcaattccaactaga

SEQ ID NO: 78 - RNA, 159 nt, Mus musculus
  source 1..159: mol_type = mRNA; note = Slc7a3 5'-UTR
gggcgcttggcttgcaaggaccctgagctgcggcattgaagcacacccaacccaactcgactgaagtcagcctcactgaaccggatctgagaatcttctctctctgggcttgccagggctctccgaacctagctagcatcctcttcaattccaactaga

SEQ ID NO: 79 - DNA, 70 bp, Homo sapiens
  source 1..70: mol_type = genomic DNA; note = RPS9 3'-UTR
gtccacctgtccctcctgggctgctggattgtctcgttttcctgccaaataaacaggatcagcgctttac

SEQ ID NO: 80 - RNA, 70 nt, Homo sapiens
  source 1..70: mol_type = mRNA; note = RPS9 3'-UTR
gtccacctgtccctcctgggctgctggattgtctcgttttcctgccaaataaacaggatcagcgctttac

SEQ ID NO: 81 - DNA, 121 bp, Homo sapiens
  source 1..121: mol_type = genomic DNA; note = CASP1 3'-UTR
aataaggaaactgtatgaatgtctgtgggcaggaagtgaagagatccttctgtaaaggtttttggaattatgtctgctgaataataaacttttttgaaataataaatctggtagaaaaatg

SEQ ID NO: 82 - RNA, 121 nt, Homo sapiens
  source 1..121: mol_type = mRNA; note = CASP1 3'-UTR
aataaggaaactgtatgaatgtctgtgggcaggaagtgaagagatccttctgtaaaggtttttggaattatgtctgctgaataataaacttttttgaaataataaatctggtagaaaaatg

SEQ ID NO: 83 - DNA, 137 bp, Homo sapiens
  source 1..137: mol_type = genomic DNA; note = COX6B1 3'-UTR
actggctgcatctccctttcctctgtcctccatccttctcccaggatggtgaagggggacctggtacccagtgatccccaccccaggatcctaaatcatgacttacctgctaataaaaactcattggaaaagtgaga

SEQ ID NO: 84 - RNA, 137 nt, Homo sapiens
  source 1..137: mol_type = mRNA; note = COX6B1 3'-UTR
actggctgcatctccctttcctctgtcctccatccttctcccaggatggtgaagggggacctggtacccagtgatccccaccccaggatcctaaatcatgacttacctgctaataaaaactcattggaaaagtgaga

SEQ ID NO: 85 - DNA, 353 bp, Mus musculus
  source 1..353: mol_type = genomic DNA; note = Gnas 3'-UTR
gaagggaacacccaaatttaattcagccttaagcacaattaattaagagtgaaacgtaattgtacaagcagttggtcacccaccatagggcatgatcaacaccgcaacctttcctttttcccccagtgattctgaaaaacccctcttcccttcagcttgcttagatgttccaaatttagtaagcttaaggcggcctacagaagaaaaagaaaaaaaaggccacaaaagttccctctcactttcagtaaataaaataaaagcagcaacagaaataaagaaataaatgaaattcaaaatgaaataaatattgtgttgtgcagcattaaaaaatcaataaaaattaaaaatgagca

SEQ ID NO: 86 - RNA, 353 nt, Mus musculus
  source 1..353: mol_type = mRNA; note = Gnas 3'-UTR
gaagggaacacccaaatttaattcagccttaagcacaattaattaagagtgaaacgtaattgtacaagcagttggtcacccaccatagggcatgatcaacaccgcaacctttcctttttcccccagtgattctgaaaaacccctcttcccttcagcttgcttagatgttccaaatttagtaagcttaaggcggcctacagaagaaaaagaaaaaaaaggccacaaaagttccctctcactttcagtaaataaaataaaagcagcaacagaaataaagaaataaatgaaattcaaaatgaaataaatattgtgttgtgcagcattaaaaaatcaataaaaattaaaaatgagca

SEQ ID NO: 87 - DNA, 133 bp, Mus musculus
  source 1..133: mol_type = genomic DNA; note = Ndufa1 3'-UTR
ggaagcattttcctggctgattaaaagaaattactcagctatggtcatctgttcctgttagaaggctatgcagcatattatatactatgcgcatgttatgaaatgcataataaaaaattttaaaaaatctaaa

SEQ ID NO: 88 - RNA, 133 nt, Mus musculus
  source 1..133: mol_type = mRNA; note = Ndufa1 3'-UTR
ggaagcattttcctggctgattaaaagaaattactcagctatggtcatctgttcctgttagaaggctatgcagcatattatatactatgcgcatgttatgaaatgcataataaaaaattttaaaaaatctaaa